SEQUENCE LISTING (51 sequences)

SEQ ID NO: 1 - DNA, 18 bp, Saccharomyces cerevisiae
  source 1..18: mol_type = genomic DNA; note = i-SceI recognition site
tagggataacagggtaat

SEQ ID NO: 2 - DNA, 3081 bp, synthetic construct
  source 1..3081: mol_type = other DNA; note = IAA activator cassette (T2A linker)
atgggcagtgtcgagctgaatctgagggagactgagctgtgtcttggtcttcccggtggagatacagtggctccggtaaccggaaacaagagagggttctcagagacggttgatctgaagctaaatctgaataatgagcctgcaaacaaggaaggatctacgactcatgacgtcgtgacttttgattccaaggagaagagtgcttgtcctaaagatccagccaaacctccggccaaggcacaagttgtgggatggccaccggtgagatcataccggaagaacggtggcggaagcggagggggatcgatgagcaccgccccccccaccgacgtgagcctgggcgacgagctgcacctggacggcgaggacgtggccatggcccacgccgacgccctggacgacttcgacctggacatgctgggcgacggcgacagccccggccccggcttcaccccccacgacagcgccccctacggcgccctggacatggccgacttcgagttcgagcagatgttcaccgacgccctgggcatcgacgagtacggcggcaggaggaagaggggaagcggagagggcaggggaagtcttctaacatgcggggacgtggaggaaaatcccggccccatgaagaacatcaagaagaaccaggtgatgaacctgggccccaacagcaagctgctgaaggagtacaagagccagctgatcgagctgaacatcgagcagttcgaggccggcatcggcctgatcctgggcagcgcctacatcaggagcagggacgagggcaagacctactgcatgcagttcgagtggaagaacaaggcctacatggaccacgtgtgcctgctgtacgaccagtgggtgctgagccccccccacaagaaggagagggtgaaccacctgggcaacctggtgatcacctggggcgcccagaccttcaagcaccaggccttcaacaagctggccaacctgttcatcgtgaacaacaagaagaccatccccaacaacctggtggagaactacctgacccccatgagcctggcctactggttcatggacgccggcggcaagtgggactacaacaagaacagcaccaacaagagcatcgtgctgaacacccagagcttcaccttcgaggaggtggagtacctggtgaagggcctgaggaacaagttccagctgaactgctacgtgaagatcaacaagaacaagcccatcatctacatcgacagcatgagctacctgatcttctacaacctgatcaagccctacctgatcccccagatgatgtacaagctgcccaacaccatcagcagcgagaccttcctgaagggtggcggaagcggagggggatcgatgacctacttccccgagaaggtggtgaagcacatcttcagcttcctgcccgcccagagggacaggaacaccgtgagcctggtgtgcaaggtgtggtacgagatcgagaggctgagcaggaggggcgtgttcgtgggcaactgctacgccgtgagggccggcagggtggccgccaggttccccaacgtgagggccctgaccgtgaagggcaagccccacttcgccgacttcaacctggtgccccccgactggggcggctacgccggcccctggatcgaggccgccgccaggggctgccacggcctggaggagctgaggatgaagaggatggtggtgagcgacgagagcctggagctgctggccaggagcttccccaggttcagggccctggtgctgatcagctgcgagggcttcagcaccgacggcctggccgccgtggccagccactgcaagctgctgagggagctggacctgcaggagaacgaggtggaggacaggggccccaggtggctgagctgcttccccgacagctgcaccagcctggtgagcctgaacttcgcctgcatcaagggcgaggtgaacgccggcagcctggagaggctggtgagcaggagccccaacctgaggagcctgaggctgaacaggagcgtgagcgtggacaccctggccaagatcctgctgaggacccccaacctggaggacctgggcaccggcaacctgaccgacgacttccagaccgagagctacttcaagctgaccagcgccctggagaagtgcaagatgctgaggagcctgagcggcttctgggacgccagccccgtgtgcctgagcttcatctaccccctgtgcgcccagctgaccggcctgaacctgagctacgcccccaccctggacgccagcgacctgaccaagatgatcagcaggtgcgtgaagctgcagaggctgtgggtgctggactgcatcagcgacaagggcctgcaggtggtggccagcagctgcaaggacctgcaggagctgagggtgttccccagcgacttctacgtggccggctacagcgccgtgaccgaggagggcctggtggccgtgagcctgggctgccccaagctgaacagcctgctgtacttctgccaccagatgaccaacgccgccctggtgaccgtggccaagaactgccccaacttcaccaggttcaggctgtgcatcctggagcccggcaagcccgacgtggtgaccagccagcccctggacgagggcttcggcgccatcgtgagggagtgcaagggcctgcagaggctgagcatcagcggcctgctgaccgacaaggtgttcatgtacatcggcaagtacgccaagcagctggagatgctgagcatcgccttcgccggcgacagcgacaagggcatgatgcacgtgatgaacggctgcaagaacctgaggaagctggagatcagggacagccccttcggcgacgccgccctgctgggcaacttcgccaggtacgagaccatgaggagcctgtggatgagcagctgcaacgtgaccctgaagggctgccaggtgctggccagcaagatgcccatgctgaacgtggaggtgatcaacgagagggacggcagcaacgagatggaggagaaccacggcgacctgcccaaggtggagaagctgtacgtgtacaggaccaccgccggcgccagggacgacgcccccaacttcgtgaagatcctgtaa

SEQ ID NO: 3 - DNA, 606 bp, synthetic construct
  source 1..606: mol_type = other DNA; note = AIDdelta34-VP16-Myc
atgggcagtgtcgagctgaatctgagggagactgagctgtgtcttggtcttcccggtggagatacagtggctccggtaaccggaaacaagagagggttctcagagacggttgatctgaagctaaatctgaataatgagcctgcaaacaaggaaggatctacgactcatgacgtcgtgacttttgattccaaggagaagagtgcttgtcctaaagatccagccaaacctccggccaaggcacaagttgtgggatggccaccggtgagatcataccggaagaacggtggcggaagcggagggggatcgatgagcaccgccccccccaccgacgtgagcctgggcgacgagctgcacctggacggcgaggacgtggccatggcccacgccgacgccctggacgacttcgacctggacatgctgggcgacggcgacagccccggccccggcttcaccccccacgacagcgccccctacggcgccctggacatggccgacttcgagttcgagcagatgttcaccgacgccctgggcatcgacgagtacggcggcggtggcggaagcggagggggatcggaacaaaaactcatctcagaagaggatctgtaa

SEQ ID NO: 4 - DNA, 587 bp, unidentified
  source 1..587: mol_type = other DNA; note = IRES
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 5 - DNA, 2610 bp, synthetic construct
  source 1..2610: mol_type = other DNA; note = 3xNLS-dI-SceI-TIR1-V5
atggccacaaccatggatccaaaaaagaagagaaaggtagatccaaaaaagaagagaaaggtagatccaaaaaagaagagaaaggtaatgaagaacatcaagaagaaccaggtgatgaacctgggccccaacagcaagctgctgaaggagtacaagagccagctgatcgagctgaacatcgagcagttcgaggccggcatcggcctgatcctgggcagcgcctacatcaggagcagggacgagggcaagacctactgcatgcagttcgagtggaagaacaaggcctacatggaccacgtgtgcctgctgtacgaccagtgggtgctgagccccccccacaagaaggagagggtgaaccacctgggcaacctggtgatcacctggggcgcccagaccttcaagcaccaggccttcaacaagctggccaacctgttcatcgtgaacaacaagaagaccatccccaacaacctggtggagaactacctgacccccatgagcctggcctactggttcatggacgccggcggcaagtgggactacaacaagaacagcaccaacaagagcatcgtgctgaacacccagagcttcaccttcgaggaggtggagtacctggtgaagggcctgaggaacaagttccagctgaactgctacgtgaagatcaacaagaacaagcccatcatctacatcgacagcatgagctacctgatcttctacaacctgatcaagccctacctgatcccccagatgatgtacaagctgcccaacaccatcagcagcgagaccttcctgaagggtggcggaagcggagggggatcgatgacctacttccccgagaaggtggtgaagcacatcttcagcttcctgcccgcccagagggacaggaacaccgtgagcctggtgtgcaaggtgtggtacgagatcgagaggctgagcaggaggggcgtgttcgtgggcaactgctacgccgtgagggccggcagggtggccgccaggttccccaacgtgagggccctgaccgtgaagggcaagccccacttcgccgacttcaacctggtgccccccgactggggcggctacgccggcccctggatcgaggccgccgccaggggctgccacggcctggaggagctgaggatgaagaggatggtggtgagcgacgagagcctggagctgctggccaggagcttccccaggttcagggccctggtgctgatcagctgcgagggcttcagcaccgacggcctggccgccgtggccagccactgcaagctgctgagggagctggacctgcaggagaacgaggtggaggacaggggccccaggtggctgagctgcttccccgacagctgcaccagcctggtgagcctgaacttcgcctgcatcaagggcgaggtgaacgccggcagcctggagaggctggtgagcaggagccccaacctgaggagcctgaggctgaacaggagcgtgagcgtggacaccctggccaagatcctgctgaggacccccaacctggaggacctgggcaccggcaacctgaccgacgacttccagaccgagagctacttcaagctgaccagcgccctggagaagtgcaagatgctgaggagcctgagcggcttctgggacgccagccccgtgtgcctgagcttcatctaccccctgtgcgcccagctgaccggcctgaacctgagctacgcccccaccctggacgccagcgacctgaccaagatgatcagcaggtgcgtgaagctgcagaggctgtgggtgctggactgcatcagcgacaagggcctgcaggtggtggccagcagctgcaaggacctgcaggagctgagggtgttccccagcgacttctacgtggccggctacagcgccgtgaccgaggagggcctggtggccgtgagcctgggctgccccaagctgaacagcctgctgtacttctgccaccagatgaccaacgccgccctggtgaccgtggccaagaactgccccaacttcaccaggttcaggctgtgcatcctggagcccggcaagcccgacgtggtgaccagccagcccctggacgagggcttcggcgccatcgtgagggagtgcaagggcctgcagaggctgagcatcagcggcctgctgaccgacaaggtgttcatgtacatcggcaagtacgccaagcagctggagatgctgagcatcgccttcgccggcgacagcgacaagggcatgatgcacgtgatgaacggctgcaagaacctgaggaagctggagatcagggacagccccttcggcgacgccgccctgctgggcaacttcgccaggtacgagaccatgaggagcctgtggatgagcagctgcaacgtgaccctgaagggctgccaggtgctggccagcaagatgcccatgctgaacgtggaggtgatcaacgagagggacggcagcaacgagatggaggagaaccacggcgacctgcccaaggtggagaagctgtacgtgtacaggaccaccgccggcgccagggacgacgcccccaacttcgtgaagatcctgggtggcggaagcggagggggatcgggtaagcctatccctaaccctctcctcggtctcgattctacgtaa

SEQ ID NO: 6 - DNA, 16 bp, Escherichia coli
  source 1..16: mol_type = genomic DNA; note = LexA Operator Sequence
ctgtatatatatacag

SEQ ID NO: 7 - DNA, 295 bp, synthetic construct
  source 1..295: mol_type = other DNA; note = 10 x i-SceI recognition site + minimal promoter
tctagaaactctagggataacagggtaatcttagggataacagggtaatcttagggataacagggtaatcttagggataacagggtaatcttagggataacagggtaattctagggataacagggtaatcttagggataacagggtaatcttagggataacagggtaatcttagggataacagggtaatcttagggataacagggtaatggggggctataaaagggggtgggggcgttcgtcctcactctagatctgcgatctaagtaagcttggcaatccggtactgttggtaa

SEQ ID NO: 8 - protein, 1026 aa, synthetic construct
  source 1..1026: note = IAA activator cassette (T2A linker)
MGSVELNLRETELCLGLPGGDTVAPVTGNKRGFSETVDLKLNLNNEPANKEGSTTHDVVTFDSKEKSACPKDPAKPPAKAQVVGWPPVRSYRKNGGGSGGGSMSTAPPTDVSLGDELHLDGEDVAMAHADALDDFDLDMLGDGDSPGPGFTPHDSAPYGALDMADFEFEQMFTDALGIDEYGGRRKRGSGEGRGSLLTCGDVEENPGPMKNIKKNQVMNLGPNSKLLKEYKSQLIELNIEQFEAGIGLILGSAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDAGGKWDYNKNSTNKSIVLNTQSFTFEEVEYLVKGLRNKFQLNCYVKINKNKPIIYIDSMSYLIFYNLIKPYLIPQMMYKLPNTISSETFLKGGGSGGGSMTYFPEKVVKHIFSFLPAQRDRNTVSLVCKVWYEIERLSRRGVFVGNCYAVRAGRVAARFPNVRALTVKGKPHFADFNLVPPDWGGYAGPWIEAAARGCHGLEELRMKRMVVSDESLELLARSFPRFRALVLISCEGFSTDGLAAVASHCKLLRELDLQENEVEDRGPRWLSCFPDSCTSLVSLNFACIKGEVNAGSLERLVSRSPNLRSLRLNRSVSVDTLAKILLRTPNLEDLGTGNLTDDFQTESYFKLTSALEKCKMLRSLSGFWDASPVCLSFIYPLCAQLTGLNLSYAPTLDASDLTKMISRCVKLQRLWVLDCISDKGLQVVASSCKDLQELRVFPSDFYVAGYSAVTEEGLVAVSLGCPKLNSLLYFCHQMTNAALVTVAKNCPNFTRFRLCILEPGKPDVVTSQPLDEGFGAIVRECKGLQRLSISGLLTDKVFMYIGKYAKQLEMLSIAFAGDSDKGMMHVMNGCKNLRKLEIRDSPFGDAALLGNFARYETMRSLWMSSCNVTLKGCQVLASKMPMLNVEVINERDGSNEMEENHGDLPKVEKLYVYRTTAGARDDAPNFVKIL

SEQ ID NO: 9 - protein, 201 aa, synthetic construct
  source 1..201: note = AIDdelta34-VP16-Myc
MGSVELNLRETELCLGLPGGDTVAPVTGNKRGFSETVDLKLNLNNEPANKEGSTTHDVVTFDSKEKSACPKDPAKPPAKAQVVGWPPVRSYRKNGGGSGGGSMSTAPPTDVSLGDELHLDGEDVAMAHADALDDFDLDMLGDGDSPGPGFTPHDSAPYGALDMADFEFEQMFTDALGIDEYGGGGGSGGGSEQKLISEEDL

SEQ ID NO: 10 - protein, 869 aa, synthetic construct
  source 1..869: note = 3xNLS-dI-SceI-TIR1-V5
MATTMDPKKKRKVDPKKKRKVDPKKKRKVMKNIKKNQVMNLGPNSKLLKEYKSQLIELNIEQFEAGIGLILGSAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDAGGKWDYNKNSTNKSIVLNTQSFTFEEVEYLVKGLRNKFQLNCYVKINKNKPIIYIDSMSYLIFYNLIKPYLIPQMMYKLPNTISSETFLKGGGSGGGSMTYFPEKVVKHIFSFLPAQRDRNTVSLVCKVWYEIERLSRRGVFVGNCYAVRAGRVAARFPNVRALTVKGKPHFADFNLVPPDWGGYAGPWIEAAARGCHGLEELRMKRMVVSDESLELLARSFPRFRALVLISCEGFSTDGLAAVASHCKLLRELDLQENEVEDRGPRWLSCFPDSCTSLVSLNFACIKGEVNAGSLERLVSRSPNLRSLRLNRSVSVDTLAKILLRTPNLEDLGTGNLTDDFQTESYFKLTSALEKCKMLRSLSGFWDASPVCLSFIYPLCAQLTGLNLSYAPTLDASDLTKMISRCVKLQRLWVLDCISDKGLQVVASSCKDLQELRVFPSDFYVAGYSAVTEEGLVAVSLGCPKLNSLLYFCHQMTNAALVTVAKNCPNFTRFRLCILEPGKPDVVTSQPLDEGFGAIVRECKGLQRLSISGLLTDKVFMYIGKYAKQLEMLSIAFAGDSDKGMMHVMNGCKNLRKLEIRDSPFGDAALLGNFARYETMRSLWMSSCNVTLKGCQVLASKMPMLNVEVINERDGSNEMEENHGDLPKVEKLYVYRTTAGARDDAPNFVKILGGGSGGGSGKPIPNPLLGLDST

SEQ ID NO: 11 - protein, 81 aa, Simplexvirus .sp
  source 1..81: note = VP16 transactivation domain
MSTAPPTDVSLGDELHLDGEDVAMAHADALDDFDLDMLGDGDSPGPGFTPHDSAPYGALDMADFEFEQMFTDALGIDEYGG

SEQ ID NO: 12 - protein, 235 aa, Saccharomyces cerevisiae
  source 1..235: note = Wild type I-SceI
MKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLK

SEQ ID NO: 13 - protein, 235 aa, synthetic construct
  source 1..235: note = Modified dI-SceI (D44S, D145A)
MKNIKKNQVMNLGPNSKLLKEYKSQLIELNIEQFEAGIGLILGSAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDAGGKWDYNKNSTNKSIVLNTQSFTFEEVEYLVKGLRNKFQLNCYVKINKNKPIIYIDSMSYLIFYNLIKPYLIPQMMYKLPNTISSETFLK

SEQ ID NO: 14 - protein, 575 aa, Oryza sativa
  source 1..575: note = Wild Type osTIR1
MTYFPEEVVEHIFSFLPAQRDRNTVSLVCKVWYEIERLSRRGVFVGNCYAVRAGRVAARFPNVRALTVKGKPHFADFNLVPPDWGGYAGPWIEAAARGCHGLEELRMKRMVVSDESLELLARSFPRFRALVLISCEGFSTDGLAAVASHCKLLRELDLQENEVEDRGPRWLSCFPDSCTSLVSLNFACIKGEVNAGSLERLVSRSPNLRSLRLNRSVSVDTLAKILLRTPNLEDLGTGNLTDDFQTESYFKLTSALEKCKMLRSLSGFWDASPVCLSFIYPLCAQLTGLNLSYAPTLDASDLTKMISRCVKLQRLWVLDCISDKGLQVVASSCKDLQELRVFPSDFYVAGYSAVTEEGLVAVSLGCPKLNSLLYFCHQMTNAALVTVAKNCPNFTRFRLCILEPGKPDVVTSQPLDEGFGAIVRECKGLQRLSISGLLTDKVFMYIGKYAKQLEMLSIAFAGDSDKGMMHVMNGCKNLRKLEIRDSPFGDAALLGNFARYETMRSLWMSSCNVTLKGCQVLASKMPMLNVEVINERDGSNEMEENHGDLPKVEKLYVYRTTAGARDDAPNFVKIL

SEQ ID NO: 15 - protein, 575 aa, synthetic construct
  source 1..575: note = Modified osTIR (E7K, E10K)
MTYFPEKVVKHIFSFLPAQRDRNTVSLVCKVWYEIERLSRRGVFVGNCYAVRAGRVAARFPNVRALTVKGKPHFADFNLVPPDWGGYAGPWIEAAARGCHGLEELRMKRMVVSDESLELLARSFPRFRALVLISCEGFSTDGLAAVASHCKLLRELDLQENEVEDRGPRWLSCFPDSCTSLVSLNFACIKGEVNAGSLERLVSRSPNLRSLRLNRSVSVDTLAKILLRTPNLEDLGTGNLTDDFQTESYFKLTSALEKCKMLRSLSGFWDASPVCLSFIYPLCAQLTGLNLSYAPTLDASDLTKMISRCVKLQRLWVLDCISDKGLQVVASSCKDLQELRVFPSDFYVAGYSAVTEEGLVAVSLGCPKLNSLLYFCHQMTNAALVTVAKNCPNFTRFRLCILEPGKPDVVTSQPLDEGFGAIVRECKGLQRLSISGLLTDKVFMYIGKYAKQLEMLSIAFAGDSDKGMMHVMNGCKNLRKLEIRDSPFGDAALLGNFARYETMRSLWMSSCNVTLKGCQVLASKMPMLNVEVINERDGSNEMEENHGDLPKVEKLYVYRTTAGARDDAPNFVKIL

SEQ ID NO: 16 - protein, 228 aa, Arabidopsis thaliana
  source 1..228: note = Wild Type AID
MGSVELNLRETELCLGLPGGDTVAPVTGNKRGFSETVDLKLNLNNEPANKEGSTTHDVVTFDSKEKSACPKDPAKPPAKAQVVGWPPVRSYRKNVMVSCQKSSGGPEAAAFVKVSMDGAPYLRKIDLRMYKSYDELSNALSNMFSSFTMGKHGGEEGMIDFMNERKLMDLVNSWDYVPSYEDKDGDWMLVGDVPWPMFVDTCKRLRLMKGSDAIGLAPRAMEKCKSRA

SEQ ID NO: 17 - protein, 94 aa, Arabidopsis thaliana
  source 1..94: note = AIDdelta34
MGSVELNLRETELCLGLPGGDTVAPVTGNKRGFSETVDLKLNLNNEPANKEGSTTHDVVTFDSKEKSACPKDPAKPPAKAQVVGWPPVRSYRKN

SEQ ID NO: 18 - DNA, 2271 bp, synthetic construct
  source 1..2271: mol_type = other DNA; note = ABA activator cassette (T2A linker)
atgagcaccgccccccccaccgacgtgagcctgggcgacgagctgcacctggacggcgaggacgtggccatggcccacgccgacgccctggacgacttcgacctggacatgctgggcgacggcgacagccccggccccggcttcaccccccacgacagcgccccctacggcgccctggacatggccgacttcgagttcgagcagatgttcaccgacgccctgggcatcgacgagtacggcggcggtggcggaagcggagggggatcgacccaggacgagttcacccagctgagccagagcatcgccgagttccacacctaccagctgggcaacggcaggtgcagcagcctgctggcccagaggatccacgccccccccgagaccgtgtggagcgtggtgaggaggttcgacaggccccagatctacaagcacttcatcaagagctgcaacgtgagcgaggacttcgagatgagggtgggctgcaccagggacgtgaacgtgatcagcggcctgcccgccaacaccagcagggagaggctggacctgctggacgacgacaggagggtgaccggcttcagcatcaccggcggcgagcacaggctgaggaactacaagagcgtgaccaccgtgcacaggttcgagaaggaggaggaggaggagaggatctggaccgtggtgctggagagctacgtggtggacgtgcccgagggcaacagcgaggaggacaccaggctgttcgccgacaccgtgatcaggctgaacctgcagaagctggccagcatcaccgaggccatgaacggcatcaggaggaagaggagcgtgagccacggaagcggagagggcaggggaagtcttctaacatgcggggacgtggaggaaaatcccggccccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgggtggcggaagcggagggggatcgatgacgcgtgtgcctttgtatggttttacttcgatttgtggaagaagacctgagatggaagctgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgagtcagttgcgccggagacggttgggtcaacgtcggtggttgccgttgttttcccgtctcacatcttcgtcgctaactgcggtgactctagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaagcatatgtcatga

SEQ ID NO: 19 - DNA, 288 bp, synthetic construct
  source 1..288: mol_type = other DNA; note = 9x Gal4 UAS recognition site + minimal promoter
gagtttctagacggagtactgtcctccgagcggagtactgtcctccgactcgagcggagtactgtcctccgagcggagtactgtcctccgagcggagtactgtcctccgagcggagtactgtcctccgagcggagtactgtcctccgagcggagtactgtcctccgaggaattccggagtactgtcctccgaagacgctagcggggggctataaaagggggtgggggcgttcgtcctcactctagatctgcgatctaagtaagcttggcaatccggtactgttggtaa

SEQ ID NO: 20 - protein, 756 aa, synthetic construct
  source 1..756: note = ABA activator cassette (T2A linker)
MSTAPPTDVSLGDELHLDGEDVAMAHADALDDFDLDMLGDGDSPGPGFTPHDSAPYGALDMADFEFEQMFTDALGIDEYGGGGGSGGGSTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNGIRRKRSVSHGSGEGRGSLLTCGDVEENPGPMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSMTRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKHMS

SEQ ID NO: 21 - protein, 202 aa, Escherichia coli
  source 1..202: note = LexA
MKALTARQQEVFDLIRDHISQTGMPPTRAEIAQRLGFRSPNAAEEHLKALARKGVIEIVSGASRGIRLLQEEEEGLPLVGRVAAGEPLLAQQHIEGHYQVDPSLFKPNADFLLRVSGMSMKDIGIMDGDLLAVHKTQDVRNGQVVVARIDDEVTVKRLKKQGNKVELLPENSEFKPIVVDLRQQSFTIEGLAVGVIRNGDWL

SEQ ID NO: 22 - protein, 147 aa, Saccharomyces cerevisiae
  source 1..147: note = Gal4 DNA binding domain
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 23 - protein, 68 aa, Arabidopsis thaliana
  source 1..68: note = mAID
KEKSACPKDPAKPPAKAQVVGWPPVRSYRKNVMVSCQKSSGGPEAAAFVKVSMDGAPYLRKIDLRMYK

SEQ ID NO: 24 - protein, 434 aa, Arabidopsis thaliana
  source 1..434: note = ABI1
MEEVSPAIAGPFRPFSETQMDFTGIRLGKGYCNNQYSNQDSENGDLMVSLPETSSCSVSGSHGSESRKVLISRINSPNLNMKESAAADIVVVDISAGDEINGSDITSEKKMISRTESRSLFEFKSVPLYGFTSICGRRPEMEDAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKPRRKLKSKPLN

SEQ ID NO: 25 - protein, 298 aa, Arabidopsis thaliana
  source 1..298: note = Modified ABI1 complementary surfaces ABIcs
VPLYGFTSICGRRPEMEDAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLK

SEQ ID NO: 26 - protein, 221 aa, Arabidopsis thaliana
  source 1..221: note = PYL1
MANSESSSSPVNEEENSQRISTLHHQTMPSDLTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMNRNNNNNNSSQVR

SEQ ID NO: 27 - protein, 177 aa, Arabidopsis thaliana
  source 1..177: note = PYL1 complementary surface (PYLcs)
TQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMN

SEQ ID NO: 28 - DNA, 17 bp, Saccharomyces cerevisiae
  source 1..17: mol_type = genomic DNA; note = Gal4 UAS recognition site
cggagtactgtcctccg

SEQ ID NO: 29 - protein, 575 aa, synthetic construct
  source 1..575: note = Modified osTIR (E7K, E10K, F74G)
MTYFPEKVVKHIFSFLPAQRDRNTVSLVCKVWYEIERLSRRGVFVGNCYAVRAGRVAARFPNVRALTVKGKPHGADFNLVPPDWGGYAGPWIEAAARGCHGLEELRMKRMVVSDESLELLARSFPRFRALVLISCEGFSTDGLAAVASHCKLLRELDLQENEVEDRGPRWLSCFPDSCTSLVSLNFACIKGEVNAGSLERLVSRSPNLRSLRLNRSVSVDTLAKILLRTPNLEDLGTGNLTDDFQTESYFKLTSALEKCKMLRSLSGFWDASPVCLSFIYPLCAQLTGLNLSYAPTLDASDLTKMISRCVKLQRLWVLDCISDKGLQVVASSCKDLQELRVFPSDFYVAGYSAVTEEGLVAVSLGCPKLNSLLYFCHQMTNAALVTVAKNCPNFTRFRLCILEPGKPDVVTSQPLDEGFGAIVRECKGLQRLSISGLLTDKVFMYIGKYAKQLEMLSIAFAGDSDKGMMHVMNGCKNLRKLEIRDSPFGDAALLGNFARYETMRSLWMSSCNVTLKGCQVLASKMPMLNVEVINERDGSNEMEENHGDLPKVEKLYVYRTTAGARDDAPNFVKIL

SEQ ID NO: 30 - DNA, 357 bp, synthetic construct
  source 1..357: mol_type = other DNA; note = alphaCaffVHH
gaggtgcagctgcaggcctccggaggaggcctggtgcaggccggagggagcctgagactgagctgtactgccagcgggaggacaggaacaatctactcaatggcctggttccgccaggcccccggcaaggaaagggagtttctggccaccgtgggctggagctctggcatcacatactacatggactcagtgaagggtaggttcaccatcagcagagacaacgccaagaattctgcctacctgcagatgaatagcctgaaaccagaggataccgccgtgtactactgtactgctacaagggcctatagtgtgggttatgattactggggacagggcacccaggtgaccgtgagccac

SEQ ID NO: 31 - protein, 119 aa, synthetic construct
  source 1..119: note = alphaCaffVHH
EVQLQASGGGLVQAGGSLRLSCTASGRTGTIYSMAWFRQAPGKEREFLATVGWSSGITYYMDSVKGRFTISRDNAKNSAYLQMNSLKPEDTAVYYCTATRAYSVGYDYWGQGTQVTVSH

SEQ ID NO: 32 - DNA, 576 bp, synthetic construct
  source 1..576: mol_type = other DNA; note = Arabidopsis thaliana PYRMandi
atgcccagcgagctgacccccgaggagaggagcgagctgaagaacagcatcgccgagttccacacctaccagctggaccccggcagctgcagcagcctgcacgcccagaggatccacgccccccccgagctggtgtggagcatcgtgaggaggttcgacaagccccagacccacaggcacttcatcaagagctgcagcgtggagcagaacttcgagatgagggtgggctgcaccagggacatcatcgtgatcagcggcctgcccgccaacaccagcaccgagaggctggacatcctggacgacgagaggagggtgaccggcgccagcatcatcggcggcgagcacaggctgaccaactacaagggcgtgaccaccgtgcacaggttcgagaaggagaacaggatctggaccgtggtgctggagagctacgtggtggacatgcccgagggcaacagcgaggacgacaccaggatgctggccgacaccgtggtgaagctgaacctgcagaagctggccaccgtggccgaggccatggccaggaacagcggcgacggcagcggcagccaggtgacctaa

SEQ ID NO: 33 - protein, 191 aa, synthetic construct
  source 1..191: note = Arabidopsis thaliana PYRMandi
MPSELTPEERSELKNSIAEFHTYQLDPGSCSSLHAQRIHAPPELVWSIVRRFDKPQTHRHFIKSCSVEQNFEMRVGCTRDIIVISGLPANTSTERLDILDDERRVTGASIIGGEHRLTNYKGVTTVHRFEKENRIWTVVLESYVVDMPEGNSEDDTRMLADTVVKLNLQKLATVAEAMARNSGDGSGSQVT

SEQ ID NO: 34 - DNA, 534 bp, synthetic construct
  source 1..534: mol_type = other DNA; note = Arabidopsis thaliana PYLcsMandi
acccaggacgagttcacccagctgagccagagcatcgccgagttccacacctaccagctgggcaacggcaggtgcagcagcctgctggcccagaggatccacgccccccccgagaccgtgtggagcgtggtgaggaggttcgacaggccccagatccacaggcacttcatcaagagctgcaacgtgagcgaggacttcgagatgagggtgggctgcaccagggacatcaacgtgatcagcggcctgcccgccaacaccagcagggagaggctggacctgctggacgacgacaggagggtgaccggcgccagcatcaccggcggcgagcacaggctgaggaactacaagggcgtgaccaccgtgcacaggttcgagaaggaggaggaggaggagaggatctggaccgtggtgctggagagctacgtggtggacgtgcccgagggcaacagcgaggaggacaccaggctgctggccgacaccgtgatcaggctgaacctgcagaagctggccagcatcaccgaggccatgaactaa

SEQ ID NO: 35 - protein, 177 aa, synthetic construct
  source 1..177: note = Arabidopsis thaliana PYLcsMandi
TQDEFTQLSQSIAEFHTYQLGNGRCSSLLAQRIHAPPETVWSVVRRFDRPQIHRHFIKSCNVSEDFEMRVGCTRDINVISGLPANTSRERLDLLDDDRRVTGASITGGEHRLRNYKGVTTVHRFEKEEEEERIWTVVLESYVVDVPEGNSEEDTRLLADTVIRLNLQKLASITEAMN

SEQ ID NO: 36 - DNA, 1038 bp, Arabidopsis thaliana
  source 1..1038: mol_type = genomic DNA; note = GID1
atggccgccagcgacgaggtgaacctgatcgagagcaggaccgtggtgcccctgaacacctgggtgctgatcagcaacttcaaggtggcctacaacatcctgaggaggcccgacggcaccttcaacaggcacctggccgagtacctggacaggaaggtgaccgccaacgccaaccccgtggacggcgtgttcagcttcgacgtgctgatcgacaggaggatcaacctgctgagcagggtgtacaggcccgcctacgccgaccaggagcagccccccagcatcctggacctggagaagcccgtggacggcgacatcgtgcccgtgatcctgttcttccacggcggcagcttcgcccacagcagcgccaacagcgccatctacgacaccctgtgcaggaggctggtgggcctgtgcaagtgcgtggtggtgagcgtgaactacaggagggcccccgagaacccctacccctgcgcctacgacgacggctggatcgccctgaactgggtgaacagcaggagctggctgaagagcaagaaggacagcaaggtgcacatcttcctggccggcgacagcagcggcggcaacatcgcccacaacgtggccctgagggccggcgagagcggcatcgacgtgctgggcaacatcctgctgaaccccatgttcggcggcaacgagaggaccgagagcgagaagagcctggacggcaagtacttcgtgaccgtgagggacagggactggtactggaaggccttcctgcccgagggcgaggacagggagcaccccgcctgcaaccccttcagccccaggggcaagagcctggagggcgtgagcttccccaagagcctggtggtggtggccggcctggacctgatcagggactggcagctggcctacgccgagggcctgaagaaggccggccaggaggtgaagctgatgcacctggagaaggccaccgtgggcttctacctgctgcccaacaacaaccacttccacaacgtgatggacgagatcagcgccttcgtgaacgccgagtgctaa

SEQ ID NO: 37 - protein, 345 aa, Arabidopsis thaliana
  source 1..345: note = GID1
MAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAEC

SEQ ID NO: 38 - DNA, 276 bp, Arabidopsis thaliana
  source 1..276: mol_type = genomic DNA; note = GAI nucleotides 1-276
atgaagagggaccaccaccaccaccaccaccaggacaagaagaccatgatgatgaacgaggaggacgacggcaacggcatggacgagctgctggccgtgctgggctacaaggtgaggagcagcgagatggccgacgtggcccagaagctggagcagctggaggtgatgatgagcaacgtgcaggaggacgacctgagccagctggccaccgagaccgtgcactacaaccccgccgagctgtacacctggctggacagcatgctgaccgacctgaac

SEQ ID NO: 39 - protein, 92 aa, Arabidopsis thaliana
  source 1..92: note = GAI amino acids 1-92
MKRDHHHHHHQDKKTMMMNEEDDGNGMDELLAVLGYKVRSSEMADVAQKLEQLEVMMSNVQEDDLSQLATETVHYNPAELYTWLDSMLTDLN

SEQ ID NO: 40 - DNA, 1542 bp, synthetic construct
  source 1..1542: mol_type = other DNA; note = Caffeine activator cassette (T2A linker)
atgaaactgctgagcagcatcgagcaggcctgtgacatctgtagactgaagaagctgaagtgcagcaaggagaagcccaaatgcgccaaatgtctgaagaacaattgggagtgcaggtactcccccaagactaaacgctcccccctgaccagagctcacctgacagaggtggagagccggctggaaaggctggagcagctgttcctgctgattttcccaagggaagacctggatatgatcctgaagatggatagcctgcaggatatcaaggctcttctgactggcctcttcgtgcaggacaatgtgaacaaggacgctgtgacagaccgactggcctccgtggagacagacatgcctctgacactccggcagcacaggatcagcgccacatcttctagcgaggagtcctctaacaaaggccaacggcagctgacagtgtctgggggcggcagcggcgggggcagcgaggtgcagctgcaggcctccggaggaggcctggtgcaggccggagggagcctgagactgagctgtactgccagcgggaggacaggaacaatctactcaatggcctggttccgccaggcccccggcaaggaaagggagtttctggccaccgtgggctggagctctggcatcacatactacatggactcagtgaagggtaggttcaccatcagcagagacaacgccaagaattctgcctacctgcagatgaatagcctgaaaccagaggataccgccgtgtactactgtactgctacaagggcctatagtgtgggttatgattactggggacagggcacccaggtgaccgtgagccacgggatccgcagaaaaagaagcgtgtctcacggctccggcgagggcagagggtccctgctgacctgtggcgatgtggaggagaaccctggccctgaggtgcagctgcaggccagtgggggcggcctggttcaggcaggcggaagtctgcggcttagctgtaccgcctccggccggaccggaactatttactcaatggcctggttccggcaggcccccgggaaagagagggagttcctggccaccgtgggatggtcctccgggattacctactacatggactccgtgaaggggaggttcaccatctctcgcgataacgccaagaatagcgcctatctgcagatgaacagcctgaaacccgaggacacagctgtgtactactgtacggccactcgggcttatagcgtgggctacgactactggggccagggcacacaggtgaccgtgtcccatggcggcggcagcggcggaggcagtatgagcaccgcccccccaaccgatgtcagcctgggcgatgagctccacctggacggcgaggacgtggccatggcccatgccgatgccctggacgacttcgatctggacatgctgggcgacggcgatagtcccggcccaggcttcacccctcatgattccgctccatacggtgccctggatatggccgacttcgagtttgagcagatgttcacagacgccctgggcatcgatgagtacggcggctaa

SEQ ID NO: 41 - protein, 513 aa, synthetic construct
  source 1..513: note = Caffeine activator cassette (T2A linker)
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSEVQLQASGGGLVQAGGSLRLSCTASGRTGTIYSMAWFRQAPGKEREFLATVGWSSGITYYMDSVKGRFTISRDNAKNSAYLQMNSLKPEDTAVYYCTATRAYSVGYDYWGQGTQVTVSHGIRRKRSVSHGSGEGRGSLLTCGDVEENPGPEVQLQASGGGLVQAGGSLRLSCTASGRTGTIYSMAWFRQAPGKEREFLATVGWSSGITYYMDSVKGRFTISRDNAKNSAYLQMNSLKPEDTAVYYCTATRAYSVGYDYWGQGTQVTVSHGGGSGGGSMSTAPPTDVSLGDELHLDGEDVAMAHADALDDFDLDMLGDGDSPGPGFTPHDSAPYGALDMADFEFEQMFTDALGIDEYGG

SEQ ID NO: 42 - DNA, 2271 bp, synthetic construct
  source 1..2271: mol_type = other DNA; note = PYLcsMandi activator cassette (T2A linker)
atgagcaccgccccccccaccgacgtgagcctgggcgacgagctgcacctggacggcgaggacgtggccatggcccacgccgacgccctggacgacttcgacctggacatgctgggcgacggcgacagccccggccccggcttcaccccccacgacagcgccccctacggcgccctggacatggccgacttcgagttcgagcagatgttcaccgacgccctgggcatcgacgagtacggcggcggtggcggaagcggagggggatcgacccaggacgagttcacccagctgagccagagcatcgccgagttccacacctaccagctgggcaacggcaggtgcagcagcctgctggcccagaggatccacgccccccccgagaccgtgtggagcgtggtgaggaggttcgacaggccccagatccacaggcacttcatcaagagctgcaacgtgagcgaggacttcgagatgagggtgggctgcaccagggacatcaacgtgatcagcggcctgcccgccaacaccagcagggagaggctggacctgctggacgacgacaggagggtgaccggcgccagcatcaccggcggcgagcacaggctgaggaactacaagggcgtgaccaccgtgcacaggttcgagaaggaggaggaggaggagaggatctggaccgtggtgctggagagctacgtggtggacgtgcccgagggcaacagcgaggaggacaccaggctgctggccgacaccgtgatcaggctgaacctgcagaagctggccagcatcaccgaggccatgaacggcatcaggaggaagaggagcgtgagccacggaagcggagagggcaggggaagtcttctaacatgcggggacgtggaggaaaatcccggccccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgggtggcggaagcggagggggatcgatgacgcgtgtgcctttgtatggttttacttcgatttgtggaagaagacctgagatggaagctgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgagtcagttgcgccggagacggttgggtcaacgtcggtggttgccgttgttttcccgtctcacatcttcgtcgctaactgcggtgactctagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaagcatatgtcataa

SEQ ID NO: 43 - protein, 756 aa, synthetic construct
  source 1..756: note = PYLcsMandi activator cassette (T2A linker)
MSTAPPTDVSLGDELHLDGEDVAMAHADALDDFDLDMLGDGDSPGPGFTPHDSAPYGALDMADFEFEQMFTDALGIDEYGGGGGSGGGSTQDEFTQLSQSIAEFHTYQLGNGRCSSLLAQRIHAPPETVWSVVRRFDRPQIHRHFIKSCNVSEDFEMRVGCTRDINVISGLPANTSRERLDLLDDDRRVTGASITGGEHRLRNYKGVTTVHRFEKEEEEERIWTVVLESYVVDVPEGNSEEDTRLLADTVIRLNLQKLASITEAMNGIRRKRSVSHGSGEGRGSLLTCGDVEENPGPMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSMTRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKHMS

SEQ ID NO: 44 - DNA, 2313 bp, synthetic construct
  source 1..2313: mol_type = other DNA; note = PYRMandi activator cassette (T2A linker)
atgagcaccgccccccccaccgacgtgagcctgggcgacgagctgcacctggacggcgaggacgtggccatggcccacgccgacgccctggacgacttcgacctggacatgctgggcgacggcgacagccccggccccggcttcaccccccacgacagcgccccctacggcgccctggacatggccgacttcgagttcgagcagatgttcaccgacgccctgggcatcgacgagtacggcggcggtggcggaagcggagggggatcgatgcccagcgagctgacccccgaggagaggagcgagctgaagaacagcatcgccgagttccacacctaccagctggaccccggcagctgcagcagcctgcacgcccagaggatccacgccccccccgagctggtgtggagcatcgtgaggaggttcgacaagccccagacccacaggcacttcatcaagagctgcagcgtggagcagaacttcgagatgagggtgggctgcaccagggacatcatcgtgatcagcggcctgcccgccaacaccagcaccgagaggctggacatcctggacgacgagaggagggtgaccggcgccagcatcatcggcggcgagcacaggctgaccaactacaagggcgtgaccaccgtgcacaggttcgagaaggagaacaggatctggaccgtggtgctggagagctacgtggtggacatgcccgagggcaacagcgaggacgacaccaggatgctggccgacaccgtggtgaagctgaacctgcagaagctggccaccgtggccgaggccatggccaggaacagcggcgacggcagcggcagccaggtgaccggcatcaggaggaagaggagcgtgagccacggaagcggagagggcaggggaagtcttctaacatgcggggacgtggaggaaaatcccggccccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgggtggcggaagcggagggggatcgatgacgcgtgtgcctttgtatggttttacttcgatttgtggaagaagacctgagatggaagctgctgtttcgactataccaagattccttcaatcttcctctggttcgatgttagatggtcggtttgatcctcaatccgccgctcatttcttcggtgtttacgacggccatggcggttctcaggtagcgaactattgtagagagaggatgcatttggctttggcggaggagatagctaaggagaaaccgatgctctgcgatggtgatacgtggctggagaagtggaagaaagctcttttcaactcgttcctgagagttgactcggagattgagtcagttgcgccggagacggttgggtcaacgtcggtggttgccgttgttttcccgtctcacatcttcgtcgctaactgcggtgactctagagccgttctttgccgcggcaaaactgcacttccattatccgttgaccataaaccggatagagaagatgaagctgcgaggattgaagccgcaggagggaaagtgattcagtggaatggagctcgtgttttcggtgttctcgccatgtcgagatccattggcgatagatacttgaaaccatccatcattcctgatccggaagtgacggctgtgaagagagtaaaagaagatgattgtctgattttggcgagtgacggggtttgggatgtaatgacggatgaagaagcgtgtgagatggcaaggaagcggattctcttgtggcacaagaaaaacgcggtggctggggatgcatcgttgctcgcggatgagcggagaaaggaagggaaagatcctgcggcgatgtccgcggctgagtatttgtcaaagctggcgatacagagaggaagcaaagacaacataagtgtggtggtggttgatttgaagcatatgtcatga

SEQ ID NO: 45 - protein, 770 aa, synthetic construct
  source 1..770: note = PYRMandi activator cassette (T2A linker)
MSTAPPTDVSLGDELHLDGEDVAMAHADALDDFDLDMLGDGDSPGPGFTPHDSAPYGALDMADFEFEQMFTDALGIDEYGGGGGSGGGSMPSELTPEERSELKNSIAEFHTYQLDPGSCSSLHAQRIHAPPELVWSIVRRFDKPQTHRHFIKSCSVEQNFEMRVGCTRDIIVISGLPANTSTERLDILDDERRVTGASIIGGEHRLTNYKGVTTVHRFEKENRIWTVVLESYVVDMPEGNSEDDTRMLADTVVKLNLQKLATVAEAMARNSGDGSGSQVTGIRRKRSVSHGSGEGRGSLLTCGDVEENPGPMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSMTRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLKHMS

SEQ ID NO: 46 - DNA, 2139 bp, synthetic construct
  source 1..2139: mol_type = other DNA; note = Gibberellin activator cassette (T2A linker)
atgagcaccgccccccccaccgacgtgagcctgggcgacgagctgcacctggacggcgaggacgtggccatggcccacgccgacgccctggacgacttcgacctggacatgctgggcgacggcgacagccccggccccggcttcaccccccacgacagcgccccctacggcgccctggacatggccgacttcgagttcgagcagatgttcaccgacgccctgggcatcgacgagtacggcggcggtggcggaagcggagggggatcgatggccgccagcgacgaggtgaacctgatcgagagcaggaccgtggtgcccctgaacacctgggtgctgatcagcaacttcaaggtggcctacaacatcctgaggaggcccgacggcaccttcaacaggcacctggccgagtacctggacaggaaggtgaccgccaacgccaaccccgtggacggcgtgttcagcttcgacgtgctgatcgacaggaggatcaacctgctgagcagggtgtacaggcccgcctacgccgaccaggagcagccccccagcatcctggacctggagaagcccgtggacggcgacatcgtgcccgtgatcctgttcttccacggcggcagcttcgcccacagcagcgccaacagcgccatctacgacaccctgtgcaggaggctggtgggcctgtgcaagtgcgtggtggtgagcgtgaactacaggagggcccccgagaacccctacccctgcgcctacgacgacggctggatcgccctgaactgggtgaacagcaggagctggctgaagagcaagaaggacagcaaggtgcacatcttcctggccggcgacagcagcggcggcaacatcgcccacaacgtggccctgagggccggcgagagcggcatcgacgtgctgggcaacatcctgctgaaccccatgttcggcggcaacgagaggaccgagagcgagaagagcctggacggcaagtacttcgtgaccgtgagggacagggactggtactggaaggccttcctgcccgagggcgaggacagggagcaccccgcctgcaaccccttcagccccaggggcaagagcctggagggcgtgagcttccccaagagcctggtggtggtggccggcctggacctgatcagggactggcagctggcctacgccgagggcctgaagaaggccggccaggaggtgaagctgatgcacctggagaaggccaccgtgggcttctacctgctgcccaacaacaaccacttccacaacgtgatggacgagatcagcgccttcgtgaacgccgagtgcggcatcaggaggaagaggagcgtgagccacggaagcggagagggcaggggaagtcttctaacatgcggggacgtggaggaaaatcccggccccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgggtggcggaagcggagggggatcgatgaagagggaccaccaccaccaccaccaccaggacaagaagaccatgatgatgaacgaggaggacgacggcaacggcatggacgagctgctggccgtgctgggctacaaggtgaggagcagcgagatggccgacgtggcccagaagctggagcagctggaggtgatgatgagcaacgtgcaggaggacgacctgagccagctggccaccgagaccgtgcactacaaccccgccgagctgtacacctggctggacagcatgctgaccgacctgaactaa

SEQ ID NO: 47 - protein, 712 aa, synthetic construct
  source 1..712: note = Gibberellin activator cassette (T2A linker)
MSTAPPTDVSLGDELHLDGEDVAMAHADALDDFDLDMLGDGDSPGPGFTPHDSAPYGALDMADFEFEQMFTDALGIDEYGGGGGSGGGSMAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAECGIRRKRSVSHGSGEGRGSLLTCGDVEENPGPMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSMKRDHHHHHHQDKKTMMMNEEDDGNGMDELLAVLGYKVRSSEMADVAQKLEQLEVMMSNVQEDDLSQLATETVHYNPAELYTWLDSMLTDLN

SEQ ID NO: 48 - protein, 533 aa, Arabidopsis thaliana
  source 1..533: note = Arabidopsis thaliana full length GAI (UniProt Q9LQT8)
MKRDHHHHHHQDKKTMMMNEEDDGNGMDELLAVLGYKVRSSEMADVAQKLEQLEVMMSNVQEDDLSQLATETVHYNPAELYTWLDSMLTDLNPPSSNAEYDLKAIPGDAILNQFAIDSASSSNQGGGGDTYTTNKRLKCSNGVVETTTATAESTRHVVLVDSQENGVRLVHALLACAEAVQKENLTVAEALVKQIGFLAVSQIGAMRKVATYFAEALARRIYRLSPSQSPIDHSLSDTLQMHFYETCPYLKFAHFTANQAILEAFQGKKRVHVIDFSMSQGLQWPALMQALALRPGGPPVFRLTGIGPPAPDNFDYLHEVGCKLAHLAEAIHVEFEYRGFVANTLADLDASMLELRPSEIESVAVNSVFELHKLLGRPGAIDKVLGVVNQIKPEIFTVVEQESNHNSPIFLDRFTESLHYYSTLFDSLEGVPSGQDKVMSEVYLGKQICNVVACDGPDRVERHETLSQWRNRFGSAGFAAAHIGSNAFKQASMLLALFNGGEGYRVEESDGCLMLGWHTRPLIATSAWKLSTN

SEQ ID NO: 49 - DNA, 1023 bp, synthetic construct
  source 1..1023: mol_type = other DNA; note = Arabidopsis thaliana full length GAI sequence (human codon optimized)
atgaagagggaccaccaccaccaccaccaccaggacaagaagaccatgatgatgaacgaggaggacgacggcaacggcatggacgagctgctggccgtgctgggctacaaggtgaggagcagcgagatggccgacgtggcccagaagctggagcagctggaggtgatgatgagcaacgtgcaggaggacgacctgagccagctggccaccgagaccgtgcactacaaccccgccgagctgtacacctggctggacagcatgctgaccgacctgaacccccccagcagcaacgccgagtacgacctgaaggccatccccggcgacgccatcctgaaccagttcgccatcgacagcgccagcagcagcaaccagggcggcggcggcgacacctacaccaccaacaagaggctgaagtgcagcaacggcgtggtggagaccaccaccgccaccgccgagagcaccaggcacgtggtgctggtggacagccaggagaacggcgtgaggctggtgcacgccctgctggcctgcgccgaggccgtgcagaaggagaacctgaccgtggccgaggccctggtgaagcagatcggcttcctggccgtgagccagatcggcgccatgaggaaggtggccacctacttcgccgaggccctggccaggaggatctacaggctgagccccagccagagccccatcgaccacagcctgagcgacaccctgcagatgcacttctacgagacctgcccctacctgaagttcgcccacttcaccgccaaccaggccatcctggaggccttccagggcaagaagagggtgcacgtgatcgacttcagcatgagccagggcctgcagtggcccgccctgatgcaggccctggccctgaggcccggcggcccccccgtgttcaggctgaccggcatcggcccccccgcccccgacaacttcgactacctgcacgaggtgggctgcaagctggcccacctggccgaggccatccacgtggagttcgagtacaggggcttcgtg

SEQ ID NO: 50 - protein, 486 aa, synthetic construct
  source 1..486: note = FMC63 scFv based CD19-targeting CAR used in examples
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 745 aa, synthetic construct
  source 1..745: note = Dual CD19/CD22 targeting CAR used in examples
MPLLLLLPLLWAGALAGSGEQKLISEEDLGGGSGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0290916 A1
(43) Pub. Date: Sep. 18, 2025

(54) Chemically Induced Proximity Systems

(71) Applicant: Antibody Analytics Limited, Motherwell, North Lanarkshire (GB)

(72) Inventors: Timothy London, Motherwell, North Lanarkshire (GB); Hannah Findlay, Motherwell, North Lanarkshire (GB); Kieran Izzett, Motherwell, North Lanarkshire (GB); Amy Graham, Motherwell, North Lanarkshire (GB); Adele Hannigan, Motherwell, North Lanarkshire (GB); Agapitos Patakas, Motherwell, North Lanarkshire (GB); Andy Upsall, Motherwell, North Lanarkshire (GB)

(73) Assignee: Antibody Analytics Limited, Motherwell, North Lanarkshire (GB)

(21) Appl. No.: 18/861,312

(22) PCT Filed: May 5, 2023
(86) PCT No.: PCT/GB2023/051204

§ 371 Date: Oct. 29, 2024

(30) Foreign Application Priority Data
May 6, 2022 (GB) 2206630.2

(51) Int. Cl. G01N 33/50; C12N 5/071; C12N 15/63; C12N 15/86

(52) U.S. Cl. CPC G01N 33/505; C12N 5/0682; C12N 5/0686; C12N 15/635; C12N 15/86; G01N 33/5052; G01N 33/5055; C12N 2510/02; C12N 2830/001; G01N 2500/10

(57) ABSTRACT

The present invention relates to methods of screening the efficacy and safety of candidate therapies/immunotherapies/cell therapies using cells containing a first and optionally a second inducible system operable to express a first and optionally a second protein of interest to the desired levels. The invention further relates to inducible systems, especially to one or more chemically induced proximity systems comprising a first plant hormone inducible proximity system and a second plant hormone inducible proximity system, and to methods of controlling the expression of proteins of interest using the systems. The invention further relates to a method of making a cell comprising the systems, and to cells comprising the system.

### FIELD OF THE INVENTION

The present invention relates to methods of screening the efficacy and safety of candidate binding molecules/immunotherapies/cell therapies using cells containing a first and optionally a second inducible system operable to express a first and optionally a second protein of interest to the desired levels. The invention further relates to inducible systems, especially to one or more chemically induced proximity systems comprising a first plant hormone inducible proximity system and a second plant hormone inducible proximity system, and to methods of controlling the expression of proteins of interest using the systems. The invention further relates to a method of making a cell comprising the systems, and to cells comprising the system.

### INTRODUCTION

The focus of drug development for cancer treatment has increasingly turned towards targeted therapies, whereby drugs are designed to target unique features of cancer cells in the hope that side-effects on healthy tissues can be avoided. The rapidly growing therapeutic area of immuno-oncology encompasses a range of modalities, some of which aim to exploit antigens on the surface of cancer cells, as a means by which to achieve the selective destruction of cancer cells. Unfortunately, very few tumour-restricted antigens (TRAs) are known, with most found expressed at low levels in one or more healthy tissues (known as tumour associated antigens or TAAs). The development of extremely promising advanced therapy modalities such as T cell engaging antibodies and chimeric antigen receptor T (CAR-T) cell therapies are encumbered by this lack of TRAs, where expression in healthy tissues of TAAs can cause serious adverse effects and even fatalities.

Several in vitro risk assessment assays are now considered essential during the development of such biologics and Advanced Therapy Medicinal Products (ATMPs), including immunogenicity and Cytokine Release Syndrome (CRS) risk assessment assays. However, to date no reliable assays exist to assess so called, ‘on-target, off tumour’ effects in respect to the TAA expression level. Most studies rely on assessing multiple cell lines with varied levels of antigen expression. This has limitations which are both logistical (requirement for acquisition of multiple cell lines per project) and scientific as the different cell lines have different genetic backgrounds and therefore will have numerous inherent differences beyond antigen expression (e.g. expression of immunomodulatory molecules, differential susceptibility to cell death etc), that will constrain data interpretation. For the assessment of multiantigen targeting formats, the use of multiple cell lines does not provide an adequate level of control over the levels of expression of each TAA.

There is a need in the art for a risk assessment assay portfolio alongside CRS and immunogenicity studies that will allow drug developers to determine the threshold (or minimum) level of TAA on the cell surface that will elicit a biological effect so as to determine such on target but off tumour effects of candidates. Clinical trials are notoriously expensive and it is estimated that the probability of success of an oncology clinical trial is as low as 3.4% (https://academic.oup.com/biostatistics/article/20/2/273/4817524). It is therefore critical for drug developers to be able to accurately assess a drug's ability to discriminate between high TAA expressing cancer cells and low level, healthy cell expression of TAAs and ideally at an early, pre-clinical stage so as to lower the risk of clinical failure due to off-tumour effects.

Chemically induced proximity (CIP) systems use membrane-permeable, small-molecule inducers to control dimerization between proteins, typically a transactivator and a DNA binding domain, when fused to the inducer-binding proteins. CIPs can be adapted to switch on transcription of a protein of interest by means of bringing a DNA binding protein into the proximity of a transactivator and placing the binding motif for a DNA binding protein upstream of the coding sequence for the protein of interest. Such CIPs can be used to provide inducible expression of any given protein including an antigen of interest. Despite the effectiveness of CIPs in inducible expression of proteins in other fields, to date, such systems have not been used in the field of risk assessment assays for screening novel biologics and ATMPs. Furthermore, the use of more than one CIP system in tandem to effectively control expression of more than one protein independently to different levels has not been achieved in such a commercial context.

The present invention aims to address one or more of the above-mentioned problems in the art.

### SUMMARY OF THE INVENTION

In a first aspect of the invention there is provided a first construct comprising a promoter operably linked to a nucleic acid sequence encoding:
a first chimeric protein, and
a second chimeric protein,
   wherein the first and second chimeric proteins each comprise binding domain and an effector domain;
   wherein the binding domain is operable to bind to an inducer; and wherein the effector domain is selected from a transactivation domain or a DNA binding domain;
   wherein the binding domain and the effector domain of the first and second chimeric proteins are different.

In an embodiment of the first aspect of the invention there is provided a first construct comprising a promoter operably linked to a nucleic acid sequence encoding:
a first chimeric protein, and
a second chimeric protein,
   wherein the first and second chimeric proteins each comprise an auxin binding domain and an effector domain;
   wherein the auxin binding domain is optionally selected from Transport Inhibitor Response 1 protein (TIR1) or Auxin/indole-3-acetic acid protein (AID); wherein the effector domain is selected from a transactivation domain or a DNA binding domain optionally selected from Gal4 DNA binding domain and a catalytically inactive I-Scel endonuclease DNA binding domain (dl-Scel);
   wherein the auxin binding domain and the effector domain of the first and second chimeric proteins are different.

In a further embodiment of the first aspect of the invention there is provided a first construct comprising a promoter operably linked to a nucleic acid sequence encoding:
a first chimeric protein, and
a second chimeric protein,
   wherein the first and second chimeric proteins each comprise a caffeine binding domain and an effector domain;
   wherein the caffeine binding domain is optionally an anti-caffeine heavy-chain antibody fragment (aCaffVHH); wherein the effector domain is selected from a transactivation domain or a DNA binding domain optionally selected from Gal4 DNA binding domain and a catalytically inactive I-Scel endonuclease DNA binding domain (dl-Scel);
   wherein the effector domain of the first and second chimeric proteins is different.

In a further first embodiment of the first aspect of the invention there is provided a first construct comprising a promoter operably linked to a nucleic acid sequence encoding:
a first chimeric protein, and
a second chimeric protein,
   wherein the first and second chimeric proteins each comprise a Mandipropamid (Mandi) binding domain and an effector domain;
   wherein the Mandipropamid binding domain is optionally selected from a modified pyrobactin receptor (PYR^{Mandi}), a modified pyrobactin-like receptor (PYLcs^{Mandi}), and abscisic acid insensitive 1 protein (ABI); wherein the effector domain is selected from a transactivation domain or a DNA binding domain optionally selected from Gal4 DNA binding domain and a catalytically inactive I-Scel endonuclease DNA binding domain (dl-Scel);
   wherein the Mandipropamid binding domain and effector domain of the first and second chimeric proteins is different.

In a further first embodiment of the first aspect of the invention there is provided a first construct comprising a promoter operably linked to a nucleic acid sequence encoding:
a first chimeric protein, and
a second chimeric protein,
   wherein the first and second chimeric proteins each comprise a gibberellin binding domain and an effector domain;
   wherein the gibberellin binding domain is optionally selected from gibberellin insensitive dwarf 1 protein (GID1) and gibberellin insensitive protein (GAI) optionally which may be modified;
   wherein the effector domain is selected from a transactivation domain or a DNA binding domain optionally selected from Gal4 DNA binding domain and a catalytically inactive I-Scel endonuclease DNA binding domain (dl-Scel);
   wherein the gibberellin binding domain and effector domain of the first and second chimeric proteins is different.

In a second aspect of the invention there is provided a second construct comprising a nucleic acid sequence encoding: one or more effector domain binding sites operably linked to a nucleic acid sequence encoding a protein of interest, wherein each effector domain binding site is a dl-Scel binding site.

In further second aspect of the invention there is also provided an alternative second construct comprising a nucleic acid sequence encoding: one or more effector domain binding sites operably linked to a nucleic acid sequence encoding a protein of interest, wherein each effector domain binding site is a Gal4 upstream activation sequence.

Suitably a second construct comprising a dl-Scel binding site is used with a first construct that comprises a catalytically inactive I-Scel endonuclease DNA binding domain (dl-Scel). Suitably a second construct comprising a Gal4 upstream activation sequence is used with a first construct that comprises a Gal4 DNA binding domain.

In a third aspect of the invention there is provided an auxin inducible proximity system comprising the first construct of the first aspect and the second construct of either of the second aspects. In a further aspect, there is provided a caffeine inducible proximity system comprising the relevant first construct as defined hereinabove and either of the second constructs defined above. In a further aspect, there is provided a Mandipropamid inducible proximity system comprising the relevant first construct as defined hereinabove and either of the second constructs defined above. In a further aspect, there is provided a gibberellin inducible proximity system comprising the relevant first construct as defined hereinabove and either of the second constructs defined above.

Suitably any of these systems may be regarded herein as an inducible system, suitably as a first inducible system.

In a fourth aspect of the invention there is provided a vector comprising the first and/or second construct of the first or second aspects or embodiments respectively.

In a fifth aspect of the invention there is provided a chimeric protein comprising a catalytically inactive I-Scel endonuclease DNA binding domain (dl-Scel) fused to an auxin binding domain optionally selected from Transport Inhibitor Response 1 protein (TIR1) or Auxin/indole-3-acetic acid protein (AID). In an alternative fifth aspect, there is provided a chimeric protein comprising a catalytically inactive I-Scel endonuclease DNA binding domain (dl-Scel) fused to an abscisic acid binding domain optionally selected from abscisic acid insensitive 1 protein (ABI1) or pyrobactin resistance-like protein (PYL1). In an alternative fifth aspect there is provided a chimeric protein comprising a catalytically inactive I-Scel endonuclease DNA binding domain (dl-Scel) fused to a caffeine binding domain optionally selected from aCaffVHH. In an fifth alternative aspect there is provided a chimeric protein comprising a catalytically inactive I-Scel endonuclease DNA binding domain (dl-Scel) fused to a Mandipropamid binding domain optionally selected from PYR^{Mandi}, PYLcs^{Mandi}, and ABI. In an alternative fifth aspect there is provided a chimeric protein comprising a catalytically inactive I-Scel endonuclease DNA binding domain (dl-Scel) optionally fused to a gibberellin binding domain selected from GID1 protein and GAI protein, which may optionally be modified.

In further aspects of the invention, there is also provided a third construct comprising a promoter operably linked to a nucleic acid sequence encoding:
a third chimeric protein, and
a fourth chimeric protein,
   wherein the third and fourth chimeric proteins each comprise an abscisic acid binding domain and an effector domain;
   wherein the abscisic acid binding domain is optionally selected from ABI1 or pyrobactin resistance-like protein PYL1; wherein the effector domain is selected from a transactivation domain or a DNA binding domain optionally selected from a catalytically inactive I-Scel endonuclease DNA binding domain (dl-Scel) and a Gal4 DNA binding domain;
   wherein the abscisic acid binding domain and the effector domain of the third and fourth chimeric proteins are different.

In further aspects of the invention there is also provided a fourth construct comprising a nucleic acid sequence encoding: one or more effector domain binding sites operably linked to a nucleic acid sequence encoding a protein of interest, wherein each effector domain binding site is a Gal4 upstream activation sequence.

In further aspects of the invention there is also provided an alternative fourth construct comprising a nucleic acid sequence encoding: one or more effector domain binding sites operably linked to a nucleic acid sequence encoding a protein of interest, wherein each effector domain binding site is a dl-Scel binding site.

In further aspects of the invention, there is also provided an abscisic acid inducible proximity system comprising the third construct and the fourth construct.

Suitably this system may be regarded as an inducible system, suitably as a second inducible system.

In further aspects of the invention, there is also provided a vector comprising the third and/or fourth construct.

In one embodiment, there is provided one or more vectors which may comprise one or more of the first, second, third or fourth constructs in any combination.

Suitably the invention may relate to any inducible system which may form the first and second inducible systems referred to herein. Suitable examples of other inducible systems that may be used in the methods of the invention are provided below. In preferred embodiments, the inducible systems referred to herein are chemically induced proximity systems, which are preferably selected from: the auxin inducible proximity system, the caffeine inducible proximity system, the Mandipropamid inducible proximity system, the gibberellin inducible proximity system, and the ABA inducible proximity system as described hereinabove.

In a sixth aspect of the invention there is provided a method of making a cell comprising a first chemically inducible proximity system and/or a second chemically inducible proximity system as defined herein, comprising:
(a) Introducing the first construct into the cell and/or the third construct into the cell;
(b) Introducing the second construct into the cell and/or the fourth construct into the cell;
(c) Integrating the first and second construct, and/or the third and fourth construct, into the genome of the cell.

In one embodiment, the chemically inducible proximity systems, and the first, second, third and fourth constructs are as defined hereinabove.

In a sixth aspect of the invention there is provided a method of making a cell comprising the auxin inducible proximity system of the third aspect and/or an abscisic acid inducible proximity system as defined herein, comprising:
(a) Introducing the first construct into the cell and/or the third construct into the cell;
(b) Introducing the second construct into the cell and/or the fourth construct into the cell;
(c) Integrating the first and second construct, and/or the third and fourth construct, into the genome of the cell.

In one embodiment, therefore, the method of the sixth aspect is a method of making a cell comprising both the auxin inducible proximity system of the third aspect, and an abscisic acid inducible proximity system as defined herein, comprising:
(a) introducing the first and third constructs into the cell;
(b) introducing the second and fourth constructs into the cell;
(c) integrating the first, second, third and fourth constructs into the genome of the cell

In one embodiment, the auxin inducible proximity system and abscisic acid inducible proximity system are as defined hereinabove, as are the first, second, third and fourth constructs thereof.

In one embodiment, the first construct and third construct may be contained upon the same construct which may be known as the Induction construct. Therefore step (a) may comprise introducing the induction construct into the cell. In one embodiment, the second construct and the fourth construct may be contained upon the same construct which may be known as a delivery construct. Therefore step (b) may comprise introducing the delivery construct into the cell. Suitably steps (a) and (b) may be in any order.

In one embodiment, step (a) may comprise introducing a viral vector comprising the first construct into the cell and/or comprising the third construct into the cell. In one embodiment, step (b) may comprise introducing a viral vector comprising the second construct into the cell and/or comprising the fourth construct into the cell. In one embodiment the method is performed by lentiviral integration, therefore the viral vectors may be viral particles, suitably lentiviral particles. In one embodiment, one viral particle may comprise the induction construct, and another viral particle may comprise the delivery construct. In one embodiment, one viral particle may comprise the first, third, second and fourth constructs.

In a seventh aspect of the invention there is provided a method of making a cell comprising a first chemically inducible proximity system and/or a second chemically inducible proximity system as defined herein, comprising:
(a) Introducing the first construct and/or the third construct into the cell;
(b) Creating an integration site in the genome of the cell, wherein the integration site comprises a first recombination site;
(c) Introducing the second construct and/or the fourth construct, and an integration construct comprising a nucleic acid sequence encoding an integrase enzyme, into the cell, wherein the second construct and/or the fourth construct further comprises a second recombination site;
(d) Integrating the second construct, and/or the fourth construct, into the genome by recombination between the first and second recombination sites using the integrase enzyme;
   wherein steps (a) and (b) may be done in any order.

In a seventh aspect of the invention there is provided a method of making a cell comprising the auxin inducible proximity system of the third aspect and/or an abscisic acid inducible proximity system as defined herein, comprising:
(a) Introducing the first construct and/or the third construct into the cell;
(b) Creating an integration site in the genome of the cell, wherein the integration site comprises a first recombination site;
(c) Introducing the second construct and/or the fourth construct, and an integration construct comprising a nucleic acid sequence encoding an integrase enzyme, into the cell, wherein the second construct and/or the fourth construct further comprises a second recombination site;
(d) Integrating the second construct, and/or the fourth construct, into the genome by recombination between the first and second recombination sites using the integrase enzyme;
   wherein steps (a) and (b) may be done in any order.

In one embodiment, the method of the seventh aspect is a method of making a cell comprising both the auxin inducible proximity system of the third aspect and an abscisic acid inducible proximity system as defined herein, which comprises:
(a) Introducing the first and third constructs into the cell;
(b) Creating an integration site in the genome of the cell, wherein the integration site comprises a first recombination site;
(c) Introducing the second and fourth constructs into the cell, and an integration construct comprising a nucleic acid sequence encoding an integrase enzyme into the cell, wherein the second and fourth constructs further comprise a second recombination site;
(d) Integrating the second construct and the fourth construct into the genome by recombination between the first and second recombination sites using the integrase enzyme;
   wherein steps (a) and (b) may be done in any order.

In one embodiment, the auxin inducible proximity system and abscisic acid inducible proximity system are as defined hereinabove, as are the first, second, third and fourth constructs.

In one embodiment, the first construct and the third construct may be contained upon the same construct which may be known as the Induction construct. Therefore step (a) may comprise introducing an induction construct comprising the first and third constructs into the cell. In one embodiment, the second construct and the fourth construct may be contained upon the same construct which may be known as a delivery construct. Therefore step (c) may comprise introducing a delivery construct comprising the second and fourth constructs into the cell wherein the delivery construct further comprises a second recombination site.

In an eighth aspect of the invention there is provided a cell comprising (a) a first chemically inducible proximity system and/or (b) a second chemically inducible proximity system,
wherein the first chemically inducible proximity system (a) comprises:
(i) a first construct comprising a promoter operably linked to a nucleic acid sequence encoding:
   a first chimeric protein, and
   a second chimeric protein,
wherein the first and second chimeric proteins each comprise a first inducer binding domain and an effector domain;
wherein each first inducer binding domain is operable to bind to a first inducer; wherein the effector domains comprise a transactivation domain and a first DNA binding domain;
wherein the effector domain of the first and second chimeric proteins is different; and (ii) a second construct comprising a nucleic acid sequence encoding: one or more first DNA binding domain binding sites operably linked to a nucleic acid sequence encoding a first protein of interest;
wherein the second chemically inducible proximity system (b) comprises:
(i) a third construct comprising a promoter operably linked to a nucleic acid sequence encoding:
   a third chimeric protein, and
   a fourth chimeric protein,
wherein the third and fourth chimeric proteins each comprise a second inducer binding domain and an effector domain;
wherein each second inducer binding domain is operable to bind to a second inducer;
wherein the effector domains comprise a transactivation domain or second DNA binding domain;
wherein the effector domain of the third and fourth chimeric proteins is different; and
(ii) a fourth construct comprising a nucleic acid sequence encoding: one or more second DNA binding domain binding sites operably linked to a nucleic acid sequence encoding a second protein of interest;
Wherein the first chemically inducible proximity system does not interact with the second chemically inducible proximity system, and wherein one of the first or second DNA binding domains is a dl-Scel DNA binding domain.

Suitably the first and second chemically inducible proximity systems may be any chemically inducible proximity system described herein.

In one embodiment, the first and second chemically inducible proximity systems may be plant hormone or plant hormone analogue inducible proximity systems as described herein. Suitably in such embodiments, the first chemically inducible proximity system comprises a first and second construct as defined herein, and the second chemically inducible proximity system comprises a third and a fourth construct as defined herein. In one embodiment, at least one of the chemically inducible proximity systems is a plant hormone inducible proximity system. In one embodiment, at least one of the chemically inducible systems is an abscisic acid inducible proximity system defined herein. In one embodiment, at least one of the chemically inducible systems is selected from a caffeine inducible proximity system defined herein, a Mandipropamid inducible proximity system defined herein, and a gibberellin inducible proximity system defined herein.

In a preferred embodiment, the first chemically inducible proximity system is selected from a caffeine inducible proximity system defined herein, a Mandipropamid inducible proximity system defined herein, and a gibberellin inducible proximity system defined herein and the second chemically inducible proximity system is an abscisic acid inducible proximity system as defined herein.

In an alternative eighth aspect of the invention there is provided a cell comprising (a) a first plant hormone inducible proximity system and/or (b) a second plant hormone inducible proximity system,
wherein the first plant hormone inducible proximity system (a) comprises:
(i) a first construct comprising a promoter operably linked to a nucleic acid sequence encoding:
   a first chimeric protein, and
   a second chimeric protein,
wherein the first and second chimeric proteins each comprise a first plant hormone inducer binding domain and an effector domain;
wherein each first plant hormone inducer binding domain is operable to bind to a first plant hormone inducer; wherein the effector domains comprise a transactivation domain and a first DNA binding domain;
wherein the first plant hormone inducer binding domain and the effector domain of the first and second chimeric proteins are different; and
(ii) a second construct comprising a nucleic acid sequence encoding: one or more first DNA binding domain binding sites operably linked to a nucleic acid sequence encoding a first protein of interest;
wherein the second plant hormone inducible proximity system (b) comprises:
(i) a third construct comprising a promoter operably linked to a nucleic acid sequence encoding:
   a third chimeric protein, and
   a fourth chimeric protein,
wherein the third and fourth chimeric proteins each comprise a second plant hormone inducer binding domain and an effector domain;
wherein each second plant hormone inducer binding domain is operable to bind to a second plant hormone inducer; wherein the effector domains comprise a transactivation domain or second DNA binding domain;
wherein the second plant hormone inducer binding domain and the effector domain of the third and fourth chimeric proteins are different; and
(ii) a fourth construct comprising a nucleic acid sequence encoding: one or more second DNA binding domain binding sites operably linked to a nucleic acid sequence encoding a second protein of interest;
Wherein the first plant hormone inducible system does not interact with the second plant hormone inducible system, and wherein one of the first or second DNA binding domains is a dl-Scel DNA binding domain.

In one embodiment the cell may comprise only the first chemically inducible proximity system, suitably only the first plant hormone inducible proximity system. In one embodiment the cell may comprise only the second chemically inducible proximity system, suitably only the second plant hormone inducible proximity system. In an embodiment where the cell comprises only one system, suitably the DNA binding domain is a dl-Scel DNA binding domain. In one embodiment the cell comprises both the first and the second chemically inducible proximity systems, suitably both the plant hormone inducible proximity systems.

In one embodiment, the first chemically inducible proximity system, suitably the first plant hormone inducible system and the second chemically inducible proximity system, suitably the second plant hormone inducible system are orthogonal, suitably therefore the first and second chemically inducible proximity systems, suitably the first and second plant hormone inducible systems operate independently of each other. In one embodiment the first inducer, suitably the first plant hormone inducer is different to the second inducer, suitably the second plant hormone inducer, therefore the first inducer binding domains, suitably the first plant hormone inducer binding domains are different to the second inducer binding domains, suitably the second plant hormone inducer binding domains. In one embodiment the first DNA binding domain and the second DNA binding domain are different to each other. In one embodiment, the first and second proteins of interest are different to each other.

In one embodiment the one or more effector domain binding sites of the second and fourth constructs comprise DNA binding domain binding sites. Suitably the one or more effector domain binding sites of the second construct comprise one or more first DNA binding domain binding sites. Suitably the one or more effector domain binding sites of the fourth construct comprise one or more second DNA binding domain binding sites.

Suitably the effector domains of either the first inducible system or the second inducible system may be selected from any transactivation domain or DNA binding domain as long as the effector domain of the first and second chimeric proteins is different, and the effector domain of the third and fourth chimeric proteins is different. Suitably the effector domains of the first and third chimeric proteins may be transactivation domains, suitably they may both be the same transactivation domain. Suitably the effector domains of the second and fourth chimeric proteins may be DNA binding domains, suitably they are different DNA binding domains. Suitably one of the first or second DNA binding domains is a dl-Scel DNA binding domain, and suitably the other DNA binding domain is a different DNA binding domain. Suitably one of the first or second DNA binding domains is a dl-Scel DNA binding domain, and suitably the other DNA binding domain is selected from a LexA binding domain or a GAL4 DNA binding domain, preferably it is a GAL4 DNA binding domain. In one embodiment, the first DNA binding domain is a dl-Scel DNA binding domain, and the second DNA binding domain is a GAL4 DNA binding domain.

In one embodiment either of the first or second chemically inducible proximity systems is a plant hormone inducible system and is selected from any inducible system in which the inducer is a plant hormone. Suitable plant hormones may be selected from auxin, abscisic acid, gibberellin, ethene, cytokinin, salicylic acid, jasmonate, brassinosteroid, peptide hormones, and caffeine for example. Suitably either of the first or second plant hormone inducible systems may be an auxin, abscisic acid, gibberellin, ethene, cytokinin, salicylic acid, jasmonate, brassinosteroid, a peptide inducible system, or a caffeine inducible system. In another embodiment, either of the first or second chemically inducible proximity systems is a plant hormone analogue inducible system and is selected from any inducible system in which the inducer is a plant hormone analogue, or a synthetic plant hormone. Suitable plant hormone analogues include Mandipropamid, for example. Suitably the use of the term ‘plant hormone’ herein encompasses plant hormone analogues.

In one embodiment either of the first or second plant hormone inducible systems is an auxin inducible proximity system as defined herein or abscisic acid inducible proximity system as defined herein in a mutually exclusive manner. Suitably the first plant hormone inducible proximity system is an auxin inducible proximity system as defined herein and a second plant hormone inducible proximity system is an abscisic acid inducible proximity system as defined herein, or vice versa. Suitably therefore the first plant hormone inducer is auxin and the second plant hormone inducer is abscisic acid, and the first plant hormone inducer binding domain is an auxin inducer binding domain and the second plant hormone inducer binding domain is an abscisic acid binding domain.

In another embodiment, either of the first or second chemically inducible proximity systems is a plant hormone or plant hormone analogue inducible proximity system. In such an embodiment, either of the first or second systems is selected from a caffeine inducible proximity system, a Mandipropamid inducible proximity system, a gibberellin inducible proximity system and an abscisic acid inducible proximity system as defined herein in a mutually exclusive manner. Suitably in some embodiments, the first plant hormone or plant hormone analogue inducible proximity system is a caffeine inducible proximity system as defined herein, and a second plant hormone inducible proximity system is an abscisic acid inducible proximity system as defined herein, or vice versa. Suitably in some embodiments, the first plant hormone or plant hormone analogue inducible proximity system is a Mandipropamid inducible proximity system as defined herein, and a second plant hormone inducible proximity system is an abscisic acid inducible proximity system as defined herein, or vice versa. Suitably in some embodiments, the first plant hormone or plant hormone analogue inducible proximity system is a gibberellin inducible proximity system as defined herein, and a second plant hormone inducible proximity system is an abscisic acid inducible proximity system as defined herein, or vice versa.

Suitably the or each auxin binding domain is selected from Transport Inhibitor Response 1 protein (TIR1) as described elsewhere herein, or Auxin/indole-3-acetic acid protein (AID) as described elsewhere herein. Suitably therefore one auxin binding domain is TIR1, suitably a first auxin binding domain is TIR1 or a fragment or derivative thereof. Suitably one auxin binding domain is AID, suitably a second auxin binding domain is AID or a fragment or derivative thereof.

Suitably the or each caffeine binding domain is anti-caffeine heavy-chain antibody fragment (aCaffVHH) as described elsewhere herein. Suitably therefore both the first and second caffeine binding domains are an Anti-caffeine heavy-chain antibody fragment (aCaffVHH), or a fragment or derivative thereof.

Suitably the or each mandipropamid binding domain is selected from: a modified pyrobactin receptor (PYR^{Mandi}), a modified pyrobactin-like receptor (PYLcs^{Mandi}), and abscisic acid insensitive 1 protein (ABI) as described elsewhere herein. Suitably one Mandipropamid binding domain is ABI, suitably a first Mandipropamid binding domain is ABI or a fragment or derivative thereof. Suitably one Mandipropamid binding domain is a modified pyrobactin receptor (PYR^{Mandi}), or a modified pyrobactin-like receptor (PYLcs^{Mandi}), suitably a second Mandipropamid binding domain is a modified pyrobactin receptor (PYR^{Mandi}) or a modified pyrobactin-like receptor (PYLcs^{Mandi}) or a fragment or derivative thereof.

Suitably the or each gibberellin binding domain is selected from gibberellin insensitive dwarf 1 (GID1) protein and gibberellin insensitive (GAI) protein optionally which may be a modified GAI protein. Suitably one gibberellin binding domain is gibberellin insensitive dwarf 1 protein (GID1), suitably a first gibberellin binding domain is gibberellin insensitive dwarf 1 protein (GID1) or a fragment or derivative thereof. Suitably one gibberellin binding domain is a gibberellin insensitive (GAI) protein or a modified GAI protein, suitably a second gibberellin binding domain is gibberellin insensitive (GAI) protein or a modified GAI protein or a fragment or derivative thereof.

Suitably the or each abscisic acid binding domain is selected from: abscisic acid insensitive 1 protein (ABI1) as described elsewhere herein or pyrobactin resistance-like protein (PYL1) as described elsewhere herein. Suitably one abscisic acid binding domain is ABI1, suitably a first abscisic acid binding domain is ABI1 or a fragment or derivative thereof. Suitably one abscisic acid binding domain is PYL1, suitably a second abscisic acid binding domain is PYL1 or a fragment or derivative thereof.

Suitably the or each transactivation domain is selected from: Gal4, Oaf1, Leu3, Rtg3, Pho4, Gln3, Gcn4 in yeast, and p53, NFAT, NF-κB, VP16 or VP34, preferably the or each transactivation domain is VP16. VP16. Suitably either of the first or second DNA binding domains may be a dl-Scel DNA binding domain or a GAL4 DNA binding domain in a mutually exclusive manner.

In one embodiment, the first chimeric protein comprises a VP16 transactivation domain and a TIR1 protein or a fragment or derivative thereof, and the second chimeric protein comprises a dl-Scel DNA binding domain and an AID protein or a fragment or derivative thereof, or vice versa. In one embodiment, an AIDΔ34 protein.

In one embodiment, the first chimeric protein comprises a VP16 transactivation domain and a aCaffVHH protein or a fragment or derivative thereof, and the second chimeric protein comprises a GAL4 DNA binding domain or a dl-Scel DNA binding domain and an aCaffVHH protein or a fragment or derivative thereof, or vice versa.

In one embodiment, the first chimeric protein comprises a VP16 transactivation domain and a PYR1 or a PYL1 protein or a fragment or derivative thereof, and the second chimeric protein comprises a GAL4 DNA binding domain or a dl-Scel DNA binding domain and an ABI1 protein or a fragment or derivative thereof, or vice versa. In one embodiment, a PYR^{Mandi} protein or a PYLcs^{Mandi} protein. In one embodiment an ABIcs protein.

In one embodiment, the first chimeric protein comprises a VP16 transactivation domain and a GID1 protein or a fragment or derivative thereof, and the second chimeric protein comprises a GAL4 DNA binding domain or a dl-Scel DNA binding domain and a GAI protein or a fragment or derivative thereof, or vice versa. In one embodiment, a modified GAI protein.

In one embodiment, the second construct comprises between 1 to 15 dl-Scel DNA binding sites, preferably ten dl-Scel DNA binding sites, preferably in tandem, or between 1 and 15 GAL4 upstream activation sequences, preferably nine GAL4 upstream activation sequences, preferably in tandem.

In one embodiment, the third chimeric protein comprises a VP16 transactivation domain and a PYL1 protein or a fragment or derivative thereof, and the fourth chimeric protein comprises a GAL4 DNA binding domain or a dl-Scel DNA binding domain and an ABI1 protein or a fragment or derivative thereof, or vice versa. In one embodiment, a PYLcs protein. In one embodiment an ABIcs protein.

In one embodiment, the fourth construct comprises between 1 and 15 GAL4 upstream activation sequences, preferably nine GAL4 upstream activation sequences, preferably in tandem, or between 1 to 15 dl-Scel DNA binding sites, preferably ten dl-Scel DNA binding sites, preferably in tandem.

Suitably if a GAL4 DNA binding domain is present in the first or third chimeric protein then the second construct or the fourth construct respectively must comprise between 1 and 15 GAL4 upstream activation sequences, preferably nine GAL4 upstream activation sequences, preferably in tandem. Suitably if a dl-Scel DNA binding domain is present in the first or third chimeric protein then the second construct or the fourth construct respectively must comprise between 1 to 15 dl-Scel DNA binding sites, preferably ten dl-Scel DNA binding sites, preferably in tandem.

Suitably, it will be appreciated that the components of the first chimeric protein and the third chimeric protein may be reversed. Suitably it will be appreciated that the components of the second chimeric protein and the fourth chimeric protein may be reversed. Suitably therefore, it will be appreciated that the second construct and the fourth construct may also be reversed.

It is envisaged that other suitable chemically inducible proximity systems, and other plant hormone or plant hormone analogue inducible systems may be used as the first and/or second plant hormone inducible proximity systems herein and combined to arrive at a cell comprising the dual chemically induced proximity system of the invention which may be used to control expression of two proteins of interest.

Suitably any references in any aspect or embodiment herein to ‘the auxin inducible proximity system’ may be replaced with a first chemically inducible proximity system, or suitably a first plant hormone or plant hormone analogue inducible proximity system, and any references to ‘the abscisic acid inducible proximity system’ may be replaced with a second chemically inducible proximity system, or suitably a second plant hormone or plant hormone analogue inducible proximity system, and the corresponding component parts of each system as defined in the eighth aspect.

In one embodiment of the eighth aspect, there is provided a cell comprising the auxin inducible proximity system of the third aspect and/or an abscisic acid inducible proximity system wherein the auxin inducible proximity system (a) comprises:
(i) a first construct comprising a promoter operably linked to a nucleic acid sequence encoding:
   a first chimeric protein, and
   a second chimeric protein,
wherein the first and second chimeric proteins each comprise an auxin binding domain and an effector domain;
wherein the auxin binding domain is selected from a Transport Inhibitor Response 1 protein (TIR1) or Auxin/indole-3-acetic acid protein (AID); wherein the effector domain is selected from a transactivation domain or a catalytically inactive I-Scel endonuclease DNA binding domain (dl-Scel);
wherein the auxin binding domain and the effector domain of the first and second chimeric proteins are different; and
(ii) a second construct comprising a nucleic acid sequence encoding: one or more effector domain binding sites operably linked to a nucleic acid sequence encoding a first protein of interest, wherein each effector domain binding site is a dl-Scel binding site;
   wherein the abscisic acid inducible proximity system (b) comprises:
(i) a third construct comprising a promoter operably linked to a nucleic acid sequence encoding:
   a third chimeric protein, and
   a fourth chimeric protein,
wherein the third and fourth chimeric proteins each comprise an abscisic acid binding domain and an effector domain;
wherein the abscisic acid binding domain is selected from an abscisic acid insensitive 1 protein (ABI1) or pyrobactin resistance-like protein (PYL1); wherein the effector domain is selected from a transactivation domain or Gal4 DNA binding domain;
wherein the abscisic acid binding domain and the effector domain of the third and fourth chimeric proteins are different; and
(ii) a fourth construct comprising a nucleic acid sequence encoding: one or more effector domain binding sites operably linked to a nucleic acid sequence encoding a second protein of interest, wherein each effector domain binding site is a Gal4 upstream activation sequence.

In one embodiment, the auxin inducible proximity system and the abscisic acid inducible proximity system are as defined hereinabove.

In one embodiment the cell may comprise only the auxin inducible proximity system. In one embodiment the cell may comprise only the abscisic acid inducible proximity system. In one embodiment, the cell comprises both the auxin inducible proximity system and the abscisic acid inducible proximity system. In one embodiment, the cell is an inducible cell, which may give rise to an inducible cell line comprising the auxin inducible proximity system of the third aspect, and/or an abscisic acid inducible proximity system described herein.

Suitably the chemically inducible proximity systems and cells comprising the systems described herein may be used in various methods. It will be appreciated however that the methods may also make use of other inducible systems, not necessarily CIP systems. Suitable other inducible systems are described herein.

Suitably the inducible systems may be used in various methods to control expression of one or more proteins of interest in a cell. Suitably such methods are useful for screening of candidate biological molecules, therapeutic agents, and/or engineered immune cells. Suitably such methods are useful for screening candidate biological molecules, therapeutic agents, and/or engineered immune cells for a biological effect, sutiably for a biological effect on the cell expressing the or each protein.

In a ninth aspect of the invention there is provided a method of controlling expression of a first and optionally a second protein of interest in a cell comprising:
(a) Providing a cell comprising a first inducible system operable to express a first protein of interest, and optionally a second inducible system operable to express a second protein of interest;
(b) Exposing the cell to an effective concentration of a first inducer to induce a desired level of expression of the first protein of interest from the first inducible system, and optionally exposing the cell to an effective concentration of a second inducer to induce a desired level of expression of the second protein of interest from the second inducible system.

Suitably the first and the second inducible system are different. In a preferred embodiment, the cell comprises both a first and second inducible system. Suitably there is substantially no crosstalk between the first and second inducible systems.

Optionally the method may comprise a step (b) of culturing the cell under conditions to express necessary components of the first and/or second inducible systems.

In some embodiments, the first and second inducible systems may be any inducible system, suitably any inducible system as described herein. In one embodiment, the first and second inducible systems may be chemically induced proximity systems (CIP systems). In one preferred embodiment, they may be plant hormone or plant hormone analogue inducible proximity systems as described herein. In some embodiments, the first inducible system may be a first plant hormone inducible proximity system of the eighth aspect. In some embodiments, the second inducible system may be a second plant hormone inducible proximity system of the eighth aspect. Suitably in such embodiments, the first inducible system comprises a first and second construct as defined herein, and the second inducible system comprises a third and a fourth construct as defined herein. In one embodiment, at least one of the inducible systems is a plant hormone inducible proximity system. In one embodiment, at least one of the inducible systems is an abscisic acid inducible proximity system defined herein. In one embodiment, at least one of the inducible systems is selected from a caffeine inducible proximity system defined herein, a Mandipropamid inducible proximity system defined herein, and a gibberellin inducible proximity system defined herein.

In a preferred embodiment, the first inducible system is selected from a caffeine inducible proximity system defined herein, a Mandipropamid inducible proximity system defined herein, and a gibberellin inducible proximity system defined herein and the second inducible system is an abscisic acid inducible proximity system as defined herein.

In an embodiment of the ninth aspect of the invention there is provided a method of controlling expression of a protein of interest in a cell comprising:
(a) Providing a cell comprising the first plant hormone inducible proximity system and/or second plant hormone inducible proximity system of the eighth aspect;
(b) Culturing the cell under conditions to express the first construct and/or the third construct;
(c) Exposing the cell to an effective concentration of a first plant hormone inducer to induce a desired level of expression of the first protein of interest from the second construct, and/or exposing the cell to an effective concentration of a second plant hormone inducer to induce a desired level of expression of the second protein of interest from the fourth construct.

In one embodiment of the ninth aspect, there is provided a method of controlling expression of a protein of interest in a cell comprising:
(a) Providing a cell comprising the auxin inducible proximity system and/or an abscisic acid inducible proximity system defined herein;
(b) Culturing the cell under conditions to express the first construct and/or the third construct;
(c) Exposing the cell to an effective concentration of auxin to induce a desired level of expression of the first protein of interest from the second construct, and/or exposing the cell to an effective concentration of abscisic acid to induce a desired level of expression of the second protein of interest from the fourth construct.

In one embodiment, the method of the ninth aspect is a method of controlling expression of a first and a second protein of interest in a cell, comprising:
(a) Providing a cell comprising the auxin inducible proximity system and abscisic acid inducible proximity system described herein;
(b) Culturing the cell under conditions to express the first construct and the third construct;
(c) Exposing the cell to an effective concentration of auxin to induce a desired level of expression of the first protein of interest from the second construct and exposing the cell to an effective concentration of abscisic acid to induce a desired level of expression of the second protein of interest from the fourth construct.

In one embodiment, the auxin inducible proximity system and abscisic acid inducible proximity system are as defined hereinabove, as are the first, second, third and fourth constructs.

In one embodiment, step (c) may comprise exposing the cell to a plurality of different concentrations of first inducer, suitably plant hormone inducer, suitably auxin, to induce a plurality of different levels of expression of the first protein of interest, which may expressed from the second construct, and/or exposing the cell to a plurality of different concentrations of second inducer, suitably plant hormone inducer, suitably abscisic acid, to induce a desired level of expression of the second protein of interest, which may be expressed from the fourth construct.

In one embodiment, step (c) may comprise exposing the cell to an effective concentration of a first inducer, suitably a plant hormone inducer, suitably auxin and an effective concentration of a second inducer, suitably a plant hormone inducer, suitably abscisic acid at the same time or at different times as explained further below.

In a tenth aspect of the invention there is provided a method of screening a candidate binding molecule for a biological effect comprising:
(a) Providing a cell comprising a first inducible system operable to express a first protein of interest, and optionally a second inducible system operable to express a second protein of interest;
(b) Exposing the cell to an effective concentration of a first inducer to induce a desired level of expression of the first protein of interest, and optionally exposing the cell to an effective concentration of a second inducer to induce a desired level of expression of the second protein of interest;
(c) Contacting the cell with a candidate binding molecule;
(d) Determining whether the candidate binding molecule enacts a biological effect on the cell expressing the first and optionally the second protein of interest.

Preferred or optional features defined above in relation to the ninth aspect, apply equally to the tenth aspect.

Suitably the term ‘enact’ herein may be used interchangeably with ‘exact’ ‘elicit’ or ‘cause’ in relation to the biological effect.

In one embodiment of the tenth aspect of the invention there is provided a method of screening a candidate binding molecule for a biological effect comprising:
(a) Providing a cell comprising the first plant hormone inducible proximity system and/or the second plant hormone inducible proximity system of the eighth aspect;
(b) Culturing the cell under conditions to express the first construct and/or the third construct;
(c) Exposing the cell to an effective concentration of a first plant hormone inducer to induce a desired level of expression of the first protein of interest from the second construct, and/or exposing the cell to an effective concentration of a second plant hormone inducer to induce a desired level of expression of the second protein of interest from the fourth construct;
(d) Contacting the cell with a candidate binding molecule;
(e) Determining whether the candidate binding molecule enacts a biological effect on the cell expressing the first and/or second protein of interest.

In one embodiment of the tenth aspect, there is provided a method of screening a candidate binding molecule for a biological effect comprising:
(a) Providing a cell comprising the auxin inducible proximity system and/or an abscisic acid inducible proximity system as defined herein;
(b) Culturing the cell under conditions to express the first construct and/or the third construct;
(c) Exposing the cell to an effective concentration of auxin to induce a desired level of expression of the first protein of interest from the second construct, and/or exposing the cell to an effective concentration of abscisic acid to induce a desired level of expression of the second protein of interest from the fourth construct;
(d) Contacting the cell with a candidate binding molecule;
(e) Determining whether the candidate binding molecule enacts a biological effect on the cell expressing the first and/or second protein of interest.

In one embodiment of the tenth aspect, the method is a method of screening a candidate binding molecule for a biological effect comprising:
(a) Providing a cell comprising the auxin inducible proximity system and an abscisic acid inducible proximity system as defined herein;
(b) Culturing the cell under conditions to express the first construct and the third construct;
(c) Exposing the cell to an effective concentration of auxin to induce a desired level of expression of the first protein of interest from the second construct, and exposing the cell to an effective concentration of abscisic acid to induce a desired level of expression of the second protein of interest from the fourth construct;
(d) Contacting the cell with a candidate binding molecule;
(e) Determining whether the candidate binding molecule enacts a biological effect on the cell expressing the first and second protein of interest.

In one embodiment, the auxin inducible proximity system and abscisic acid inducible proximity system are as defined hereinabove, as are the first, second, third and fourth constructs.

In one embodiment, the biological effect comprises binding to the first and/or second protein of interest. In one embodiment, step (d) may comprise determining whether the candidate binding molecule binds to both the first and second protein of interest.

In one embodiment, step (c) may comprise exposing the cell to a plurality of different concentrations of first inducer, suitably a plant hormone inducer, suitably auxin, to induce a plurality of different levels of expression of the first protein of interest, which may be expressed from the second construct, and/or exposing the cell to a plurality of different concentrations of second inducer, suitably a plant hormone inducer, suitably abscisic acid, to induce a desired level of expression of the second protein of interest, which may be expressed from the fourth construct.

In one embodiment, step (c) may comprise exposing the cell to an effective concentration of a first inducer, suitably a plant hormone inducer, suitably auxin and an effective concentration of a second inducer, suitably a plant hormone inducer, suitably abscisic acid at the same time or at different times as explained further below.

In one embodiment, step (d) may comprise determining the minimum level of expression of the first and/or second protein of interest at which the candidate binding molecule enacts a biological effect on the cell expressing the first and/or second protein of interest.

In a further aspect, there is therefore provided a method determining the minimum level of expression of at least one protein of interest in a cell at which a candidate binding molecule enacts a biological effect, the method comprising:
(a) Providing a cell comprising a first inducible system operable to express a first protein of interest, and optionally a second inducible system operable to express a second protein of interest;
(b) Exposing the cell comprising the first inducible system, and optionally the second inducible system, to a plurality of different concentrations of a first inducer to induce a plurality of different levels of expression of the first protein of interest, and optionally exposing the cell to a plurality of different concentrations of a second inducer to induce a plurality of different levels of expression of the second protein of interest;
(c) Contacting the cell with the candidate binding molecule;
(d) Determining whether the candidate binding molecule enacts a biological effect on the cell expressing the first and optionally the second protein of interest at each level of expression of the first and optionally the second protein of interest; and
(e) Determining the minimum level of expression of the first and optionally the second protein of interest at which the candidate binding molecule enacts a biological effect on the cell expressing the first and optionally the second protein of interest

In one embodiment of such an aspect, there is provided a method of determining the minimum level of expression of at least one protein of interest in a cell at which a candidate binding molecule enacts a biological effect, the method comprising:
(a) Providing a cell comprising the first plant hormone inducible proximity system and/or second plant hormone inducible proximity system of the eighth aspect;
(b) Culturing the cell comprising the first plant hormone inducible proximity system and/or the second plant hormone inducible proximity system under conditions to express the first construct and the third construct;
(c) Exposing the cell comprising the first plant hormone inducible proximity system, and/or the second plant hormone inducible proximity system, to a plurality of different concentrations of a first plant hormone inducer to induce a plurality of different levels of expression of the first protein of interest from the second construct, and/or exposing the cell to an effective concentration of a second plant hormone inducer to induce a desired level of expression of the second protein of interest from the fourth construct;
(d) Contacting the cell with the candidate binding molecule;
(e) Determining whether the candidate binding molecule enacts a biological effect on the cell expressing the first and/or second protein of interest at each level of expression of the first and/or second protein of interest; and
(f) Determining the minimum level of expression of the first and/or second protein of interest at which the candidate binding molecule enacts a biological effect on the cell expressing the first and/or second protein of interest.

In one embodiment of the further aspect, there is provided a method of determining the minimum level of expression of at least one protein of interest in a cell at which a candidate binding molecule enacts a biological effect, the method comprising:
(a) Providing a cell comprising the auxin inducible proximity system and/or an abscisic acid inducible proximity system as defined herein;
(b) Culturing the cell comprising the auxin inducible proximity system and/or the abscisic acid inducible proximity system under conditions to express the first construct and the third construct;
(c) Exposing the cell comprising the auxin inducible proximity system, and/or the abscisic acid inducible proximity system, to a plurality of different concentrations of auxin to induce a plurality of different levels of expression of the first protein of interest from the second construct, and/or exposing the cell to an effective concentration of abscisic acid to induce a desired level of expression of the second protein of interest from the fourth construct;
(d) Contacting the cell with the candidate binding molecule;
(e) Determining whether the candidate binding molecule enacts a biological effect on the cell expressing the first and/or second protein of interest at each level of expression of the first and/or second protein of interest; and
(f) Determining the minimum level of expression of the first and/or second protein of interest at which the candidate binding molecule enacts a biological effect on the cell expressing the first and/or second protein of interest.

In one embodiment of the further aspect, the method is a method of determining the minimum level of expression of a first and a second protein of interest in a cell at which a candidate binding molecule enacts a biological effect, the method comprising:
(a) Providing a cell comprising the auxin inducible proximity system and an abscisic acid inducible proximity system as defined herein;
(b) Culturing the cell comprising the auxin inducible proximity system and the abscisic acid inducible proximity system under conditions to express the first construct and the third construct;
(c) Exposing the cell comprising the auxin inducible proximity system, and the abscisic acid inducible proximity system, to a plurality of different concentrations of auxin to induce a plurality of different levels of expression of the first protein of interest from the second construct, and exposing the cell to an effective concentration of abscisic acid to induce a desired level of expression of the second protein of interest from the fourth construct;
(d) Contacting the cell with the candidate binding molecule;
(e) Determining whether the candidate binding molecule enacts a biological effect on the cell expressing the first and second protein of interest at each level of expression of the first and second protein of interest; and
(f) Determining the minimum level of expression of the first and/or second protein of interest at which the candidate binding molecule enacts a biological effect on the cell expressing the first and second protein of interest.

In one embodiment, the auxin inducible proximity system and abscisic acid inducible proximity system are as defined hereinabove, as are the first, second, third and fourth constructs.

In one embodiment, step (b) or (c) as appropriate may comprise exposing the cell to a concentration of auxin and a concentration of abscisic acid at the same time or at different times as explained further below.

In one embodiment, the biological effect comprises binding to the first and/or second protein of interest. In one embodiment, step (d) or (e) as appropriate may comprise determining whether the candidate binding molecule binds to both the first and/or second protein of interest at each level of expression of the first and/or second protein of interest.

In one embodiment, step (d) or (e) as appropriate may comprise determining whether the candidate binding molecule enacts a biological effect on the cell expressing the first and second protein of interest at each level of expression of both the first and second protein of interest.

In one embodiment, step (e) or (f) as appropriate may comprise determining the minimum level of expression of both the first and second protein of interest at which the candidate binding molecule enacts a biological effect on the cell expressing both the first and second protein of interest.

In one embodiment, the minimum level of expression of the first and/or second protein of interest at which the candidate binding molecule enacts a biological effect on the cell expressing the first and/or second protein of interest may be the level of expression at which a biological effect higher than the background biological effect is achieved. Suitably at which a biological effect of at least 3 standard deviation above the background biological effect is achieved, suitably at least 4 standard deviations above the background biological effect is achieved, suitably at least 5 standard deviations above the background biological effect is achieved, suitably at least 6 standard deviations above the background biological effect is achieved, suitably at least 7 standard deviations above the background biological effect is achieved, suitably at least 8 standard deviations above the background biological effect is achieved, suitably at least 9 standard deviations above the background biological effect is achieved, suitably at least 10 standard deviations above the background biological effect is achieved. Suitably wherein the background biological effect is the biological effect of the candidate binding molecule on a control cell. Suitably wherein the control cell is a cell which does not contain an inducible system as described herein. Suitably the control cell does not express the or each protein of interest. Suitably wherein the control cells is a wild type cell. Alternatively, the control cell may be a cell that has been modified to prevent expression of the or each protein of interest, suitably by ‘knocking out’ the gene encoding the or each protein of interest which may be achieved by known modification techniques such as RNA interference, RNA silencing, CRISPRi, zinc finger nucleases, TALENs etc.

In one embodiment, the minimum level of expression of the first and/or second protein of interest at which the candidate binding molecule enacts a biological effect on the cell expressing the first and/or second protein of interest may be the activation threshold of the first and/or second protein of interest. Suitably this may be determined using receiver operator characteristic (ROC) curve analysis, suitably using Youden's index or Youden's J statistic.

In one embodiment, the candidate binding molecule is selected from: a fusion protein, an antibody (e.g. a monoclonal antibody, an antibody drug conjugate, a nanobody, scFv, di-scFv, Fab, sdAb, F(ab)2, a 27glycol-engineered antibody) or a binding fragment thereof, a fusion protein, an antibody-drug conjugate, an aptamer, an ankyrin, a designed ankyrin repeat protein (DARPin), a peptide, a bicyclic peptide, a vaccine, a cytokine, a chemokine, a hormone, an Oncolytic virus, and a bacterium, preferably the binding molecule is an immunotherapy.

In an eleventh aspect of the invention there is provided a method of screening a candidate therapeutic agent for a biological effect comprising:
(a) Providing a cell comprising first inducible system operable to express a first protein of interest, and optionally a second inducible system operable to express a second protein of interest, and an immune cell;
(b) Exposing the cell comprising the first inducible system, and optionally the second inducible system, to an effective concentration of a first inducer to induce a desired level of expression of the first protein of interest, and optionally exposing the cell to an effective concentration of a second inducer to induce a desired level of expression of the second protein of interest;
(c) Contacting the immune cell with the candidate therapeutic agent; and
(d) Determining whether the contacted immune cell enacts a biological effect on the cell expressing the first and optionally the second protein of interest.

Preferred or optional features defined above in relation to the ninth aspect, apply equally to the eleventh aspect.

Optionally the method may further comprise a step of: contacting or exposing the cell expressing the first and optionally the second protein of interest with/to the contacted immune cell. Suitably prior to step (d).

In one embodiment of the eleventh aspect of the invention there is provided a method of screening a candidate therapeutic agent for a biological effect comprising:
(a) Providing a cell comprising the first plant hormone inducible proximity system and/or second plant hormone inducible proximity system of the eighth aspect, and an immune cell;
(b) Culturing the cell comprising the first plant hormone inducible proximity system and/or the second plant hormone inducible proximity system under conditions to express the first construct and/or the third construct;
(c) Exposing the cell comprising the first plant hormone inducible proximity system, and/or the second plant hormone inducible proximity system, to an effective concentration of a first plant hormone inducer to induce a desired level of expression of the first protein of interest from the second construct, and/or exposing the cell to an effective concentration of a second plant hormone inducer to induce a desired level of expression of the second protein of interest from the fourth construct;
(d) Contacting the immune cell with the candidate therapeutic agent;
(e) Determining whether the contacted immune cell enacts a biological effect on the cell expressing the first and/or second protein of interest

In one embodiment of the eleventh aspect, there is provided a method of screening a candidate therapeutic agent for a biological effect comprising:
(a) Providing a cell comprising the auxin inducible proximity system and/or an abscisic acid inducible proximity system as defined herein, and an immune cell;
(b) Culturing the cell comprising the auxin inducible proximity system and/or the abscisic acid inducible proximity system under conditions to express the first construct and/or the third construct;
(c) Exposing the cell comprising the auxin inducible proximity system, and/or the abscisic acid inducible proximity system, to an effective concentration of auxin to induce a desired level of expression of the first protein of interest from the second construct, and/or exposing the cell to an effective concentration of abscisic acid to induce a desired level of expression of the second protein of interest from the fourth construct;
(d) Contacting the immune cell with the candidate therapeutic agent;
(e) Determining whether the contacted immune cell enacts a biological effect on the cell expressing the first and/or second protein of interest

In one embodiment of the eleventh aspect of the invention, the method is a method of screening a candidate therapeutic agent for a biological effect comprising:
(a) Providing a cell comprising the auxin inducible proximity system and an abscisic acid inducible proximity system as defined herein, and an immune cell;
(b) Culturing the cell comprising the auxin inducible proximity system and abscisic acid inducible proximity system under conditions to express the first construct and the third construct;
(c) Exposing the cell comprising the auxin inducible proximity system to an effective concentration of auxin to induce a desired level of expression of a first protein of interest from the second construct, and exposing the cell to an effective concentration of abscisic acid to induce a desired level of expression of a second protein of interest from the fourth construct;
(d) Contacting the immune cell with the candidate therapeutic agent;
(e) Determining whether the contacted immune cell enacts a biological effect on the cell expressing the first and second protein of interest

In one embodiment, the auxin inducible proximity system and abscisic acid inducible proximity system are as defined hereinabove, as are the first, second, third and fourth constructs.

In one embodiment, step (b) or (c) as appropriate may comprise exposing the cell to a plurality of different concentrations of first inducer, suitably a plant hormone inducer, suitably auxin, to induce a plurality of different levels of expression of the first protein of interest, which may be expressed from the second construct, and/or exposing the cell to a plurality of different concentrations of second inducer, suitably plant hormone inducer, suitably abscisic acid, to induce a desired level of expression of the second protein of interest, which may be expressed from the fourth construct.

In one embodiment, step (b) or (c) as appropriate may comprise exposing the cell to an effective concentration of a first inducer, suitably a plant hormone inducer, suitably auxin and an effective concentration of a second inducer, suitably a plant hormone inducer, suitably abscisic acid at the same time or at different times as explained further below.

In one embodiment, step (d) or (e) as appropriate may comprise determining whether the contacted immune cell enacts a biological effect on the cell expressing both the first and second protein of interest. In one embodiment, step (d) or (e) as appropriate may comprise determining whether the contacted immune cell enacts a biological effect on the cell expressing the first and/or second protein of interest at each level of expression of the first and/or second protein of interest.

In one embodiment, the method may further comprise a step (e) or (f) as appropriate of determining the minimum expression levels of the first and/or second protein of interest at which the contacted immune cell is enacting a biological effect in the presence of the therapeutic agent.

In one embodiment, step (e) or (f) as appropriate may comprise determining the minimum expression levels of both the first and second protein of interest at which the contacted immune cell is enacting a biological effect in the presence of the therapeutic agent.

In one embodiment, the minimum level of expression of the first and/or second protein of interest at which the contacted immune cell enacts a biological effect on the cell expressing the first and/or second protein of interest may be the level of expression at which a biological effect higher than the background biological effect is achieved. Suitably at which a biological effect of at least 3 standard deviation above the background biological effect is achieved, suitably at least 4 standard deviations above the background biological effect is achieved, suitably at least 5 standard deviations above the background biological effect is achieved, suitably at least 6 standard deviations above the background biological effect is achieved, suitably at least 7 standard deviations above the background biological effect is achieved, suitably at least 8 standard deviations above the background biological effect is achieved, suitably at least 9 standard deviations above the background biological effect is achieved, suitably at least 10 standard deviations above the background biological effect is achieved. Suitably wherein the background biological effect is the biological effect of the candidate binding molecule on a control cell. Suitably wherein the control cell is a cell which does not contain an inducible system as described herein. Suitably the control cell does not express the or each protein of interest. Suitably wherein the control cells is a wild type cell. Alternatively, the control cell may be a cell that has been modified to prevent expression of the or each protein of interest, suitably by ‘knocking out’ the gene encoding the or each protein of interest which may be achieved by known modification techniques such as RNA interference, RNA silencing, CRISPRi, zinc finger nucleases, TALENs etc.

In one embodiment, the minimum level of expression of the first and/or second protein of interest at which the contacted immune cell enacts a biological effect on the cell expressing the first and/or second protein of interest may be the activation threshold of the first and/or second protein of interest. Suitably this may be determined using receiver operator characteristic (ROC) curve analysis, suitably using Youden's index or Youden's J statistic.

In one embodiment, the candidate therapeutic agent is a biologic, preferably the candidate therapeutic agent is an immunotherapy, preferably selected from a fusion protein, an antibody (e.g. a monoclonal antibody, an antibody drug conjugate, a nanobody, scFv, di-scFv, Fab, sdAb, F(ab)₂, a 31glycol-engineered antibody) or a binding fragment thereof, a fusion protein, an antibody-drug conjugate, an aptamer, an ankyrin, a designed ankyrin repeat protein (DARPin), a peptide, a bicyclic peptide, a vaccine, a cytokine, a chemokine, a hormone, an Oncolytic virus, and a bacterium.

In one embodiment, the immune cell is selected from a T cell, an NK cell, a B cell, a lymphocyte, a dendritic cell, and a mesenchymal cell, or immortalised cells thereof, or immortalised cells thereof.

In a twelfth aspect of the invention there is provided a method of determining the minimum level of expression of at least one protein of interest in a cell at which an immune cell enacts a biological effect in the presence of a candidate therapeutic agent the method comprising:
(a) Providing a cell comprising a first inducible system operable to express a first protein of interest, and optionally a second inducible system operable to express a second protein of interest, and an immune cell;
(b) Exposing the cell comprising the first inducible system and optionally the second inducible system to a plurality of different concentrations of a first inducer to induce a plurality of different levels of expression of the first protein of interest, and optionally exposing the cell to a plurality of different concentrations of a second inducer to induce a plurality of different levels expression of the second protein of interest;
(c) Contacting the immune cell with the candidate therapeutic agent;
(d) Determining whether the contacted immune cell enacts a biological effect on the cell expressing the first and optionally the second protein of interest at each level of expression of the first and optionally the second protein of interest;
(e) Determining the minimum level of expression of the first and optionally the second protein of interest at which the contacted immune cell enacts a biological effect on the cell expressing the first and optionally the second protein of interest.

Preferred or optional features defined above in relation to the ninth aspect, apply equally to the twelfth aspect.

Optionally the method may further comprise a step of: contacting or exposing the cell expressing the first and optionally the second protein of interest with/to the contacted immune cell. Suitably prior to step (d).

In an embodiment of the twelfth aspect of the invention there is provided a method of determining the minimum level of expression of at least one protein of interest in a cell at which an immune cell enacts a biological effect in the presence of a candidate therapeutic agent the method comprising:
(a) Providing a cell comprising the first plant hormone inducible proximity system and/or second plant hormone inducible proximity system of the eighth aspect, and an immune cell;
(b) Culturing the cell comprising the first plant hormone inducible proximity system and/or the second plant hormone inducible proximity system, under conditions to express the first construct and/or the third construct;
(c) Exposing the cell comprising the first plant hormone inducible proximity system and/or the second plant hormone inducible proximity system to a plurality of different concentrations of a first plant hormone inducer to induce a plurality of different levels of expression of the first protein of interest from the second construct, and/or exposing the cell to a plurality of different concentrations of a second plant hormone inducer to induce a plurality of different levels of expression of the second protein of interest from the fourth construct;
(d) Contacting the immune cell with the candidate therapeutic agent;
(e) Determining whether the contacted immune cell enacts a biological effect on the cell expressing the first and/or second protein of interest at each level of expression of the first and/or second protein of interest
(f) Determining the minimum level of expression of the first and/or second protein of interest at which the contacted immune cell enacts a biological effect on the cell expressing the first and/or second protein of interest

In one embodiment of the twelfth aspect, there is provided a method of determining the minimum level of expression of at least one protein of interest in a cell at which an immune cell enacts a biological effect in the presence of a candidate therapeutic agent the method comprising:
(a) Providing a cell comprising the auxin inducible proximity system and/or an abscisic acid inducible proximity system as defined herein, and an immune cell;
(b) Culturing the cell comprising the auxin inducible proximity system and/or the abscisic acid inducible proximity system, under conditions to express the first construct and/or the third construct;
(c) Exposing the cell comprising the auxin inducible proximity system and/or the abscisic acid inducible proximity system to a plurality of different concentrations of auxin to induce a plurality of different levels of expression of the first protein of interest from the second construct, and/or exposing the cell to a plurality of different concentrations of abscisic acid to induce a a plurality of different levels of expression of the second protein of interest from the fourth construct;
(d) Contacting the immune cell with the candidate therapeutic agent;
(e) Determining whether the contacted immune cell enacts a biological effect on the cell expressing the first and/or second protein of interest at each level of expression of the first and/or second protein of interest
(f) Determining the minimum level of expression of the first and/or second protein of interest at which the contacted immune cell enacts a biological effect on the cell expressing the first and/or second protein of interest

In one embodiment of the twelfth aspect, the method is a method of determining the minimum level of expression of a first and a second protein of interest in a cell at which an immune cell enacts a biological effect in the presence of a candidate therapeutic agent the method comprising:
(a) Providing a cell comprising the auxin inducible proximity system and an abscisic acid inducible proximity system as defined herein, and an immune cell;
(b) Culturing the cell comprising the auxin inducible proximity system and the abscisic acid inducible proximity system, under conditions to express the first construct and the third construct;
(c) Exposing the cell comprising the auxin inducible proximity system and the abscisic acid inducible proximity system to a plurality of different concentrations of auxin to induce a plurality of different levels of expression of the first protein of interest from the second construct, and exposing the cell to a plurality of different concentrations of abscisic acid to induce a plurality of different levels of expression of the second protein of interest from the fourth construct;
(d) Contacting the immune cell with the candidate therapeutic agent;
(e) Determining whether the contacted immune cell enacts a biological effect on the cell expressing the first and/or second protein of interest at each level of expression of the first and/or second protein of interest;
(f) Determining the minimum level of expression of the first and/or second protein of interest at which the contacted immune cell enacts a biological effect on the cell expressing the first and second protein of interest

In one embodiment, the auxin inducible proximity system and abscisic acid inducible proximity system are as defined hereinabove, as are the first, second, third and fourth constructs.

In one embodiment, step (b) or (c) as appropriate may comprise exposing the cell to a concentration of a first inducer, suitably a plant hormone inducer, suitably auxin and a concentration of a second inducer, suitably a plant hormone inducer, suitably abscisic acid at the same time or at different times as explained further below.

In one embodiment, step (d) or (e) as appropriate may comprise determining whether the contacted immune cell enacts a biological effect on the cell expressing the first and second protein of interest at each level of expression of both the first and second protein of interest.

In one embodiment, step (e) or (f) as appropriate may comprise determining the minimum level of expression of both the first and second protein of interest at which the contacted immune cell enacts a biological effect on the cell expressing the first and second protein of interest.

In one embodiment, determining the minimum level of expression of a first and/or a second protein of interest in a cell comprises determining the threshold level of expression of a first and/or a second protein of interest in a cell.

In one embodiment, the minimum level of expression of the first and/or second protein of interest at which the contacted immune cell enacts a biological effect on the cell expressing the first and/or second protein of interest may be the level of expression at which a biological effect higher than the background biological effect is achieved. Suitably at which a biological effect of at least 3 standard deviation above the background biological effect is achieved, suitably at least 4 standard deviations above the background biological effect is achieved, suitably at least 5 standard deviations above the background biological effect is achieved, suitably at least 6 standard deviations above the background biological effect is achieved, suitably at least 7 standard deviations above the background biological effect is achieved, suitably at least 8 standard deviations above the background biological effect is achieved, suitably at least 9 standard deviations above the background biological effect is achieved, suitably at least 10 standard deviations above the background biological effect is achieved. Suitably wherein the background biological effect is the biological effect of the candidate binding molecule on a control cell. Suitably wherein the control cell is a cell which does not contain an inducible system as described herein. Suitably the control cell does not express the or each protein of interest. Suitably wherein the control cells is a wild type cell. Alternatively, the control cell may be a cell that has been modified to prevent expression of the or each protein of interest, suitably by ‘knocking out’ the gene encoding the or each protein of interest which may be achieved by known modification techniques such as RNA interference, RNA silencing, CRISPRi, zinc finger nucleases, TALENs etc.

In one embodiment, the minimum level of expression of the first and/or second protein of interest at which the contacted immune cell enacts a biological effect on the cell expressing the first and/or second protein of interest may be the activation threshold of the first and/or second protein of interest. Suitably this may be determined using receiver operator characteristic (ROC) curve analysis, suitably using Youden's index or Youden's J statistic.

In one embodiment, the candidate therapeutic agent is a biologic, preferably the candidate therapeutic agent is an immunotherapy, preferably selected from a fusion protein, an antibody (e.g. a monoclonal antibody, an antibody drug conjugate, a nanobody, scFv, di-scFv, Fab, sdAb, F(ab)₂, a 35glycol-engineered antibody) or a binding fragment thereof, a fusion protein, an antibody-drug conjugate, an aptamer, an ankyrin, a designed ankyrin repeat protein (DARPin), a peptide, a bicyclic peptide, a vaccine, a cytokine, a chemokine, a hormone, an Oncolytic virus, and a bacterium.

In one embodiment, the immune cell is selected from a T cell, an NK cell, a B cell, a lymphocyte, a dendritic cell, and a mesenchymal cell, or immortalised cells thereof.

In a thirteenth aspect of the invention there is provided a method of screening a candidate engineered immune cell for a biological effect comprising:
(a) Providing a cell comprising a first inducible system operable to express a first protein of interest, and optionally a second inducible system operable to express a second protein of interest;
(b) Exposing the cell comprising the first inducible system and optionally the second inducible system to an effective concentration of a first inducer to induce a desired level of expression of the first protein of interest, and optionally exposing the cell to an effective concentration of a second inducer to induce a desired level of expression of the second protein of interest;
(c) Contacting the cell with the candidate engineered immune cell;
(d) Determining whether the candidate engineered immune cell enacts a biological effect on the cell expressing the first and optionally the second protein of interest.

Preferred or optional features defined above in relation to the ninth aspect, apply equally to the thirteenth aspect.

In an embodiment of the thirteenth aspect of the invention there is provided a method of screening a candidate engineered immune cell for a biological effect comprising:
(a) Providing a cell comprising the first plant hormone inducible proximity system and/or second plant hormone inducible proximity system of the eighth aspect;
(b) Culturing the cell comprising the first plant hormone inducible proximity system and/or the second plant hormone inducible proximity system under conditions to express the first construct and/or the third construct;
(c) Exposing the cell comprising the first plant hormone inducible proximity system and/or the second plant hormone inducible proximity system to an effective concentration of a first plant hormone inducer to induce a desired level of expression of the first protein of interest from the second construct, and/or exposing the cell to an effective concentration of a second plant hormone inducer to induce a desired level of expression of the second protein of interest from the fourth construct;
(d) Contacting the cell with the candidate engineered immune cell;
(e) Determining whether the candidate engineered immune cell enacts a biological effect on the cell expressing the first and/or second protein of interest.

In one embodiment of the thirteenth aspect, there is provided a method of screening a candidate engineered immune cell for a biological effect comprising:
(a) Providing a cell comprising the auxin inducible proximity system and/or an abscisic acid inducible proximity system as defined herein;
(b) Culturing the cell comprising the auxin inducible proximity system and/or the abscisic acid inducible proximity system under conditions to express the first construct and/or the third construct;
(c) Exposing the cell comprising the auxin inducible proximity system and/or the abscisic acid inducible proximity system to an effective concentration of auxin to induce a desired level of expression of the first protein of interest from the second construct, and/or exposing the cell to an effective concentration of abscisic acid to induce a desired level of expression of the second protein of interest from the fourth construct;
(d) Contacting the cell with the candidate engineered immune cell;
(e) Determining whether the candidate engineered immune cell enacts a biological effect on the cell expressing the first and/or second protein of interest.

In one embodiment of the thirteenth aspect, the method is a method of screening a candidate engineered immune cell for a biological effect comprising:
(a) Providing a cell comprising the auxin inducible proximity system and an abscisic acid inducible proximity system as defined herein;
(b) Culturing the cell comprising the auxin inducible proximity system and the abscisic acid inducible proximity system under conditions to express the first construct and the third construct;
(c) Exposing the cell comprising the auxin inducible proximity system and the abscisic acid inducible proximity system to an effective concentration of auxin to induce a desired level of expression of a first protein of interest from the second construct, and exposing the cell to an effective concentration of abscisic acid to induce a desired level of expression of a second protein of interest from the fourth construct;
(d) Contacting the cell with the candidate engineered immune cell;
(e) Determining whether the candidate engineered immune cell enacts a biological effect on the cell expressing the first and second protein of interest.
(f) Determining the expression levels of the first and/or second protein of interest at which the contacted engineered immune cell is enacting a biological effect

In one embodiment, the auxin inducible proximity system and abscisic acid inducible proximity system are as defined hereinabove, as are the first, second, third and fourth constructs.

In one embodiment, step (b) or (c) as appropriate may comprise exposing the cell to a plurality of different concentrations of first inducer, suitably a plant hormone inducer, suitably auxin, to induce a plurality of different levels of expression of the first protein of interest, which may be expressed from the second construct, and/or exposing the cell to a plurality of different concentrations of second inducer, suitably a plant hormone inducer, suitably abscisic acid, to induce a desired level of expression of the second protein of interest, which may be expressed from the fourth construct.

In one embodiment, step (b) or (c) as appropriate may comprise exposing the cell to an effective concentration of a first inducer, suitably a plant hormone inducer, suitably auxin and an effective concentration of a second inducer, suitably a plant hormone inducer, suitably abscisic acid at the same time or at different times as explained further below.

In one embodiment, step (d) or (e) as appropriate may comprise determining whether the candidate engineered immune cell enacts a biological effect on the cell expressing both the first and second protein of interest. In one embodiment, enacting a biological effect may comprise targeting the cell expressing the first and/or second protein of interest.

In one embodiment, the method may further comprise a step (e) or (f) as appropriate of determining the minimum expression levels of the first and/or second protein of interest at which the contacted engineered immune cell is enacting a biological effect.

In one embodiment, step (e) or (f) as appropriate may comprise determining the minimum expression levels of both the first and second protein of interest at which the candidate engineered immune cell is enacting a biological effect.

In one embodiment, the minimum level of expression of the first and/or second protein of interest at which the candidate engineered immune cell enacts a biological effect may be the level of expression at which a biological effect higher than the background biological effect is achieved. Suitably at which a biological effect of at least 3 standard deviation above the background biological effect is achieved, suitably at least 4 standard deviations above the background biological effect is achieved, suitably at least 5 standard deviations above the background biological effect is achieved, suitably at least 6 standard deviations above the background biological effect is achieved, suitably at least 7 standard deviations above the background biological effect is achieved, suitably at least 8 standard deviations above the background biological effect is achieved, suitably at least 9 standard deviations above the background biological effect is achieved, suitably at least 10 standard deviations above the background biological effect is achieved. Suitably wherein the background biological effect is the biological effect of the candidate binding molecule on a control cell. Suitably wherein the control cell is a cell which does not contain an inducible system as described herein. Suitably the control cell does not express the or each protein of interest. Suitably wherein the control cells is a wild type cell. Alternatively, the control cell may be a cell that has been modified to prevent expression of the or each protein of interest, suitably by ‘knocking out’ the gene encoding the or each protein of interest which may be achieved by known modification techniques such as RNA interference, RNA silencing, CRISPRi, zinc finger nucleases, TALENs etc.

In one embodiment, the minimum level of expression of the first and/or second protein of interest at which the candidate engineered immune cell enacts a biological effect may be the activation threshold of the first and/or second protein of interest. Suitably this may be determined using receiver operator characteristic (ROC) curve analysis, suitably using Youden's index or Youden's J statistic.

In one embodiment, the candidate engineered immune cell is selected from a cell expressing a CAR or a T-cell receptor (TCR), preferably selected from a CAR T-cell, a TCR T cell, a CAR NK cell, a CAR macrophage, and a CAR B cell.

In a fourteenth aspect of the invention there is provided a method of determining the minimum level of expression of at least one protein of interest in a cell at which a candidate engineered immune cell enacts a biological effect, the method comprising:
(a) Providing a cell comprising a first inducible system operable to express a first protein of interest, and optionally a second inducible system operable to express a second protein of interest;
(b) Exposing the cell comprising the first inducible system, and optionally the second inducible system, to a plurality of different concentrations of a first inducer to induce a plurality of different levels of expression of the first protein of interest, and optionally exposing the cell to a plurality of different concentrations of a second inducer to induce a plurality of different levels of expression of the second protein of interest;
(c) Contacting the cell with the candidate engineered immune cell;
(d) Determining whether the candidate engineered immune cell enacts a biological effect on the cell expressing the first and optionally the second protein of interest at each level of expression of the first and optionally the second protein of interest; and
(e) Determining the minimum level of expression of the first and optionally the second protein of interest at which the candidate engineered immune cell enacts a biological effect on the cell expressing the first and optionally the second protein of interest.

Preferred or optional features defined above in relation to the ninth aspect, apply equally to the fourteenth aspect.

In a fourteenth aspect of the invention there is provided a method of determining the minimum level of expression of at least one protein of interest in a cell at which a candidate engineered immune cell enacts a biological effect, the method comprising:
(a) Providing a cell comprising the first plant hormone inducible proximity system and/or second plant hormone inducible proximity system of the eighth aspect;
(b) Culturing the cell comprising the first plant hormone inducible proximity system and/or the second plant hormone inducible proximity system under conditions to express the first construct and the third construct;
(c) Exposing the cell comprising the first plant hormone inducible proximity system, and/or the second plant hormone inducible proximity system, to a plurality of different concentrations of a first plant hormone inducer to induce a plurality of different levels of expression of the first protein of interest from the second construct, and/or exposing the cell to an effective concentration of a second plant hormone inducer to induce a desired level of expression of the second protein of interest from the fourth construct;
(d) Contacting the cell with the candidate engineered immune cell;
(e) Determining whether the candidate engineered immune cell enacts a biological effect on the cell expressing the first and/or second protein of interest at each level of expression of the first and/or second protein of interest; and
(f) Determining the minimum level of expression of the first and/or second protein of interest at which the candidate engineered immune cell enacts a biological effect on the cell expressing the first and/or second protein of interest.

In one embodiment of the fourteenth aspect, there is provided a method of determining the minimum level of expression of at least one protein of interest in a cell at which a candidate engineered immune cell enacts a biological effect, the method comprising:
(a) Providing a cell comprising the auxin inducible proximity system and/or an abscisic acid inducible proximity system as defined herein;
(b) Culturing the cell comprising the auxin inducible proximity system and/or the abscisic acid inducible proximity system under conditions to express the first construct and the third construct;
(c) Exposing the cell comprising the auxin inducible proximity system, and/or the abscisic acid inducible proximity system, to a plurality of different concentrations of auxin to induce a plurality of different levels of expression of the first protein of interest from the second construct, and/or exposing the cell to an effective concentration of abscisic acid to induce a desired level of expression of the second protein of interest from the fourth construct;
(d) Contacting the cell with the candidate engineered immune cell;
(e) Determining whether the candidate engineered immune cell enacts a biological effect on the cell expressing the first and/or second protein of interest at each level of expression of the first and/or second protein of interest
(f) Determining the minimum level of expression of the first and/or second protein of interest at which the candidate engineered immune cell enacts a biological effect on the cell expressing the first and/or second protein of interest

In one embodiment of the fourteenth aspect, the method is a method of determining the minimum level of expression of a first and a second protein of interest in a cell at which a candidate engineered immune cell enacts a biological effect, the method comprising:
(a) Providing a cell comprising the auxin inducible proximity system and an abscisic acid inducible proximity system as defined herein;
(b) Culturing the cell comprising the auxin inducible proximity system and the abscisic acid inducible proximity system under conditions to express the first construct and the third construct;
(c) Exposing the cell comprising the auxin inducible proximity system, and the abscisic acid inducible proximity system, to a plurality of different concentrations of auxin to induce a plurality of different levels of expression of the first protein of interest from the second construct, and exposing the cell to an effective concentration of abscisic acid to induce a desired level of expression of the second protein of interest from the fourth construct;
(d) Contacting the cell with the candidate engineered immune cell;
(e) Determining whether the candidate engineered immune cell enacts a biological effect on the cell expressing the first and second protein of interest at each level of expression of the first and second protein of interest
(f) Determining the minimum level of expression of the first and/or second protein of interest at which the candidate engineered immune cell enacts a biological effect on the cell expressing the first and second protein of interest

In one embodiment, the auxin inducible proximity system and abscisic acid inducible proximity system are as defined hereinabove, as are the first, second, third and fourth constructs.

In one embodiment, step (b) or (c) as appropriate may comprise exposing the cell to a concentration of a first inducer, suitably a plant hormone inducer, suitably auxin and a concentration of a second inducer, suitably a plant hormone inducer, suitably abscisic acid at the same time or at different times as explained further below.

In one embodiment, step (d) or (e) as appropriate may comprise determining whether the candidate engineered immune cell enacts a biological effect on the cell expressing the first and second protein of interest at each level of expression of both the first and second protein of interest.

In one embodiment, step (e) or (f) as appropriate may comprise determining the minimum level of expression of both the first and second protein of interest at which the candidate engineered immune cell enacts a biological effect on the cell expressing both the first and second protein of interest.

In one embodiment, the minimum level of expression of the first and/or second protein of interest at which the candidate engineered immune cell enacts a biological effect on the cell expressing the first and/or second protein of interest may be the level of expression at which a biological effect higher than the background biological effect is achieved. Suitably at which a biological effect of at least 3 standard deviation above the background biological effect is achieved, suitably at least 4 standard deviations above the background biological effect is achieved, suitably at least 5 standard deviations above the background biological effect is achieved, suitably at least 6 standard deviations above the background biological effect is achieved, suitably at least 7 standard deviations above the background biological effect is achieved, suitably at least 8 standard deviations above the background biological effect is achieved, suitably at least 9 standard deviations above the background biological effect is achieved, suitably at least 10 standard deviations above the background biological effect is achieved. Suitably wherein the background biological effect is the biological effect of the candidate binding molecule on a control cell. Suitably wherein the control cell is a cell which does not contain an inducible system as described herein. Suitably the control cell does not express the or each protein of interest. Suitably wherein the control cells is a wild type cell. Alternatively, the control cell may be a cell that has been modified to prevent expression of the or each protein of interest, suitably by ‘knocking out’ the gene encoding the or each protein of interest which may be achieved by known modification techniques such as RNA interference, RNA silencing, CRISPRi, zinc finger nucleases, TALENs etc.

In one embodiment, the minimum level of expression of the first and/or second protein of interest at which the candidate engineered immune cell enacts a biological effect on the cell expressing the first and/or second protein of interest may be the activation threshold of the first and/or second protein of interest. Suitably this may be determined using receiver operator characteristic (ROC) curve analysis, suitably using Youden's index or Youden's J statistic.

In one embodiment, the candidate engineered immune cell is selected from a cell expressing a CAR or a T-cell receptor (TCR), preferably selected from a CAR T-cell, a TCR T cell, a CAR NK cell, a CAR macrophage, and a CAR B cell.

In a fifteenth aspect of the invention there is provided a method of inducing a first chemically inducible proximity system such as an auxin inducible proximity system comprising:
(a) Providing a cell comprising first chemically inducible proximity system such as the auxin inducible proximity system of the eighth aspect;
(b) Culturing the cell under conditions to express the first and second chimeric proteins from the first construct;
(c) Exposing the cell to an effective concentration of a first inducer, such as auxin to cause the first and second chimeric proteins to associate.

In such an embodiment, the first chemically inducible proximity system such as the auxin inducible proximity system, first and second constructs are as defined hereinabove.

In a further embodiment of the invention there is provided a method of inducing a second chemically inducible proximity system such as an abscisic acid inducible proximity system comprising:
(a) Providing a cell comprising a second chemically inducible proximity system such as an abscisic acid inducible proximity system of the eighth aspect;
(b) Culturing the cell under conditions to express the third and fourth chimeric proteins from the third construct;
(c) Exposing the cell to an effective concentration of a second inducer such as abscisic acid to cause the third and fourth chimeric proteins to associate.

In such an embodiment, the second chemically inducible proximity system such as the abscisic acid inducible proximity system, third and fourth constructs are as defined hereinabove.

In a further embodiment of the invention there is provided a method of inducing a first and/or a second plant hormone inducible proximity system comprising:
(a) Providing a cell comprising a first and/or a second plant hormone inducible proximity system of the eighth aspect;
(b) Culturing the cell under conditions to express the first and second chimeric proteins from the first construct and/or the third and fourth chimeric proteins from the third construct;
(c) Exposing the cell to an effective concentration of a first plant hormone inducer to cause the first and second chimeric proteins to associate and/or exposing the cell to an effective concentration of a second plant hormone inducer to cause the third and fourth chimeric proteins to associate.

In such an embodiment, the cell may be the cell of the eighth aspect.

In a further embodiment of the invention there is provided a method of inducing an auxin inducible proximity system and an abscisic acid inducible proximity system comprising:
(a) Providing a cell comprising the auxin inducible proximity system and the abscisic acid inducible proximity system as defined herein;
(b) Culturing the cell under conditions to express the first and second chimeric proteins from the first construct, and the third and fourth chimeric proteins from the third construct;
(c) Exposing the cell to an effective concentration of auxin to cause the first and second chimeric proteins to associate, and exposing the cell to an effective concentration of abscisic acid to cause the third and fourth chimeric proteins to associate.

In such an embodiment, the auxin inducible proximity system, abscisic acid inducible proximity system, first, second, third and fourth constructs are as defined hereinabove.

In one embodiment, the methods of inducing the proximity system/s may comprise exposing the cell to a plurality of different concentrations of first plant hormone inducer, suitably auxin, to induce a plurality of different levels of expression of the first protein of interest from the second construct, and/or exposing the cell to a plurality of different concentrations of second plant hormone inducer, suitably abscisic acid, to induce a desired level of expression of the second protein of interest from the fourth construct.

In one embodiment, step (c) may comprise exposing the cell to an effective concentration of auxin and an effective concentration of abscisic acid at the same time or at different times as explained further below.

In such an embodiment, the cell may be the cell of the eighth aspect.

In a sixteenth aspect of the invention there is provided a method of determining whether a first compound such as an auxin compound is present in a sample, the method comprising:
(a) Providing a cell comprising the first chemically inducible proximity system such as auxin inducible proximity system of the eighth aspect, wherein the protein of interest of the second construct is a reporter;
(b) Culturing the cell under conditions to express the first and second chimeric proteins from the first construct;
(c) contacting the sample with the cell; and
(d) evaluating expression of the reporter to determine whether the compound such as an auxin compound is present in the sample.

In such an embodiment, the first chemically inducible proximity system such as the auxin inducible proximity system, first and second constructs are as defined hereinabove.

In a seventeenth aspect, there is provided a method of determining whether a second compound such as an abscisic acid compound is present in a sample, the method comprising:
(a) Providing a cell comprising a second chemically inducible proximity system such as an abscisic acid inducible proximity system, wherein the protein of interest of the fourth construct is a reporter;
(b) Culturing the cell under conditions to express the third and fourth chimeric proteins from the third construct;
(c) contacting the sample with the cell; and
(d) evaluating expression of the reporter to determine whether the second compound such as an abscisic acid compound is present in the sample.

In such an embodiment, the second chemically inducible proximity system such as the abscisic acid inducible proximity system, third and fourth constructs are as defined hereinabove.

In a further embodiment, there is provided a method of determining whether a first compound and/or a second compound is present in a sample, the method comprising:
(a) Providing a cell comprising a first inducible system operable to express a first protein of interest, and/or a second inducible system operable to express a second protein of interest, wherein the first protein of interest is a first reporter and the second protein of interest is a second reporter;
(b) contacting the sample with the cell; and
(c) evaluating expression of the first and/or second reporter to determine whether the first and/or second compound is present in the sample.

Suitable inducible systems are defined herein. In one embodiment, the first and second inducible systems may be chemically induced proximity systems (CIP systems). In one preferred embodiment, they may be plant hormone or plant hormone analogue inducible systems as described herein. In some embodiments, the first inducible system may be a first plant hormone inducible proximity system of the eighth aspect. In some embodiments, the second inducible system may be a second plant hormone inducible proximity system of the eighth aspect. Suitably in such embodiments, the first inducible system comprises a first and second construct as defined herein, and the second inducible system comprises a third and a fourth construct as defined herein. In one embodiment, at least one of the inducible systems is a plant hormone inducible proximity system. In one embodiment, at least one of the inducible systems is an abscisic acid inducible proximity system defined herein. In one embodiment, at least one of the inducible systems is selected from a caffeine inducible proximity system defined herein, a Mandipropamid inducible proximity system defined herein, and a gibberellin inducible proximity system defined herein.

In a preferred embodiment, the first inducible system is selected from a caffeine inducible proximity system defined herein, a Mandipropamid inducible proximity system defined herein, and a gibberellin inducible proximity system defined herein and the second inducible system is an abscisic acid inducible proximity system as defined herein.

In a further embodiment, there is provided a method of determining whether a first compound and/or a second compound is present in a sample, the method comprising:
(a) Providing a cell comprising a first plant hormone inducible proximity system and second plant hormone inducible proximity system, wherein the protein of interest of the second construct is a first reporter and the protein of interest of the fourth construct is a second reporter;
(b) Culturing the cell under conditions to express the first and second chimeric proteins from the first construct, and the third and fourth chimeric proteins from the third construct;
(c) contacting the sample with the cell; and
(d) evaluating expression of the first and/or second reporter to determine whether the first and/or second compound is present in the sample.

In such an embodiment, the cell may be the cell of the eighth aspect.

In a further embodiment, there is provided a method of determining whether an auxin compound and/or an abscisic acid compound is present in a sample, the method comprising:
(a) Providing a cell comprising an auxin inducible proximity system and an abscisic acid inducible proximity system, wherein the protein of interest of the second construct is a first reporter and the protein of interest of the fourth construct is a second reporter;
(b) Culturing the cell under conditions to express the first and second chimeric proteins from the first construct, and the third and fourth chimeric proteins from the third construct;
(c) contacting the sample with the cell; and
(d) evaluating expression of the first and/or second reporter to determine whether the auxin and/or abscisic acid compound is present in the sample.

In such an embodiment, the auxin inducible proximity system, abscisic acid inducible proximity system, first, second, third and fourth constructs are as defined hereinabove.

In one embodiment, step (d) may comprise evaluating expression of both the first and second reporter to determine whether the auxin and abscisic acid compound is present in the sample.

In an eighteenth aspect of the invention there is provided a kit comprising: a first inducible proximity system of the third aspect, such as the auxin inducible proximity system of the third aspect, the vector of the fourth aspect, or the cell comprising said system as defined above; and a first inducer compound such as an auxin compound.

In a nineteenth aspect of the invention, there is provided a kit comprising: a second inducible proximity system such as the abscisic acid inducible proximity system defined hereinabove, the vector comprising said system, or the cell comprising said system as defined hereinabove, and a second inducer compound such as an abscisic acid compound.

In one embodiment, the kit may comprise the components for both first compound and second compound detection such as both auxin detection and abscisic acid detection.

Suitably any references in the above aspects to the first construct is a reference to the first construct according to the first aspect. Suitably any reference in the above aspects to the second construct is a reference to the second construct according to the second aspect. Suitably any references in the above aspects to the auxin inducible proximity system is a reference to the auxin inducible proximity system according to the third aspect. Suitably any references in the above aspects to the third construct is a reference to the third construct as defined hereinabove. Suitably any reference in the above aspects to the fourth construct is a reference to the fourth construct as defined hereinabove. Suitably any references in the above aspects to the abscisic acid inducible proximity system is a reference to the abscisic acid inducible proximity system as defined hereinabove.

Suitably any references in the above aspects to an auxin inducible proximity system and/or an abscisic acid inducible proximity system may encompass one of these two systems, or both of these two systems. Suitably, such references may encompass an auxin inducible proximity system optionally combined with an abscisic acid inducible proximity system and any corresponding constructs thereof optionally in combination.

Suitably it will be appreciated by the skilled person that the cells and methods described herein may contain third, fourth, fifth, sixth or a plurality of inducible systems operable to express a plurality of proteins of interest at different controllable levels using the same constructs, steps and techniques described herein for the first and second inducible systems.

### BRIEF DESCRIPTION OF THE DRAWINGS

FIG. 1 is a cartoon diagram showing an exemplary auxin (indole-3-acetic acid (IAA)) controlled, chemically induced proximity (CIP) for titratable expression of a first protein of interest, which may be a first target antigen of interest (TAOI expression). Panel A1) shows the IAA activator cassette comprises the simplex virus VP16 transactivation domain (VP16AD) fused to TIR1 and a catalytically inactive I-Scel homing endonuclease (dl-Scel) fused to AIDΔ34. Panel A2) shows the expression construct employs the 18 bp recognition sequence for I-Scel downstream of which the target antigen of interest (TAOI) is placed. Panel B) shows the dl-Scel associates with its recognition sequence but cannot activate transcription as it lacks a transactivator domain. In the presence of IAA, the AIDΔ34 associates with TIR1 and brings the VP16 transactivator domain into proximity of the upstream region of the TAOI, activating transcription.

FIG. 2 shows cytotoxicity of IAA on HEK293 and CHO-K1 cells, analysed by flow cytometry. HEK293 and CHO-K1 cells were treated with the indicated concentrations of IAA for 24 hours before being stained with eFluor780 fixable viability dye and analysed by flow cytometry. Viability is reported as the percentage of cells which do not take up the dye

FIG. 3 shows graphs demonstrating the effect of IAA on proliferation and activation of T cells. PBMCs from two donors were stimulated with Human T-Activator CD3/CD28 beads in the presence of a concentration series of IAA. Proliferation of CD4+ (panel A) and CD8+ (panel B) T cells were assessed by flow cytometry using a proliferation dye. IFNγ production was assessed by ELISA (panel C). n=3, graphs report medium±SD.

FIG. 4 demonstrates the expression of the IAA activator cassette in HEK293 (A) and CHO-K1 (B) cells as determined using an anti-VP16 detection antibody. Stained parental HEK293 or CHO-K1 cells are shown in dark grey, cells transduced with IAA activator cassettes are shown in light grey.

FIG. 5 demonstrates IAA treatment of IAA activator HEK293 (A) and CHO-K1 (B) cells transduced with EGFP reporter constructs containing varying numbers of i-Scel binding sites as indicated. Analysis was performed by flow cytometry after 24 hours incubation at the indicated concentrations of IAA.

FIG. 6 demonstrates the induction of EGFP expression with IAA HEK293 (A) and CHO-K1 (B) cells containing the IAA activator cassette and the 10× and 5× i-Scel reporter constructs were seeded overnight in 96 well plates. IAA was added at the indicated concentration and the cells were incubated for a further 24 hours. EGFP expression was detected by flow cytometry analysis.

FIG. 7 shows IAA dose dependent induction of EGFP expression. CHO-K1 (A) and HEK293 (B) cells containing the IAA activator cassette and the 10× i-Scel reporter construct were seeded overnight in 96 well plates. IAA was added at the indicated concentrations and the cells were incubated for a further 24 hours. EGFP expression was detected by flow cytometry analysis.

FIG. 8 is a cartoon diagram showing Abscisic acid (ABA) controlled, chemically induced proximity (CIP) for titratable expression of a second protein of interest, which may be a second target antigen of interest (TAO2 expression). Panel A1) shows the ABA activator cassette comprises yeast Gal4 DNA binding domain (Gal4DBD) fused to ABIcs and the herpes simplex virus VP16 transactivation domain (VP16AD) fused to PYLcs. Expression of these two fusion proteins is operably linked by inclusion of a Thosea asigna 2A (T2A) self-cleaving peptide. Panel A2) shows the expression construct employs a 9× repeat of the Gal4 upstream activation sequence (UAS) under the control of which the target antigen of interest (TAOI) is placed. Panel B) shows the Gal4 DNA binding domain associates with the Gal4 UAS but cannot activate transcription as it lacks a transactivator domain. In the presence of ABA, the PYLcs associates with ABIcs and brings the VP16 transactivator domain into proximity of the upstream region of the target antigen (gene) of interest, activating transcription.

FIG. 9 shows Cytotoxicity of ABA on HEK293 and CHO-K1 cells analysed by flow cytometry. HEK293 and CHO-K1 cells were treated with the indicated concentrations of ABA for 24 hours before being stained with eFluor780 fixable viability dye and analysed by flow cytometry. Viability is reported as the percentage of cells which do not take up the dye

FIG. 10 shows graphs demonstrating the effect of ABA on proliferation and activation of T cells. PBMCs from two donors were stimulated with Human T-Activator CD3/CD28 in the presence of a concentration series of IAA. Proliferation of CD4+ (Panel A) and CD8+ (Panel B) T cells was assessed by flow cytometry using a proliferation dye. IFNγ production was assessed by ELISA (Panel C). n=3, graphs report medium±SD.

FIG. 11 shows the expression of the ABA activator cassette in HEK293 (A) and CHO-K1 (B) cells as determined using an anti-VP16 detection antibody. Stained parental HEK293 or CHO-K1 cells are shown in dark grey, cells transduced with ABA activator cassette are shown in light grey.

FIG. 12 shows ABA treatment of ABA activator CHO-K1 and HEK293 cells transduced with tagBFP (A) and CD19 (B) reporter constructs. The cells were treated with 1000 μM ABA for 24 hours before being analysed by flow cytometry for the expression of tagBFP and CD19.

FIG. 13 shows ABA dose dependent induction of CD19 expression. CHO-K1 cells containing the ABA activator cassette and the 9×Gal4 UAS CD19 reporter construct were seeded overnight in 96 well plates. ABA was added at the indicated concentrations and the cells were incubated for a further 48 hours. CD19 expression was detected by flow cytometry analysis and the number of CD19 receptors per cell was quantified using a Quantibrite PE bead fluorescence quantification kit (BD Biosciences). The CD19 receptors per cell for Ramos, Raji and B cells isolated from PBMCs are shown by way of comparison.

FIG. 14 is a cartoon showing (A) the constructs used in the examples to prepare the auxin inducible CIP system and (B) the constructs used in the examples to prepare the abscisic acid inducible CIP system.

FIG. 15 shows quantification of IAA dose dependent expression of CD19. CHO-K1 cells containing the IAA activator cassette and a 10× i-Scel CD19 reporter construct were seeded overnight in 96 well plates. IAA was added at the indicated concentrations and the cells were incubated for a further 72-hours. CD19 expression was assess by flow cytometry and quantified for receptor number per cell. By means of comparison, the CD19 expression level of the CD19 positive B cells found in peripheral blood mononuclear cells (PBMCs) and Ramos cells were calculated in the same experiment.

FIG. 16 shows validation of the orthogonality of the ABA and IAA CIP systems. CHO-K1 ABA activator CD19 reporter cells and CHO-K1 IAA activator CD19 reporter cells were each treated with (A) 1000 μM ABA and (B) 1000 μM IAA separately for 24 hours. Following this incubation, the cells were analysed for CD19 expression by flow cytometry.

FIG. 17 shows independent T-cell activation by CD19 expressing CHO-K1 ABA or IAA CIP systems assessed using Jurkat NFAT T cell activation reporter cells. CHO-K1 IAA or ABA activator CD19 reporter cells were plated with a concentration series of IAA or ABA (7.18 μM to 500 μM) to induce CD19 expression over 24 hours. After 24 hours, IAA and ABA were removed from the system and Jurkat NFAT luciferase reporter cells were added with the CD19×CD3 bispecific Blincyto at 10 ng/ml and 25 ng/mL. NFAT activation was determined by luminescence after 24 hours (48 Hours from assay start) and plotted against CD19 expression quantified by staining with PE anti-CD19 and plotted on a curve generated using Quanti-BriteQuantibrite PE receptor quantification kit. n=3, graphs report mean±SD.

FIG. 18 shows CHO-K1 cells containing ABA-inducible CD22 and IAA-inducible CD19 CIP systems were treated with a concentration series of IAA or ABA, and CD22 and CD19 expression were measured by flow cytometry.

FIG. 19 shows ABA-induced HER2 expression to screen the biological activity of anti-HER2 ADCs Trastuzumab Emtansin or Trastumuzab Deruxtecan. A) HER2 expression in CHO-K1 ABA HER2 cells treated with a concentration series of ABA. B) Target cell cytolysis in HER2 expressing CHO-K1 ABA HER2 cells treated with anti-HER2 ADCs Trastuzumab Emtansin or Trastumuzab Deruxtecan. n=3, graphs report mean±SD.

FIG. 20 shows T cell-dependent cellular cytotoxicity (TDCC) assays using CHO-K1 ABA activator CD19 cells and a CD19×CD3 bispecific T cell engager (TCE). CD19 expression was induced with a concentration series of ABA, then CD8+ T cells isolated from four donor PBMCs were added at a 3:1 ratio in the presence of 225 μM CD19×CD3 bispecific TCE. A) Target cell death after 48 hours. B) CD8+ T cell proliferation as measured by Ki-67 staining. C) IFN-γ production by CD8+ T cells after 48-hour. For all panels, n=3, graphs report mean±SD.

FIG. 21 compares the cytotoxicity of two CD19×CD3 bispecific T cell engagers (TCEs) across a range of CD19 receptor expression levels achieved by treating CHO-K1 ABA CD19 cells with a concentration series of ABA. T cells were isolated from donor PBMCs (n=10) and used in conjunction with TCE 1 or TCE 2. A and C). T cell mediated cytotoxicity (A) and IFN-gamma production (C). Each data point represents a single T cell donor. B and D) Estimation of the minimum number of receptors for which a biologic effect in the forms of cytotoxicity (B) and IFN-gamma production (D) was observed for each bispecific TCE. This was defined as the minimum number of receptors at which an effect higher than the mean background effect +3 standard deviations was observed, where background was the biological effect observed when wild type CHOs were used as target cells which were assumed to have 0 number of target CD19 receptors. Data represented as median, bars and confidence intervals. The Wilcoxon matched-pairs signed rank test was employed to compare the two treatments. E and F) Receiver operating characteristic (ROC) curves for both TCE 1 and TCE 2 to determine the threshold of activation for the 10-donor cohort for cytolysis (E) and IFN-gamma production (F). The Youden's Index for each molecule and readout was employed to determine the receptor activation thresholds which are depicted in the graphs. For classification of the results as positive for each level of receptor expression and for each donor, the same threshold of background+3 standard deviations cut-off value was employed.

FIG. 22 shows CD19 CAR-T cell activity against target CHO-K1 ABA activator CD19 cells. A) Schematic representation of the CD19 CAR construct used in this experiment, consisting of a FMC63 anti-CD19 scFv (CD19VL and CD19VH), CD8 stalk and transmembrane domain, and 41BB and CD3Z intracellular domains. B) Expression of the CD19 CAR construct after transduction into T cells from three donors, detected in CD3 positive T cells using an anti-FMC63 scFv antibody via flow cytometry. C) The cytolytic activity of CD19 CAR-T cells against CHO-K1 ABA CD19 cells expressing a series of CD19 levels through treatment with a concentration series of ABA. D) IFN-γ release by the effector CD19 CAR-T cells after 24 incubation with target cells. For C and D, n=3, graphs report mean±SD.

FIG. 23 shows dual CD19/CD22 CAR Jurkat NFAT luciferase reporter cell activity against target CHO-K1 cells expressing ABA inducible CD22 and IAA inducible CD19 CIP systems. A) Schematic representation of the CD19/CD22 targeting CAR used in this experiment, consisting of the FMC63 anti-CD19 scFv (CD19VL, CD19VH), the m971 anti-CD22 scFv (CD22VL, CD22VH), CD8 stalk and transmembrane domains, and 41BB and CD3Z intracellular domains. B) Luciferase activity measured in relative luminescence units (RLUs) of CD19/CD22 CAR Jurkat NFAT luciferase reporter (effector) cells after being added to target CHO-K1 ABA CD22 IAA CD19 cells which have been induced with a concentration series of either IAA, ABA, or both.

FIG. 24 shows the activity of dual CD19/CD22 CAR-T cells, made from three donor PBMC-derived primary T cells, against target CHO-K1 cells containing ABA inducible CD22 and IAA inducible CD19 CIP systems. A) CD19/CD22 CAR expression detected in transduced CD3 positive T cells from three donors. B) CD19 and CD22 expression in CHO-K1 IAA CD19 ABA CD22 target cells after induction either separately or together with concentration series of IAA or ABA. C) Target CHO-K1 IAA CD19 ABA CD22 cell cytolysis induced by CD19/CD22 CAR positive T cells. For C, n=3, error bars report mean±SD.

FIG. 25 assesses the impact of HER2 expression, induced by treating CHO-K1 ABA HER2 cells with a concentration series of ABA, on the antibody-dependent cellular cytotoxicity (ADCC) activity of trastuzumab, pertuzumab or a combination of the two. PBMC from three healthy donors were employed (each represented by a data point) at an effector to target ratio of 10:1 and the cocultures were treated with 20 μg/mL of the individual or combination agents. Target cell cytolysis was assessed with the xCELLIgence RTCA system. Data represented as mean±SD.

FIG. 26 shows a caffeine controlled inducible expression system. A1) A caffeine activator cassette comprising yeast Gal4 DNA binding domain (Gal4DBD) and the Herpes simplex virus VP16 transactivation domain (VP16AD), each fused to the aCaffVHH nanobody isolated from Llama (Lama glama)⁶. Expression of these two fusion proteins is operably linked by inclusion of a Thosea asigna 2A (T2A) self-cleaving peptide. A2) The target antigen of interest (TAOI) under the control of a 9× repeat of the Gal4 upstream activation sequence (UAS). B) The Gal4 DNA binding domain associates with the Gal4 UAS but cannot activate transcription as it lacks a transactivator domain. In the presence of caffeine, the aCaffVHH nanobody dimerizes and brings the VP16 transactivator domain into proximity of the upstream region of the TAOI, activating transcription of the TAOI.

FIG. 27 shows a Mandi controlled inducible expression system. A) The chemical structure of Mandipropramid (Mandi). B1 and D1) The Mandi activator cassette comprises VP16AD fused to PYR^{Mandi} (B1) or PYLcs^{Mandi} (D1) and Gal4DBD fused to ABI. Expression of these two fusion proteins is operably linked by inclusion T2A self-cleaving peptide. B2 and D2) The target antigen of interest (TAOI) under the control of a 9× repeat of the Gal4 UAS. C and E) The Gal4DBD associates with the Gal4 UAS but cannot activate transcription as it lacks a transactivator domain. In the presence of Mandi, PYR^{Mandi} (C) and PYLcs^{Mandi} (E) associate with ABI and brings the VP16 transactivator domain into proximity of the upstream region of the TAOI, activating transcription of the TAOI.

FIG. 28 shows a gibberellin controlled inducible expression system. A1) The Gibberellin activator cassette comprises VP16AD fused to GID1 and Gal4DBD fused to GAI. Expression of these two fusion proteins is operably linked by inclusion of a T2A self-cleaving peptide. A2) The target antigen of interest (TAOI) under the control of a 9× repeat of the Gal4 UAS. B) The Gal4DBD associates with the Gal4 UAS but cannot activate transcription as it lacks a transactivator domain. In the presence of Gibberellin, GID1 associates with GAI and brings the VP16 transactivator domain into proximity of the upstream region of the TAOI, activating transcription of the TAOI.

### DETAILED DESCRIPTION OF THE INVENTION

The inventors have surprisingly overcome the problems in the art with in vitro risk assessment of candidate therapeutics such as immunotherapies by establishment of an inducible platform which allows the independent control of expression of one or two different target antigens in a titratable manner, which can express any target antigen of interest to any desired level.

The inventors have found that expression of a first and optionally a second target antigen of interest (TAOI1 and TAOI2), can be precisely controlled via inducible systems, specifically by chemically induced proximity (CIP) systems which may be derived from plant hormone signalling systems, specifically the auxin plant signalling pathway or the abscisic acid plant signalling pathway, the caffeine signalling pathway, or the gibberellin plant signalling pathway. The inventors have found that development of engineered cell lines with the simultaneous inducible and titratable expression of up to two different antigens of interest TAOI1 and/or TAOI2 using at least one inducible system is possible. Advantageously, the use of plant hormone inducible systems means that these systems do not have unwarranted effects on animal cells, and can be induced using plant hormones or analogues thereof which are small non-toxic molecules. The chemically induced proximity (CIP) systems derived from plant hormone signalling systems can also be based on plant hormone analogues such as Mandipropamid (Mandi).

In its preferred embodiment, the invention provides a modified CIP system comprising a first auxin inducible proximity system, a caffeine inducible proximity system, a Mandipropamid inducible proximity stem, or a gibberellin inducible proximity system and/or a second abscisic acid inducible proximity system providing a finely titratable induction of a TAOI1 and/or TAOI2, with a large dynamic range and compatibility with both immortalised cell lines and primary human cells.

However the advantages of the system apply equally to methods using any inducible systems. Together, these features make the cells comprising the inducible system such as the modified CIP systems suitable for the in vitro characterisation of candidate targeted therapies using cell-based bioassays, and complex co-cultures of primary human immune cells and immortalised cell lines. The system provides an in vitro risk assessment assay that will allow drug developers to determine the threshold (or minimum) level of antigen on the cell surface that will elicit a biological effect and thereby determine efficacy and safety, especially the on target and off tumour effects of a candidate therapeutic. It will also allow this assessment to be conducted for up to two different antigens simultaneously, thereby better reflecting the in vivo tumour environment and enabling development of candidate therapies which can target more than one antigen. This information is valuable to inform candidate drug selection and allow for safer therapeutics to enter clinic, thus increasing the chances of success.

The invention will now be further described with reference to the following headed sections. Any of the features described in any of the sections may be applied to any of the aspects of the invention in any workable combination.

### Inducible Systems

As described above, the methods of the invention may make use of any inducible systems which are operable to control expression of a protein of interest in a cell, such that the cell can be used to screen biological molecules, candidate therapeutics or engineered immune cells for their activity against said protein. Such inducible systems may be used individually or in pairs to express two or more different antigens, optionally at different levels, in the same cell for such screening methods. In preferred embodiments, the inducible systems used in the methods may be chemically inducible proximity systems based on plant hormone signalling pathways.

Suitably inducible systems which may be used in the methods of the invention include systems induced by the presence of an inducer, the absence of an repressor, or any other suitable physical or chemical change.

By way of non-limiting example, an inducible system for use in embodiments of the invention may be a forskolin-inducible system, a hypoxia-inducible system, a tetracycline-regulatable (e.g. inducible or repressible) system, an alcohol-inducible system, a steroid-inducible system, a mifepristone (RU486)-inducible system, an ecdysone-inducible system, a rapamycin-inducible system, a metallothionein-inducible system, a hormone-inducible system, a plant-hormone or analogue inducible system, a cumate-inducible system, a temperature-inducible system, a pH-inducible system and a metal-inducible system.

As will be discussed further below, various suitable inducible system have been described in the art and other are discussed herein. The person skilled in the art will be able to select one or more suitable inducible system for use in the various embodiments of the invention.

### Hypoxia Inducible Systems

In some embodiments the inducible system is a hypoxia-inducible system.

In some embodiments the hypoxia-inducible system comprises a hypoxia-inducible promoter operably linked to a sequence encoding the protein of interest. In some embodiments the hypoxia-inducible promoter comprises at least one hypoxia-responsive element (HRE) that is capable of being bound and activated by a hypoxia-inducible factor (HIF).

HIF is a family of transcription factors which are activated by decrease in the oxygen level in a cell. Under normal oxygen conditions, HIF is degraded following hydroxylation. Hypoxic conditions stabilise HIF and prevent its degradation. This allows HIF to translocate to the nucleus, bind to the HRE and activate HRE-responsive genes.

The hypoxia-inducible promoter typically comprises an HRE that is capable of being bound and activated by HIF operably linked to a minimal promoter. The particular promoter associated with the HRE can be selected depending on the circumstances, but typically minimal promoters are preferred, especially when it is desired to minimise background expression levels.

HREs are generally composed of multimers of short conserved sequences, termed HIF-binding sites (HBSs). As the name suggests, HBSs are bound by HIF, whereupon the HRE is activated to drive transcription of a gene encoding a protein of interest. Suitably the HRE comprises a plurality of HBS. Suitably the spacing between adjacent core consensus sequences in adjacent HBSs is from 3 to 50 nucleotides.

Suitable descriptions of a hypoxia inducible system can be found for example in US20110158947A1, which is incorporated herein by reference. Accordingly, in some embodiments the hypoxia-inducible promoter can be selected from the group consisting of: Adenosine A2B receptor (A2BR) promoter, Plasminogen activator receptor (uPAR) VEGF receptors (VEGFR1 and VEGFR2) promoter, Platelet-derived endothelial cell growth factor/thymidine phosphorylase (PDECGF/TP) promoter, nitric oxide synthase (NOS) promoter, Phosphoglycerate kinase-1 (PGK-1) promoter, Pyruvate kinase M (PK-M) promoter, Glucose transporter 1 (GLUT1) promoter, Hypoxia-inducible factor (HIF-1) promoter, Early growth response 1 (Egr-1) promoter, Nuclear factor kB (NFkB) promoter, Hepatocyte growth factor activator (HGFA) promoter, Vascular endothelial growth factor (VEGF) promoter, CXCL8 promoter, CCL11 promoter, Transforming growth factor-beta (TGF-β) promoter, procollagen promoter, Integrin-linked kinase (ILK) promoter, K1PDC1 promoter, Erythropoietin (EPO) promoter, Serine/Threonine Kinase-15 (STK15) promoter, the histone demethylase Jumonji domain containing 1A (JMJD1A) promoter, Endothelin-2 (EDN2) promoter, choline kinase (Chk) promoter, Sphingosine kinase 1 promoter, Carcinoembryonic antigen (CEA, ceacam5) promoter, Monocyte chemoattractant protein-1 (MCP-1/CCL2) promoter, MCP-5 (Ccl1 2) promoter, prostate-specific antigen (PSA) promoter, c-Met promoter, Matrix metalloproteinases Class III beta-tubulin (TUBB3) promoter, Glutamine: fructose-6-phosphate amidotransferase (GFAT) promoter, Protein phosphatase 1 nuclear targeting subunit beta-secretase (BACE1) promoter and Plasminogen activator inhibitor-1 (PAI-1) promoter.

Other naturally-occurring hypoxia-inducible promoters are described in WO2016/146819, which is incorporated herein by reference. See, for example, Table 4.

Hypoxia responsive elements have been described in L. Marignol, M. Lawler, M. Coffey & D. Hollywood (2005) Achieving hypoxia inducible gene expression in tumours, Cancer Biology & Therapy, 4:4, 365-370; U.S. Pat. No. 6,218,179; Madan et al, PNAS 90:3928, 1993; JP2005095173A, US 2006/0099709; and WO1999/048916. A murine hypoxia response element is disclosed in U.S. Pat. No. 5,942,434.

### Forskolin-Inducible Systems

In some embodiments the inducible system is a forskolin-inducible system.

In some embodiments the forskolin-inducible system comprises a forskolin-inducible promoter operably linked to a sequence encoding the protein of interest. In some embodiments the forskolin-inducible promoter comprises a forskolin-inducible cis-regulatory element (CRE) that is capable of being bound by CREB and/or AP1.

While the CRE/promoter is referred to as forskolin-inducible, it may also be induced by other agents. The mechanism of induction by forskolin is via the activation of adenylyl cyclase and the resultant increase of intracellular cAMP. Accordingly, the CRE/promoter is also inducible by other activators of adenylyl cyclase or factors that increase intracellular cAMP.

Preferably the CRE comprises at least 2, more preferably at least 3, transcription factor binding sites (TFBS) for CREB and/or AP1.

Preferably the forskolin-inducible promoter comprises a CRE as discussed above linked to a minimal promoter or a proximal promoter, preferably a minimal promoter.

The minimal promoter can be any suitable minimal promoter. A wide range of minimal promoters are known in the art. Without limitation, suitable minimal promoters include CMV minimal promoter (CMV-MP), YB-TATA minimal promoter (YB-TABA), HSV thymidine kinase minimal promoter (MinTK), SV40 minimal promoter (SV40-MP), or G6PC-MP (which is a liver-derived non-TATA box MP)

cAMP response elements are also described in U.S. Pat. No. 8,986,937, which is incorporated herein by reference. Exemplary naturally occurring CAMP-inducible promoters described therein include the PEPCK promoter (Roesler et al. (1998) The Journal of Biological Chemistry, 273, 14950-14957); promoters containing the CAMP-responsive element (CRE) that is located at position-294 with respect to the translation initiation site of the human cyclin D2 promoter (Muñiz et al. (2006) Biology of Reproduction 75 (2): 279-288); and promoters containing the CAMP-responsive element (CRE) of the lactate dehydrogenase A subunit promoter (Welfeld et al. (1989) J. Biol. Chem. 264 (12): 6941-7. Exemplary CAMP-inducible promoters comprise a 236-nucleotide glycoprotein hormone alpha subunit promoter, which contains a cyclic AMP (cAMP) regulatory element (CRE) (AF401991), as described in U.S. Pat. Appl. Publication no. US2008-0187942, published on Aug. 7, 2008, which is incorporated herein by reference. Such elements can be used in forskolin-inducible promoters as described above.

U.S. Pat. No. 9,060,310, which is incorporated herein by reference, describes further CAMP response elements, e.g. various CRE-palindromes and hairpins of SEQ ID NOs: 2, 3, 8, 9, 10 and 11. Such cAMP response elements can be used in forskolin-inducible promoters as described above.

US20070036810 and Mayr B, Montminy M., Nat Rev Nat Rev Mol Cell Biol 2001 August; 2 (8): 599-609 (both incorporated by reference) disclose cAMP responses elements which include a palindromic sequence of TGACGTCA or asymmetric variations which include a CRE half site with the core sequence TGAC. Such elements can be used in forskolin-inducible promoters as described above.

### Temperature Inducible Systems

The inducible system may be a temperature inducible system, for example it may be induced by reduction of temperature.

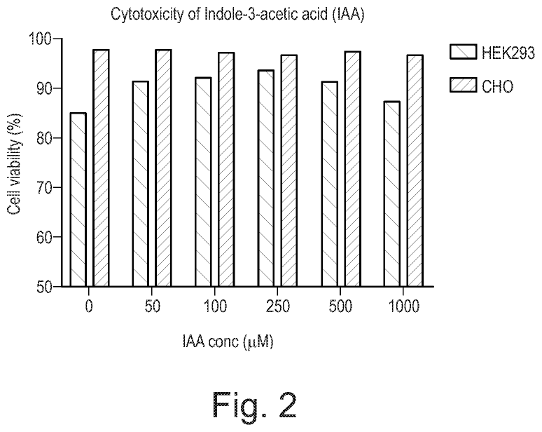

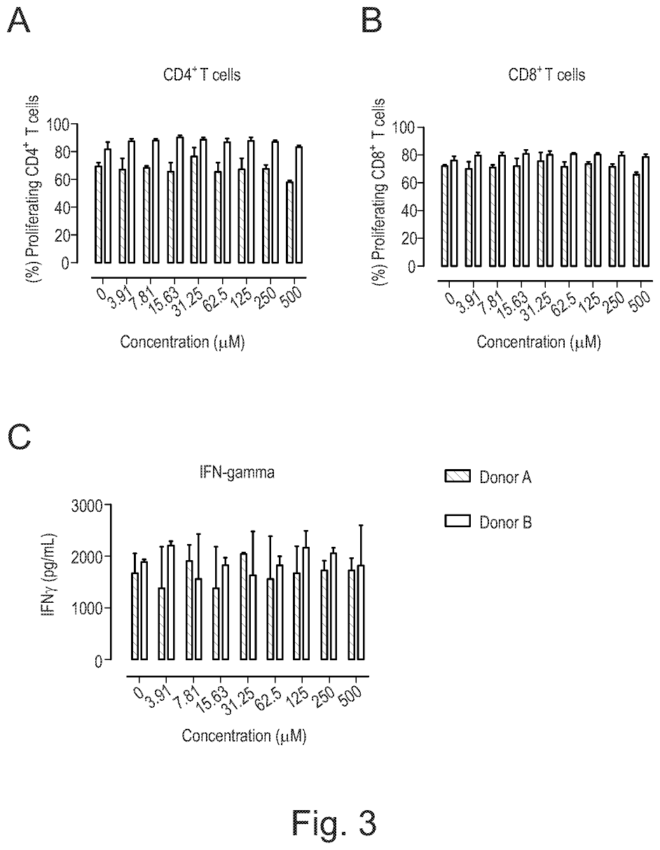

In some embodiments, the temperature inducible system comprises a temperature sensitive promoter operably linked to a sequence encoding the protein of interest. Suitably the promoter is a synthetic cold-shock responsive promoter derived from the S1006a gene (calcyclin) of CHO cells. The temperature sensitivity of the S1006a gene (calcyclin) promoter was identified by (Thaisuchat et al., 2011), which is incorporated herein by reference. In some embodiments, the inducible promoter is one of the synthetic cold-shock responsive promoters shown in FIG. 2 of (Thaisuchat et al., 2011). These promoters are induced by decrease of temperature as shown in FIG. 3 of (Thaisuchat et al., 2011). Most of these synthetic promoter constructs show expression similar to the known promoter SV40 at 37° C. and are induced by 2-3 times when the temperature is reduced to 33° C. In some preferred embodiments, the inducible promoter is sps5 from FIG. 2 of (Thaisuchat et al., 2011). In some preferred embodiments, the inducible promoter is sps8 from FIG. 2 of (Thaisuchat et al., 2011).

### pH Inducible Systems

The inducible system may be a pH inducible system, for example it may be induced by reduction or increase of pH to which cells comprising the inducible system are exposed.

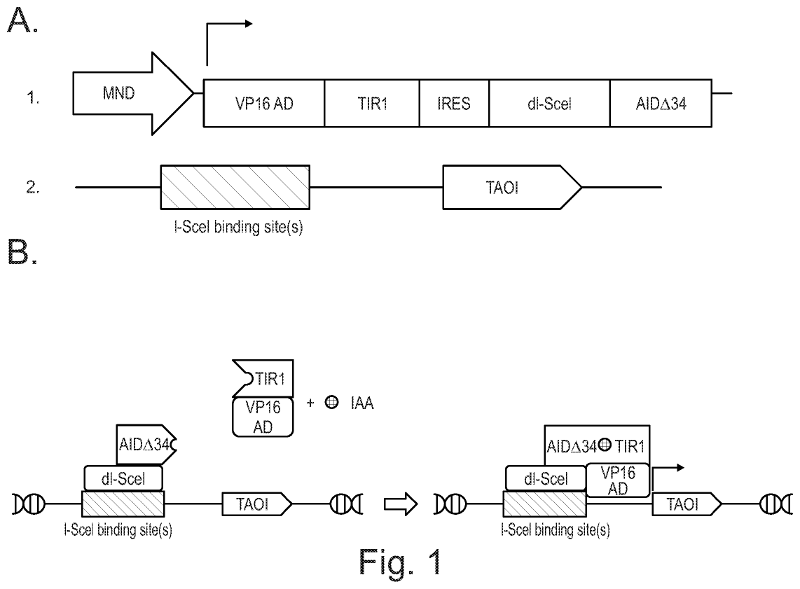

Suitably, the inducible system may comprise a pH sensitive promoter operably linked to a sequence encoding the protein of interest. Suitably the pH sensitive promoter may be induced by reduction of pH, i.e. a promoter inducible under acidic conditions. Suitable acid-inducible promoters are described in (Hou et al., 2016), which is incorporated herein by reference. In some embodiments, the inducible promoter is a synthetic promoter inducible under acidic conditions derived from the YGP1 gene or the CCW14 gene. The inducibility by acidic conditions of the YGP1 gene or the CCW14 gene was studied and improved by modifying transcription factor binding sites by (Rajkumar et al., 2016), which is incorporated herein by reference. In some embodiments, the inducible promoter is one of the synthetic promoter inducible under acidic conditions in FIGS. 1A, 2A, 3A and 4A of (Rajkumar et al., 2016). These promoters are induced by decrease of pH as shown in FIGS. 1B, 2B, 3B and 4B of (Rajkumar et al., 2016). Most of these synthetic promoters are induced by up to 10-15 times when the reduced from pH 6 to pH 3. In some preferred embodiments, the inducible promoter is YGP1pr from FIG. 1 of (Rajkumar et al., 2016). In other preferred embodiments, the inducible promoter is YGP1pr from FIG. 1 of (Rajkumar et al., 2016

### Osmolarity Induction

The inducible system may be an osmolarity-induced system.

Suitably, the osmolarity-induced system may comprise a osmolarity sensitive promoter operably linked to a sequence encoding the protein of interest. Suitably promoters induced by osmolarity are described in Zhang et al https://doi.org/10.1007/s11033-012-1566-3, which is incorporated herein by reference.

### Carbon Source Induction

The inducible system may be a carbon source inducible system, suitably which may be induced by addition of a specific carbon source, e.g. a non-sugar carbon source. Alternatively, the inducible promoter may be induced by withdrawal or the absence of a carbon source. Suitably, the carbon source inducible system may comprise a carbon source inducible promoter operably linked to a sequence encoding the protein of interest. Suitable promoters induced by the presence or absence of various carbon sources are described in (Weinhandl et al., 2014) which is incorporated herein by reference.

Alcohol (e.g. Ethanol) Induction

The inducible system may be an alcohol inducible system, suitably which is induced by addition of ethanol. Suitably, the alcohol inducible system may comprise an alcohol inducible promoter operably linked to a sequence encoding the protein of interest. Suitable promoters induced by ethanol are described in Matsuzawa et al https://doi.org/10.1007/s00253-013-4812-2 which is incorporated herein by reference.

### Amino Acid Induction

The inducible system may be an amino acid inducible system, suitably induced by addition of one or more amino acids. Suitably, the amino acid inducible system may comprise an amino acid inducible promoter operably linked to a sequence encoding the protein of interest. Suitably, the amino acid may be an aromatic amino acid. Suitably, the amino acid may be GABA (gamma aminobutyric acid), which is also a neurotransmitter. Suitable promoter induced by aromatic amino acids and GABA are described in Kim et al https://doi.org/10.1007/s00253-014-6303-5 which is incorporated herein by reference.

### Ecdysone Induction

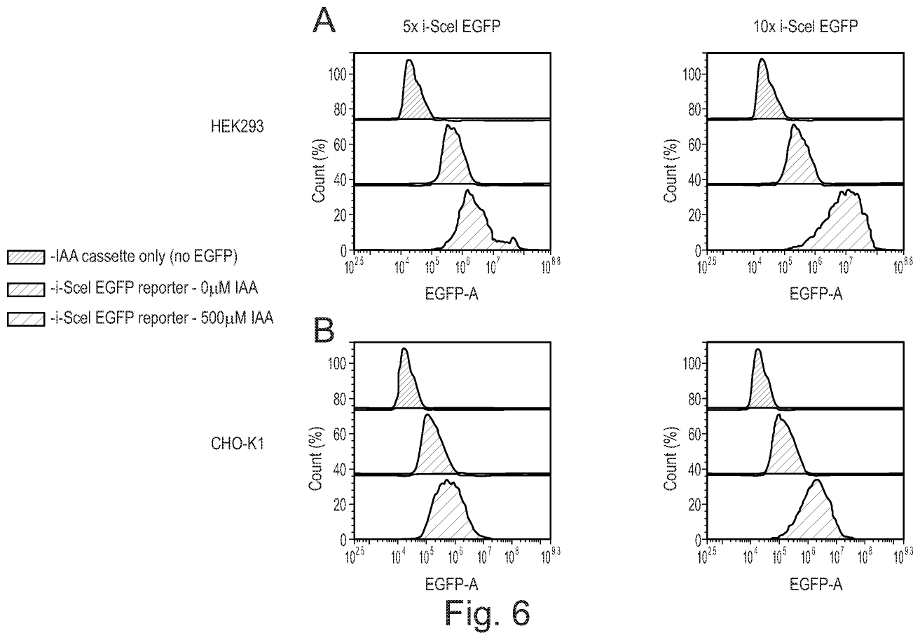

The inducible system may be a steroid hormone inducible system, suitably induced by a steroid hormone. Suitably, the steroid hormone inducible system may comprise a steroid hormone inducible promoter operably linked to a sequence encoding the protein of interest. Suitably, the steroid hormone may be ecdysone. A mammalian ecdysone-inducible system was created by No, Yao and Evans (No, Yao and Evans, 1996), which is incorporated herein by reference. Expression of a modified ecdysone receptor in mammalian cells allows expression from an ecdysone responsive promoter to be induced upon addition of ecdysone as shown in FIG. 2 of (No, Yao and Evans, 1996). This system showed lower basal activity and higher inducibility than the tetracycline-inducible system as shown in FIG. 6 of (No, Yao and Evans, 1996). A suitable commercially available inducible system is available from Agilent technologies and is described in (Agilent Technologies, 2015), which is incorporated herein by reference.

### Tetracycline-Regulated Induction

The inducible system may be a Tetracycline inducible system. Suitably, the tetracycline inducible system may comprise a promoter induced by the presence or absence of tetracycline or its derivatives operably linked to a sequence encoding the protein of interest.

In some embodiments, the promoter may be induced by the presence or absence of tetracycline or its derivatives. Suitable promoter induced by absence of tetracycline or its derivatives is the promoter in the tet-OFF system. In the tet-OFF system, tetracycline-controlled transactivator (tTA) allows transcriptional activation of a tTA-dependent promoter in the absence of tetracycline or its derivatives. tTA and the tTA-dependent promoter were initially created by (Gossen and Bujard, 1992), which is incorporated herein by reference. tTA was created by fusion of the tetracycline resistance operon (tet repressor) encoded in Tn10 of Escherichia coli with the activating cycline-controlled transactivator (tTA) and the tTA-dependent promoter was created by combining the tet operator sequence and a minimal promoter from the human cytomegalovirus promoter IE (hCMV-IE) (Gossen and Bujard, 1992). When tetracycline or its derivatives are added, tTA can no longer bind its target sequence within the tTA-dependent promoter and there is no expression from the tTA-dependent promoter. This is shown in FIG. 1A and explained on page 96 of (Jaisser, 2000), which is incorporated herein by reference. The mechanism of the conformational change brought by binding of tetracycline or its derivatives to tTA is described in Orth et al https://doi.org/10.1038/73324, which is incorporated herein by reference.

A suitable promoter induced by presence of tetracycline or its derivatives is the promoter in the tet-ON system. In the tet-ON system, a reverse tetracycline-controlled transactivator (rtTA) allows transcriptional activation of a tTA-dependent promoter in the presence of tetracycline or its derivatives as described in Gossen et al DOI: 10.1126/science.7792603, which is incorporated herein by reference. In the absence of tetracycline or its derivatives, tTA can no longer bind its target sequence within the tTA-dependent promoter and there is no expression from the tTA-dependent promoter. This is This is shown in FIG. 1B and explained on page s96 of (Jaisser, 2000). Suitably, an improved variant of the reverse tetracycline-controlled transactivator (rtTA) may be used.

Suitable improved variants are described in table 1 of (Urlinger et al., 2000), which is incorporated herein by reference. Variants rtTA-S2 and rtTA-M2 were shown to have lower basal activity in FIG. 3 of (Urlinger et al., 2000) which indicates minimal background expression from the tTA-dependent promoter in the absence of tetracycline or its derivatives. Additionally rtTA-M2 showed an increased sensitivity towards tetracycline and its derivatives as shown in in FIG. 3 of (Urlinger et al., 2000) and functions at 10 fold lower concentrations than rtTA. In some preferred embodiments, the improved variant of rtTA is rtTA-M2 from of (Urlinger et al., 2000).

Alternative improved variants are described in Table 1 of (Zhou et al., 2006). The majority of these variants were shown to have higher transcriptional activity and doxycycline sensitivity than rtTA as described in FIG. 3 of (Zhou et al., 2006). The highest performing variants were seven-fold more active and 100 times more sensitive to doxycycline. In some preferred embodiments, the improved variant of rtTA is V14, V15 or V16 from (Zhou et al., 2006). Suitable commercially available tetracycline-inducible system is the T-Rex system from (Life-Technologies, 2014).

### Cumate Induction

In some embodiments, the inducible system may be a cumate inducible system, suitably wherein the promoter may be induced by the presence or the absence of cumate. Suitably, the cumate inducible system may comprise a promoter induced by the presence or absence of cumate operably linked to a sequence encoding the protein of interest.

In the cumate switch system from (Mullick et al., 2006), which is incorporated herein by reference, a repressor CymR blocks transcription from a promoter comprising CuO sequence placed downstream of the promoter. Once cumate is added, the CymR repressor is unable to bind to CuO and transcription from a promoter comprising CuO can proceed. This is shown in FIG. 1B and FIG. 2 from (Mullick et al., 2006).

In an alternative cumate switch system, a chimeric transactivator (cTA) created from the fusion of CymR with the activation domain of VP16 does not prevent transcription from a promoter comprising CuO sequence upstream of a promoter in the presence of cumate. In the absence of cumate, the chimeric transactivator (cTA) binds to the CuO sequence and prevents transcription. This is shown in FIG. 1C and FIG. 3 from (Mullick et al., 2006).

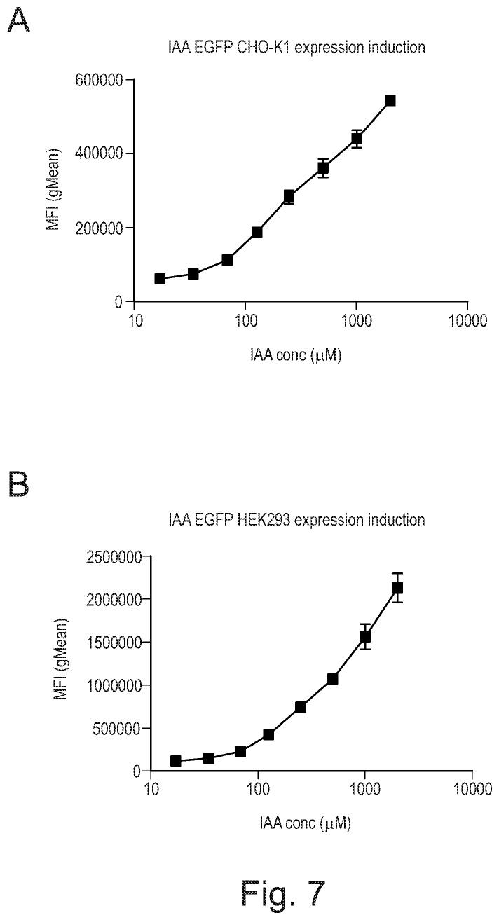

In a third configuration, a reverse chimeric transactivator (rcTA) prevents transcription from a promoter comprising CuO sequence upstream of a promoter in the absence of cumate. In the presence of cumate, the rcTA binds to the CuO sequence and transcription from the promoter comprising CuO sequence can proceed. This is shown in FIG. 1D and FIG. 7 from (Mullick et al., 2006).

Suitable commercially available cumate-inducible systems is found from SBI Biosciences (SBI, 2020), which is incorporated herein by reference.

### 4-Hydroxytamoxifen (OHT) Induction

The inducible system may be a 4-hydroxytamoxifen (OHT) inducible system suitably comprising a promoter which may be induced by 4-hydroxytamoxifen (OHT). Suitably the inducible system may comprise a promoter induced by the presence of 4-hydroxytamoxifen (OHT) operably linked to a sequence encoding the protein of interest. Suitable 4-hydroxytamoxifen inducible promoters are described by Feil et al https://doi.org/10.1006/bbrc. 1997.7124 which is incorporated herein by reference.

### Gas Induction

The inducible system may be a gas inducible system, suitably comprising a promoter which may be a gas-inducible promoter, e.g. acetaldehyde-inducible. Suitably the inducible system may comprise a promoter induced by the presence of a gas, operably linked to a sequence encoding the protein of interest. Suitable gas-inducible promoters are described in Weber et al https://doi.org/10.1038/nbt1021, which is incorporated herein by reference.

### Riboswitches, Ribozymes and Aptazyme Induction

The inducible system may be a riboswitch, ribozyme or an aptazyme inducible system, suitably which may be induced by the presence or absence of a ribozyme. The ribozyme can, in turn be induced by a ligand. Suitably the inducible system may comprise a promoter induced by the presence of a ribozyme, operably linked to a sequence encoding the protein of interest.

The inducible promoter may be induced in the absence of a metabolite. In some embodiments, the metabolite may be glucosamine-6-phosphate-responsive. Suitable ribozyme which acts as a glucosamine-6-phosphate-responsive gene repressor is described by Winkler et al https://doi.org/10.1038/nature02362 which is incorporated herein by reference.

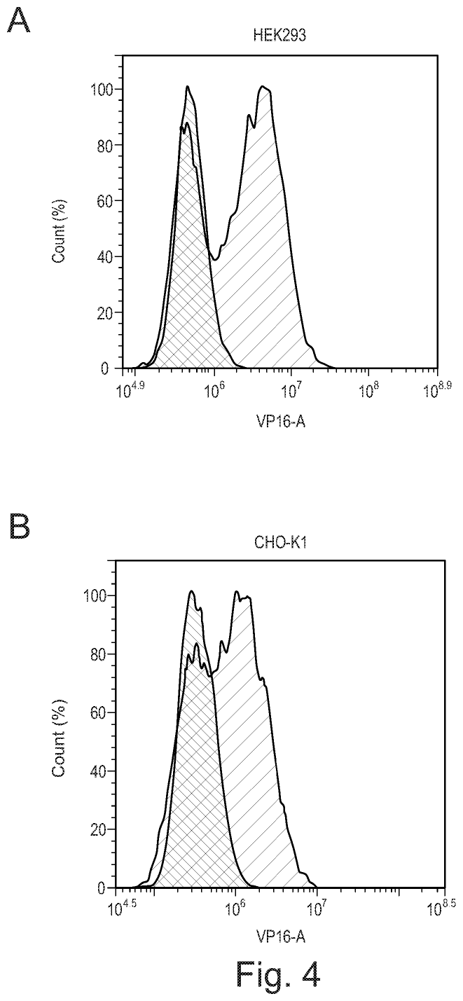

Protein expression can also be downregulated by ligand-inducible aptazyme. Protein expression can be downregulated by aptazyme which downregulate protein expression by small molecule-induced self-cleavage of the ribozyme resulting in mRNA degradation (Zhong et al., 2016) which is incorporated herein by reference. Suitable aptazymes are shown in FIG. 4A of (Zhong et al., 2016). These apraztymes reduce relative expression of a gene of interest as shown in FIG. 4 of (Zhong et al., 2016).

On the other hand, protein expression can also be upregulated by a small-molecule dependent ribozyme. The ribozyme may be tetracycline-dependent. Suitable tetracycline-dependent ribozymes which can switch on protein expression by preventing ribozyme cleavage which otherwise cleaves mRNA in the absence of ligand is described in Beilstein et al https://doi.org/10.1021/sb500270h which is incorporated herein by reference.

Protein expression can also be regulated by a guanine dependent aptazyme as described by Nomura et al https://doi.org/10.1039/C2CC33140C, which is incorporated herein by reference.

Additionally, an RNA architecture that combines a drug-inducible allosteric ribozyme with a microRNA precursor analogue that allows chemical induction of RNAi in mammalian cells is described in Kumar et al https://doi.org/10.1021/ja905596t, which is incorporated herein by reference.

### Metallothionein Induction

The inducible system may be a Metallothionein-inducible system, suitably comprising Metallothionein-inducible promoters that have been described in the literature. Suitably the inducible system may comprise a promoter induced by the presence of Metallothionein, operably linked to a sequence encoding the protein of interest. See for example Shinichiro Takahashi “Positive and negative regulators of the metallothionein gene” Molecular Medicine Reports Mar. 9, 2015, P795-799, which is incorporated herein by reference.

### Rapamycin Induction

The inducible system may be a rapamycin inducible system. Suitably the inducible system may be comprise a promoter induced by the presence of rapamycin, operably linked to a sequence encoding the protein of interest. The inducible promoter may be induced by a small molecule drug such as rapamycin. A humanized system for pharmacologic control of gene expression using rapamycin is described in Rivera et al Nature Medicine volume 2, pages 1028-1032 (1996) https://doi.org/10.1038/nm0996-1028, which is incorporated herein by reference.

### Rheoswitch

The inducible system may be a rheoswitch. Suitably the system may comprise a promoter which may be induced by small synthetic molecules. In some embodiments, these small synthetic molecules may be diacylhydrazine ligands. Suitable such systems for inducible up- and down-regulation of gene expression is described in Cress et al https://cancerres.aacrjournals.org/content/66/8_Supplement/27.2 which is incorporated herein by reference.

### CRISPR Induction

Gene expression may be induced by CRISPR-based transcription regulators. A nuclease-deficient Cas9 can be directed to a sequence of interest by designing its associated single guide RNA (sgRNA) and it can modulate the gene expression by tethering of effector domains on the sgRNA-Cas9 complex as shown in FIG. 1A of (Ferry, Lyutova and Fulga, 2017) which is incorporated herein by reference. Suitably the inducible system may be an inducible CRISPR-TR platform. Suitable versatile inducible-CRISPR-TR platform based on minimal engineering of the sgRNA is described in (Ferry, Lyutova and Fulga, 2017).

The CRISPR-based transcriptional regulation may in turn be induced by drugs. Suitable drug inducible CRISPR-based transcription regulators systems are shown in (Zhang et al., 2019).

### Chemically-Induced Proximity Induction

The inducible system may be a chemically induced proximity system. Suitable such small molecule-based systems for controlling protein abundance or activities is described in Liang et al 10.1126/scisignal.2001449 which is incorporated herein by reference.

Gene expression may be induced chemically by induced proximity caused by a chemical molecule combining two protein binding surfaces as shown in (Belshaw et al., 1996) which is incorporated herein by reference. Transcriptional activation of a gene of interest by chemically induced proximity by a molecule combining two protein binding surfaces is shown in FIG. 3 of (Belshaw et al., 1996).

As discussed hereinabove, in preferred embodiments of the invention, the or each inducible system is a chemically inducible proximity system. In particular, a plant hormone or plant hormone analogue inducible proximity system.

Suitably therefore in preferred embodiments of the invention, the inducible systems used in the methods of the invention are chemically inducible proximity systems, more preferably plant hormone or plant hormone analogue inducible proximity systems.

Suitably therefore in preferred embodiments of the methods of the invention, the cell which is provided may comprise the cell of the eighth aspect of the invention. Suitably the cell provided comprises a first and optionally a second chemically inducible proximity system.

In one embodiment, the first and second chemically inducible proximity systems may be plant hormone or plant hormone analogue inducible proximity systems as described herein. Suitably in such embodiments, the first chemically inducible proximity system comprises a first and second construct as defined herein, and the second chemically inducible proximity system comprises a third and a fourth construct as defined herein. In one embodiment, at least one of the chemically inducible proximity systems is a plant hormone inducible proximity system. In one embodiment, at least one of the chemically inducible systems is an abscisic acid inducible proximity system defined herein. In one embodiment, at least one of the chemically inducible systems is selected from a caffeine inducible proximity system defined herein, a Mandipropamid inducible proximity system defined herein, and a gibberellin inducible proximity system defined herein.

In a preferred embodiment, the first chemically inducible proximity system is selected from a caffeine inducible proximity system defined herein, a Mandipropamid inducible proximity system defined herein, and a gibberellin inducible proximity system defined herein and the second chemically inducible proximity system is an abscisic acid inducible proximity system as defined herein.

Details of the preferred plant hormone or plant hormone analogue inducible proximity systems for use in the present invention are described below.

### Components of Chemically Inducible Proximity Systems

### Effector Domain

The first construct of the first inducible system in accordance with the invention comprises two effector domains, one in the first chimeric protein and one in the second chimeric protein. The third construct of the second inducible system also comprises two effector domains, one in the third chimeric protein and one in the fourth chimeric protein.

Suitably the effector domains of either the first inducible system or the second inducible system may be selected from any transactivation domain or DNA binding domain as long as the effector domain of the first and second chimeric proteins is different, and the effector domain of the third and fourth chimeric proteins is different. Suitably the effector domains of the first and third chimeric proteins may be transactivation domains, suitably they may both be the same transactivation domain. Suitably the effector domains of the second and fourth chimeric proteins may be DNA binding domains, suitably they are different DNA binding domains.

Suitably the transactivation domains of the first and third chimeric proteins may be any transactivation domain. Suitably any transactivation domain selected from Gal4, Oaf1, Leu3, Rtg3, Pho4, Gln3, Gcn4 in yeast, and p53, NFAT, NF-κB, VP16 and VP34, as explained further below.

Suitably the DNA binding domains of the second and fourth chimeric proteins may be any DNA binding domain. Suitably any DNA binding domain selected from: LexA, dl-Scel and Gal4, as explained further below.

Suitably, each effector domain of the first construct is selected from a transactivation domain or a DNA binding domain. Suitably any DNA binding domain or transactivation domain may be used in the first construct. Suitably therefore any transactivation domain may be used in the first chimeric protein. Suitably therefore any DNA binding domain may be used in the second chimeric protein. Suitably, for example, the transactivation domain of the first construct, suitably of the first chimeric protein, may be selected from Gal4, Oaf1, Leu3, Rtg3, Pho4, Gln3, Gcn4 in yeast, and p53, NFAT, NF-κB, VP16 or VP34. Suitably, for example, the DNA binding domain of the first construct, suitably of the second chimeric protein, may be selected from: LexA, dl-Scel and Gal4. In one embodiment, the transactivation domain of the first construct, suitably of the first chimeric protein is VP16. In one embodiment the DNA binding domain of the first construct, suitably of the second chimeric protein, is a catalytically inactive I-Scel endonuclease DNA binding domain (dl-Scel).

Suitably, each effector domain of the third construct is selected from a transactivation domain or DNA binding domain. Suitably any DNA binding domain or transactivation domain may be used in the third construct. Suitably therefore any transactivation domain may be used in the third chimeric protein. Suitably, therefore, any DNA binding domain may be used in the fourth chimeric protein. Suitably, for example, the transactivation domain of the third construct, suitably of the third chimeric protein, may be selected from Gal4, Oaf1, Leu3, Rtg3, Pho4, Gln3, Gcn4 in yeast, and p53, NFAT, NF-κB, VP16 or VP34. Suitably, for example, the DNA binding domain of the third construct, suitably of the fourth chimeric protein, may be selected from: LexA, dl-Scel and Gal4. In one embodiment, the transactivation domain of the third construct, suitably of the third chimeric protein is VP16. In one embodiment the DNA binding domain of the third construct, suitably of the fourth chimeric protein, is a Gal4 DNA binding domain.

In some embodiments, as noted above, the effector domain is a transactivation domain.

Suitably, the transactivation domain of the first or third construct is brought into proximity with the dl-Scel DNA binding domain. Suitably, the proximity of the transactivation domain with the dl-Scel DNA binding domain in accordance with the present invention induces transcription from a dl-Scel binding site.

Suitably, the transactivation domain of the first or third construct may associate with the dl-Scel DNA binding domain. Suitably, the transactivation domain may bind to the dl-Scel DNA binding domain.

Suitably, the transactivation domain of the first or third construct is brought into proximity with the Gal4 DNA binding domain. Suitably, the proximity of the transactivation domain with the Gal4 DNA binding domain in accordance with the present invention induces transcription from a Gal4 upstream activation sequence.

Suitably, the transactivation domain of the first or third construct may associate with the Gal4 DNA binding domain. Suitably, the transactivation domain may bind to the Gal4 upstream activation sequence.

It would be understood by those skilled in the art that the transactivation domain in accordance with the present invention may be any transactivation domain. Merely by way of example, the transactivation domain may be Gal4, Oaf1, Leu3, Rtg3, Pho4, Gln3, Gcn4 in yeast, and p53, NFAT, NF-κB, VP16 or VP34.

Preferably, the transactivation domain is VP16.

More preferably, the transactivation domain is Herpes Simplex virus VP16.

Suitably the VP16 transactivation domain comprises a sequence according to SEQ ID NO: 11 or a functional fragment thereof, or a sequence having at least 70%, at least 75%, at least 80%, at least 85%, at least 90%, at least 95%, at least 96%, at least 97%, at least 98%, at least 99% identity thereto.

Suitably the VP16 transactivation domain consists of a sequence according to SEQ ID NO: 11 In some embodiments, as noted above, the effector domain is a DNA binding domain.

It would be understood by those skilled in the art that the DNA binding domain in accordance with the present invention may be any DNA binding domain. Merely by way of example, the DNA binding domain may be LexA, dl-Scel or Gal4.

Preferably the DNA binding domain is either dl-Scel or Gal4 as described hereinbelow.

In the context of the present invention, there is provided a modified endonuclease, which has been modified such that the endonuclease is catalytically inactive whilst retaining the ability to bind DNA.

In some embodiments, the effector domain is a modified I-Scel endonuclease DNA binding domain. Suitably in embodiments of the first or third construct.

In a suitable embodiment, the modified endonuclease is I-Scel endonuclease, which has been modified such that the I-Scel endonuclease is catalytically inactive whilst retaining the ability to bind DNA. An I-Scel endonuclease that is catalytically inactive whilst retaining DNA binding function may be referred to herein as ‘dead’I-Scel (dl-Scel).

It will be appreciated that an endonuclease (such as I-Scel) may be modified by any means or at any location that effectively removes the endonuclease function but retains DNA binding such that the modified endonucleases is catalytically inactive, leaving only DNA binding function. Suitably the modification may comprise one or more mutations.

A modified mutant or variant should be taken as a protein or nucleic acid sequence that shares sequence identity with the original sequence (or with a particular fragment of the original sequence) but that includes at least one modification (for example, a substitution, addition, or deletion) as compared to the original sequence.

In a suitable embodiment, the dl-Scel has at least one mutation within its active site.

Suitably, the mutation may be a substitution, an addition, or a deletion.

Suitably, the mutation is a substitution. Suitably the mutation is a non-conservative substitution. By ‘non-conservative’ it is meant that the substitution does not retain the characteristics of the original amino acid residue at the listed position.

In a suitable embodiment, a modified I-Scel includes at least one modification (for example, a substitution, addition, or deletion) as compared to the original sequence. Suitably, a modified I-Scel includes at least 2 modifications, at least 3 modifications, at least 4 modifications, at least 5 modifications, at least 6 modifications, at least 7 modifications, at least 8 modifications, at least 9 modifications, at least 10 modifications, at least 11 modifications, at least 12 modifications, at least 13 modifications, at least 14 modifications, at least 15 modifications, at least 16 modifications, at least 17 modifications, at least 18 modifications, at least 19 modifications, at least 20 modifications, at least 21 modifications, at least 22 modifications, at least 23 modifications, at least 24 modifications, at least 25 modifications, at least 26 modifications, at least 27 modifications, at least 28 modifications, at least 29 modifications or at least 30 modification.

Suitably, a modified I-Scel includes at least 35 modifications, at least 40 modifications, at least 45 modifications, at least 50 modifications, at least 55 modifications, at least 60 modifications, at least 75 modifications, at least 80 modifications, at least 85 modifications, at least 90 modifications, at least 95 modifications, or at least 100 modifications as compared to the original sequence.

Suitably, a modified I-Scel includes at least 120 modifications, at least 140 modifications, at least 160 modifications, at least 180 modifications, at least 200 modifications, at least 220 modifications, at least 240 modifications, at least 260 modifications, at least 280 modifications or at least 300 modifications as compared to the original sequence.

In a suitable embodiment, the dl-Scel comprises at least two mutations, suitably within its active site. Suitably at least two non-conservative substitutions within its active site. Suitably at least two alanine substitutions in its active site.

Suitably, the dl-Scel comprises a substitution at position 44 and/or position 145 of SEQ ID NO: 12. Suitably the dl-Scel comprises a substitution at position 44 and at position 145 of SEQ ID NO: 12. Suitably the dl-Scel comprises the substitution Asp44Ser and/or Asp145Ala in SEQ ID NO: 12. Suitably the dl-Scel comprises the substitution Asp44Ser and Asp 145Ala in SEQ ID NO: 12.

Suitably, the DNA recognition region of dl-Scel remains unchanged (as compared to the original sequence) and thus its DNA binding function is retained.

Suitably the dl-Scel DNA binding domain may comprise a sequence according to SEQ ID NO: 12, or a functional fragment thereof, or a sequence having at least 70%, at least 75%, at least 80%, at least 85%, at least 90%, at least 95%, at least 96%, at least 97%, at least 98%, at least 99% identity thereto, and which comprises a substitution at position 44 and/or position 145 as described above, or corresponding positions thereto.

By ‘position corresponding thereto’ as used herein it is meant the same position but in a orthologous or homologous sequence to that referred to, for example the same position in the same protein derived from a different organism. Corresponding positions may be determined by the skilled person, by alignment of the reference sequence with the orthologous or homologous sequence. Suitable alignment tools are available and well known in the art such as BLAST.

Suitably the dl-Scel DNA binding domain consists of a sequence according to SEQ ID NO: 13.

Alternatively the DNA binding domain may be LexA. Preferably LexA from E. coli.

Suitably the LexA DNA binding domain may comprise a sequence according to SEQ ID NO:21 or a functional fragment thereof, or a sequence having at least 70%, at least 75%, at least 80%, at least 85%, at least 90%, at least 95%, at least 96%, at least 97%, at least 98%, at least 99% identity thereto.

Suitably the LexA DNA binding domain consists of SEQ ID NO:21

Alternatively the DNA binding domain may be Gal4. Preferably Gal4 from S. cerevisiae.

Suitably the Gal4 DNA binding domain may comprise a sequence according to SEQ ID NO: 22 or a functional fragment thereof, or a sequence having at least 70%, at least 75%, at least 80%, at least 85%, at least 90%, at least 95%, at least 96%, at least 97%, at least 98%, at least 99% identity thereto.

Suitably the Gal4 DNA binding domain consists of SEQ ID NO: 22

### Auxin Binding Domain

The first or equally the third nucleic acid construct in accordance with the invention may comprise two auxin binding domains as a plant hormone inducer binding domain, one in the first or third chimeric protein and one in the second or fourth chimeric protein. Suitably, the auxin binding domain is selected from F-box transport inhibitor response 1 (TIR1) protein and transcriptional corepressor auxin/indole-3-acetic acid (AUX/IAA) proteins (termed AIDs), or fragments or derivatives thereof.

By ‘fragments or derivatives thereof’ as used herein it is meant modified forms of the protein, such as truncated forms of the protein, or mutated forms of the protein, suitable types truncations and mutations are described elsewhere herein. References to any protein component of the systems herein, may also refer to fragments or derivatives of said protein, some of which are specifically described herein.

In accordance with the present invention, the F-box transport inhibitor response 1 (TIR1) protein and transcriptional corepressor auxin/indole-3-acetic acid (AUX/IAA) protein (termed AID) heterodimerise in the presence of Auxin.

In one embodiment, the auxin binding domain is F-box transport inhibitor response 1 protein (TIR1).

TIR1 is a member of the F-box family of proteins. It is known that the F-box is a protein motif of approximately 50 amino acids that functions to mediate protein-protein interactions.

Suitably, the F-box protein is TIR1. Suitably, TIR1 in accordance with the present invention may originate from any plant, for example: Amborella trichopoda, Arabidopsis thaliana, Brachypodium distachyon, Brassica rapa, Brassica napus, Carica papaya, Cicer arietinum, Citrus, Cucumis sativus, Eucalyptus grandis, Glycine max, Gossypium raimondii, Marchantia polymorpha, Medicago truncatula, Oryza sativa, Petunia hybrida, Phalaenopsis equestris, Physcomitrella patens, Picea abies, Populus trichocarpa, Prunus persica, Ricinus communis, Selaginella moellendorffii, Solanum lycopersicum, Solanum melongena, Solanum tuberosum, Sorghum bicolor, Triticum aestivum, Triticum Urartu, Utricularia gibba, Vitis vinifera, Zea mays.

In a preferred embodiment, the TIR1 protein is Oryza sativa TIR1 (osTIR1).

Suitably, the Oryza sativa TIR1 (osTIR1) has been modified. Suitably therefore the osTIR1 is a modified mutant or variant of a reference osTIR1.

A modified mutant or variant should be taken as a protein or nucleic acid sequence that shares sequence identity with the original sequence (or with a particular fragment of the reference sequence) but that includes at least one modification (for example, a substitution, addition, or deletion) as compared to the reference sequence.

Suitably the modification may comprise one or more mutations. Suitably, the mutation may be a substitution, an addition, or a deletion.

The term “reference sequence” as used herein means the entire native or wild type sequence or fragment thereof of the same protein or nucleic acid from the same organism. Suitably the reference sequence is unmodified.

In a suitable embodiment, a modified TIR1 includes at least one modification (for example, a substitution, addition, or deletion) as compared to the reference sequence. Suitably, a modified mutant of TIR1 includes at least 2 modifications, at least 3 modifications, at least 4 modifications, at least 5 modifications, at least 6 modifications, at least 7 modifications, at least 8 modifications, at least 9 modifications, at least 10 modifications, at least 11 modifications, at least 12 modifications, at least 13 modifications, at least 14 modifications, at least 15 modifications, at least 16 modifications, at least 17 modifications, at least 18 modifications, at least 19 modifications, at least 20 modifications, at least 21 modifications, at least 22 modifications, at least 23 modifications, at least 24 modifications, at least 25 modifications, at least 26 modifications, at least 27 modifications, at least 28 modifications, at least 29 modifications or at least 30 modification as compared to the reference sequence.

Suitably, a modified TIR1 includes at least 35 modifications, at least 40 modifications, at least 45 modifications, at least 50 modifications, at least 55 modifications, at least 60 modifications, at least 75 modifications, at least 80 modifications, at least 85 modifications, at least 90 modifications, at least 95 modifications, or at least 100 modifications as compared to the reference sequence.

Suitably, a modified TIR1 includes at least 120 modifications, at least 140 modifications, at least 160 modifications, at least 180 modifications, at least 200 modifications, at least 220 modifications, at least 240 modifications, at least 260 modifications, at least 280 modifications or at least 300 modifications as compared to the reference sequence.

In a suitable embodiment, the modified TIR1 comprises at least two mutations. Suitably the modified TIR1 comprises at least two substitutions. Suitably the modified TIR1 comprises a substitution at position 7 and/or at position 10 of SEQ ID NO: 14. Suitably the modified TIR1 comprises a substitution at position 7 and at position 10 of SEQ ID NO: 14. Suitably the modified TIR1 comprises the substitution E7K and/or E10K in SEQ ID NO: 14. Suitably the modified TIR1 comprises the substitution E7K and E10K in SEQ ID NO: 14.

Suitably the TIR1 may comprise a sequence according to SEQ ID NO: 14, or a functional fragment thereof, or a sequence having at least 70%, at least 75%, at least 80%, at least 85%, at least 90%, at least 95%, at least 96%, at least 97%, at least 98%, at least 99% identity thereto, and which comprises a substitution at position 7 and/or at position 10 as described above, or at a corresponding position thereto.

Suitably the TIR1 consists of a sequence according to SEQ ID NO: 15.

Optionally the modified TIR1 may further comprise a mutation at position 74 of SEQ ID NO: 14. Optionally therefore the modified TIR1 may comprise a mutation at position 7 and/or 10 and/or 74 of SEQ ID NO: 14. Optionally therefore the modified TIR1 may comprise a mutation at position 7 and 10 and 74 of SEQ ID NO: 14. Suitably the modification at position 74 may be a substitution. Suitably the substitution is F74G. Optionally the modified TIR1 comprises the substitution E7K and/or E10K and/or F74G in SEQ ID NO: 14. Suitably the modified TIR1 comprises the substitution E7K and E10K and F74G in SEQ ID NO: 14.

Suitably the TIR1 may comprise a sequence according to SEQ ID NO: 14, or a functional fragment thereof, or a sequence having at least 70%, at least 75%, at least 80%, at least 85%, at least 90%, at least 95%, at least 96%, at least 97%, at least 98%, at least 99% identity thereto, and which comprises a substitution at position 7 and/or at position 10 and/or at position 74 as described above, or at a corresponding position thereto.

Suitably the TIR1 consists of a sequence according to SEQ ID NO:22. Suitably the TIR1 having a further modification at position 74 is used when the auxin is 5-Ph-IAA. Advantageously, constructs using this modified TIR1 show no detectable leaky degradation, and require a much lower ligand concentration.

As previously discussed, transcriptional corepressor auxin/indole-3-acetic acid (AUX/IAA) proteins (termed AIDs) heterodimerize with TIR1 in the presence of Auxin.

In one embodiment the auxin binding protein is therefore an AID protein.

Suitably, the AID protein may from any of the following plant species: Amborella trichopoda, Arabidopsis thaliana, Brachypodium distachyon, Brassica rapa, Brassica napus, Carica papaya, Cicer arietinum, Citrus, Cucumis sativus, Eucalyptus grandis, Glycine max, Gossypium raimondii, Marchantia polymorpha, Medicago truncatula, Oryza sativa, Petunia hybrida, Phalaenopsis equestris, Physcomitrella patens, Picea abies, Populus trichocarpa, Prunus persica, Ricinus communis, Selaginella moellendorffii, Solanum lycopersicum, Solanum melongena, Solanum tuberosum, Sorghum bicolor, Triticum aestivum, Triticum Urartu, Utricularia gibba, Vitis vinifera, Zea mays.

Suitably, the AID is a truncated version of an AID protein.

In the context of the present invention, a truncated version of AID is a truncated version of the reference sequence (i.e. not the full length sequence), but sharing full sequence identity with a corresponding portion of the reference sequence.

In one embodiment, the AID protein is a truncation of the sequence according to SEQ ID NO: 16. Suitably the truncation may comprise up to 100 amino acids, up to 90 amino acids, up to 80 amino acids, up to 70 amino acids, up to 60 amino acids, up to 50 amino acids, up to 40 amino acids, up to 30 amino acids. Suitably the truncation is from the N-terminal end or the C-terminal end of the amino acid sequence.

In a suitable embodiment of the present invention the truncated version of AID is a 133 amino acid truncated version, removing C-terminal domains 3 and 4, otherwise known as a Delta34 mutant (AIDΔ34). Suitably, AIDΔ34 is a truncated version of the reference sequence. Suitably AIDΔ34 is a C-terminal truncation of the reference sequence.

Suitably the AIDΔ34 comprises a sequence as set out in SEQ ID NO: 17, or a sequence having at least 70%, at least 75%, at least 80%, at least 85%, at least 90%, at least 95%, at least 96%, at least 97%, at least 98%, at least 99% identity thereto.

Suitably the AIDΔ34 consists of the sequence as set out in SEQ ID NO: 17.

In an alternative embodiment of the present invention, the truncated version of AID has a truncation of 63 amino acids at the N-terminus and a truncation of 97 amino acids at the C-terminus, otherwise known as the mAID mutant. Suitably mAID is a truncated version of the reference sequence.

Suitably mAID comprises a sequence as set out in SEQ ID NO:23, or a sequence having at least 70%, at least 75%, at least 80%, at least 85%, at least 90%, at least 95%, at least 96%, at least 97%, at least 98%, at least 99% identity thereto.

Suitably the mAID consists of the sequence set out in SEQ ID NO:23.

### Abscisic Acid Binding Domain

The first or equally the third nucleic acid construct in accordance with the invention may comprise two abscisic acid binding domains as a plant hormone inducer binding domain, one in the third or first chimeric protein and one in the fourth or second chimeric protein. Suitably, the abscisic acid binding domain is selected from abscisic acid insensitive 1 protein (ABI1) or pyrobactin resistance-like protein (PYL1), or a fragment or derivative thereof.

In accordance with the present invention, the ABI1 and PYL1 proteins heterodimerise in the presence of Abscisic Acid.

In one embodiment, the abscisic acid binding domain is ABI1.

ABI1 is a member of the 2C class of protein serine/threonine phosphatases (PP2Cs).

Suitably, the PP2C protein is ABI1. Suitably, ABI1 in accordance with the present invention may originate from any plant, for example: Amborella trichopoda, Arabidopsis thaliana, Brachypodium distachyon, Brassica rapa, Brassica napus, Carica papaya, Cicer arietinum, Citrus, Cucumis sativus, Eucalyptus grandis, Glycine max, Gossypium raimondii, Marchantia polymorpha, Medicago truncatula, Oryza sativa, Petunia hybrida, Phalaenopsis equestris, Physcomitrella patens, Picea abies, Populus trichocarpa, Prunus persica, Ricinus communis, Selaginella moellendorffii, Solanum lycopersicum, Solanum melongena, Solanum tuberosum, Sorghum bicolor, Triticum aestivum, Triticum Urartu, Utricularia gibba, Vitis vinifera, Zea mays.

In a preferred embodiment, the ABI1 protein is the Arabidopsis thaliana ABI1.

Suitably, the ABI1 protein has been modified. Suitably therefore the ABI1 protein is a modified mutant or variant of a reference ABI1 protein.

A modified mutant or variant should be taken as a protein or nucleic acid sequence that shares sequence identity with the original sequence (or with a particular fragment of the reference sequence) but that includes at least one modification (for example, a substitution, addition, or deletion) as compared to the reference sequence.

Suitably the modification may comprise one or more mutations. Suitably, the mutation may be a substitution, an addition, or a deletion.

Suitable such modifications are defined hereinabove in relation to the auxin binding domain.

The term “reference sequence” as used herein means the entire native or wild type sequence or fragment thereof of the same protein or nucleic acid from the same organism. Suitably the reference sequence is unmodified.

Suitably the ABI1 protein is modified by a deletion. Suitably by a truncation which leaves only the complementary surface of the ABI1 protein which is operable to interact with PYL1 protein. Suitably the ABI1 protein is modified to consist of the ABI1 protein complementary surface.

In one embodiment the ABI1 protein comprises a truncation at its N terminus and its C terminus. In one embodiment the ABI1 protein comprises a truncation of amino acids 1-125 from its N-terminus, suitably 125 amino acids. In one embodiment, the ABI1 protein comprises a truncation of amino acid residues 424-434 from its C-terminus, suitably 10 amino acids. Suitably the ABI1 protein comprises amino acids 126 to 423 of SEQ ID NO:24.

In one embodiment, the ABI1 protein consists of amino acids 126 to 423 of SEQ ID NO:24, otherwise known as ‘ABI1cs’. In one embodiment, the ABIcs protein consists of SEQ ID NO:25

As previously discussed, PYL1 protein heterodimerises with ABI1 protein in the presence of abscisic acid

In one embodiment the abscisic acid binding protein is therefore a PYL1 protein.

Suitably, the PYL1 protein may from any of the following plant species: Amborella trichopoda, Arabidopsis thaliana, Brachypodium distachyon, Brassica rapa, Brassica napus, Carica papaya, Cicer arietinum, Citrus, Cucumis sativus, Eucalyptus grandis, Glycine max, Gossypium raimondii, Marchantia polymorpha, Medicago truncatula, Oryza sativa, Petunia hybrida, Phalaenopsis equestris, Physcomitrella patens, Picea abies, Populus trichocarpa, Prunus persica, Ricinus communis, Selaginella moellendorffii, Solanum lycopersicum, Solanum melongena, Solanum tuberosum, Sorghum bicolor, Triticum aestivum, Triticum Urartu, Utricularia gibba, Vitis vinifera, Zea mays.

In a preferred embodiment, the PYL1 protein is the Arabidopsis thaliana PYL1.

Suitably, the PYL1 protein has been modified. Suitably therefore the PYL1 protein is a modified mutant or variant of a reference PYL1 protein.

A modified mutant or variant should be taken as a protein or nucleic acid sequence that shares sequence identity with the original sequence (or with a particular fragment of the reference sequence) but that includes at least one modification (for example, a substitution, addition, or deletion) as compared to the reference sequence.

Suitably the modification may comprise one or more mutations. Suitably, the mutation may be a substitution, an addition, or a deletion.

Suitable such modifications are defined hereinabove in relation to the auxin binding domain.

The term “reference sequence” as used herein means the entire native or wild type sequence or fragment thereof of the same protein or nucleic acid from the same organism. Suitably the reference sequence is unmodified.

Suitably the PYL1 protein is modified by a deletion. Suitably by a truncation which leaves only the complementary surface of the PYL1 protein which is operable to interact with ABI1 protein. Suitably the PYL1 protein is modified to consist of the PYL1 protein complementary surface.

In one embodiment the PYL1 protein comprises a truncation at its N terminus and its C terminus. In one embodiment the PYL1 protein comprises a truncation of amino acids 1-32 from its N-terminus, suitably 32 amino acids. In one embodiment, the PYL1 protein comprises a truncation of amino acid residues 210-221 from its C-terminus, suitably 12 amino acids. Suitably the PYL1 protein comprises amino acids 33 to 209 of SEQ ID NO:26.

In one embodiment, the PYL1 protein consists of amino acids 33 to 209 of SEQ ID NO:26, otherwise known as ‘PYLcs’. In one embodiment, the PYLcs protein consists of SEQ ID NO:27

### Caffeine Binding Domain

The first or equally the third nucleic acid construct in accordance with the invention may comprise two caffeine binding domains as an inducer binding domain, one in the first or third chimeric protein and one in the second or fourth chimeric protein.

Suitably, the caffeine binding domain may be an anti-caffeine antibody, or fragments or derivatives thereof.

By ‘fragments or derivatives thereof’ as used herein it is meant modified forms of the protein, such as truncated forms of the protein, Fab domains, scFvs, minibodies, camelid heavy chain antibodies, VHH domains, single variable domains, or mutated forms of the protein, suitable types truncations and mutations are described elsewhere herein. References to any protein component of the systems herein may also refer to fragments or derivatives of said protein, some of which are specifically described herein.

In one embodiment, the caffeine binding domain is an anti-caffeine antibody heavy-chain fragment, preferably an anti-caffeine antibody VHH domain, (aCaffVHH).

In accordance with the present invention, two aCaffVHH domains homodimerise in the presence of caffeine.

Suitably any two identical anti-caffeine antibodies or functional binding fragments or derivatives thereof may also homodimerize in the presence of caffeine and may also be used as the caffeine binding domains.

Suitably, the caffeine binding domain in accordance with the present invention may originate from any camelid. In a preferred embodiment, originating from Lama glama.

Suitably the caffeine binding domain is an anti-caffeine antibody heavy-chain fragment, preferably an anti-caffeine antibody VHH domain, (aCaffVHH) originating from a camelid, suitably from Lama glama. In one embodiment the caffeine binding domain may comprise a sequence according to SEQ ID NO:31.

Suitably the aCaffVHH domain may comprise a sequence according to SEQ ID NO:31, or a functional fragment thereof, or a sequence having at least 70%, at least 75%, at least 80%, at least 85%, at least 90%, at least 95%, at least 96%, at least 97%, at least 98%, at least 99% identity thereto. Suitably the caffeine binding domain may consist of SEQ ID NO:31. Suitably the aCaffVHH domain may consist of SEQ ID NO:31.

### Mandipropamid (Mandi) Binding Domain

The first or equally the third nucleic acid construct in accordance with the invention may comprise two Mandipropamid (Mandi) binding domains as an inducer binding domain, one in the third or first chimeric protein and one in the fourth or second chimeric protein.

Suitably, the Mandipropamid binding domain is selected from abscisic acid insensitive 1 protein (ABI1), PYR^{Mandi} (a hextuple mutant of the Arabidopsis thaliana ABA receptor PYR110), PYLcs^{Mandi} (a hextuple mutant of the complementary surface region of Arabidopsis thaliana PYR1-like, PYL1cs, with the mutations transposed from PYR^{Mandi}), or a fragment or derivative thereof.

By ‘fragments or derivatives thereof’ as used herein it is meant modified forms of the protein, such as truncated forms of the protein, or mutated forms of the protein, suitable types truncations and mutations are described elsewhere herein. References to any protein component of the systems herein, may also refer to fragments or derivatives of said protein, some of which are specifically described herein.

In accordance with the present invention, the ABI1 and PYR^{Mandi} proteins, and ABI1 and PYLcs^{Mandi} proteins, heterodimerise in the presence of Mandi.

In one embodiment, the Mandipropamid binding domain is ABI1.

ABI1 is a member of the 2C class of protein serine/threonine phosphatases (PP2Cs).

Suitably, the PP2C protein is ABI1. Suitably, ABI1 in accordance with the present invention may originate from any plant, for example: Amborella trichopoda, Arabidopsis thaliana, Brachypodium distachyon, Brassica rapa, Brassica napus, Carica papaya, Cicer arietinum, Citrus, Cucumis sativus, Eucalyptus grandis, Glycine max, Gossypium raimondii, Marchantia polymorpha, Medicago truncatula, Oryza sativa, Petunia hybrida, Phalaenopsis equestris, Physcomitrella patens, Picea abies, Populus trichocarpa, Prunus persica, Ricinus communis, Selaginella moellendorffii, Solanum lycopersicum, Solanum melongena, Solanum tuberosum, Sorghum bicolor, Triticum aestivum, Triticum Urartu, Utricularia gibba, Vitis vinifera, Zea mays.

In a preferred embodiment, the ABI1 protein is the Arabidopsis thaliana ABI1. Suitably comprising of SEQ ID NO:24. Suitably consisting of SEQ ID NO:24.

Suitably, the ABI1 protein has been modified. Suitably therefore the ABI1 protein is a modified mutant or variant of a reference ABI1 protein.

A modified mutant or variant should be taken as a protein or nucleic acid sequence that shares sequence identity with the original sequence (or with a particular fragment of the reference sequence) but that includes at least one modification (for example, a substitution, addition, or deletion) as compared to the reference sequence.

Suitably the modification may comprise one or more mutations. Suitably, the mutation may be a substitution, an addition, or a deletion.

Suitable such modifications are defined hereinabove in relation to the auxin binding domain.

The term “reference sequence” as used herein means the entire native or wild type sequence or fragment thereof of the same protein or nucleic acid from the same organism. Suitably the reference sequence is unmodified.

Suitably the ABI1 protein is modified by a deletion. Suitably by a truncation which leaves only the complementary surface of the ABI1 protein which is operable to interact with PYL1 protein. Suitably the ABI1 protein is modified to consist of the ABI1 protein complementary surface.

In one embodiment the ABI1 protein comprises a truncation at its N terminus and its C terminus. In one embodiment the ABI1 protein comprises a truncation of amino acids 1-125 from its N-terminus, suitably 125 amino acids. In one embodiment, the ABI1 protein comprises a truncation of amino acid residues 424-434 from its C-terminus, suitably 10 amino acids. Suitably the ABI1 protein comprises amino acids 126 to 423 of SEQ ID NO:24.

In one embodiment, the ABI1 protein consists of amino acids 126 to 423 of SEQ ID NO:24, otherwise known as ‘ABI1cs’. In one embodiment, the ABIcs protein consists of SEQ ID NO: 25.

As previously discussed, PYR^{Mandi} protein heterodimerises with ABI1 protein in the presence of Mandi.

In one embodiment the Mandipropamid binding protein is therefore a PYR^{Mandi} protein.

Suitably, the PYR^{Mandi} protein may originate from any of the following plant species: Amborella trichopoda, Arabidopsis thaliana, Brachypodium distachyon, Brassica rapa, Brassica napus, Carica papaya, Cicer arietinum, Citrus, Cucumis sativus, Eucalyptus grandis, Glycine max, Gossypium raimondii, Marchantia polymorpha, Medicago truncatula, Oryza sativa, Petunia hybrida, Phalaenopsis equestris, Physcomitrella patens, Picea abies, Populus trichocarpa, Prunus persica, Ricinus communis, Selaginella moellendorffii, Solanum lycopersicum, Solanum melongena, Solanum tuberosum, Sorghum bicolor, Triticum aestivum, Triticum Urartu, Utricularia gibba, Vitis vinifera, Zea mays.

In a preferred embodiment, the PYR^{Mandi} protein is the Arabidopsis thaliana PYR^{Mandi} protein. Suitably comprising SEQ ID NO:33, or a functional fragment thereof, or a sequence having at least 70%, at least 75%, at least 80%, at least 85%, at least 90%, at least 95%, at least 96%, at least 97%, at least 98%, at least 99% identity thereto. In one embodiment, the PYR^{Mandi} protein consists of SEQ ID NO:33.

As previously discussed, PYLcs^{Mandi} protein heterodimerises with ABI1 protein in the presence of Mandipropamid.

In one embodiment the Mandipropamid binding protein is therefore a PYL1cs^{Mandi} protein.

Suitably, the PYL1cs^{Mandi} protein may originate from any of the following plant species: Amborella trichopoda, Arabidopsis thaliana, Brachypodium distachyon, Brassica rapa, Brassica napus, Carica papaya, Cicer arietinum, Citrus, Cucumis sativus, Eucalyptus grandis, Glycine max, Gossypium raimondii, Marchantia polymorpha, Medicago truncatula, Oryza sativa, Petunia hybrida, Phalaenopsis equestris, Physcomitrella patens, Picea abies, Populus trichocarpa, Prunus persica, Ricinus communis, Selaginella moellendorffii, Solanum lycopersicum, Solanum melongena, Solanum tuberosum, Sorghum bicolor, Triticum aestivum, Triticum Urartu, Utricularia gibba, Vitis vinifera, Zea mays.

In a preferred embodiment, the PYL1cs^{Mandi} protein is the Arabidopsis thaliana derived PYL1cs^{Mandi} Suitably comprising SEQ ID NO: 35, or a functional fragment thereof, or a sequence having at least 70%, at least 75%, at least 80%, at least 85%, at least 90%, at least 95%, at least 96%, at least 97%, at least 98%, at least 99% identity thereto. In one embodiment, the PYL1cs^{Mandi} protein consists of SEQ ID NO: 35.

### Gibberellin Binding Domain

The first or equally the third nucleic acid construct in accordance with the invention may comprise two gibberellin binding domains as a plant hormone inducer binding domain, one in the first or third chimeric protein and one in the second or fourth chimeric protein.

Suitably, the gibberellin binding domain is selected from gibberellin insensitive dwarf 1 (GID1) protein, or gibberellin insensitive (GAI) protein or fragments or derivatives thereof.

By ‘fragments or derivatives thereof’ as used herein it is meant modified forms of the protein, such as truncated forms of the protein, or mutated forms of the protein, suitable types truncations and mutations are described elsewhere herein. References to any protein component of the systems herein, may also refer to fragments or derivatives of said protein, some of which are specifically described herein.

In accordance with the present invention, the gibberellin insensitive dwarf 1 (GID1) protein and the gibberellin insensitive (GAI) protein heterodimerise in the presence of gibberellin.

In some embodiments the GAI protein may be modified. Suitably it may be modified by truncation, at either the C or N terminus, suitably at the C terminus. Suitably the modified GAI protein may consist of amino acids 1-92 (or nucleotides 1-276) of the full-length GAI protein. Suitably references herein to ‘modified GAI protein’ or ‘GAI protein which may be modified’ or ‘GAI protein fragment’ refer to this truncated form.

Suitably therefore, also in accordance with the invention, the gibberellin insensitive dwarf 1 (GID1) protein and amino acids 1-92 of gibberellin insensitive (GAI) protein, i.e. the modified GAI protein, heterodimerise in the presence of gibberellin.

In one embodiment, the gibberellin binding domain is gibberellin insensitive dwarf 1 (GID1) protein.

Suitably, GID1 in accordance with the present invention may originate from any plant, for example: Amborella trichopoda, Arabidopsis thaliana, Brachypodium distachyon, Brassica rapa, Brassica napus, Carica papaya, Cicer arietinum, Citrus, Cucumis sativus, Eucalyptus grandis, Glycine max, Gossypium raimondii, Marchantia polymorpha, Medicago truncatula, Oryza sativa, Petunia hybrida, Phalaenopsis equestris, Physcomitrella patens, Picea abies, Populus trichocarpa, Prunus persica, Ricinus communis, Selaginella moellendorffii, Solanum lycopersicum, Solanum melongena, Solanum tuberosum, Sorghum bicolor, Triticum aestivum, Triticum Urartu, Utricularia gibba, Vitis vinifera, Zea mays.

In a preferred embodiment, the GID1 protein is the Arabidopsis thaliana GID1. Suitably GID1 may comprise a sequence according to SEQ ID NO:37, or a functional fragment thereof, or a sequence having at least 70%, at least 75%, at least 80%, at least 85%, at least 90%, at least 95%, at least 96%, at least 97%, at least 98%, at least 99% identity thereto. In one embodiment, the GID1 protein consists of SEQ ID NO:37.

In one embodiment the gibberellin binding protein is gibberellin insensitive (GAI) protein.

In a preferred embodiment, the GAI protein is the Arabidopsis thaliana GAI protein. Suitably GAI may comprise a sequence according to SEQ ID NO:48, or a functional fragment thereof, or a sequence having at least 70%, at least 75%, at least 80%, at least 85%, at least 90%, at least 95%, at least 96%, at least 97%, at least 98%, at least 99% identity thereto. In one embodiment, the GAI protein consists of SEQ ID NO:48.

Suitably, the GAI protein may originate from any of the following plant species: Amborella trichopoda, Arabidopsis thaliana, Brachypodium distachyon, Brassica rapa, Brassica napus, Carica papaya, Cicer arietinum, Citrus, Cucumis sativus, Eucalyptus grandis, Glycine max, Gossypium raimondii, Marchantia polymorpha, Medicago truncatula, Oryza sativa, Petunia hybrida, Phalaenopsis equestris, Physcomitrella patens, Picea abies, Populus trichocarpa, Prunus persica, Ricinus communis, Selaginella moellendorffii, Solanum lycopersicum, Solanum melongena, Solanum tuberosum, Sorghum bicolor, Triticum aestivum, Triticum Urartu, Utricularia gibba, Vitis vinifera, Zea mays.

In one embodiment the gibberellin binding protein is a modified GAI protein, suitably nucleotides 1-276 (amino acids 1-92) of SEQ ID NO: 48. In one embodiment, the modified GAI protein consists of amino acids 1-92 of SEQ ID NO:48.

Suitably the modified GAI protein, i.e. nucleotides 1-276 (amino acids 1-92) of the full length GAI protein, comprises a sequence as set out in SEQ ID NO:39, or a sequence having at least 70%, at least 75%, at least 80%, at least 85%, at least 90%, at least 95%, at least 96%, at least 97%, at least 98%, at least 99% identity thereto. In one embodiment, the modified GAI protein consists of SEQ ID NO:39.

### Auxin

Auxins are a class of plant hormones that control growth and development in plants. It is known that Auxins regulate transcription by promoting the degradation of the family of transcriptional repressors AIDs. In the context of the present invention, Auxin acts as a plant hormone inducer for the chemically induced proximity system of the invention. Suitably, Auxin induces heterodimerization of the auxin binding proteins TIR1 and AID.

Suitably, the Auxin may be any Auxin, or precursor or mimics thereof.

Suitably, the Auxin may be, indole-3-acetic acid, 4-chloroindole-3-acetic acid, phenylacetic acid, 5-phenyl-indole-3-acetic acid (5-Ph-IAA), indole-3-butyric acid, or indole-3-propionic acid. Suitably the auxin may be a synthetic auxin such as 1-naphthaleneacetic acid, or 2,4-dichlorophenoxyacetic acid (2,4-D).

In a suitable embodiment, the Auxin is indole-3-acetic acid (IAA) which is used in the invention.

Suitably, the IAA may be IAA1, or IAA2.

### Abscisic Acid

Abscisic Acid (ABA) is an isoprenoid plant hormone which functions in plant developmental processed including seed and bud dormancy, and the control of stomatal closure. It is also important for response to environmental stresses. In the context of the present invention, Abscisic acid acts as a plant hormone inducer for the chemically induced proximity system of the invention. Suitably, Abscisic acid induces heterodimerization of the abscisic acid binding proteins ABI1 and PYL1.

Suitably the term abscisic acid may also encompass abscisic acid precursors such as zeaxanthin, xanthin, or abscisic aldehyde, or mimics such as pyrobactin. Suitably the abscisic acid may be synthetic.

In a suitable embodiment abscisic acid (ABA) is used in the invention.

### Caffeine

Caffeine is a xanthine alkaloid, which acts as a natural pesticide, serving as a toxic substance that deters herbivores and insects from consuming the plant's leaves, seeds, and other parts. Caffeine also plays a role in enhancing the plant's reproductive success by attracting pollinators such as bees and butterflies. Caffeine can be considered to be a plant hormone. Caffeine acts as an inducer for the chemically induced proximity system of the invention. Suitably, caffeine induces homodimerization of the caffeine binding protein aCaffVHH.

In a suitable embodiment caffeine is used in the invention

### Mandipropamid (Mandi)

Mandipropamid is a fungicide extensively used in agriculture. Mandipropamid acts as an inducer for the chemically induced proximity system of the invention. Suitably in a similar manner to that of ABA, effectively Mandipropamid induces heterodimerization of the Mandipropamid binding proteins ABI1 and PYR^{Mandi} and heterodimerization of ABI1 and PYLcs^{Mandi}. Suitably therefore Mandipropamid may function when used with the ABA inducible proximity system described herein. Suitably Mandipropamid is a plant hormone analogue.

In a suitable embodiment Mandipropamid is used in the invention.

### Gibberellin

Gibberellins are plant hormones that play a crucial role in regulating various aspects of plant growth and development and a wide range of physiological processes in plants, including seed germination, stem elongation, leaf expansion, flowering and fruit development. Gibberellin acts as a plant hormone inducer for the chemically induced proximity system of the invention. Suitably, gibberellin induces heterodimerization of the gibberellin binding proteins GID1 and GAI, and heterodimerization of GID1 and a modified GAI protein (i.e. nucleotides 1-276 (amino acids 1-92) of the full length GAI protein).

Suitably the term gibberellin may encompass any compound of the gibberellin family, or precursors or mimics thereof.

Suitably the gibberellin may be selected from any one of: GA1, GA3, GA4, and GA7, for example.

In a suitable embodiment gibberellin GA3 is used in the invention.

### Protein of Interest

In the context of the present invention, a protein of interest may be any protein expressed or presented intracellularly, within a cell, or on the surface of a cell. Suitably, in some embodiments, the protein of interest is expressed or presented on the surface of a cell.

Suitably, the protein of interest may be an antigen. Suitably an antigen may be regarded as any protein typically expressed or presented on the surface of a cell. Such an antigen may be described herein as target antigen of interest i.e. ‘TAO1’ or ‘TAOI2’.

Suitably, the antigen may be any immunostimulatory antigen. Suitably the antigen may be any antigen to which a therapeutic agent or drug will bind. Such therapeutic agents or drugs are defined herein.

Suitably, a therapeutic agent or drug, such as an immunotherapy, that binds to an antigen of interest may be selected from: a fusion protein, an antibody (e.g. a monoclonal antibody, a bispecific antibody, a multi-specific antibody, an antibody drug conjugate, a nanobody, scFv, di-scFv, Fab, sdAb, F(ab)₂, a glyco-engineered antibody) or a binding fragment thereof, an antibody-like molecule, a fusion protein, an aptamer, an ankyrin, an ankyrin repeat protein (DARPin), a peptide, a bicycle peptide, a small molecule, a vaccine, a T-Cell, a Natural Killer (NK) cell, a cell expressing a CAR such as a CAR T-cell or a CAR NK cell, an Oncolytic virus, a cytokine, a chemokine, a hormone, a bacterium, a tumour infiltrating lymphocyte, a dendritic cell, a macrophage or a mesenchymal cell.

Suitably the therapeutic agent or drug is a biologic. Suitably the therapeutic or drug is an immunotherapy. Suitably, an immunotherapy that binds to an antigen of interest. In some embodiments, the immunotherapy may be an antibody or binding fragment thereof.

In other embodiments, an immunotherapy that binds to an antigen of interest may be an immune cell, such as for example a T-cell, an NK cell, a B-cell, a tumour infiltrating lymphocyte, a dendritic cell, a macrophage, a mesenchymal cell, or immortalised cells thereof. Suitably an immunotherapy which is an immune cell may also be an engineered immune cell. Suitably an engineered immune cell may express a CAR. Suitably therefore an engineered immune cell may be selected from a CAR T-cell, TCR T-cell, CAR B-cell, CAR-macrophage or a CAR NK cell.

In a suitable embodiment, the antigen of interest may be any antigen which is a therapeutic target. By way of example, the antigen may be, CD19, BCMA, CD123, mesothelin, GD2, CD20, CD33, CD47, HER2, CD22, CD13, PSMA, EGFR vIII, EGFR, CD38, EpCAM, PSCA, CEA, HIV, Glypican-3, FLT3, NKG2D, claudin 18.2, DLL3, CS1, MUC16, CD3, PD-L1, 4-1BB, PD-1, LAG3, CTLA-4, MUC1, 5T4, CD40, CD155, OX-40, NY-ESO, ROR1, TROP2, VEGFR1, VEGFRII, CLL, CD30, CD70, CD133, TIM-3, L1CAM, ICOS, DLL4, FRalpha, WT1, IL13Ralpha, Lewis-Y or cMET.

In one embodiment, the first antigen of interest may be CD19 and the second antigen of interest may be CD22.

In one embodiment, the first antigen of interest may be CD38 and the second antigen of interest may be BCMA.

In one embodiment, the first antigen of interest may be PD-L1 and the second antigen of interest may be HER2.

In one embodiment, the first antigen of interest may be HER2 and the second antigen of interest may be HER3.

In one embodiment, the first antigen of interest may be CD13 and the second antigen of interest may be TIM3.

In one embodiment, the first antigen of interest may be CD155 and the second antigen of interest may be PD-L1.

In one embodiment, the first antigen of interest may be CD19 and the second antigen of interest may be CD20.

In one embodiment, the first antigen of interest may be EGFR and the second antigen of interest may be MET.

In one embodiment, the first antigen of interest may be PD-1 and the second antigen of interest may be ICOS.

Suitably, therefore, the immunotherapy may be an antibody selected from an: anti-CD19, anti-BCMA, anti-CD123, anti-mesothelin, anti-GD2, anti-CD20, anti-CD33, anti-HER2, anti-CD22, anti-CD30, anti-PSMA, anti-EGFR vIII, anti-EGFR, anti-CD38, anti-EpCAM, anti-PSCA, anti-CEA, anti-HIV, anti-Glypican-3, anti-FLT3, anti-NKG2D, anti-claudin 18.2, anti-DLL3, anti-CS1, anti-MUC16, anti-CD3, anti-PD-L1, anti-4-1BB, anti-PD-1, anti-LAG3, anti-CTLA-4, anti-MUC1, anti-5T4, anti-CD40, anti-OX-40, anti-NY-ESO, anti-ROR1, anti-TROP2, anti-VEGFRII, anti-CLL, anti-CD30, anti-CD70, anti-CD133, anti-TIM-3, anti-L1CAM, anti-ICOS, anti-DLL4, anti-FRalpha, anti-WT1, anti-IL13Ralpha, anti-Lewis-Y or anti-cMET antibody, or binding fragment thereof.

In some embodiments therefore, the immunotherapy may be one or more antibodies directed towards one or both of the first and second antigens in the pairs listed above.

Suitably the antigen is an antigen associated with a disease or disorder.

Suitably the antigen may be associated with any disease or disorder, for example infectious diseases, autoimmune diseases, inflammatory diseases, cancers, hereditary or genetic diseases.

Suitable infectious diseases may include viral, bacterial, fungal, or protozoan infections.

Suitable viral infections include: COVID-19, SARS, MERS, influenza, common cold, respiratory syncytial virus infection, adenovirus infection, parainfluenza virus infection, norovirus infection, rotavirus infection, astrovirus infection, measles, mumps, rubella, chickenpox, shingles, roseola, smallpox, fifth disease, chikungunya virus infection, HPV infection, Hepatitis A, B, C, D or E, warts, herpes, molluscum contagiosum, ebola, lassa fever, dengue fever, yellow fever, Marburg hemorrhagic fever, Crimean-Congo hemorrhagic fever, polio, viral meningitis, viral encephalitis, rabies, zika virus infection, west nile virus infection, HIV/AIDS, Hantavirus infection, HPS.

Suitable bacterial infections include: urinary tract infections, cystitis, impetigo, bacterial food poisoning, campylobacteriosis, C. difficile infection, bacterial cellulitis, MRSA, CRPA, VRSA, sepsis, erysipelas, necrotising fasciitis, bacterial folliculitis, gonorrhoea, chlamydia, syphilis, Mycoplasma genitalium, bacterila vaginosis, pelvic inflammatory disease, tuberculosis, whooping cough, Haemophilus influenzae disease, pneumonia, bacterial meningitis, lyme disease, cholera, botulism, tetanus, anthrax, Cryptosporidiosis, Diphtheria, E. coli infection, Legionnaires Disease, Leptospirosis, Listeriosis, salmonella infections, Shigellosis gastroenteritis, Staphylococcal infections, Streptococcal infections, TSS, typhoid fever, Yersenia infection.

Suitable autoimmune diseases include: asthma, psoriasis, MS, rheumatoid arthritis, reactive arthritis, lupus, inflammatory bowel syndrome/disease, type 1 diabetes, Guillain-Barre syndrome, demyelinating polyneuropathy, Graves' disease, Hashimo's thyroiditis, Myasthenia gravis, vasculitis, pernicious anemia, ulcerative colitis, antiphospholipid syndrome, Kawasaki disease, alopecia, vitiligo, scleroderma, Sjogren's syndrome, crohn's disease, coeliac disease, Addison's disease, narcolepsy.

Suitable cardiovascular diseases include: angina, heart attack, heart failure, coronary heart disease, stroke, transient ischemic attack, peripheral arterial disease, aortic disease, atherosclerosis, hypertension, cerebrovascular disease, renal artery stenosis, aneurysm, cardiomyopathy, pulmonary heart disease, arrythmia, dysrhythmia, endocarditis, cardiomegaly, myocarditis, valvular heart disease, congenital heart disease, rheumatic heart disease.

Suitable inflammatory diseases may include any of the above infections or autoimmune diseases. Suitable inflammatory diseases may include include: arthritis, asthma, tuberculosis, periodontis, chronic ulcers, sinusitis, hepatitis, glomerulonephritis, inflammatory bowel syndrome/disease, preperfusion injury, transplant rejection, sickle cell disease, allergies, cardiovascular disease, psoriasis, cytokine-mediated pruritus, COPD, diabetes, bronchitis, Crohn's disease, atherosclerosis, dermatitis, arteritis, lupus.

Suitable cancers include: breast cancer, liver cancer, lung cancer, pancreatic cancer, brain cancer, prostate cancer, bowel cancer, rectal cancer, bone cancer, leukemia, bladder cancer, cervical cancer, endometrial cancer, eye cancer, retinoblastoma, ewing sarcoma, gallbladder cancer, head and neck cancer, kaposi's sarcoma, kidney cancer, laryngeal cancer, mesothelioma, myeloma, lymphoma, ovarian cancer, oesophageal cancer, mouth cancer, nasopharyngeal cancer, nose and sinus cancer, skin cancer, sarcoma, stomach cancer, testicular cancer, thyroid cancer, uterine cancer, vaginal cancer, penile cancer, vulval cancer.

Suitably the antigen may be associated with a disease in any system of the body, for example: a neurological disease, cardiovascular disease, blood disease, skin disease, gastrointestinal disease, muscular disease, skeletal disease, respiratory disease, reproductive disease, urinary disease, or endocrine disease.

Suitably the antigen is an antigen associated with cancer. Suitably the antigen is an antigen associated with tumours, suitably with cancerous tumours. Suitably therefore the antigen may be a tumour associated antigen (TAA) or a tumour restricted antigen (TRA).

In one embodiment, the antigen is a tumour associated antigen (TAA). As described hereinabove, TAAs are associated not only with tumour cells but also with healthy cells.

Suitably the present invention provides a means to assess binding of candidate therapeutic agents, which may be any of those listed above, to TAAs, and thereby their binding and biological activity on both tumour cells and healthy cells. In some embodiments, the first and second proteins of interest may be first and second TAAs respectively.

### First Construct

A first construct in accordance with the present invention comprises a promoter operably linked to a nucleic acid sequence encoding a first chimeric protein, and a second chimeric protein. Suitably therefore the first construct is a first nucleic acid construct.

Suitably, the promotor may be a mammalian cell promoter. In a suitable embodiment of the invention, the mammalian cell promotor is selected from: MND, CAG EF1-α, CMV, MSCV, SV40, mouse PGK, human PGK or UBC. In one embodiment the promoter is MND.

Suitably, the promoter of the first construct of the invention is operably linked to the nucleic acid sequence encoding a first chimeric protein, and is operably linked to the nucleic acid sequence encoding a second chimeric protein.

By “operably linked” as used herein, it is meant that the indicated elements are functionally related to each other, and are also generally physically related. Thus, the term “operably linked” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably linked with a nucleotide sequence if the promoter effects the transcription or expression of said nucleotide sequence. Those skilled in the art will appreciate that the control sequences (e.g., promoter) need not be contiguous with the nucleotide sequence to which it is operably linked, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

Operably linked” means joined as part of the same nucleic acid molecule, suitably positioned and oriented for transcription to be initiated from the promoter. DNA operably linked to a promoter is under transcriptional initiation regulation of the promoter or in functional combination therewith.

Suitably therefore the first construct is a bicistronic construct in that it comprises a first and a second nucleic acid sequence which may each be regarded as a cistron.

In some embodiments, the first construct in accordance with the present invention may further comprise a cleavable linker.

Suitably, the cleavable linker links the nucleic acid sequence encoding the first chimeric protein to the nucleic acid sequence encoding the second chimeric protein. Suitably the cleavable linker is located between the nucleic acid sequence encoding the first chimeric protein and the nucleic acid sequence encoding the second chimeric protein

Suitably, the cleavable linker is a nucleic acid sequence encoding a self-cleaving peptide. Suitably the self-cleaving peptide links the first chimeric protein and the second chimeric protein. Suitably when the first construct is transcribed, the linker is automatically cleaved such that the first chimeric protein and the second chimeric protein are separated.

Suitably, the cleavable linker may be a 2A self-cleaving peptide. Suitably the 2A self cleaving peptide may be a teschovirus-1 2A (P2A) self-cleaving peptide, a foot and mouth (F2A) self-cleaving peptide, an equine rhinitis (E2A) self cleaving peptide, or a thosea asigna (T2A) self-cleaving peptide. Suitably the self cleaving peptide may be derived from any specific virus of the groups listed above. In one embodiment, the cleavable linker is a porcine teschovirus P2A self-cleaving peptide.

In an alternative embodiment, the first nucleic acid construct in accordance with the present invention may further comprise an IRES.

Suitably the IRES is positioned between the nucleic acid sequence encoding the first chimeric protein and the nucleic acid sequence encoding the second chimeric protein. Suitably the IRES ensures that the first nucleic acid sequence is translated separately from the second nucleic acid sequence to form separate first and second chimeric proteins.

Suitably the IRES sequence may be selected from any suitable viral or cellular IRES, such as of the encephalomyocarditis virus (EMCV).

In one embodiment, the IRES comprises a sequence according to SEQ ID NO:4. In one embodiment, the IRES consists of a sequence according to SEQ ID NO:4.

Suitably, the first chimeric protein and the second chimeric protein encoded by the nucleic acid sequence of the first construct of the invention each comprise an auxin binding domain and an effector domain. Thus, the first construct comprises a nucleic acid sequence encoding two auxin binding domains and two effector domains.

Suitably, the auxin binding domain of either of the first and second chimeric proteins encoded by the nucleic acid sequence of the first construct of the invention may be Transport inhibitor response 1 protein (TIR1), or fragment or derivative thereof. Thus, the first construct comprises a nucleic acid sequence encoding a TIR1 protein, or fragment or derivative thereof.

Suitably, the auxin binding domain of the first or second chimeric proteins encoded by the nucleic acid sequence of the first construct of the invention may be Auxin/indole-3-acetic acid protein (AID). Thus the first construct comprises a nucleic acid sequence encoding an AID protein, or fragment or derivative thereof.

Alternatively, the first chimeric protein and the second chimeric protein encoded by the nucleic acid sequence of the first construct of the invention each comprise a caffeine binding domain and an effector domain. Thus, the first construct comprises a nucleic acid sequence encoding two caffeine binding domains and two effector domains.

Suitably, the caffeine binding domain of both of the first and second chimeric proteins encoded by the nucleic acid sequence of the first construct of the invention may be an anti-caffeine heavy-chain antibody fragment (aCaffVHH) or fragment or derivative thereof. Thus, the first construct comprises a nucleic acid sequence encoding two aCaffVHH proteins, or fragments or derivatives thereof.

Alternatively, the first chimeric protein and the second chimeric protein encoded by the nucleic acid sequence of the first construct of the invention each comprise a mandipropamid binding domain and an effector domain. Thus, the first construct comprises a nucleic acid sequence encoding two Mandipropamid binding domains and two effector domains.

Suitably, the Mandipropamid binding domain of either of the first and second chimeric proteins encoded by the nucleic acid sequence of the first construct of the invention may be a PYR^{Mandi} or a PYLcs^{Mandi} protein, or a fragment or derivative thereof. Thus, the first construct comprises a nucleic acid sequence encoding a PYR^{Mandi}, or a PYLcs^{Mandi} protein, or fragments or derivatives thereof.

Suitably, the mandipropamid binding domain of the first or second chimeric proteins encoded by the nucleic acid sequence of the first construct of the invention may be ABI1. Thus the first construct comprises a nucleic acid sequence encoding an ABI1 protein, or fragment or derivative thereof.

Alternatively, the first chimeric protein and the second chimeric protein encoded by the nucleic acid sequence of the first construct of the invention each comprise a gibberellin binding domain and an effector domain. Thus, the first construct comprises a nucleic acid sequence encoding two gibberellin binding domains and two effector domains.

Suitably, the gibberellin binding domain of either of the first and second chimeric proteins encoded by the nucleic acid sequence of the first construct of the invention may be a GID1 protein, or a fragment or derivative thereof. Thus, the first construct comprises a nucleic acid sequence encoding a GID1 protein, or fragments or derivatives thereof.

Suitably, the gibberellin binding domain of the first or second chimeric proteins encoded by the nucleic acid sequence of the first construct of the invention may be GAI protein or a modified GAI protein. Thus the first construct comprises a nucleic acid sequence encoding an GAI protein, or fragment or derivative thereof.

Suitably, the effector domain of either of the first and second chimeric proteins encoded by the nucleic acid sequence of the first construct of the invention may be a transactivation domain. Thus, the first construct comprises a nucleic acid sequence encoding transactivation domain.

Suitably, the effector domain of either of the first and second chimeric proteins encoded by the nucleic acid sequence of the first construct of the invention may be a catalytically inactive I-Scel endonuclease DNA binding domain (dl-Scel). Thus, the first construct comprises a nucleic acid sequence encoding dl-Scel DNA binding domain.

In the context of the present invention, the auxin binding domain and the effector domain of the first and second chimeric proteins are different.

Suitably, therefore, the nucleic acid sequence encoding the transactivation domain and the nucleic acid sequence encoding dl-Scel DNA binding domain are each linked to one of the nucleic acid sequences encoding TIR1 or AID proteins, or the other chemical binding domains noted above, in a mutually exclusive manner to produce the first and a second chimeric proteins.

In one embodiment, the nucleic acid sequence encoding the transactivation domain is linked to the nucleic acid sequence encoding a TIR1 protein, the aCaffVHH protein, the PYR^{Mandi} the PYLcs^{Mandi} protein, or the GID1 protein. These linked nucleic acid sequences encode a first chimeric protein comprising a transactivation domain fused to a TIR1 protein, an aCaffVHH protein, a PYR^{Mandi}, a PYLcs^{Mandi} protein, or a GID1 protein.

In one embodiment, the nucleic acid sequence encoding the dl-Scel DNA binding domain is linked to the nucleic acid sequence encoding an AID protein, the aCaffVHH protein, the ABI1 protein, or the GAI protein or a modified GAI protein. These linked nucleic acid sequences encode a second chimeric protein comprising the dl-Scel DNA binding domain fused to an AID protein, αCaffVHH protein, ABI1 protein, or GAI protein or a modified GAI protein.

Suitably in such an embodiment, the first chimeric protein comprises a transactivation domain and a TIR1 protein, and the second chimeric protein comprises the dl-Scel DNA binding domain and an AID protein.

In alternative embodiments, the first chimeric protein comprises a transactivation domain and a aCaffVHH protein, a PYR^{Mandi} protein, a PYLcs^{Mandi} protein, or a GID1 protein, and the second chimeric protein comprises the dl-Scel DNA binding domain and an aCaffVHH protein, an ABI1 protein, or a GAI protein or a modified GAI protein.

Alternatively, in another embodiment, the nucleic acid sequence encoding the transactivation domain is linked to the nucleic acid sequence encoding an AID protein. These linked nucleic acid sequences encode a first chimeric protein comprising a transactivation domain fused to an AID protein.

Alternatively, in another embodiment, the nucleic acid sequence encoding the dl-Scel DNA binding domain is linked to the nucleic acid sequence encoding a TIR1 protein. These linked nucleic acid sequences encode a second chimeric protein comprising the dl-Scel DNA binding domain fused to a TIR1 protein.

Suitably in such an embodiment, the first chimeric protein comprises a transactivation domain and an AID protein, and the second chimeric protein comprises the dl-Scel DNA binding domain and a TIR1 protein.

Equally therefore, the nucleic acid sequence encoding the transactivation domain may be linked to the nucleic acid sequence encoding the aCaffVHH protein, the ABI1 protein, or the GAI protein or a modified GAI protein. Equally therefore, the nucleic acid sequence encoding the dl-Scel DNA binding domain may be linked to the nucleic acid sequence encoding the aCaffVHH protein, the PYR^{Mandi}, the PYLcs^{Mandi} protein, or the GID1 protein.

It will be appreciated, as explained elsewhere herein that the DNA binding domains used in the first construct and the third construct, and consequently their binding sites in the second and fourth constructs are interchangeable. In some embodiments, the first construct suitably the second chimeric protein thereof may comprise a GAL4 DNA binding domain, and the second construct may comprise one or more GAL4 upstream activation sequences.

In the context of the present invention, the first and second chimeric proteins dimerize in the presence of auxin, or in the presence of caffeine, mandipropamid, or gibberellin. Suitably, the presence of auxin, or the other compounds, allows the transactivation domain to associate with the dl-Scel DNA binding domain, or the GAL4 DNA binding domain if this is used.

Suitably, dimerization of the first and second chimeric proteins of the invention stimulates transcription. Suitably transcription of the second construct. Suitably transcription of the second construct from the effector domain binding site, suitably from the dl-Scel binding site, or from the GAL4 UAS if this is used. Suitably, dimerization of the first and second chimeric proteins of the invention thereby stimulates transcription of the first protein of interest, suitably transcription of the first protein of interest from the second construct.

It will be appreciated that a nucleic acid construct in accordance with the invention may comprise DNA or RNA. It will be appreciated that a suitable nucleic acid construct may essentially consist of DNA, may essentially consist of RNA, or may comprise a combination of DNA and RNA.

### Second Construct

A second construct in accordance with the present invention comprises a nucleic acid sequence encoding one or more effector domain binding sites and a nucleic acid sequence encoding a protein of interest. Suitably therefore the second construct is a second nucleic acid construct.

Suitably, the nucleic acid sequence encoding one or more effector domain binding sites is operably linked to the nucleic acid sequence encoding a protein of interest. The term ‘operably linked’ is defined hereinabove.

Suitably, the one or more effector domain binding sites is suitable for the chosen effector domain to bind thereto. Suitably, given that the preferred effector domain is a dl-Scel, the effector domain binding site is an IScel DNA binding site.

Suitably, each IScel DNA binding site comprises the sequence; TAGGGATAACAGGGTAAT (SEQ ID NO: 1).

Suitably, each dl-Scel DNA binding site is an 18 bp sequence consisting of the sequence; TAGGGATAACAGGGTAAT (SEQ ID NO: 1).

Suitably, the second (or fourth) construct may comprise one, or more than one effector domain binding sites. Suitably more than one IScel DNA binding sites, suitably a plurality of IScel DNA binding sites.

Suitably, the more than one effector domain binding sites, preferably IScel DNA binding sites, are in tandem.

Suitably, the second (or fourth) construct comprises between 1 to 15 effector domain binding sites, suitably arranged in tandem. Suitably the second construct comprises one IScel DNA binding site, two IScel DNA binding sites, three IScel DNA binding sites, four IScel DNA binding sites, five IScel DNA binding sites, six IScel DNA binding sites, seven IScel DNA binding sites, eight IScel DNA binding sites, nine IScel DNA binding sites, ten IScel DNA binding sites, eleven IScel DNA binding sites, twelve IScel DNA binding sites, thirteen IScel DNA binding sites, fourteen IScel DNA binding sites or fifteen IScel DNA binding sites. Suitably in tandem.

In a preferred embodiment, the second (or fourth) construct comprises ten IScel DNA binding sites in tandem.

Suitably the second construct may further comprise a promoter. Suitably the second construct may comprise a promoter operably linked to the effector domain binding site, suitably downstream of the effector domain binding site. Suitably the promoter is a minimal promoter. Suitably the minimal promoter may be selected from a minimal TATA-box promoter, a minimal Adenovirus late promoter, a minimal herpes simplex virus (HSV) thymidine kinase promoter or a minimal c-fos promoter (consisting of nucleotides −53 to +42), for example.

Suitably therefore the effector domain binding site may comprise a sequence according to SEQ ID NO:7. Suitably therefore the effector domain binding site may consist of a sequence according to SEQ ID NO:7. Suitably therefore, the second construct may comprise a sequence according to SEQ ID NO: 7.

A second construct in accordance with the present invention may further optionally comprise a recombination site.

Suitably, the recombination site is positioned upstream of the one or more effector domain binding sites.

In a suitable embodiment, the recombination site is selected from any serine recombinase site. In one embodiment, the recombination site is an attB site.

Suitably the second construct comprises a nucleic acid sequence encoding a protein of interest. A suitable protein of interest is as defined elsewhere in this description. Suitably the nucleic acid sequence encoding a protein of interest is downstream of the one or more effector domain binding sites. Suitably downstream of the one or more IScel DNA binding sites.

In the context of the present invention, a dl-Scel DNA binding domain binds to each of the one or more dl-Scel DNA binding sites. Suitably in some embodiments, the dl-Scel DNA binding domains which are each fused to an auxin binding domain bind to each of the one or more dl-Scel DNA binding sites. Suitably therefore either a first or second chimeric protein comprising the dl-Scel DNA binding domain fused to an auxin binding domain is bound to each of the one or more dl-Scel DNA binding sites.

Suitably, in preferred embodiments, in the presence of auxin, association of the auxin binding domains of the first and second chimeric proteins brings the transactivation domain in proximity to the dl-Scel DNA binding domain bound to the I-Scel binding site in the second construct, and thereby stimulates transcription of the downstream nucleic acid encoding the protein of interest.

It will be appreciated that the effector binding domains of the second and fourth constructs are interchangeable. Suitably therefore the second construct may instead comprise one or more GAL4 upstream activation sequences which interact with a GAL4 DNA binding domain as described hereinbelow in relation to the fourth construct.

It will be appreciated that a nucleic acid construct in accordance with the invention may comprise DNA or RNA. It will be appreciated that a suitable nucleic acid construct may essentially consist of DNA, may essentially consist of RNA, or may comprise a combination of DNA and RNA.

### Third Construct

A third construct in accordance with the present invention comprises a promoter operably linked to a nucleic acid sequence encoding a third chimeric protein, and a fourth chimeric protein. Suitably therefore the third construct is a third nucleic acid construct.

Suitably, the promotor may be a mammalian cell promoter. In a suitable embodiment of the invention, the mammalian cell promotor is selected from: MND, CAG EF1-α, CMV, MSCV, SV40, mouse PGK, human PGK or UBC. In one embodiment the promoter is EF1-α.

Suitably, the promoter of the third construct of the invention is operably linked to the nucleic acid sequence encoding a third chimeric protein, and is operably linked to the nucleic acid sequence encoding a fourth chimeric protein.

The term ‘operably linked’ is as defined above in relation to the first construct.

Suitably therefore the third construct is a bicistronic construct in that it comprises a first and a second nucleic acid sequence which may each be regarded as a cistron.

In some embodiments, the third construct in accordance with the present invention may further comprise a cleavable linker.

Suitably, the cleavable linker links the nucleic acid sequence encoding the third chimeric protein to the nucleic acid sequence encoding the fourth chimeric protein. Suitably the cleavable linker is located between the nucleic acid sequence encoding the third chimeric protein and the nucleic acid sequence encoding the fourth chimeric protein

Suitably, the cleavable linker is a nucleic acid sequence encoding a self-cleaving peptide. Suitably the self-cleaving peptide links the third chimeric protein and the fourth chimeric protein. Suitably when the third construct is transcribed, the linker is automatically cleaved such that the third chimeric protein and the fourth chimeric protein are separated.

Suitably, the cleavable linker may be a 2A self-cleaving peptide. Suitably the 2A self cleaving peptide may be a teschovirus-1 2A (P2A) self-cleaving peptide, a foot and mouth (F2A) self-cleaving peptide, an equine rhinitis (E2A) self cleaving peptide, or a thosea asigna (T2A) self-cleaving peptide. Suitably the self cleaving peptide may be derived from any specific virus of the groups listed above. In one embodiment, the cleavable linker is a porcine teschovirus P2A self-cleaving peptide.

In an alternative embodiment, the third nucleic acid construct in accordance with the present invention may further comprise an IRES, suitably the IRES may be as described above in relation to the first construct.

Suitably, the third chimeric protein and the fourth chimeric protein encoded by the nucleic acid sequence of the third construct of the invention each comprise an abscisic acid binding domain and an effector domain. Thus, the third construct comprises a nucleic acid sequence encoding two abscisic acid binding domains and two effector domains.

Suitably, the abscisic acid binding domain of either of the third and fourth chimeric proteins encoded by the nucleic acid sequence of the third construct of the invention may be an ABI1 protein, or fragment or derivative thereof. Thus, the third construct comprises a nucleic acid sequence encoding an ABI1 protein, or fragment or derivative thereof.

Suitably, the abscisic acid binding domain of the third or fourth chimeric proteins encoded by the nucleic acid sequence of the third construct of the invention may be a PYL1 protein, or fragment or derivative thereof. Thus the third construct comprises a nucleic acid sequence encoding a PYL1 protein, or fragment or derivative thereof.

Suitably, the effector domain of either of the third and fourth chimeric proteins encoded by the nucleic acid sequence of the third construct of the invention may be a transactivation domain. Thus, the third construct comprises a nucleic acid sequence encoding transactivation domain.

Suitably, the effector domain of either of the third and fourth chimeric proteins encoded by the nucleic acid sequence of the third construct of the invention may be a GAL4 DNA binding domain. Thus, the third construct comprises a nucleic acid sequence encoding a GAL4 DNA binding domain.

In the context of the present invention, the abscisic acid binding domain and the effector domain of the third and fourth chimeric proteins are different.

Suitably, therefore, the nucleic acid sequence encoding the transactivation domain and the nucleic acid sequence encoding GAL4 DNA binding domain are each linked to one of the nucleic acid sequences encoding an ABI1 or a PYL1 protein in a mutually exclusive manner to produce the third and fourth chimeric proteins.

In one embodiment, the nucleic acid sequence encoding the transactivation domain is linked to the nucleic acid sequence encoding a PYL1 protein. These linked nucleic acid sequences encode a third chimeric protein comprising a transactivation domain fused to a PYL1 protein.

In one embodiment, the nucleic acid sequence encoding the GAL4 DNA binding domain is linked to the nucleic acid sequence encoding an ABI1 protein. These linked nucleic acid sequences encode a fourth chimeric protein comprising the GAL4 DNA binding domain fused to an ABI1 protein.

Suitably in such an embodiment, the third chimeric protein comprises a transactivation domain and a PYL1 protein, and the fourth chimeric protein comprises the GAL4 DNA binding domain and an ABI1 protein.

Alternatively, in another embodiment, the nucleic acid sequence encoding the transactivation domain is linked to the nucleic acid sequence encoding an ABI1 protein. These linked nucleic acid sequences encode a third chimeric protein comprising a transactivation domain fused to an ABI1 protein.

Alternatively, in another embodiment, the nucleic acid sequence encoding the GAL4 DNA binding domain is linked to the nucleic acid sequence encoding a PYL1 protein. These linked nucleic acid sequences encode a fourth chimeric protein comprising the GAL4 DNA binding domain fused to a PYL1 protein.

Suitably in such an embodiment, the third chimeric protein comprises a transactivation domain and an ABI1 protein, and the fourth chimeric protein comprises the GAL4 DNA binding domain and a PYL1 protein.

It will be appreciated, as explained elsewhere herein that the DNA binding domains used in the first construct and the third construct, and consequently their binding sites in the second and fourth constructs are interchangeable. In some embodiments, the third construct, suitably the fourth chimeric protein thereof, may comprise a dl-Scel DNA binding domain, and the fourth construct may comprise one or more dl-Scel binding sites.

In the context of the present invention, the third and fourth chimeric proteins dimerize in the presence of abscisic acid. Suitably, the presence of abscisic acid allows the transactivation domain to associate with the GAL4 DNA binding domain.

Suitably, dimerization of the third and fourth chimeric proteins of the invention stimulates transcription. Suitably transcription of the fourth construct. Suitably transcription of the fourth construct from the effector domain binding site, suitably from the GAL4 upstream activation sequence. Suitably, dimerization of the third and fourth fusion proteins of the invention thereby stimulates transcription of the protein of interest, suitably transcription of the protein of interest from the fourth construct.

It will be appreciated that a nucleic acid construct in accordance with the invention may comprises DNA or RNA. It will be appreciated that a suitable nucleic acid construct may essentially consist of DNA, may essentially consist of RNA, or may comprise a combination of DNA and RNA.

### Fourth Construct

A fourth construct in accordance with the present invention comprises a nucleic acid sequence encoding one or more effector domain binding sites and a nucleic acid sequence encoding a protein of interest. Suitably therefore the fourth construct is a fourth nucleic acid construct.

Suitably, the nucleic acid sequence encoding one or more effector domain binding sites is operably linked to the nucleic acid sequence encoding a protein of interest. The term ‘operably linked’ is defined hereinabove.

Suitably, the one or more effector domain binding sites is suitable for the chosen effector domain to bind thereto. Suitably, given that the preferred effector domain is a GAL4 DNA binding domain, the effector domain binding site is an GAL4 DNA binding site, otherwise known as a GAL4 upstream activation sequence.

Suitably, each GAL4 upstream activation sequence comprises the sequence: CGGAGTACTGTCCTCCG (SEQ ID NO:28)

Suitably, each is GAL4 upstream activation sequence a 17 bp sequence consisting of the sequence; CGGAGTACTGTCCTCCG (SEQ ID NO:28)

Suitably, the fourth (or second) construct may comprise one, or more than one effector domain binding sites. Suitably more than one GAL4 upstream activation sequence, suitably a plurality of GAL4 upstream activation sequences.

Suitably, the more than one effector domain binding sites, preferably GAL4 upstream activation sequences, are in tandem.

Suitably, the fourth (or second) construct comprises between 1 to 15 effector domain binding sites, suitably arranged in tandem. Suitably the second or fourth construct comprises one GAL4 upstream activation sequence, two GAL4 upstream activation sequences, three GAL4 upstream activation sequences, four GAL4 upstream activation sequences, five GAL4 upstream activation sequences, six GAL4 upstream activation sequences, seven GAL4 upstream activation sequences, eight GAL4 upstream activation sequences, nine GAL4 upstream activation sequences, ten GAL4 upstream activation sequences, eleven GAL4 upstream activation sequences, twelve GAL4 upstream activation sequences, thirteen GAL4 upstream activation sequences, fourteen GAL4 upstream activation sequences or fifteen GAL4 upstream activation sequences. Suitably in tandem.

In a preferred embodiment, the fourth (or second) construct comprises nine GAL4 upstream activation sequences in tandem.

Suitably the fourth construct may further comprise a promoter. Suitably the fourth construct may comprise a promoter operably linked to the effector domain binding site, suitably downstream of the effector domain binding site. Suitably the promoter is a minimal promoter. Suitably the minimal promoter may be selected from a minimal TATA-box promoter, a minimal Adenovirus late promoter, a minimal herpes simplex virus (HSV) thymidine kinase promoter or a minimal c-fos promoter (consisting of nucleotides-53 to +42), for example.

Suitably therefore the effector domain binding site may comprise a sequence according to SEQ ID NO: 19. Suitably therefore the effector domain binding site may consist of a sequence according to SEQ ID NO:19. Suitably therefore, the second construct may comprise a sequence according to SEQ ID NO: 19.

A fourth construct in accordance with the present invention may further optionally comprise a recombination site.

Suitably, the recombination site is positioned upstream of the one or more effector domain binding sites.

In a suitable embodiment, the recombination site is selected from any serine recombinase site. In one embodiment, the recombination site is an attB site.

Suitably the fourth construct comprises a nucleic acid sequence encoding a protein of interest. A suitable protein of interest is as defined elsewhere in this description. Suitably the nucleic acid sequence encoding a protein of interest is downstream of the one or more effector domain binding sites. Suitably downstream of the one or more GAL4 upstream activation sequences.

In the context of the present invention, a GAL4 DNA binding domain binds to each of the one or more GAL4 upstream activation sequences. Suitably in one embodiment, the GAL4 DNA binding domains which are each fused to an abscisic acid binding domain bind to each of the one or more GAL4 upstream activation sequences. Suitably therefore, in a preferred embodiment either a third or fourth chimeric protein comprising the GAL4 DNA binding domain fused to an abscisic acid binding domain is bound to each of the one or more GAL4 upstream activation sequences.

Suitably, in preferred embodiments in the presence of abscisic acid, association of the abscisic acid binding domains of the third and fourth chimeric proteins brings the transactivation domain in proximity to the GAL4 DNA binding domain bound to the GAL4 upstream activation sequence in the fourth construct, and thereby stimulates transcription of the downstream nucleic acid encoding the protein of interest.

It will be appreciated that the effector binding domains of the second and fourth constructs are interchangeable. Suitably therefore the fourth construct may comprise one or more dl-Scel binding sites which interact with a dl-Scel DNA binding domain as described hereinabove in relation to the second construct.

It will be appreciated that a nucleic acid construct in accordance with the invention may comprises DNA or RNA. It will be appreciated that a suitable nucleic acid construct may essentially consist of DNA, may essentially consist of RNA, or may comprise a combination of DNA and RNA.

### Cells

The first, second, third and fourth constructs according to the invention may be introduced into any cell. Suitably therefore the invention relates to a cell comprising the first, second, third and/or fourth constructs, preferably all of the first, second, third and fourth constructs.

Suitably therefore the invention relates to a cell comprising a first chemically inducible proximity system, and/or a second chemically inducible proximity system, preferably a first plant hormone or hormone analogue inducible system, preferably the auxin inducible system of the invention, the caffeine inducible system, or the mandipropamid inducible system, or the gibberellin inducible system, and/or a second plant hormone or hormone analogue inducible system, preferably the abscisic acid inducible system of the invention. Suitably in accordance with the eighth aspect of the invention.

Suitably the cell may be regarded as a host cell.

Suitably, the cell comprising the nucleic acid constructs according to the invention may be an insect, animal, plant, fungal, bacterial, or archaeon cell. Suitably the cell is an animal cell. Suitably the cell is a mammalian cell. Suitably the cell may be a human cell or a non-human cell. Suitably the cell may be a monkey, dog, cat, mouse, rat, pig, or other animal cell.

Suitably the cell may be an immortalised cell or a primary cell. Suitably the cell may be an immortalised mammalian cell. Suitably the cell may be an immortalised human or monkey cell. Suitably, the cell comprising the nucleic acid constructs according to the invention may be any mammalian cell line but preferably a HEK293 or CHO-K1 cell.

Suitably, the cell comprising the nucleic acid constructs according to the invention may mimic in vivo healthy cells or healthy tissue.

Suitably, the cell comprising the nucleic acid constructs according to the invention may mimic in vivo diseased cells or diseased tissue.

### Vectors

A construct of the invention may be provided or introduced into a cell in the form of a vector. Suitably, the first, second, third and/or fourth constructs of the invention may be provided or introduced upon a vector.

Generally, the term “vector” herein refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art.

Suitably therefore a further aspect of the invention is a vector comprising the first and/or second construct, or the first plant hormone inducible system, preferably the Auxin inducible system, or the caffeine inducible system, or the mandipropamid inducible system, or the gibberellin inducible system described herein. Suitably therefore further provided herein is a vector comprising the third and/or fourth construct, or the second plant hormone inducible system, preferably the Abscisic Acid inducible system described herein.

Suitably one or more vectors may comprise the first second, third and/or fourth constructs.

In one embodiment, there is provided a vector comprising the first and third constructs which may be referred to as the Induction vector.

In one embodiment there is provided a vector comprising the second and fourth constructs which may be referred to as the Delivery vector.

Some vectors are able to direct expression of genes to which they are operatively-linked. Such vectors are “expression vectors” and there will usually be regulatory elements, which may be selected on the basis of the host cells in which the expression takes place. This means the nucleic acid to be expressed is operably linked to the regulatory elements thereby resulting in expression of the nucleotide sequence whether in an in vitro transcription/translation system or in a host cell when the vector is introduced into the host cell.

The term “operably linked” is described elsewhere in the specification.

Suitably or each vector comprising one or more of the constructs of the invention further comprise one or more regulatory sequences. Suitably the regulatory sequences are operably linked to the nucleic acid sequences comprised within the or each constructs.

Suitable regulatory sequences control expression of a nucleic acid sequence with the construct and may include promoters, enhancers, terminators, internal ribosomal entry sites (IRES), and other expression control elements (e.g., transcription termination signals, such as polyadenylation signals and poly-U sequences) UTRs, ITRs, introns etc For more information the average skilled person would refer to, for example, in Goeddel, (1990), Gene Expression Technology in Methods in Enzymology vol 185, Academic Press. Regulatory elements include those giving direct constitutive expression in many types of host cell and those that direct expression of the nucleotide sequence only in certain cells (i.e., tissue-specific regulatory sequences).

A tissue-specific promoter directs expression primarily in a desired tissue of interest, such as blood, specific organs (e.g., liver, pancreas), or particular cell types. Regulatory elements may also direct expression in a temporal-dependent manner, such as in a cell-cycle dependent or developmental stage-dependent manner, which may or may not also be tissue or cell-type specific. A promoter useful with this invention can include, but is not limited to, constitutive, inducible, developmentally regulated, tissue-specific/preferred-promoters, and the like, as described herein.

A regulatory element as used herein can be endogenous or heterologous. In some embodiments, an endogenous regulatory element derived from a subject cell can be inserted into a genetic context in which it does not naturally occur (e.g., a different position in the genome than as found in nature), thereby producing a recombinant or modified nucleic acid. In some embodiments, promoters useful with the constructs described herein may be any combination of heterologous and/or endogenous promoters.

In some embodiments, inducible promoters can be used. Examples of inducible promoters include, but are not limited to, tetracycline repressor system promoters, Lac repressor system promoters, copper-inducible system promoters, salicylate-inducible system promoters (e.g., the PR1a system), glucocorticoid-inducible promoters, and ecdysone-inducible system promoters.

Suitably, the promotor operably linked to the first construct, i.e. operably linked to a nucleic acid sequence encoding a first chimeric protein, and a second chimeric protein is: MND, CAG EF1-α, CMV, MSCV, SV40, mouse PGK, human PGK or UBC. In one embodiment the promoter is MND. as described above.

Suitably the promoter operably linked to the third construct, i.e. operably linked to a nucleic acid sequence encoding a third chimeric protein and a fourth chimeric protein is: MND, CAG EF1-α, CMV, MSCV, SV40, mouse PGK, human PGK or UBC. In one embodiment the promoter is EF1-α.

Suitably the promoter operably linked to the second construct is a minimal promoter. Suitably the minimal promoter may be selected from a minimal TATA-box promoter, a minimal Adenovirus late promoter, a minimal herpes simplex virus (HSV) thymidine kinase promoter or a minimal c-fos promoter (−53 to +42) as described above.

Suitably the promoter operably linked to the fourth construct is a minimal promoter. Suitably the minimal promoter may be selected from a minimal TATA-box promoter, a minimal Adenovirus late promoter, a minimal herpes simplex virus (HSV) thymidine kinase promoter or a minimal c-fos promoter (−53 to +42) as described above.

As well as promoters, regulatory elements may include enhancer elements, such as WPRE; CMV enhancers; the R-U5′ segment in LTR of HTLV-I; SV40 enhancer; and the intron sequence between exons 2 and 3 of rabbit β-globin. It will be appreciated by those skilled in the art that the design of the expression vector can depend on such factors as the choice of the host cell to be transformed, the level of expression desired, etc.

Suitably the vector may also optionally include a transcriptional and/or translational termination region (i.e., termination region) that is functional in the selected host cell. A variety of transcriptional terminators are available and are responsible for the termination of transcription beyond the heterologous nucleotide sequence of interest and correct mRNA polyadenylation. The termination region may be native to the transcriptional initiation region, may be native to the operably linked nucleic acid sequence, may be native to the host cell, or may be derived from another source (i.e., foreign or heterologous to the promoter, to the nucleic acid sequence, to the host, or any combination thereof).

Suitably the vector may also include a nucleotide sequence for a selectable marker, which can be used to select a transformed host cell. As used herein, “selectable marker” means a nucleotide sequence that when expressed imparts a distinct phenotype to the host cell expressing the marker and thus allows such transformed cells to be distinguished from those that do not have the marker. Such a nucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Of course, many examples of suitable selectable markers are known in the art and can be used in the construct described herein.

In some embodiments, a selectable marker useful with this invention includes polynucleotide encoding a polypeptide conferring resistance to an antibiotic. Non-limiting examples of antibiotics useful with this invention include blasticidin, puromycin, hycromycin, and/or erythromycin, for example. Thus, in some embodiments, a polynucleotide encoding a gene for resistance to an antibiotic may be introduced into the cell, thereby conferring resistance to the antibiotic to that cell.

Non-limiting examples of general classes of vectors include but are not limited to a viral vector, a plasmid vector, a phage vector, a phagemid vector, a cosmid vector, a fosmid vector, a bacteriophage, an artificial chromosome, or an Agrobacterium binary vector in double or single stranded linear or circular form which may or may not be self-transmissible or mobilizable. A vector as defined herein can transform a host cell either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). A plasmid may be vector in accordance with this description, which is a circular double stranded DNA loop into which additional DNA segments can be inserted, such as by standard molecular cloning techniques.

Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

Suitably, in some embodiments of the invention, the vector is a viral vector. In some embodiments, the first, second, third and fourth constructs of the invention may be comprised on one or more lentiviral vectors. Suitably comprised in one or more lentiviral particles.

Suitably in accordance with the sixth aspect of the invention, a viral particle comprising the first construct and optionally comprising the third construct may be used, and a viral particle comprising the second construct and optionally comprising the fourth construct may be used. Suitably therefore the induction vector and the delivery vector may be comprised in a viral particle. Suitably said viral particles are lentiviral particles.

In other cases, suitably one or more of the constructs of the invention may be comprised upon a different type of vector, for example a plasmid. Suitably therefore the methods of the invention may use a mix of different vectors to introduce the constructs into a cell.

Suitably, in accordance with the seventh aspect of the invention, the first and third constructs of the invention may be comprised on a Lentiviral vector. Suitably therefore the induction vector may be a lentiviral vector. Suitably said lentiviral vector may be a lentiviral particle. Suitably however the second and fourth constructs of the invention may be comprised on a plasmid. Suitably therefore the delivery vector may be a plasmid.

Suitably the invention, particularly the methods of the invention, may make use of several vectors to modify cells such that they comprise and express the system of the invention. Suitably the methods of the invention may make use of a plurality of vectors.

Suitably in methods of the invention which make use of integration sites within the genome and recombination techniques, further accessory vectors may be required to facilitate such integration.

Suitably in such methods, a vector may be required for generating an integration site or ‘landing pad’ in the genome of a cell ready for insertion of a construct of the invention. Suitably such a vector may be a lentiviral vector. Suitably such a vector comprises a first recombination site which may pair with a second recombination site present in a vector comprising a construct to be inserted in the genome, such as the delivery vector described above.

Suitably such recombination may be catalysed by a recombinase or integrase enzyme, such as a serine integrase. Suitably therefore the recombination sites may be attP and attB.

Suitably in such methods a vector may further be required to express the recombinase or integrase enzyme to catalyse said recombination steps, such as BXB1 integrase.

Method of Making a Cell Comprising the First Plant Hormone Inducible Proximity System and/or the Second Plant Hormone Inducible Proximity System

As discussed elsewhere in the specification, in the context of the present invention, Auxin may act as a plant hormone inducer for a chemically induced proximity system of the invention. Suitably, Auxin induces heterodimerization of TIR1 and AID proteins. Equally, caffeine may act as a plant hormone inducer for a chemically induced proximity system of the invention. Suitably caffeine induces homodimerization of two αCaffVHH proteins. Equally Mandipropamid may act as a plant hormone analogue inducer for a chemically induced proximity system of the invention. Suitably, Mandipropamid induces heterodimerization of PYR^{Mandi} or PYLcs^{Mandi} with ABI1 protein. Equally gibberellin may act as a plant hormone inducer for a chemically induced proximity system of the invention. Suitably, gibberellin induces heterodimerization of GID1 protein and GAI protein, or a fragment thereof.

In an aspect of the invention, there is provided a method of making a cell comprising the first plant hormone or plant hormone analogue inducible proximity system of the invention, suitably an auxin inducible proximity system, a caffeine inducible proximity system, a mandipropamid inducible proximity system, or a gibberellin inducible proximity system.

In an embodiment of the invention the first and/or second construct of the invention are introduced into a cell via one or more vectors.

Furthermore, as described herein abscisic acid may also act as a plant hormone inducer for a chemically induced proximity system of the invention. Suitably abscisic acid induces heterodimerization of ABI1 and PYL1 proteins.

In an aspect of the invention, there is provided a method of making a cell comprising the second plant hormone inducible proximity system of the invention, suitably an abscisic acid inducible system.

In one embodiment, the third and/or fourth construct of the invention are introduced into a cell via one or more vectors.

There is further provided a method of making a cell comprising both the first and second plant hormone inducible systems, suitably both the Auxin inducible proximity system of the invention and the Abscisic Acid inducible proximity system of the invention. Equally both a caffeine inducible proximity system, a Mandipropamid inducible proximity system, or a gibberellin inducible proximity system of the invention, and the Abscisic Acid inducible proximity system of the invention.

In one embodiment, the first, second, third and/or fourth construct of the invention are introduced into a cell via one or more vectors.

Introducing “, “introduce”, “introduced” (and grammatical variations thereof) in the context of a construct of the present invention and a cell means presenting the construct of interest to the cell (e.g., host cell) in such a manner that the construct gains access to the interior of a cell and includes such terms as transformation,” “transfection,” and/or “transduction.” The terms “transformation,” “transfection,” and “transduction” as used herein refer to the introduction of a construct into a cell. Such introduction into a cell may be stable or transient. Thus, in some embodiments, a host cell is stably transformed with the construct. In other embodiments, a host cell is transiently transformed with the construct.

In some embodiments, the first, second, third and/or fourth constructs of the invention may be stably or transiently introduced into a cell.

In some embodiments, the introduction of the first, second, third and/or fourth constructs into the cells is by transient transfection.

Suitably, the nucleic acid sequences of the first, second, third and/or fourth constructs introduced by transient transfection may exist in a cell for a limited time.

Suitably, the nucleic acid sequences of the constructs introduced by transient transfection may exist in a cell for up to 6 hours, up to 12 hours up to 18 hours, up to 24 hours, up to 30 hours, up to 36 hours, up to 42 hours, up to 48 hours or more in the cell.

Suitably, the nucleic acid sequences of the constructs introduced by transient transfection may exist in a cell for up to 1 day, up to 2 days, up to 3 days, up to 4 days, up to 5 days, up to 6 days, up to 7 days, up to 8 days or more in the cell.

Suitably, in such an embodiment, the constructs of the invention are introduced into a cell on vectors which remain extrachromosomal. Suitably, the nucleic acids comprised in the constructs of the invention are not integrated into the genome of the host cell. Suitably the constructs of the invention are expressed directly from the vectors.

The term “genome,” as used herein, refers to both chromosomal and non-chromosomal elements (i.e., extrachromosomal (e.g., mitochondrial, plasmid, and/or extrachromosomal circular DNA (eccDNA)) of a target cell. As used herein, “extrachromosomal” refers to nucleic acid from a mitochondrion, a plasmid, and/or an extrachromosomal circular DNA (eccDNA)).

In other more preferred embodiments, the introduction of the first, second, third and/or fourth constructs may be stably introduced or stably transformed.

As used herein, the term “stably introduced” or “stably transformed” means that the nucleic acid sequence is stably incorporated into the genome of the cell, and thus the cell is stably transformed with the constructs. When a construct is stably transformed and therefore integrated into a cell, the integrated nucleic acid of the construct is capable of being inherited by the progeny thereof, more particularly, by the progeny of multiple successive generations.

Suitably, the first, second, third and/or fourth construct of the invention may be introduced into a cell by stable transformation. Suitably, the first, second, third and/or fourth construct of the invention may be integrated into the genome of the cell.

Suitably introduction of the constructs of the invention into a cell may be achieved by any delivery method known in the art e.g. standard transfection, electroporation, viral-mediated delivery, transposons, gene editing etc.

Suitably, any one or more of the first, second, third and/or fourth constructs of the invention is integrated into the genome of the cell, by using any known technique. Suitably by use of a lentiviral integration or by recombination.

In one embodiment, the constructs of the invention are introduced into the genome by lentiviral integration.

Suitably therefore the method of making a cell comprising the first and/or second plant hormone inducible proximity systems, such as the Auxin inducible proximity system and/or the abscisic acid inducible proximity system, comprises a) introducing a viral vector comprising the first construct, and/or the third construct into the cell, b) introducing a viral vector comprising the second construct, and/or the fourth construct into the cell b) integrating the first and second constructs, and/or the third and fourth constructs into the genome of the cell.

In one embodiment, the method of making a cell comprises both the first plant hormone inducible proximity system, such as the auxin inducible proximity system of the third aspect, and three second plant hormone inducible proximity system, such as an abscisic acid inducible proximity system, comprising:
(a) introducing a viral vector comprising the first and third constructs into the cell;
(b) introducing a viral vector comprising the second and fourth constructs into the cell;
(c) integrating the first, second, third and fourth construct into the genome of the cell

In one embodiment, the first construct and third construct may be contained upon the same construct which may be known as the Induction construct. Therefore step (a) may comprise introducing a viral vector comprising the induction construct into the cell. In one embodiment, the second construct and the fourth construct may be contained upon the same construct which may be known as a delivery construct. Therefore step (b) may comprise introducing a viral vector comprising the delivery construct into the cell.

In one embodiment, one single viral vector may comprise the first, third, second and fourth constructs. Suitably the constructs may be comprised in the same viral vector. Suitably the or each viral vector is a lentivirus. Suitably a lentivirus particle.

Suitably the viral particles are generated by known techniques in the art for generating lentiviral particles. Suitably the lentiviral particles are manufactured in producer cells. Suitably the producer cells are transfected with one or more vectors comprising the constructs and one or more vectors comprising essential viral proteins, and cultured under suitable conditions to form viral particles comprising the constructs. Suitably the one or more vectors comprising essential viral proteins may comprise an envelope vector and a packaging vector. Suitably the envelope vector may encode VSV-G. Suitably the packaging vector may encode Gag, Pol, Rev and Tat. Suitably therefore the method may comprise an earlier step of manufacturing a viral particle comprising the first construct and a viral particle comprising the second construct, and/or manufacturing a viral particle comprising a third construct and a fourth construct.

Suitably the cell is then transduced with the or each viral particle. Suitably the cell is transduced with the viral particles under conditions which promote uptake of the viral particles, for example in the presence of a polycation. Suitably upon transduction the viral particles release the constructs into the cell which are then transcribed and integrated into the genome. Suitably the constructs may then be expressed from the genome.

In another embodiment, the constructs of the invention are introduced into the genome by recombination.

Suitably therefore the method of making a cell comprising first and/or second plant hormone inducible proximity systems, such as the auxin inducible proximity system, and/or the abscisic acid inducible proximity system, may comprise:
(a) Introducing the first construct and/or the third construct into the cell;
(b) Creating an integration site in the genome of the cell, wherein the integration site comprises a first recombination site;
(c) Introducing the second construct, and/or the fourth construct, and an integration construct comprising a nucleic acid sequence encoding an integrase enzyme into the cell, wherein the second construct and/or the fourth construct further comprises a second recombination site;
(d) Integrating the second construct and/or the fourth construct into the genome by recombination between the first and second recombination sites using the integrase enzyme;
   wherein steps (a) and (b) may be done in any order.

In one embodiment, the method is a method of making a cell comprising both the first and the second plant hormone inducible proximity systems, such as both of the auxin inducible proximity system of the third aspect and an abscisic acid inducible proximity system, comprising:
(a) Introducing the first and third constructs into the cell;
(b) Creating an integration site in the genome of the cell, wherein the integration site comprises a first recombination site;
(c) Introducing the second and fourth constructs into the cell, and an integration construct comprising a nucleic acid sequence encoding an integrase enzyme into the cell, wherein the second and fourth constructs further comprise a second recombination site;
(d) Integrating the second construct and the fourth construct into the genome by recombination between the first and second recombination sites using the integrase enzyme;
   wherein steps (a) and (b) may be done in any order.

Suitably the first and/or the third construct may be introduced into the cell at step (a) by any known technique such as standard transfection, electroporation, viral-mediated delivery, transposons, gene editing etc. Suitably in one preferred embodiment, the first and third constructs are introduced into the cell by viral-mediated delivery, suitably lentiviral delivery, suitably in a Lentivirus particles explained hereinabove. In one embodiment, the first construct and third construct may be contained upon the same construct which may be known as the Induction construct. Therefore step (a) may comprise introducing an induction construct comprising the first and third constructs into the cell.

Suitably, the rest of the method of this embodiment is based on recombinase mediated integration.

Suitably, the process of integrating the second and/or the fourth construct into the genome of the cell may involve one or more recombination steps.

Merely by way of example, integrating the second construct and/or the fourth construct into the genome may comprise three steps. It will be appreciated by the skilled person that recombination based techniques for integration of nucleic acids are known and any suitable method may be used. The number of steps are for illustrative purposes and the processes described herein may be combined into one or more “steps”.

Suitably, the first step (b) of creating an integration site in the genome of the cell (termed a “landing pad”) comprises the insertion of a first recombination site into the genome of the cell. Suitably the first recombination site is an integrase site, suitably a serine recombinase site. In one embodiment, the first recombination site is an attP site.

In an embodiment, a nucleotide sequence for a selectable marker is also integrated into the genome of the cell at the integration site. Suitably therefore the integration site comprises a first recombination site and a selectable marker.

Suitably, the selectable marker may be any marker described elsewhere in the specification.

Suitably therefore after step (b) the method may comprise a step of screening for cells that comprise a landing pad in the genome. Suitably such a step of screening comprises exposing the cells to an effective amount of a selection agent, and selecting those cells that express the selectable marker. In some embodiments the selection agent may be an antibiotic and the selectable marker may be an antibiotic resistance gene.

Suitably, a nucleic acid sequence encoding the recombination site and/or a nucleic acid sequence encoding a selectable marker is delivered into the cell by any suitable means.

Suitably by lentiviral Integration. Lentiviral integration is described hereinabove. Suitably therefore a viral particle, suitably a lentiviral particle, is introduced into the cell comprising a construct, suitably a nucleic acid construct, which encodes the recombination site and/or a nucleotide sequence for a selectable marker.

In another embodiment, the nucleic acid sequence encoding the recombination site and/or a nucleic acid sequence for a selectable marker may be delivered into the cell by a CRISPR-Cas system. Suitably the CRISPR-Cas system comprises a Cas nuclease such as Cas9 or Cas13 which is operable to cleave the genomic DNA, in combination with a guide RNA which is operable to bind to a target region of genomic DNA. Suitably the Cas protein and the guide RNA are introduced into the cell together with a construct, suitably a nucleic acid construct, encoding the recombination site and/or a nucleotide sequence for a selectable marker. Suitably the guide RNA directs the Cas protein to cleave the genomic DNA at a target region, and the nucleic acid construct encoding the recombination site and/or a nucleotide sequence for a selectable marker is introduced at the cleavage site by HDR.

The second step (c) of introducing the second construct and/or the fourth construct, and an integration construct into the cell comprises co-transfecting the cell with the second construct, the fourth construct, and an integration construct, wherein the integration construct encodes an integrase enzyme.

Suitably the integration construct comprises a plasmid encoding the integrase enzyme, suitably a serine integrase. Suitably therefore the integrase enzyme is a serine recombinase, suitably a phage derived serine recombinase.

Suitably, the phage derived serine recombinase may be selected from any of: C31, Bxb1, φBT1, φC1, MR11, TP901-1, R4, A118, φRV, TG1, φ370.1, Wβ, BL3, SPBc and K38. In one embodiment, the phage derived serine recombinase is BXB1.

Suitably the second construct and/or the fourth construct are also comprised upon a vector. Suitably a delivery vector may be used to integrate the second and/or the fourth construct into the genome of a cell. Suitably, the delivery vector may be a plasmid. Suitably the delivery vector further comprises, in addition to the second construct, and/or the fourth construct, a second recombination site. Suitably the second recombination site is an integrase site, suitably a serine recombinase site. In one embodiment, the second recombination site is an attB site.

In one embodiment, the second construct and the fourth construct may be contained upon the same construct which may be known as a delivery construct. Therefore step (c) may comprise introducing a delivery construct comprising the second and fourth constructs into the cell wherein the delivery construct further comprises a second recombination site. Suitably, in some embodiments, the same construct may also comprise the integrase enzyme.

Suitably methods of co-transfection may include calcium-phosphate mediated, electroporation, liposome mediated, exosome mediated, gene gun, microinjection, agrobacterium mediated transfection, for example. Suitable methods for carrying out such transfection will be known to a person skilled in the art.

The third step (d) of integrating the second construct and/or the fourth construct into the genome at the integration site comprises, expressing the integrase enzyme such that it catalyses recombination between the second construct, and/or the fourth construct, and the integration site. Suitably the integrase enzyme may be expressed from the integration vector.

Suitably, the integrase catalyses recombination between the first and second recombination sites. Suitably, the site of recombination is between the attP recombination site present at the integration site in the genome, and attB recombination site present in delivery vector comprising the second construct and/or the fourth construct.

Suitably, upon recombination, the second construct and/or the fourth construct is inserted into the genome of the cell. Suitably the second construct and/or the fourth construct is inserted at the integration site within the genome. Suitably within the first recombination site in the integration site.

Suitably, cells having successful integration of the second construct and/or the fourth construct into the genome, may be selected by expression of a selective marker. Suitably therefore one or more of the above constructs may comprise a selectable marker. Suitably therefore the method may further comprise one or more steps of selecting the cells that have successfully been transformed. In an embodiment of the invention where the selective marker is an antibiotic resistant gene, cells may be selected by exposure to an effective amount of certain antibiotics.

Suitably once the first, second, third and fourth constructs are within the cell, then the cell comprises the components of the first plant hormone inducible proximity system and the second plant hormone inducible proximity system of the invention ready to be induced and used for screening methods as described hereinbelow.

Suitably it will be understood that when making a cell comprising other inducible systems, which are indicated for use in the methods of the invention, the methods of making the cells is substantially the same as those described above. The cells may be produced by inserting into the cell, by transduction, transformation or otherwise as is known in the art a construct encoding the first and/or second inducible system. Suitably by lentiviral induction of the cell. Lentiviral integration is described hereinabove. Suitably therefore a viral particle, suitably a lentiviral particle, is introduced into the cell comprising a construct, suitably a nucleic acid construct, which encodes the first and/or second inducible systems. Suitably any features above may equally apply to the methods of making a cell comprising a first inducible system operable to express a first protein of interest, and/or a second inducible system operable to express a second protein of interest.

### Controlling Expression of a Protein of Interest in a Cell Using the Chemically Induced Proximity Systems

In an aspect of the present invention, there is provided a method of controlling expression of a protein of interest in a cell. Suitably the inducible systems may be used in various methods to control expression of one or more proteins of interest in a cell. Suitably such methods are useful for screening of candidate biological molecules, therapeutic agents, and/or engineered immune cells. Suitably such methods are useful for screening candidate biological molecules, therapeutic agents, and/or engineered immune cells for a biological effect, suitably for a biological effect on the cell expressing the or each protein.

Suitably the method comprises (a) Providing a cell comprising a first inducible system operable to express a first protein of interest, and optionally a second inducible system operable to express a second protein of interest; (b) Exposing the cell to an effective concentration of a first inducer to induce a desired level of expression of the first protein of interest from the first inducible system, and optionally exposing the cell to an effective concentration of a second inducer to induce a desired level of expression of the second protein of interest from the second inducible system.

In some embodiments, the first and second inducible systems may be any inducible system, suitably any inducible system as described herein. In one embodiment, the first and second inducible systems may be chemically induced proximity systems (CIP systems). In one preferred embodiment, they may be plant hormone or plant hormone analogue inducible proximity systems as described herein. In some embodiments, the first inducible system may be a first plant hormone inducible proximity system of the eighth aspect. In some embodiments, the second inducible system may be a second plant hormone inducible proximity system of the eighth aspect. Suitably in such embodiments, the first inducible system comprises a first and second construct as defined herein, and the second inducible system comprises a third and a fourth construct as defined herein. In one embodiment, at least one of the inducible systems is a plant hormone inducible proximity system. In one embodiment, at least one of the inducible systems is an abscisic acid inducible proximity system defined herein. In one embodiment, at least one of the inducible systems is selected from a caffeine inducible proximity system defined herein, a Mandipropamid inducible proximity system defined herein, and a gibberellin inducible proximity system defined herein.

In a preferred embodiment, the first inducible system is selected from a caffeine inducible proximity system defined herein, a mandipropamid inducible proximity system defined herein, and a gibberellin inducible proximity system defined herein and the second inducible system is an abscisic acid inducible proximity system as defined herein.

Suitably in some embodiments the method comprises, a) providing a cell comprising the first plant hormone inducible proximity system and/or the second plant hormone inducible proximity system; b) culturing the cell under conditions to express the first construct and/or the third construct; c) exposing the cell to an effective concentration of a first plant hormone to induce a desired level of expression of a first protein of interest from the second construct, and/or exposing the cell to an effective concentration of a second plant hormone to induce a desired level of expression of a second protein of interest from the fourth construct.

Suitably the cell of step (a) is produced as described above. Suitably step (b) comprises expressing the first construct and/or the third construct in the cell. Suitably therefore the first and second chimeric proteins of the first construct are expressed in the cell, and/or the third and fourth chimeric proteins of the third construct are expressed in the cell.

Suitably, once expressed, the chimeric protein which comprises the catalytically inactive I-Scel homing endonuclease (dl-Scel) binds to the dl-Scel binding site. Suitably, in some embodiments, the chimeric protein comprising a catalytically inactive I-Scel homing endonuclease (dl-Scel) fused to an AID protein binds to the dl-Scel binding site. Suitably, in some embodiments, the chimeric protein comprising a catalytically inactive I-Scel homing endonuclease (dl-Scel) fused to aCaffVHH domain, ABI1 protein, GAI protein or a fragment thereof, binds to the dl-Scel binding site.

Suitably, once expressed, the chimeric protein which comprises the GAL4 DNA binding domain binds to the GAL4 upstream activation sequence. Suitably, in some embodiments, the chimeric protein comprising a GAL4 DNA binding domain fused to an ABI1 protein binds to the GAL4 upstream activation sequence.

As described elsewhere in the specification, the first and second chimeric proteins can dimerize in the presence of Auxin, or another plant hormone or analogue thereof such as caffeine, Mandipropamid, or gibberellin, allowing the effector domain of the first chimeric protein to associate with the effector domain of the second chimeric protein.

As described elsewhere in the specification, the third and fourth chimeric proteins can dimerize in the presence of Abscisic acid, allowing the effector domain of the third chimeric protein to associate with the effector domain of the fourth chimeric protein.

Suitably therefore step (c) comprises exposing the cell to an effective concentration of auxin, another plant hormone or analogue thereof such as caffeine, mandipropamid, or gibberellin, to allow the first chimeric protein to associate with the second chimeric protein. Suitably as defined in the fifteenth aspect of the invention. Suitably to allow the plant hormone binding domain, such as the auxin binding domain, of the first chimeric protein to bind to the plant hormone binding domain, such as the auxin binding domain of the second chimeric protein. Suitably allowing the effector domain of the first chimeric protein to associate with the effector domain of the second chimeric protein.

Suitably therefore step (c) may optionally comprise exposing the cell to an effective concentration of abscisic acid to allow the third chimeric protein to associate with the fourth chimeric protein. Suitably as defined in the fifteenth aspect of the invention. Suitably to allow the abscisic acid binding domain of the third chimeric protein to bind to the abscisic acid binding domain of the fourth chimeric protein. Suitably allowing the effector domain of the third chimeric protein to associate with the effector domain of the fourth chimeric protein.

In an embodiment of the invention, the first chimeric protein comprises herpes simplex virus VP16 transactivation domain (VP16AD) fused to a TIR1 protein. In an embodiment of the invention, the first chimeric protein comprises herpes simplex virus VP16 transactivation domain (VP16AD) fused to aCaffVHH domain. In an embodiment of the invention, the first chimeric protein comprises herpes simplex virus VP16 transactivation domain (VP16AD) fused to PYR^{Mandi} protein or PYLcs^{Mandi} protein. In an embodiment of the invention, the first chimeric protein comprises herpes simplex virus VP16 transactivation domain (VP16AD) fused to GID1 protein.

In an embodiment of the invention, the second chimeric protein comprises a catalytically inactive I-Scel homing endonuclease (dl-Scel) fused to an AID protein. In an embodiment of the invention, the second chimeric protein comprises a catalytically inactive I-Scel homing endonuclease (dl-Scel) fused to aCaffVHH domain. In an embodiment of the invention, the second chimeric protein comprises a catalytically inactive I-Scel homing endonuclease (dl-Scel) fused to ABI1 protein. In an embodiment of the invention, the second chimeric protein comprises a catalytically inactive I-Scel homing endonuclease (dl-Scel) fused to GAI protein or a fragment thereof.

Suitably, in this embodiment of the invention, the auxin binding domains TIR1 and AID heterodimerize in the presence of Auxin. Alternatively the caffeine binding domains aCaffVHH homodimerize in the presence of caffeine. Alternatively the Mandipropamid binding domains PYR^{Mandi} protein or PYLcs^{Mandi} protein and ABI1 protein heterodimerize in the presence of Mandipropamid. Alternatively the gibberellin binding domains GID1 and GAI, or a fragment thereof, heterodimerize in the presence of gibberellins.

Suitably, association of the plant hormone binding domains, such as TIR1 and AID in the presence of the relevant plant hormone or analogue, such as Auxin, associates the effector domains. Suitably the binding of the plant hormone binding domains, such as TIR1 and AID, causes the effector domains to be brought into proximity with each other. Suitably the VP16 transactivation domain is brought into proximity with the catalytically inactive I-Scel homing endonuclease (dl-Scel) bound at the dl-Scel binding site (or the GAL4 DNA binding domain at the GAL4 UAS if this is used). Suitably this stimulates transcription of the protein of interest, which may be a first protein of interest. Suitably the transactivation domain VP16 stimulates transcription from the dl-Scel binding site (or the GAL4 UAS). Suitably, this transcription results in the expression of the downstream nucleic acid sequence encoding the protein of interest.

In an embodiment of the invention, the third chimeric protein comprises herpes simplex virus VP16 transactivation domain (VP16AD) fused to a PYL1 protein.

In an embodiment of the invention, the third chimeric protein comprises a GAL4 DNA binding domain fused to an ABI1 protein.

Suitably, in this embodiment of the invention, the abscisic acid binding domains PYL1 and ABI1 heterodimerize in the presence of abscisic acid.

Suitably, association of PYL1 and ABI1 in the presence of abscisic acid associates the effector domains. Suitably the binding of PYL1 and ABI1 causes the effector domains to be brought into proximity with each other. Suitably the VP16 transactivation domain is brought into proximity with the GAL4 DNA binding domain bound at the GAL4 upstream activation site (or the catalytically inactive I-Scel homing endonuclease (dl-Scel) bound at the dl-Scel binding site if this is used). Suitably this stimulates transcription of the protein of interest, which may be a second protein of interest. Suitably the transactivation domain VP16 stimulates transcription from the GAL4 upstream activation site (or the dl-Scel binding site). Suitably, this transcription results in the expression of the downstream nucleic acid sequence encoding the protein of interest.

Suitably the or each protein of interest may be expressed intracellularly, within the cell, or on the surface of the cell comprising the first inducible system which may be a plant hormone inducible proximity system and/or the second inducible system which may be a plant hormone inducible proximity system. In one embodiment, the or each protein of interest is expressed on the surface of the cell.

In the context of the present invention, expression of the or each protein of interest is proportional to the concentration of first and/or second inducer, which may be a plant hormone or analogue such as Auxin and/or Abscisic acid that the cell is exposed to. Thus, the expression of the or each protein of interest is titratable between a low level of expression and a high level of expression depending on the concentration of inducer, e.g. plant hormones, the cell is exposed to.

Suitably, the expression of a first protein of interest may be controlled by the concentration of first inducer, for example auxin, the cell is exposed to. Suitably expression of the first protein of interest is induced from the second construct as explained above. Suitably, the expression of a second protein of interest may be controlled by the concentration of a second inducer, for example abscisic acid, the cell is exposed to. Suitably expression of the second protein of interest is induced from the fourth construct as explained above.

Suitably, an increase in the first or second inducer concentration, such as the Auxin or abscisic acid concentration, in the cell increases expression of the or each protein of interest. Suitably, a reduction in the first or second inducer concentration, such as the Auxin or abscisic acid concentration, in the cell decreases expression of the or each protein of interest.

In a suitable embodiment, the cell may be exposed to a concentration of first and/or second inducer, such as Auxin and/or abscisic acid, of between 0.001 μM to 2000 μM, suitably between 0.01 μM to 2000 μM. Suitably the cell may be exposed to a concentration of first and/or second inducer, such as auxin and abscisic acid simultaneously or sequentially. Suitably the cell may be exposed to a concentration of first inducer, such as auxin, first and a concentration of second inducer, such as abscisic acid, second. Suitably the cell may be exposed to a concentration of a first inducer such as abscisic acid first and a concentration of a second inducer such as auxin second. Alternatively, the cell may be exposed to the first and second inducers, such as auxin and abscisic acid, intermittently. Suitably the exposure may alternate.

Suitably the cell may be exposed to the first and second inducers, such as auxin and abscisic acid, in any order with a period of time therebetween, suitably the period of time may be 1 second, 2 seconds, 5 seconds, 10 seconds, 30 seconds, 1 minute, 5 minutes, 10 minutes, 30 minutes, 1 hour, 2 hours, 5 hours, 10 hours, 1 day, 2 days, 5 days, 10 days, 1 month, for example.

In some embodiments, the cell may be exposed to an effective concentration of first and/or second inducer, such as Auxin and/or abscisic acid, to induce a high level of expression of the or each protein of interest, suitably from the second construct and/or the fourth construct. In one embodiment, the cell may be exposed to an effective concentration of the first inducer, such as Auxin, to induce a high level of expression of the first protein of interest, suitably from the second construct. In one embodiment, the cell may be exposed to an effective concentration of the second inducer, such as Abscisic acid, to induce a high level of expression of the second protein of interest, suitably from the fourth construct.

It will be appreciated that a cell that expresses a high level of a protein of interest intracellularly, or on its surface is representative of a cell in a diseased state as compared to a healthy reference cell. Suitable high concentrations of first or second inducer such as auxin or abscisic acid may be 50 μM-2000 μM

In some embodiments, the cell may be exposed to an effective concentration of first and/or second inducer, such as Auxin and/or abscisic acid to induce a low level of expression of the protein of interest, suitably from the second construct, and/or the fourth construct. In one embodiment, the cell may be exposed to an effective concentration of a first inducer such as Auxin to induce a low level of expression of the first protein of interest, suitably from the second construct. In one embodiment, the cell may be exposed to an effective concentration of a second inducer, such as abscisic acid to induce a low level of expression of the second protein of interest, sutiably from the fourth construct.

It will also be appreciated that a cell that expresses a low level of a protein of interest intracellularly, or on its surface is representative of a cell in a healthy i.e. reference state. Suitable low concentrations of first or second inducer, such as auxin or abscisic acid, may be 0.001 μM-50μ, or 0.01 μM-50 μM.

Suitably the cell may be exposed to different concentrations of first and/or second inducer, such as auxin and/or abscisic acid, or the same concentration of first and/or second inducer, such as auxin and/or abscisic acid, suitably any combination of concentrations may be used.

Suitably the cell may be exposed to a plurality of different concentrations of first and/or second inducer, such as auxin and/or abscisic acid, suitably selected from any one or more of: 0.001 μM, 0.002 μM, 0.003 μM, 0.004 μM, 0.005 μM, 0.006 μM, 0.007 μM, 0.008 μM, 0.009 μM 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 10 μM, 50 μM, 100 μM, 200 μM, 500 μM, 750 μM, 1000 μM, and 2000 μM. Suitably the cell may be sequentially exposed to a plurality of increasing or decreasing concentrations of first and/or second inducer, such as auxin and/or abscisic acid, suitably within this range. Suitably the cell may be sequentially exposed to a plurality of increasing or decreasing concentrations of first and/or second inducer, such as auxin and/or abscisic acid selected from: 0.001 μM, 0.002 μM, 0.003 μM, 0.004 μM, 0.005 μM, 0.006 μM, 0.007 μM, 0.008 μM, 0.009 μM, 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 10 μM, 50 μM, 100 μM, 200 μM, 500 μM, 750 μM, 1000 μM, and 2000 μM.

Suitably the cell may be exposed to a high concentration of first inducer, such as auxin, and a low concentration of second inducer, such as abscisic acid, or vice versa. Suitably the cell may be exposed to a high concentration of first inducer, such as auxin and a high concentration of second inducer such as abscisic acid. Suitably the cell may be exposed to a low concentration of first inducer, such as auxin and a low concentration of second inducer such as abscisic acid.

Suitably the concentration of the first inducer such as auxin and optionally the second inducer such as abscisic acid is chosen to induce the desired level of expression of the or each protein of interest under the control of the first inducible system, which may be an auxin inducible system or the second inducible system, which may be the abscisic acid inducible system respectively. Suitably to reflect an in vivo cellular environment for testing the candidate binding molecules, therapeutic agents, immune cells in accordance with methods of the invention.

Advantageously, the level of expression of the or each protein of interest in the cell may be finely controlled to any desired level by selecting the first or second inducer, such as auxin or abscisic acid, concentration to which the cell exposed. The same cell comprising the same first inducible system and/or second inducible system, such as an auxin inducible system and/or an abscisic acid inducible system, may be used to mimic diseased expression or healthy expression of the or each protein of interest.

### Screening Candidate Binding Molecules, Therapeutic Agents, or Engineered Immune Cells Using the Chemically Induced Proximity Systems

Several aspects of the present invention relate to a method of screening a candidate binding molecule, a candidate therapeutic agent or a candidate engineered immune cell for binding to a protein of interest expressed by the cell comprising the first inducible system, which may be a first plant hormone inducible proximity system and/or the second inducible system, which may be a second plant hormone inducible proximity system. Suitably for binding directly or indirectly to a cell comprising the first inducible system, such as a plant hormone inducible proximity system of the invention and/or the second inducible system, such as a plant hormone inducible proximity system.

Suitably all of these methods use a cell comprising the first inducible system, such as a plant hormone inducible system of the invention and/or the second inducible system, such as a plant hormone inducible proximity system. Suitably the methods may comprise providing a first inducible system operable to express a first protein of interest, and optionally a second inducible system operable to express a second protein of interest.

Suitably the methods in some embodiments comprise providing a cell comprising the Auxin inducible proximity system of the invention and/or the abscisic acid inducible proximity system, as described elsewhere in the specification. Suitably the methods in some embodiments comprise providing a cell comprising the caffeine inducible proximity system of the invention and/or the abscisic acid inducible proximity system, as described elsewhere in the specification. Suitably the methods in some embodiments comprise providing a cell comprising the Mandipropamid inducible proximity system of the invention and/or the abscisic acid inducible proximity system, as described elsewhere in the specification. Suitably the methods in some embodiments comprise providing a cell comprising the gibberellin inducible proximity system of the invention and/or the abscisic acid inducible proximity system, as described elsewhere in the specification.

Suitably the methods may further comprise culturing the cell to express the component parts of the system. Suitably, in embodiments in which the cell comprises a first and/or a second chemically inducible proximity system, culturing the cell to express the first and second chimeric proteins of the first construct, and/or the third and fourth chimeric proteins of the third construct. Suitably, therefore the cell comprising the first plant hormone inducible proximity system is cultured under conditions suitable to express the first construct of the invention. Suitably, therefore the cell comprising the second plant hormone inducible proximity system is cultured under conditions suitable to express the third construct of the invention. Optionally, the cell may be cultured under conditions suitable to select only those cells which successfully express the or each desired construct. Suitably the cells may be cultured in the presence of an antibiotic.

Suitably, the cell is then exposed to an effective concentration of the first and/or second inducers, which may be first and/or second plant hormone inducers, suitably to Auxin and/or abscisic acid to induce a desired level of expression of a protein of interest, suitably from the second construct and/or the fourth construct. Preferably this occurs, as explained above, by the auxin binding proteins, the caffeine binding proteins, the Mandipropamid binding proteins or the gibberellin binding proteins associating and/or the abscisic acid binding proteins associating, which then brings the effector domains into proximity such that the transactivation domain can activate transcription from the dl-Scel binding site and optionally the GAL4 upstream activation sequence, which in turn causes transcription of the or each downstream protein of interest.

Once the cell has been induced to express the or each protein of interest to any desired level of expression, the cell may then be used in screening methods of the invention. Suitably therefore the methods of the invention then further comprise the step of contacting the cell with a candidate binding molecule, candidate therapeutic agent or candidate engineered immune cell.

Suitably therefore the methods may comprise a step of contacting the cell with a candidate binding molecule or candidate engineered immune cell, and suitably a step of determining whether the candidate binding molecule or engineered immune cell enacts a biological effect on the cell expressing the protein of interest.

Suitably, whether the candidate binding molecule or engineered immune cell binds to the protein of interest expressed by the cells may be determined.

Suitably, a candidate binding molecule may be any binding molecule.

As described elsewhere in the specification, a protein of interest may be an antigen. In such an embodiment, the binding molecule may be a molecule that binds to an antigen.

Suitably, the binding molecule is a biologic. Suitably the binding molecule is an immunotherapy.

Suitably, the binding molecule is an immunotherapy that binds to a protein of interest, which may be an antigen of interest, expressed by the cell. Suitably, the binding molecule may be a fusion protein, an antibody (e.g. a monoclonal antibody, an antibody drug conjugate, a nanobody, scFv, di-scFv, Fab, sdAb, F(ab)₂, a 124glycol-engineered antibody) or a binding fragment thereof, a fusion protein, an antibody-drug conjugate, an aptamer, an ankyrin, a designed ankyrin repeat protein (DARPin), a peptide, a bicyclic peptide, a vaccine, a cytokine, a chemokine, a hormone, an Oncolytic virus, a bacterium, and the like.

Suitably the engineered immune cell may be a cell expressing a CAR or a T-cell receptor (TCR) such as a CAR T-cell, a TCR T cell, a CAR NK cell, a CAR macrophage, a CAR B cell.

In a further aspect of the methods of the invention, the method may be used to screen candidate therapeutic agents or drugs, such as immunotherapies, indirectly. Suitably in such methods the screening determines whether an immune cell contacted with a therapeutic agent or drug such as an immunotherapy enacts a biological effect on a cell expressing the or each protein of interest.

Suitably the immune cell may be a reference or native immune cell that has not been modified. Suitable immune cells may be a T cell, an NK cell, a B cell, a lymphocyte, a dendritic cell, or a mesenchymal cell, or immortalised cells thereof.

Suitably the immune cell may be an immortalised cell. Suitable the immune cell may express a receptor signalling pathway reporter. These employ response elements of specific transcription factors that drive expression of reporter gene. Examples of such transcription factors may be but are not limited to NFAT, NFkappaB, STAT3, STAT4, STAT5, STAT6, SRE, SRF, CRE/CREB, FOXO1. Examples of such reporter genes may be but are not limited to Firefly Luciferase, Luc2 (Humanized firefly luciferase), MetLuc (Metridia luciferase), Rluc (Renilla luciferase), Nluc (Nano luciferase), beta-galactosidase, EGFP, ZsGreen, mCherry, TurboGFP, TagBFP.

Suitably in such methods, there is a prior step of contacting the immune cell with a candidate therapeutic agent or drug such as an immunotherapy, and then subsequently determining whether the contacted immune cell enacts a biological effect on, or targets, the cell expressing the or each protein of interest, which may be an antigen. Suitable candidate therapeutic agents or drugs are as defined hereinabove.

Suitable methods of determining whether a binding molecule, engineered immune cell and/or therapeutic agent binds to a protein of interest on the surface of a cell would be known to the skilled person. Merely by way of example, these methods may include (but are not limited to) flow cytometry, fluorescence microscopy or ELISA.

Suitable methods of determining whether a binding molecule, therapeutic agent or engineered immune cell enacts a biological effect on the cell expressing the or each protein of interest would be known to the skilled person, these methods may include but are not limited to flow cytometry, imaging flow cytometry, fluorescence microscopy, ELISA.

Suitably, a biological effect may be binding, engulfing, endocytosis, phagocytosis, Antibody-Dependent Cellular Cytotoxicity (ADCC), T-cell mediated cytolysis, Antibody Dependent Cellular Phagocytosis (ADCP), perforation, cytotoxicity, cytokine/chemokine activity or release, proliferation, upregulation or downregulation of surface receptors for example.

Suitable methods to measure biological effects may be, but are not limited to, flow cytometry, ELISA, bead-based immunoassays, electrochemiluminescence, bioluminescence or fluorescence, or colorimetric measurement, electrical impedance monitoring using the xCELLigence RTCA instrument, cell avidity measurement using a cell avidity analyser, for example. Suitably these effects may be measured by measuring a signal from a reporter gene/protein expressed by the cell. Suitably the signal may be electrochemiluminescence, bioluminescence, fluorescence, or colorimetric.

Suitably determining whether a binding molecule, engineered immune cell and/or contacted immune cell after contact with a therapeutic agent binds to a protein of interest on the surface of a cell may further comprise determining the expression level of the protein of interest at which the binding molecule, engineered immune cell and/or contacted immune cell binds. Likewise, determining whether a binding molecule, therapeutic agent or engineered immune cell enacts a biological effect on the cell expressing the or each protein of interest may further comprise determining the expression level of the or each protein of interest at which the binding molecule, therapeutic agent, or engineered immune cell is enacting a biological effect, or at which the contacted immune cell is enacting a biological effect in the presence of the therapeutic agent.

Suitably therefore, further aspects of the invention relate to methods of determining the minimum level of expression of a protein of interest in a cell at which a binding molecule enacts a biological effect. Further aspects of the invention relate to determining the minimum level of expression of the protein of interest in a cell at which an immune cell enacts a biological effect in the presence of a candidate therapeutic agent or drug such as an immunotherapy. Further aspects of the invention relate to determining the minimum level of expression of a protein of interest in a cell at which a candidate engineered immune cell enacts a biological effect.

Suitably in such methods, the cell is exposed to a plurality of different first and/or second inducer concentrations, such as plant hormone inducer, suitably auxin and/or abscisic acid, concentrations. Suitably a plurality of first inducer concentrations, such as hormone, auxin, concentrations suitably which will induce a plurality of different levels of expression of the protein of interest from the second construct. Suitably a plurality of second inducer concentrations, such as hormone, abscisic acid, concentrations suitably which will induce a plurality of different levels of expression of the protein of interest from the fourth construct. Suitably the cell may be exposed to first and/or second inducer concentrations, such as a first and/or second plant hormone, suitably auxin and/or abscisic acid, concentrations ranging from low to high, suitably ranging from 0.001 μM to 2000 μM, suitably ranging from 0.01 μM to 2000 μM. Suitably the cell may be exposed to first and/or second inducer concentrations, such as auxin and/or abscisic acid concentrations selected from: 0.001 μM, 0.002 μM, 0.003 μM, 0.004 μM, 0.005 μM, 0.006 μM, 0.007 μM, 0.008 μM, 0.009 μM 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 10 μM, 50 μM, 100 μM, 200 μM, 500 μM, 750 μM, 1000 μM, 2000 μM, or any values therebetween. Suitably the cell is exposed to at least 2 different first inducer concentrations, such as plant hormone, auxin, and optionally 2 different second plant hormone, abscisic acid, concentrations across a range of values, suitably at least a low first inducer, such as auxin, and optionally a low second inducer, such as abscisic acid, concentration within the range of 0.001 μM to 50 μM, suitably 0.01-50 μM, and a high auxin and optionally a high abscisic acid concentration within the range of 50-2000 μM.

Suitably as explained above, the cell may be exposed to the same or different concentrations of inducers, such as plant hormone inducers such as auxin and abscisic acid, at the same or different times, to induce the described level of expression of the first and second proteins of interest when using both inducible systems, which may be chemically induced proximity systems.

Suitably the cell is first exposed to the lowest inducer concentration, suitably which may be a plant hormone inducer, suitably auxin and/or abscisic acid, concentration in order to induce the lowest level of expression of the or each protein of interest. Suitably the cell is then exposed to increasing concentrations of the or each inducer, which may be plant hormone inducers, suitably auxin and/or abscisic acid, to progressively increase the level of expression of the or each protein of interest.

Suitably at each concentration of inducer, suitably plant hormone inducers, suitably auxin or abscisic acid, it is determined whether the candidate binding molecule, candidate engineered immune cell, or contacted immune cell enacts a biological effect on the cell expressing the or each protein of interest.

Suitably it can then be determined at what concentration of the or ach inducer, suitably plant hormone inducers, suitably auxin and/or abscisic acid, a biological effect is seen on the cell expressing the or each protein of interest, and by extrapolation, at what level of expression of the or each protein of interest the candidate binding molecule, candidate engineered immune cell, or contacted immune cell will have a biological effect.

Suitably, the minimum level of expression of a protein of interest in a cell at which a binding molecule, contacted immune cell, or engineered immune cell, enacts a biological effect may be determined as the level of expression, of the protein of interest, at which a biological effect higher than the background biological effect is achieved. Suitably at which a biological effect of at least 3 standard deviation above the background biological effect is achieved, suitably at least 4 standard deviations above the background biological effect is achieved, suitably at least 5 standard deviations above the background biological effect is achieved, suitably at least 6 standard deviations above the background biological effect is achieved, suitably at least 7 standard deviations above the background biological effect is achieved, suitably at least 8 standard deviations above the background biological effect is achieved, suitably at least 9 standard deviations above the background biological effect is achieved, suitably at least 10 standard deviations above the background biological effect is achieved.

Suitably wherein the background biological effect is the biological effect of the candidate binding molecule, candidate engineered immune cell, or contacted immune cell on a control cell. Suitably wherein the control cell is a cell which does not contain an inducible system as described herein. Suitably the control cell does not express the or each protein of interest. Suitably wherein the control cells is a wild type cell. Alternatively, the control cell may be a cell that has been modified to prevent expression of the or each protein of interest, suitably by ‘knocking out’ the gene encoding the or each protein of interest which may be achieved by known modification techniques such as RNA interference, RNA silencing, CRISPRi, zinc finger nucleases, TALENs etc.

In one embodiment, the minimum level of expression of the first and/or second protein of interest at which the candidate binding molecule, candidate engineered immune cell, or contacted immune cell enacts a biological effect on the cell expressing the first and/or second protein of interest may be the activation threshold of the first and/or second protein of interest. Suitably the activation threshold is the number of proteins of interest that must be activated to produce a biological effect. Suitably activation may be achieved by binding the protein of interest, suitably by the candidate binding molecule, candidate engineered immune cell, or contacted immune cell. Suitably the activation threshold is the number of proteins of interest that must be bound to produce a biological effect. Suitably many of the proteins of interest described herein are receptors. Therefore suitably, the activation threshold is the number of receptors of interest that must be activated (i.e. bound) to produce a biological effect, suitably by the candidate binding molecule, candidate engineered immune cell, or contacted immune cell.

Suitably this may be determined using receiver operator characteristic (ROC) curve analysis, suitably using Youden's index, or Youden's J statistic.

Suitably the ROC curve formula may comprise:

Sensitivity=true positives/(true positives+false negatives)

Specificity=true negatives/(true negatives+false positives)

Suitably the Youden's Index formula may comprise:

J=sensitivity+specificity−1

Suitably if the candidate binding molecule, candidate engineered immune cell, or contacted immune cell enact a biological effect on the cell at a low concentration of inducer, which may be a plant hormone inducer, such as auxin and/or abscisic acid, then the candidate binding molecule, candidate engineered immune cell, or contacted immune cell will enact a biological effect in cells expressing low levels of the or each protein of interest. Suitably this may indicate that the candidate binding molecule, candidate engineered immune cell, or contacted immune cell will enact biological effects on healthy cells. Suitably this may indicate that the candidate binding molecule, candidate engineered immune cell, or contacted immune cell will enact undesirable effects, which may be considered ‘off-target’ effects.

Suitably if the candidate binding molecule, candidate engineered immune cell, or contacted immune cell enact a biological effect on the cell at a high concentration of inducer, which may be a plant hormone inducer, such as auxin and/or abscisic acid, then the candidate binding molecule, candidate engineered immune cell, or contacted immune cell will enact a biological effect in cells expressing high levels of the or each protein of interest. Suitably this may indicate that the candidate binding molecule, candidate engineered immune cell, or contacted immune cell will enact biological effects on diseased cells, specifically tumour cells. Suitably this may indicate that the candidate binding molecule, candidate engineered immune cell, or contacted immune cell will enact desirable effects, which be considered ‘on-target’ effects.

Suitably if a candidate binding molecule, candidate engineered immune cell, or contacted immune cell enacts a biological effect on the cell only at a high concentration of inducer, such as a plant hormone inducer, such as auxin and/or abscisic acid, then it is desirable, and the candidate may be selected. Suitably the methods of the invention may comprise a step of selecting a candidate binding molecule, candidate engineered immune cell, or therapeutic agent if it enacts a biological effect on the cell at a high concentration of an inducer, suitably which may be a plant hormone inducer, and optionally does not enact a biological effect on the cell at a low concentration of an inducer, suitably which may be a plant hormone inducer. Suitably the methods of the invention may comprise a step of selecting a candidate binding molecule, candidate engineered immune cell, or therapeutic agent if it enacts a biological effect on the cell at a high concentration of both first and second inducers, suitably which may be plant hormone inducers, and optionally does not enact a biological effect on the cell at a low concentration of both first and second inducers, which suitably may be plant hormone inducers.

Suitably if a candidate binding molecule, candidate engineered immune cell, or contacted immune cell enacts a biological effect on the cell only at a low concentration of an inducer, suitably a plant hormone inducer, such as auxin and/or abscisic acid, then it is undesirable, and the candidate may not be selected.

The following statements are numbered aspects and embodiments relating to the invention:
1. A cell comprising (a) a first plant hormone inducible proximity system and/or (b) a second plant hormone inducible proximity system,
   wherein the first plant hormone inducible proximity system (a) comprises:
   (i) a first construct comprising a promoter operably linked to a nucleic acid sequence encoding:
      a first chimeric protein, and
      a second chimeric protein,
   wherein the first and second chimeric proteins each comprise a first plant hormone inducer binding domain and an effector domain;
   wherein each first plant hormone inducer binding domain is operable to bind to a first plant hormone inducer; wherein the effector domains comprise a transactivation domain and a first DNA binding domain;
   wherein the first plant hormone inducer binding domain and the effector domain of the first and second chimeric proteins are different; and
   (ii) a second construct comprising a nucleic acid sequence encoding: one or more first DNA binding domain binding sites operably linked to a nucleic acid sequence encoding a first protein of interest;
   wherein the second plant hormone inducible proximity system (b) comprises:
   (i) a third construct comprising a promoter operably linked to a nucleic acid sequence encoding:
      a third chimeric protein, and
      a fourth chimeric protein,
   wherein the third and fourth chimeric proteins each comprise a second plant hormone inducer binding domain and an effector domain;
   wherein each second plant hormone inducer binding domain is operable to bind to a second plant hormone inducer; wherein the effector domains comprise a transactivation domain and a second DNA binding domain;
   wherein the second plant hormone inducer binding domain and the effector domain of the third and fourth chimeric proteins are different; and
   (ii) a fourth construct comprising a nucleic acid sequence encoding: one or more second DNA binding domain binding sites operably linked to a nucleic acid sequence encoding a second protein of interest;
   Wherein the first plant hormone inducible system does not interact with the second plant hormone inducible system, and wherein one of the first or second DNA binding domains is a dl-Scel DNA binding domain.
2. A cell according to paragraph 1, wherein the first plant hormone inducible proximity system is an auxin inducible proximity system and the second plant hormone inducible proximity system is an abscisic acid inducible proximity system.
3. A cell according to paragraph 2, wherein the first plant hormone inducer is auxin and the second plant hormone inducer is abscisic acid.
4. A cell according to paragraph 2 or 3 wherein each first plant hormone inducer binding domain is an auxin binding domain, and each second plant hormone inducer binding domain is an abscisic acid binding domain.
5. A cell according to paragraph 4 wherein each auxin binding domain is selected from Transport Inhibitor Response 1 protein (TIR1) or Auxin/indole-3-acetic acid protein (AID) in a mutually exclusive manner.
6. A cell according to paragraph 4 or 5 wherein each abscisic acid binding domain is selected from abscisic acid insensitive 1 protein (ABI1) or pyrobactin resistance-like protein (PYL1) in a mutually exclusive manner.
7. A cell according to any preceding paragraph wherein the or each transactivation domain is selected from: Gal4, Oaf1, Leu3, Rtg3, Pho4, Gln3, Gcn4 in yeast, and p53, NFAT, NF-κB, VP16 or VP34, preferably wherein the or each transactivation domain is VP16.
8. A cell according to any preceding paragraph wherein one of the first or second DNA binding domains is a LexA binding domain or a GAL4 DNA binding domain, preferably one of first or second DNA binding domains is a Gal4 DNA binding domain.
9. A cell according to any preceding paragraph, wherein the dl-Scel DNA binding domain comprises a sequence according to SEQ ID NO: 12, or a functional fragment thereof, or a sequence having at least 70%, at least 75%, at least 80%, at least 85%, at least 90%, at least 95%, at least 96%, at least 97%, at least 98%, at least 99% identity thereto, and which comprises a substitution at position 44 and/or position 145 of SEQ ID NO: 12 or at a corresponding position thereto, preferably which comprises an Asp44Ser and/or Asp145Ala substitution, more preferably wherein the dl-Scel DNA binding domain consists of a sequence according to SEQ ID NO: 13.
10. A cell according to any of paragraphs 5-9 wherein the TIR1 protein comprises a sequence according to SEQ ID NO: 14, or a functional fragment thereof, or a sequence having at least 70%, at least 75%, at least 80%, at least 85%, at least 90%, at least 95%, at least 96%, at least 97%, at least 98%, at least 99% identity thereto, and which comprises a substitution at position 7 and/or at position 10 and/or at position 74 of SEQ ID NO:14 or at a corresponding position thereto, preferably which comprises an E7K and/or E10K and/or F74G substitution, more preferably wherein the TIR1 protein consists of a sequence according to SEQ ID NO:29.
11. A cell according to any of paragraphs 5-10 wherein the AID protein is a truncation of the sequence according to SEQ ID NO: 16, preferably wherein the AID protein is truncated by up to 100 amino acids, up to 90 amino acids, up to 80 amino acids, up to 70 amino acids, up to 60 amino acids, up to 50 amino acids, up to 40 amino acids, up to 30 amino acid from the C-terminus of SEQ ID NO: 16, preferably wherein the AID protein is truncated by 34 amino acids from the C terminus of SEQ ID NO: 16, more preferably wherein the AID protein consists of the sequence as set out in SEQ ID NO: 17 or as set out in SEQ ID NO:23.
12. A cell according to any of paragraphs 6-11 wherein the ABI1 protein is a truncation of the sequence according to SEQ ID NO:24, preferably wherein the ABI1 protein comprises a truncation at both the N and C terminus of SEQ ID NO:24, more preferably wherein the truncation at the N terminus is 125 amino acids and wherein the truncation at the C terminus is 10 amino acids, still more preferably wherein the ABI1 protein consists of the sequence as set out in SEQ ID NO:25.
13. A cell according to any of paragraphs 6-12 wherein the PYL1 protein is a truncation of the sequence according to SEQ ID NO:26, preferably wherein the PYL1 protein comprises a truncation at both the N and C terminus of SEQ ID NO:26, more preferably wherein the truncation at the N terminus is 32 amino acids and wherein the truncation at the C terminus is 12 amino acids, still more preferably wherein the PYL1 protein consists of the sequence as set out in SEQ ID NO:27.
14. A cell according to any preceding paragraph wherein the first construct and the third construct each further comprise a nucleic acid encoding a cleavable linker, preferably wherein the nucleic acid encoding the cleavable linker of the first construct is located between the nucleic acid sequence encoding the first chimeric protein and the nucleic acid sequence encoding the second chimeric protein, and the nucleic acid encoding the cleavable linker of the third construct is located between the nucleic acid sequence encoding the third chimeric protein and the nucleic acid sequence encoding the fourth chimeric protein, preferably wherein the cleavable linker is a 2A self-cleaving peptide.
15. A cell according to any preceding paragraph wherein the first chimeric protein comprises a VP16 transactivation domain and a TIR1 protein, and the second chimeric protein comprises the dl-Scel DNA binding domain and an AID protein.
16. A cell according to any preceding paragraph wherein the second construct comprises between 1 to 15 I-Scel DNA binding sites, preferably ten I-Scel DNA binding sites, preferably wherein the I-Scel DNA binding sites are in tandem.
17. A cell according to any preceding paragraph, wherein the third chimeric protein comprises a VP16 transactivation domain and a PYL1 protein, and the fourth chimeric protein comprises a GAL4 DNA binding domain and an ABI1 protein.
18. A cell according to any preceding paragraph wherein the fourth construct comprises between 1 and 15 GAL4 upstream activation sequences, preferably nine GAL4 upstream activation sequences, preferably wherein the GAL4 upstream activation sequences are in tandem.
19. A cell according to any preceding paragraph wherein the cell is a mammalian cell, preferably a HEK293 or CHO-K1 cell.
20. A method of controlling expression of a protein of interest in a cell comprising:
   (a) Providing a cell according to any one of paragraphs 1-19;
   (b) Culturing the cell under conditions to express the first construct and/or the third construct; and
   (c) Exposing the cell to an effective concentration of a first plant hormone inducer to induce a desired level of expression of the first protein of interest from the second construct, and/or exposing the cell to an effective concentration of a second plant hormone inducer to induce a desired level of expression of the second protein of interest from the fourth construct.
21. A method of screening a candidate binding molecule for a biological effect comprising:
   (a) Providing a cell according to any one of paragraphs 1-19;
   (b) Culturing the cell under conditions to express the first construct and/or the third construct;
   (c) Exposing the cell to an effective concentration of a first plant hormone inducer to induce a desired level of expression of the first protein of interest from the second construct, and/or exposing the cell to an effective concentration of a second plant hormone inducer to induce a desired level of expression of the second protein of interest from the fourth construct;
   (d) Contacting the cell with a candidate binding molecule; and
   (e) Determining whether the candidate binding molecule enacts a biological effect on the cell expressing the first and/or second protein of interest.
22. A method of determining the minimum level of expression of a protein of interest in a cell at which a candidate binding molecule enacts a biological effect, comprising:
   (a) Providing a cell according to any one of paragraphs 1-19;
   (b) Culturing the cell under conditions to express the first construct and/or the third construct;
   (c) Exposing the cell comprising the first plant hormone inducible proximity system and/or the second plant hormone inducible proximity system to a plurality of different concentrations of a first plant hormone inducer to induce a plurality of different levels of expression of the first protein of interest from the second construct, and/or exposing the cell to a plurality of different concentrations of second plant hormone inducer to induce a desired level of expression of the second protein of interest from the fourth construct;
   (c) Contacting the cell with a candidate binding molecule;
   (d) Determining whether the candidate binding molecule enacts a biological effect on the cell expressing the first and/or second protein of interest; and
   (e) Determining the minimum level of expression of the first and/or second protein of interest at which the candidate binding molecule enacts a biological effect on the cell expressing the first and/or second protein of interest.
23. A method according to paragraphs 21 or 22 wherein the candidate binding molecule is selected from: a fusion protein, an antibody (e.g. a monoclonal antibody, an antibody drug conjugate, a nanobody, scFv, di-scFv, Fab, sdAb, F(ab)2, a 135glycol-engineered antibody) or a binding fragment thereof, an aptamer, an ankyrin, a designed ankyrin repeat protein (DARPin), a peptide, a bicyclic peptide, a vaccine, a cytokine, a chemokine, a hormone, an oncolytic virus, and a bacterium, preferably the binding molecule is an immunotherapy.
24. A method of screening a candidate therapeutic agent for a biological effect comprising:
   (a) Providing a cell according to any one of paragraphs 1-19, and an immune cell;
   (b) Culturing the cell comprising the first plant hormone inducible proximity system and/or the second plant hormone inducible proximity system under conditions to express the first construct and/or the third construct;
   (c) Exposing the cell comprising the first plant hormone inducible proximity system, and/or the second plant hormone inducible proximity system, to an effective concentration of a first plant hormone inducer to induce a desired level of expression of the first protein of interest from the second construct, and/or exposing the cell to an effective concentration of a second plant hormone inducer to induce a desired level of expression of the second protein of interest from the fourth construct;
   (d) Contacting the immune cell with the candidate therapeutic agent; and
   (e) Determining whether the contacted immune cell enacts a biological effect on the cell expressing the first and/or second protein of interest.
25. A method of determining the minimum level of expression of a protein of interest in a cell at which an immune cell enacts a biological effect in the presence of a candidate therapeutic agent, the method comprising:
   (a) Providing a cell according to any one of paragraphs 1-19, and an immune cell;
   (b) Culturing the cell comprising the first plant hormone inducible proximity system and/or the second plant hormone inducible proximity system, under conditions to express the first construct and/or the third construct;
   (c) Exposing the cell comprising the first plant hormone inducible proximity system and/or the second plant hormone inducible proximity system to a plurality of different concentrations of a first plant hormone inducer to induce a plurality of different levels of expression of the first protein of interest from the second construct, and/or exposing the cell to a plurality of different concentrations of a second plant hormone inducer to induce a desired level of expression of the second protein of interest from the fourth construct;
   (d) Contacting the immune cell with the candidate therapeutic agent;
   (e) Determining whether the contacted immune cell enacts a biological effect on the cell expressing the first and/or second protein of interest at each level of expression of the first and/or second protein of interest; and
   (f) Determining the minimum level of expression of the first and/or second protein of interest at which the contacted immune cell enacts a biological effect on the cell expressing the first and/or second protein of interest.
26. A method according to paragraph 24 or 25 wherein the candidate therapeutic agent is a biologic, preferably the candidate therapeutic agent is an immunotherapy, preferably selected from a fusion protein, an antibody (e.g. a monoclonal antibody, an antibody drug conjugate, a nanobody, scFv, di-scFv, Fab, sdAb, F(ab)₂, a 136glycol-engineered antibody) or a binding fragment thereof, an aptamer, an ankyrin, a designed ankyrin repeat protein (DARPin), a peptide, a bicyclic peptide, a vaccine, a cytokine, a chemokine, a hormone, an oncolytic virus, and a bacterium.
27. A method according to any of paragraphs 24-26 wherein the immune cell is selected from a T cell, an NK cell, a B cell, a lymphocyte, a dendritic cell, and a mesenchymal cell, or immortalised cells thereof.
28. A method of screening a candidate engineered immune cell for biological effects comprising:
   (a) Providing a cell according to any one of paragraphs 1-19;
   (b) Culturing the cell comprising the first plant hormone inducible proximity system and/or the second plant hormone inducible proximity system under conditions to express the first construct and/or the third construct;
   (c) Exposing the cell comprising the first plant hormone inducible proximity system and/or the second plant hormone inducible proximity system to an effective concentration of a first plant hormone inducer to induce a desired level of expression of the first protein of interest from the second construct, and/or exposing the cell to an effective concentration of a second plant hormone inducer to induce a desired level of expression of the second protein of interest from the fourth construct;
   (d) Contacting the cell with the candidate engineered immune cell; and
   (e) Determining whether the candidate engineered immune cell enacts a biological effect on the cell expressing the first and/or second protein of interest.
29. A method of determining the minimum level of expression of a protein of interest in a cell at which a candidate engineered immune cell enacts a biological effect, the method comprising:
   (a) Providing a cell according to any one of paragraphs 1-19;
   (b) Culturing the cell comprising the first plant hormone inducible proximity system and/or the second plant hormone inducible proximity system under conditions to express the first construct and the third construct;
   (c) Exposing the cell comprising the first plant hormone inducible proximity system, and/or the second plant hormone inducible proximity system, to a plurality of different concentrations of first plant hormone inducer to induce a plurality of different levels of expression of the first protein of interest from the second construct, and/or exposing the cell to a plurality of different concentrations of second plant hormone inducer to induce a desired level of expression of the second protein of interest from the fourth construct;
   (d) Contacting the cell with the candidate engineered immune cell;
   (e) Determining whether the candidate engineered immune cell enacts a biological effect on the cell expressing the first and/or second protein of interest at each level of expression of the first and/or second protein of interest; and
   (f) Determining the minimum level of expression of the first and/or second protein of interest at which the candidate engineered immune cell enacts a biological effect on the cell expressing the first and/or second protein of interest.
30. A method according to paragraphs 28 or 29 wherein the candidate engineered immune cell is selected from a cell expressing a CAR or a T-cell receptor (TCR), preferably selected from a CAR T-cell, a TCR T cell, a CAR NK cell, a CAR macrophage, and a CAR B cell.
31. A method according to any of paragraphs 20, 21, 23, 24, 26-28 or 30, wherein the effective concentration of first or second plant hormone inducer comprises between 0.01 μM to 2000 μM of auxin or abscisic acid.
32. A method according to any of paragraphs 20, 21, 23, 24, 26-28, 30 or 31 comprising exposing the cell to a plurality of different concentrations of first plant hormone inducer to induce a plurality of different levels of expression of the first protein of interest from the second construct, and/or exposing the cell to a plurality of different concentrations of second plant hormone inducer to induce a desired level of expression of the second protein of interest from the fourth construct.
34. A method according to any of paragraphs 22, 23, 25, 26, 29, 30, or 32 wherein the plurality of different concentrations of first and/or second plant hormone inducer range from 0.1 μM to 2000 μM, preferably the plurality of different concentrations of first and/or second plant hormone inducer are selected from: 0.01 μM, 0.1 μM, 1 μM, 2 μM, 5 μM, 10 μM, 50 μM, 100 μM, 200 μM, 500 μM, 750 μM, 1000 μM, and 2000 μM.
35. A method according to any of paragraphs 22, 23, 25, 26, 29, 30, 32, or 34 wherein the cell is exposed to at least two different concentrations of first and/or second plant hormone inducer acid, preferably at least a low concentration within the range of 0.01-50M and a high concentration in the range of 50-2000 μM.
36. A method according to any of paragraphs 20-35 wherein the biological effect is selected from: binding, engulfing, trogocytosis, endocytosis, phagocytosis, Antibody-Dependent Cellular Cytotoxicity (ADCC), T-cell mediated cytolysis, Antibody Dependent Cellular Phagocytosis (ADCP), perforation, cytotoxicity, cytokine/chemokine activity or release, proliferation, cell activation, upregulation or downregulation of surface receptors.
37. A method according to any of paragraphs 22-23, 25-27 or 29-36, wherein the minimum level of expression of the first and/or second protein of interest at which the candidate enacts a biological effect on the cell may be determined using receiver operator characteristic (ROC) curve analysis, preferably using Youden's Index.

The invention will now be further described with reference to the following Examples.

### EXAMPLES

### Example 1: Auxin Inducible System

### 1 CIP System Based on the Plant Phytohormone Auxin Signalling Pathway for Controlling Titratable Expression of a Target Antigen of Interest (TAOI)

The inventors describe the use of a CIP system based on the plant phytohormone auxin signalling pathway for controlling titratable expression of a TAOI. In this signalling pathway, auxins such as Indole-3-acetic acid (IAA) induce heterodimerization between F-box protein transport inhibitor response 1 (TIR1) and transcriptional corepressor auxin/indole-3-acetic acid (AUX/IAA) proteins (termed AIDs)¹. It is known that Arabidopsis thaliana TIR1 does not possess thermal stability under the physiological conditions in mammalian systems. As a result, Oryza sativa TIR1 (osTIR1) is utilized in place of auxin-induced protein degradation in mammalian cells². Based on the work of Zhao et al (2018), an E7K/E10K osTIR1 mutant and a 34 amino acid truncated version of AID (AIDΔ34) will be employed as the IAA dependent heterodimerization component³ The herpes simplex virus VP16 transactivation domain will be fused to one of the heterodimerization components, in this case, TIR1.

To facilitate induction of expression in the presence of the small molecule (IAA), a DNA binding protein with a unique recognition sequence which is placed upstream of the TAOI is required to localise the transactivator domain and induce expression. Intron-encoded endonuclease I-Sce-I is a homing endonuclease with an 18 bp recognition sequence (TAG GGATAACAGGGTAAT SEQ ID NO:1). This recognition sequence is sufficiently large that it would require a genome approximately 20 times the size of the human genome for it to occur by chance but is also small enough that multiple copies can be used, potentially enhancing the dynamic range of expression induction. By substituting amino acids at the active site of I-Scel (Asp44Ser and Asp145Ala) it is possible to create a catalytically inactive mutant (dl-Scel) that is competent in specific DNA binding but not cleavage⁴. Therefore, the inventors employed a dl-Scel fused to AIDΔ34 as part of the construct shown in FIG. 1, panel A1. The 18 bp binding I-Scel binding site is placed upstream of the TAOI (FIG. 1, panel A2). When these two constructs are introduced into a cell, transcription of the antigen TAOI is only induced in the presence of IAA (FIG. 1, panel B).

The inventors have demonstrated an indole-3-acetic acid (IAA) controlled, chemically induced proximity (CIP) for titratable TAOI expression. As shown in FIG. 1 panel A1), the IAA activator cassette comprises the herpes simplex virus VP16 transactivation domain (VP16AD) fused to TIR1 and a catalytically inactive I-Scel homing endonuclease (dl-Scel) fused to AIDΔ34. Expression of these two fusion proteins is operably linked by inclusion of a porcine teschovirus-1 2A (P2A) self-cleaving peptide. As demonstrated in FIG. 1 panel A2, the expression construct employs the 18 bp recognition sequence for I-Scel downstream of which the target antigen of interest (TAOI) is placed. FIG. 1 penal B) shows the dl-Scel associates with its recognition sequence but cannot activate transcription as it lacks a transactivator domain. In the presence of IAA, the AIDΔ34 associates with TIR1 and brings the VP16 transactivator domain into proximity of the upstream region of the TAOI, activating transcription.

### 2 Assessment of Cytotoxicity of IAA on HEK293 and CHO-K1 Cells

Before IAA was used as an expression inducer for the transduced cell lines, it was assessed for any cytotoxic effects on CHO-K1 and HEK293 cells. Cell viability in both HEK293 and CHO-K1 cells was measured by flow cytometry 24 hours post addition of IAA over the concentration range indicated.

HEK293 and CHO-K1 cells were treated with the indicated concentrations of IAA for 24 hours before being stained with eFluor780 fixable viability dye and analysed by flow cytometry. FIG. 2 shows viability reported as the percentage of cells which do not take up the dye. The inventors found that no decrease in cell viability was observed over increasing concentrations of IAA.

### 3 Effect of IAA on Proliferation of T Cells

One of the main considerations for application of the CIP system in immunology assays using primary human immune cells, is the effect that the small molecules (e,g. IAA) may have on cell viability and normal function. For example, one of the main limitations of one of the rapamycin-CIP systems is the high immunoregulatory effect of rapamycin on immune cells that limits its applicability in immunology assays. The inventors assessed the effect of IAA in the function of immune cells. PBMCs from two donors were stimulated with Human T-Activator CD3/CD28 targeting Dynabeads to stimulate T cell activation and proliferation, in the presence of a increasing concentration series of IAA. T cell proliferation and IFNγ production was analysed 96 hrs post stimulation to determine any negative effects on T cells. The concentration series of IAA was selected to reflect the concentration expected to be used in assays. The results show that IAA had little or no effect on proliferation of both CD4+ and CD8+ T cells or on the ability of immune cells to produce IFNγ in the two human donors tested. Proliferation of CD4+ (A) and CD8+ (B) T cells was assessed by flow cytometry using a proliferation dye. The results are shown in FIG. 3, IFNγ production was assessed by ELISA (panel C), n=3, graphs report medium+SD.

### 4 Generation of IAA Activator Cassette Pools

HEK293 and CHO-K1 cells were transduced with lentivirus expressing the IAA activator cassettes and selected using G418. Expression of the activator cassette was confirmed via intracellular staining with an anti-VP16AD antibody and flow cytometry analysis. The results of this analysis are shown in FIG. 4, in which stained parental HEK293 or CHO-K1 cells are shown in dark grey, cells transduced with IAA activator cassettes are shown in light grey. The expression profile shows that whilst some cells are transduced and expressing the activator cassette (as evidenced by the detection of VP16 expression), there are some cells that overlay the parental cells, suggesting a non-transduced population. With sufficient numbers of activator cassette expressing cells, the stable pools were transduced with the reporter cassette, followed by cloning of the resulting cell pools. Clones were selected on a balance of induction of expression vs background expression.

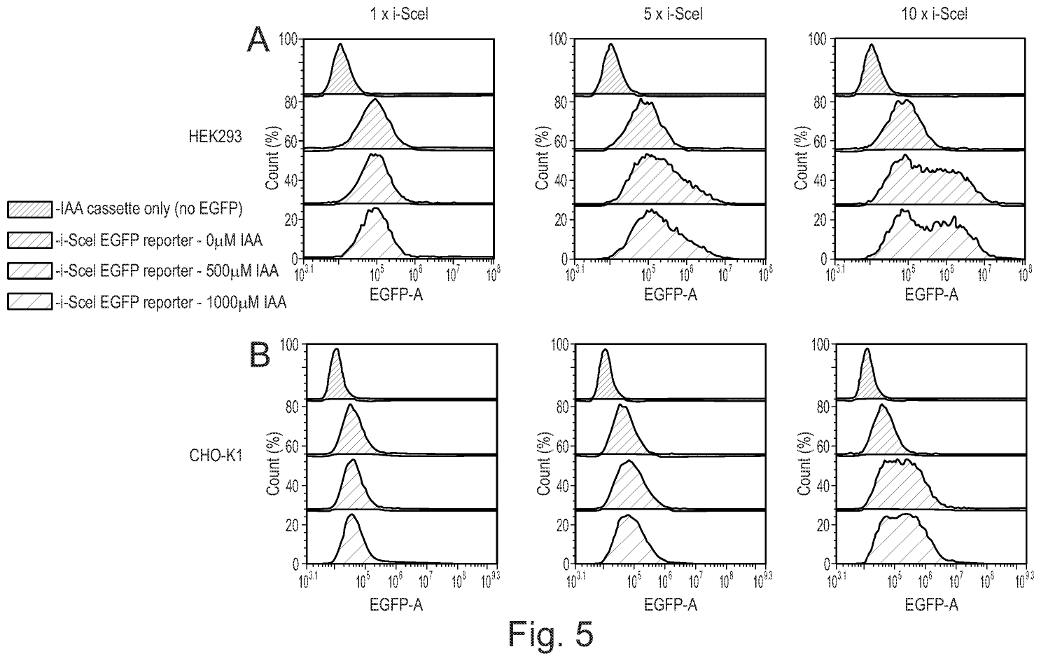

As can be seen in FIG. 5, the inventors have demonstrated successful induction of reporter gene expression with the IAA CIP system, whereby induction of expression of EGFP can clearly be seen in the presence of IAA in both the HEK293 and CHO-K1 cell backgrounds. The levels of expression correlate with the number of i-Scel sites, with negligible induction observed at 1× i-Scel and the highest induction over background observed with the 10× i-Scel sites reporter. There is no evidence of additional induction at 1000 μM IAA compared to 500 μM suggesting that the IAA concentration is not a limiting factor at 500 μM. However, the inventors predict, that the inclusion of higher numbers of i-Scel binding sites in the reporter/TAOI cassette, would result in higher levels of induction of the reporter/TAOI.

5 Induction of EGFP Expression with IAA

The inventors hypothesised that due to the non-clonal nature of the reporter construct pools, potentially increased induction could be obtained by performing single cell dilution cloning and analysing clones on the basis of both background expression and expression of EGFP following induction with IAA. Therefore, the 5× and 10× i-Scel binding site pools for HEK293 and CHO-K1 cells were single cell dilution cloned, and colonies picked from each cell pool and then analysed for induction of expression following IAA treatment. FIG. 6 shows the 4 best performing clones with and without 500 μM IAA treatment. The HEK293 and CHO-K1 cells containing the IAA activator cassette and the 10× and 5× i-Scel reporter constructs were seeded overnight in 96 well plates. IAA was added at the indicated concentration and the cells were incubated for a further 24 hours. EGFP expression was detected by flow cytometry analysis.

The selected clones showed greater induction of expression of EGFP following treatment with 500 μM IAA than the stable pools. Following the successful demonstration of the induction of EGFP expression in the 10× and 5× i-Scel reporter clones, the 10× i-Scel EGFP reporter clones of HEK293 and CHO-K1 were taken forward for analysis of IAA dose dependent expression.

The HEK293 and CHO-K1 cells containing the IAA activator cassette and the 10× i-Scel reporter construct were seeded overnight in 96 well plates. IAA was added at the indicated concentrations and the cells were incubated for a further 24 hours. EGFP expression was detected by flow cytometry analysis.

As can be seen in FIG. 7, both the CHO-K1 and HEK293 10× i-Scel EGFP clones show an IAA dose-dependent induction of EGFP expression, demonstrating a finely titratable induction of reporter gene expression in the presence of incremental changes in the quantity of IAA.

6 Induction of CD19 Expression with IAA

To test the system in a therapeutically relevant target, a construct expressing CD19 under the control of 10× i-Scel binding sites was introduced into the IAA activator cassette containing CHO-K1 cells and clones isolated. As the purpose of the system is to provide titratable expression of a given receptor (usually a tumour associated antigen) within a biologically relevant range, the CD19 expression was quantified using a Quantibrite bead fluorescence quantification kit which contains multiple bead populations each conjugated with set numbers of fluorophore molecules (PE in this case) per bead. Assuming the fluorophore conjugation to the detection antibody is at a 1:1 ratio (which was confirmed by the manufacture) and antibody binding to the receptor of interest (CD19) occurs at a 1:1 ratio, the number of CD19 receptors per cell can be calculated. By means of comparison, the CD19 expression level of the CD19 positive cells found in peripheral blood mononuclear cells (PBMCs), e.g. B cells, and Ramos cells were calculated in the same experiment.

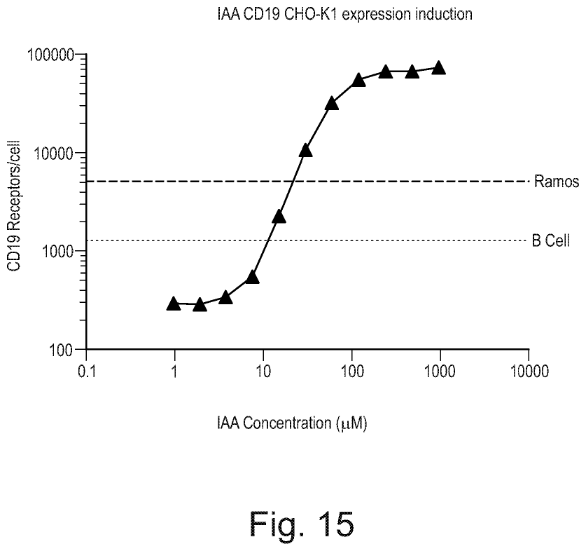

FIG. 15 shows one of the best performing clones induced across a range of concentrations of IAA. As can be seen in FIG. 15, a dose dependent range of CD19 expression has been achieved which is titratable at both higher and lower levels than both Ramos and B cells. This will allow the expression level of TAOIs to be matched to both tumour and healthy cell levels.

### 7 Summary

The inventors have demonstrated the generation of HEK293 and CHO-K1 cells expressing the necessary molecular components of a novel IAA CIP system, that is, the so-called IAA activator cassette which comprises the herpes simplex virus VP16 transactivation domain (VP16AD) fused to TIR1 and a catalytically inactive I-Scel homing endonuclease (dl-Scel) fused to AIDΔ34.

Following the introduction of reporter constructs containing EGFP downstream of varying numbers of the i-Scel binding site, it was determined that expression of EGFP could be induced by the addition of IAA to the cell culture media for 24 hours, with the 10× i-Scel binding site reporter providing the largest level of expression induction. Using clones derived from these transductions, we have demonstrated finely titratable, IAA dose dependent expression of EGFP, representative of any desired antigen (TAO1), in both HE293 and CHO-K1 cell backgrounds.

### Example 2: Abscisic Acid Inducible System

### 1 CIP System Based on the Plant Phytohormone Abscisic Acid Signalling Pathway for Controlling Titratable Expression of an Antigen (TAO2)

The inventors describe the use of a CIP system based on the plant phytohormone phytohormone S-(+)-abscisic acid (ABA) signalling pathway for controlling titratable expression of a target antigen of interest (TAOI). The ABA signalling pathway mediates stress responses and developmental decisions in plants. ABA produces its effects by binding to the pyrabactin resistance (PYR)/PYR1-like (PYL)/regulatory component of ABA receptor (RCAR) family of intracellular receptors, and the resulting complexes inhibit the activity of protein phosphatase type 2Cs (PP2Cs), which leads to the activation or repression of downstream target genes 4. Based on the crystal structure of the PYL1-ABA-ABI15, Liang et al 5 postulated that the interacting complementary surfaces (CSs) of PYL1 (PYLcs, amino acids 33 to 209) and ABI1 (ABIcs, amino acids 126 to 423) would confer ABA-induced proximity to proteins fused to these fragments.

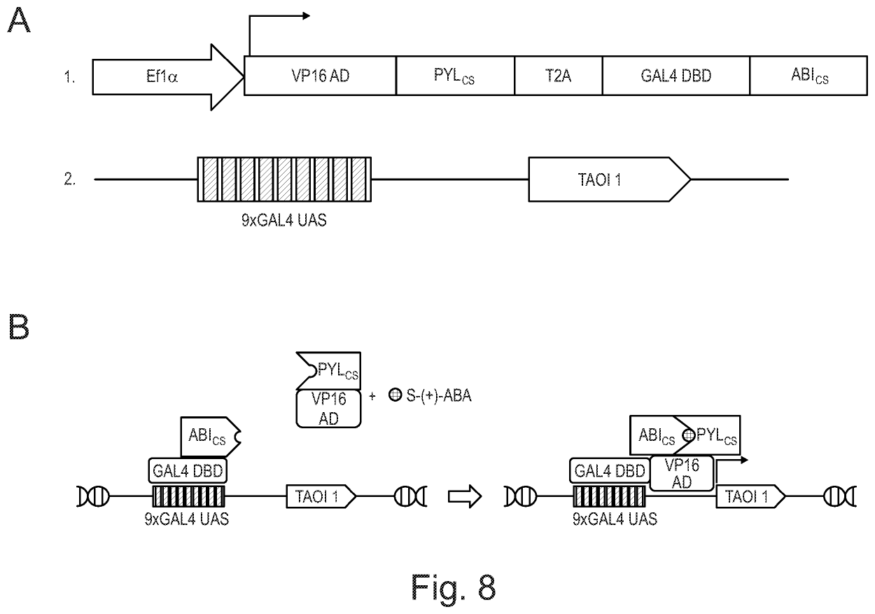

As described in Liang et al 5, an ABA-activator cassette will be generated by fusing the yeast Gal4 DNA binding domain (Gal4DBD) to ABIcs and the herpes simplex virus VP16 transactivation domain (VP16AD) to PYLcs. The Gal4DBD binds specifically to the Gal4 upstream activation sequence (UAS) but cannot activate transcription without a transactivation domain. The VP16AD, when tethered to DNA binding domains, strongly activates transcription but cannot act without proximity to a DNA binding domain. The Gal4DBD-ABIcs and VP16AD-PYLcs fusion proteins can be expressed together at stoichiometric ratios by employing a porcine teschovirus-1 2A (P2A) self-cleaving peptide between the two polypeptides as shown in FIG. 8A. In this system, the TAOI is placed under the control of the Gal4 UAS, as shown in FIG. 8B where a 9× repeat of the UAS sequence is employed. When these two constructs are introduced into a cell, the Gal4 DNA binding domain will associate with the Gal4 UAS sequences upstream of the TAOI. Expression of the TAOI is only induced in the presence of ABA, which, by causing association of ABICS and PYLCS, brings the fused VP16 transactivation domain into proximity of the upstream region of the TAOI, initiating transcription.

As shown in FIG. 8: the inventors provide an Abscisic acid (ABA) controlled, chemically induced proximity (CIP) for titratable TAOI expression. FIG. 8A1 shows the ABA activator cassette comprises yeast Gal4 DNA binding domain (Gal4DBD) fused to ABIcs and the herpes simplex virus VP16 transactivation domain (VP16AD) fused to PYLcs. Expression of these two fusion proteins is operably linked by inclusion of a Thosea asigna 2A (T2A) self-cleaving peptide. FIG. 8A2 shows the expression construct employs a 9× repeat of the Gal4 upstream activation sequence (UAS) under the control of which the target antigen of interest (TAOI) is placed. FIG. 8B shows the Gal4 DNA binding domain associates with the Gal4 UAS but cannot activate transcription as it lacks a transactivator domain. In the presence of ABA, the PYLcs associates with ABIcs and brings the VP16 transactivator domain into proximity of the upstream region of the target antigen (gene) of interest, activating transcription.

### 2 ABA Dose Response Assessment

CHO-K1 cells containing the ABA activator cassette and the CD19 reporter constructs were seeded in 96 well plates. Following overnight incubation, the media was replaced with fresh media containing the indicated concentrations of ABA and the cells were incubated for a further 48 hours. CD19 expression was detected by anti-CD19 staining (Biolegend #302254) and flow cytometry analysis. The number of CD19 receptors per cell was quantified using a Quantibrite PE bead fluorescence quantification kit (BD Biosciences #340495).

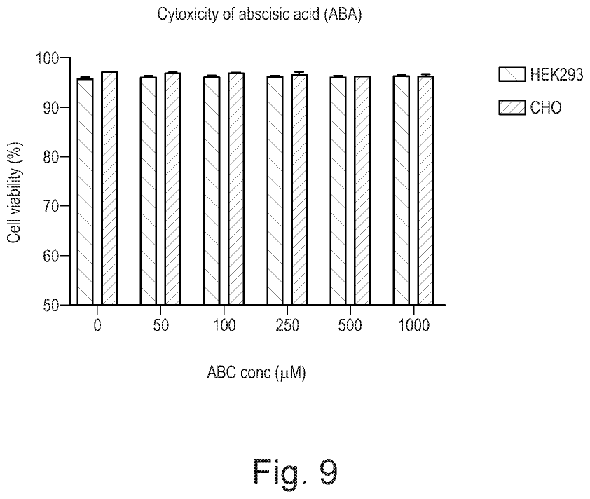

FIG. 9 shows HEK293 and CHO-K1 cells that were treated with the indicated concentrations of ABA for 24 hours before being stained with eFluor780 fixable viability dye and analysed by flow cytometry. Viability is reported as the percentage of cells which do not take up the dye. No decrease in cell viability was observed over increasing concentrations of ABA.

### 3 Effect of ABA on Proliferation of T Cells

One of the main consideration of CIP system for use in immunology assays is the effect that the small molecules (e,g. ABA) will have on the function of immune cells. For example one of the main limitation of one of the rapamycin-CIP system is the high immunoregulatory effect of rapamycin on immune cells that limits its applicability in immunology assays. In order to assess the effect of ABA in the function of immune cell PBMCs from two donors were stimulated with CD3/CD28 targeting Dynabeads in the presence of a concentration series of ABA and proliferation and IFNγ production was analysed 96 hrs post stimulation. The concentration series of ABA was selected to reflect the concentration expected using in assays. ABA had minimal effects on proliferation of both CD4+ and CD8+ T cells or on the ability of immune cells to produce IFNγ.

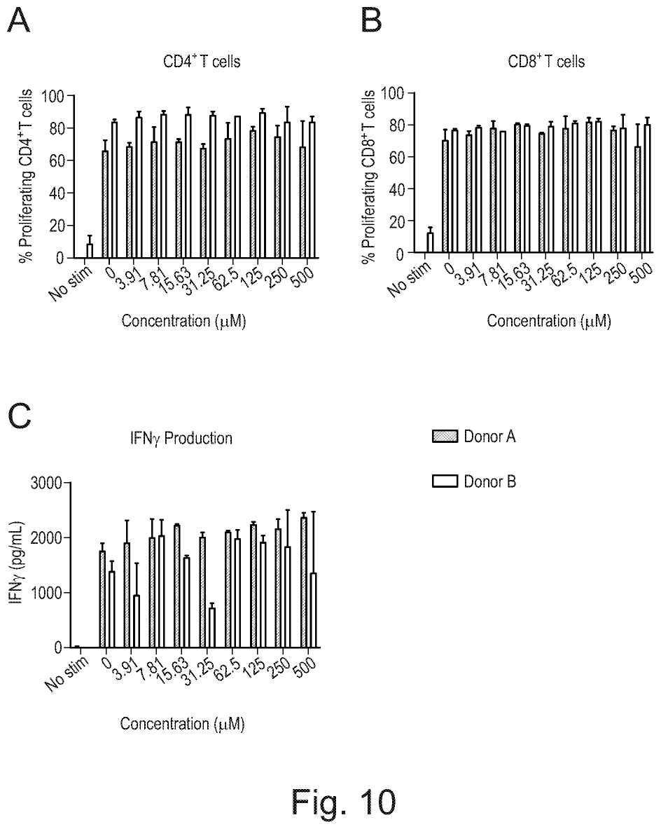

FIG. 10 shows the proliferation of CD4+ (A) and CD8+ (B) T cells assessed by flow cytometry using a proliferation dye. IFNγ production was assessed by ELISA (C). n=3, graphs report medium±SD.

### 4 Generation of ABA Activator Cassette Pools

HEK293 and CHO-K1 cells were transduced with lentivirus expressing the ABA activator cassette and selected using puromycin. Expression of the activator cassette was confirmed via intracellular staining with an anti-VP16AD antibody and flow cytometry analysis

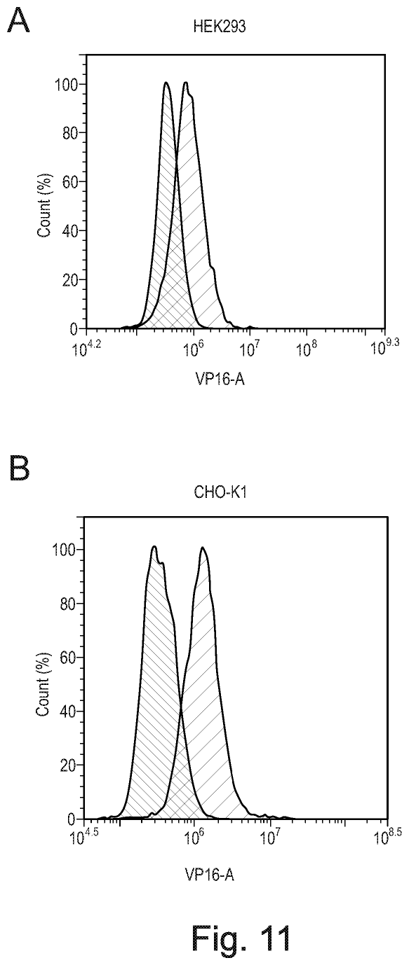

FIGS. 11A and 11B shows expression of the ABA activator cassette in HEK293 and CHO-K1 cells. Stained parental HEK293 or CHO-K1 cells are shown in dark grey, cells transduced with ABA activator cassette are shown in light grey.

The expression profile shows that whilst some cells are transduced and expressing the activator cassette, as evidenced by the detection of VP16 expression, there are some cells that overlay the parental cells. In the interests of time, it was decided to proceed with the transduction of the reporter cassette, then clone out the resulting cell pools. Clones would be selected on a balance of induction of expression vs background expression.

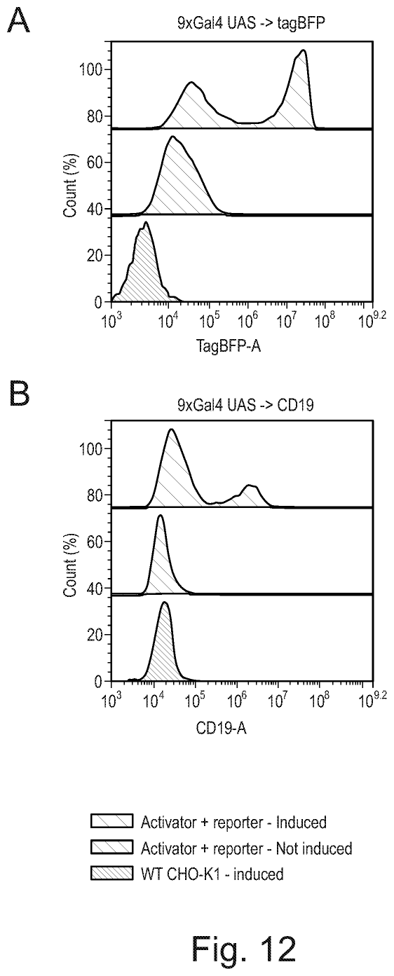

FIG. 12 shows ABA treatment of ABA activator CHO-K1 and HEK293 cells transduced with tagBFP (A) and CD19 (B) reporter constructs. The cells were treated with 1000 μM ABA for 24 hours before being analysed by flow cytometry for the expression of tagBFP and CD19.

As can be seen in FIG. 12, expression of both tagBFP and CD19 was obtained upon addition of ABA to the cell culture medium. It was hypothesised that due to the non-clonal nature of the reporter construct pools, potentially increased induction could be obtained by performing single cell dilution cloning and analysing clones on the basis of both background expression and expression of CD19 or tagBFP following induction with IAA.

5 Induction of CD19 Expression with ABA

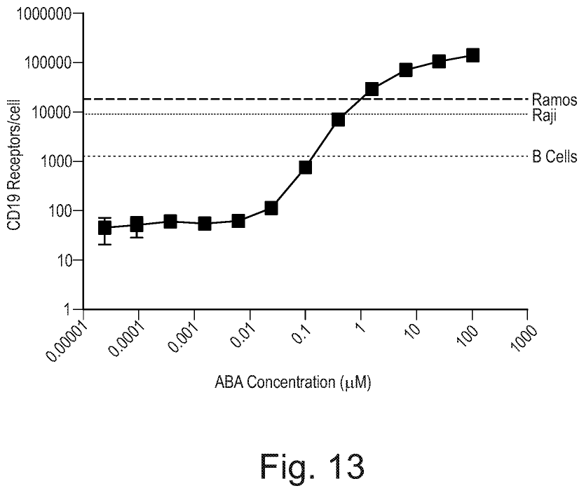

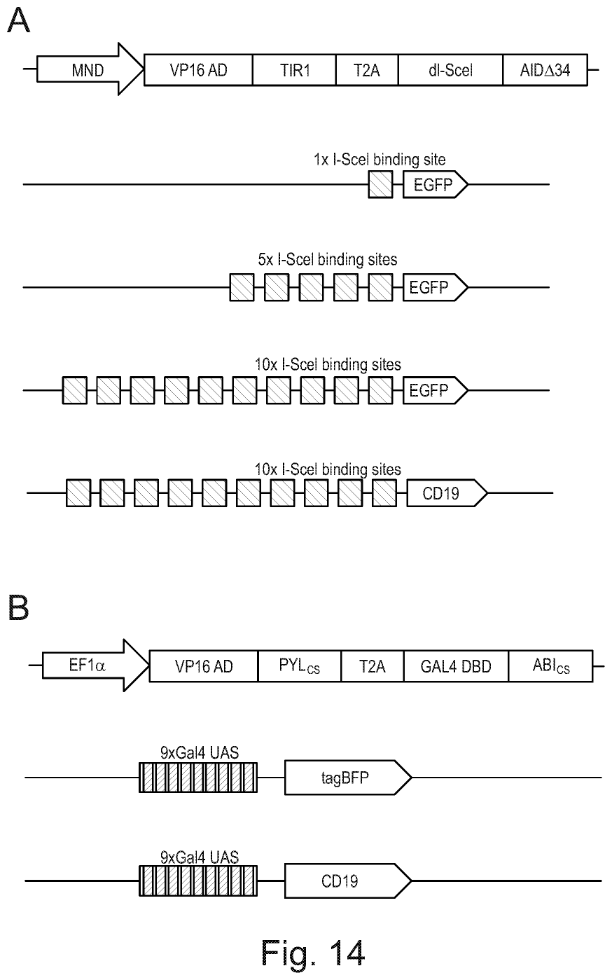

As CD19 was the more therapeutically relevant target, the 9×Gal4 UAS->CD19 CHO-K1 cells were single cell dilution cloned, and colonies picked for induction analysis following ABA treatment. FIG. 13 shows one of the best performing clones induced across a range of concentrations of ABA.

CHO-K1 cells containing the ABA activator cassette and the 9×Gal4 UAS CD19 reporter construct were seeded overnight in 96 well plates. ABA was added at the indicated concentrations and the cells were incubated for a further 48 hours. CD19 expression was detected by flow cytometry analysis and the number of CD19 receptors per cell was quantified using a Quantibrite PE bead fluorescence quantification kit (BD Biosciences). The CD19 receptors per cell for Ramos, Raji and B cells isolated from PBMCs are shown by way of comparison.

### Example 3: Concurrent Use of the IAA and ABA CIP Systems

To assess if the ABA and IAA system are compatible for use in the same cell line with no cross-talk, the CHO-K1 CD19 reporter clones generated for each system were used to test the effect of IAA on the ABA CIP system and the effect of ABA on the IAA CIP system.

CHO-K1 ABA activator CD19 reporter cells and CHO-K1 IAA activator CD19 reporter cells were each treated with 1000 μM IAA and 1000 μM ABA separately for 24 hours. Following this incubation, the cells were analysed for CD19 expression by flow cytometry.

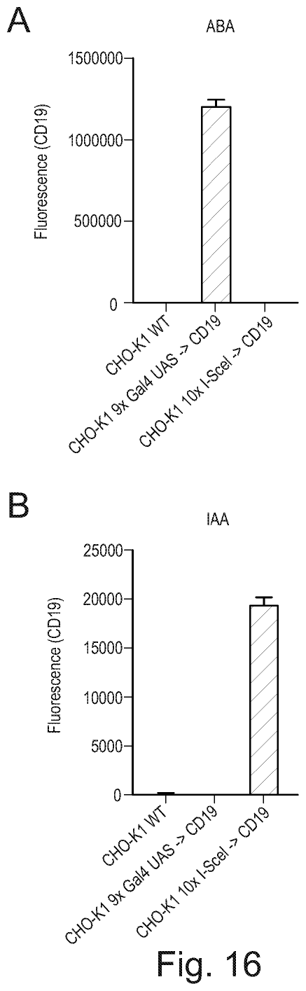

As can be seen in FIG. 16, whilst the ABA system has higher over expression than the IAA system, IAA does not induce expression from the ABA CIP system and ABA does not induce expression from the IAA system.

To provide the initial proof of concept of the use of the ABA and IAA CIP systems in an assay setting, it was decided to utilise an in-house developed Jurkat NFAT luciferase reporter system as a marker for T cell activation in a T cell engager assay employing the CD19×CD3 targeting bispecific Blincyto. ABA or IAA CIP CD19 cells are used as the target cells in this scenario. This represents a typical assay set up for measuring the effect of a bispecific against cells expressing its target antigen.

CHO-K1 IAA or ABA activator CD19 reporter cells were plated with a concentration series of IAA or ABA (7.18 μM to 500 μM) to induce CD19 expression over 24 hours. After 24 hours, IAA and ABA were removed from the system and Jurkat NFAT luciferase reporter cells were added with the CD19×CD3 bispecific Blincyto at 10 ng/mL and 25 ng/mL. NFAT activation was determined by luminescence after 24 hours (48 Hours from assay start) and plotted against CD19 expression quantified by staining with PE anti-CD19 and plotted on a curve generated using Quantibrite PE receptor quantification kit. n=3, graphs report mean±SD.

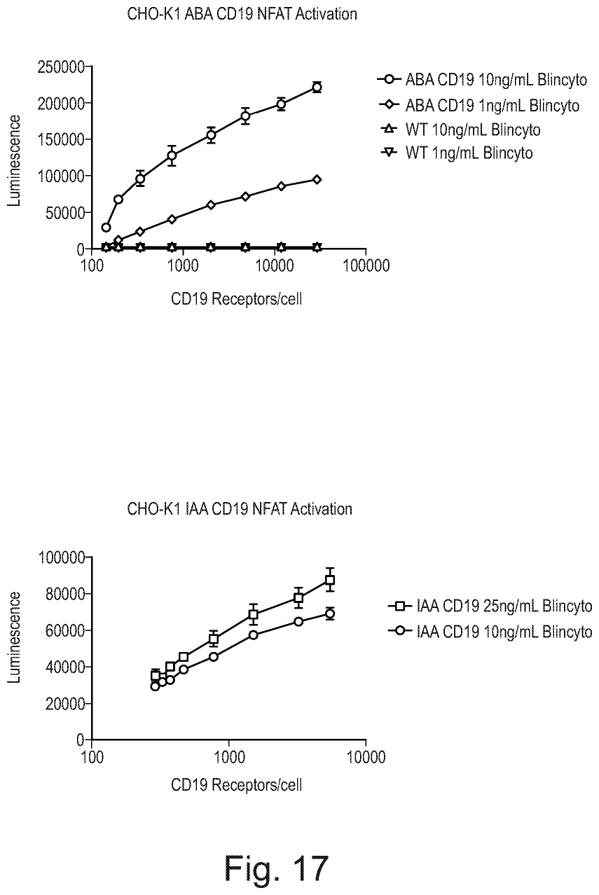

As can be seen in FIG. 17, an ABA or IAA concentration dependent increase in the number of CD19 receptors causes a concurrent increase in luciferase signal from the Jurkat NFAT luciferase reporter cells. This signal is higher but remains IAA and ABA dose dependent when a higher concentration of the CD×CD19 bispecific Blincyto is used.

### Example 4: Further Concurrent Use of the IAA and ABA CIP Systems

To demonstrate the use of both IAA and ABA CIP systems in a single cell, the inventors introduced both CIP systems into a single cell line. To this end, we employed a lentiviral vector approach to introduce the two systems in a single cell, using already established CIP systems described hereinabove and specifically the previously used CHO-K1 IAA activator CD19 clone, where CD19 is under the expression of 10×I-Scel recognition sites, was transduced with an ABA activator cassette as described above, and a corresponding 9×Gal4 upstream activator sequence (UAS)-driven CD22 reporter lentivirus concurrently. Transduced cells were dual selected with hygromycin and puromycin to select for the CD22 reporter and ABA activator cassette, respectively. A successfully transduced cell line was then induced with a concentration series of ABA and IAA, and the expression of CD19 and CD22 was analysed by flow cytometry.

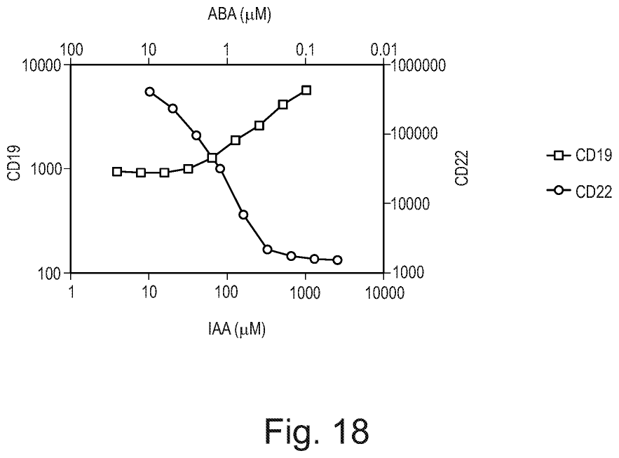

As can be seen in FIG. 18, the two CIP systems (IAA and ABA) are capable of functioning independently in the same cell line. Addition of IAA does not induce expression from the ABA system and addition of ABA does not induce expression of the IAA CIP system.

### Example 5: Assays and Screening/Determination Methods Using the CIP System

In this example, the inventors have demonstrated the use of the chemically inducible proximity (CIP) system in bioassays for screening a candidate binding molecule, a therapeutic agent, and an engineered immune cell for a biological effect, and for determining the minimum level of expression of a target antigen of interest (TAOI) in a target cell required for the biological effects of a candidate binding molecule or therapeutic agent.

The inventors assessed the impact of the concentration of a target antigen, that is, a protein of interest, expressed using the CIP system, on the efficacy of biological molecules including antibody drug conjugates (ADCs), T-cell engagers (TCEs), and chimeric antigen receptor (CAR)-T cells.

### 1 CIP Assay for Screening Antibody Drug Conjugate (ADC) Efficacy

To test the impact of target antigen density on the cytolytic efficacy of antibody drug conjugate (ADCs), two ADCs were used, both comprising the HER2 targeting antibody Trastuzumab but carrying different cytotoxic payloads. Trastuzumab Emtansine is conjugated with the tubulin inhibitor Mertansine, which, following HER2 mediated internalisation of the carrier antibody (Trastuzumab), binds to intracellular tubulin, thus causing mitotic arrest and cell death. Trastuzumab Deruxtecan is conjugated with the topoisomerase I inhibitor Deruxtecan (a derivative of Exatecan). Following HER2 mediated carrier antibody (Trastuzumab) internalization, Deruxtecan inhibits topoisomerase I activity, preventing DNA re-ligation during transcription and replication and thus inducing lethal DNA strand breaks and ultimately cell death.

ABA inducible HER2 CHO-K1 cells were created. These were created by using the ABA activator cassette is as described in FIG. 8 and sequence of SEQ ID NO: 18. The Her2 construct is shown in SEQ ID NO: 19 with the sequence of human Her2 (UniProt P04626) as the reporter gene. CHO-K1 cells were transduced lentivirus expressing the ABA activator cassette (SEQ ID NO: 18) and the 9×Gal4 UAS reporter construct (SEQ ID NO: 19) containing Her2 as the target antigen of interest (TAOI). The resulting 9×Gal4 UAS->Her2 CHO-K1 cells were single cell dilution cloned, and colonies picked for induction analysis following ABA treatment. The clone with the best Her2 expression profile was picked. In particular, the ABA inducible HER2 CHO-K1 cells were seeded in 96 well plates, and treated with a concentration series of ABA for approximately 24 hours. HER2 expression was quantified via immunolabeling and flow cytometry by staining with PE anti-Her2 and then converted to HER2 receptors per cell using the Quantibrite PE receptor quantification kit. HER2 expressing cells were then treated with 50 μg/mL of the anti-Her2 ADCs either Trastuzumab Emtansin or Trastuzumab Deruxtecan for a further 96 hours, and target cell cytolysis measured.

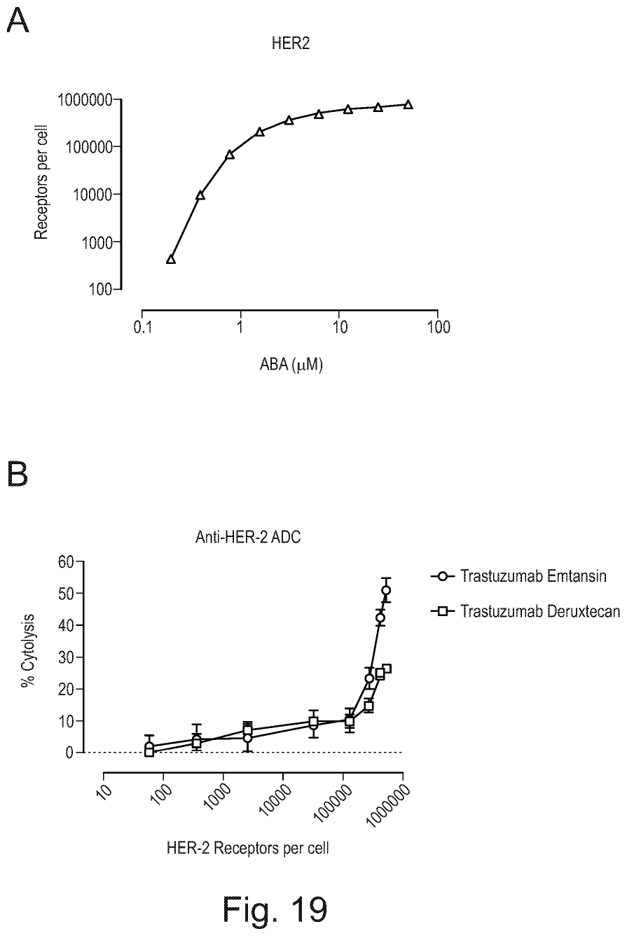

As can be seen in FIG. 19, a difference in the cytotoxicity of the anti-HER2 ADCs is apparent with Trastuzumab Emtansin killing approximately 50% of cells at the highest level of expression of HER2, while at the same level Trastuzumab Deruxtecan kills approximately 25% of cells. Such information can inform their therapeutical potential. In addition, it can be seen that the cytolytic (i.e., biological) activity of ADCs is dependent on HER2 expression.

### 2 CIP Assay for Screening T Cell Engager (TCE) Efficacy and Determining Minimum Levels of Target Antigen Expression Required for TCEs to Enact a Biological Effect

T cell engagers (TCEs) retarget T cell function in an MHC-independent manner against cells that express a specific target antigen of interested (TAOI, CD19 in this example). Usually, these molecules are assessed in bioassays that employ an effector cell, usually T cells, and a target cell that expresses the antigen of interest.

The inventors used a T cell dependent cellular cytotoxicity (TDCC) assay that employed primary immune cells, namely primary T cells, as effector cells. T cell effector functions were monitored, including target cell cytolysis and cytokine production, which are considered the primary mechanisms of action of TCE therapeutics. For this bioassay the xCELLigence RCTA system was employed as in the case of the ADC example above to measure target cell cytolysis. CHO-K1 ABA activator CD19 cells produced as described hereinabove, were seeded and induced with a concentration series of ABA for 24 hours to induce CD19 expression, and CD19 expression was quantified by staining with PE anti-CD19 and plotted on a curve generated using the Quantibrite PE receptor quantification kit. Effector CD8+ T cells isolated from the peripheral blood mononuclear cells (PBMCs) of four donors were then added to the CD-19 expressing target cells at a 3:1 ratio in the presence of 225 pM CD19×CD3 bispecific TCE.

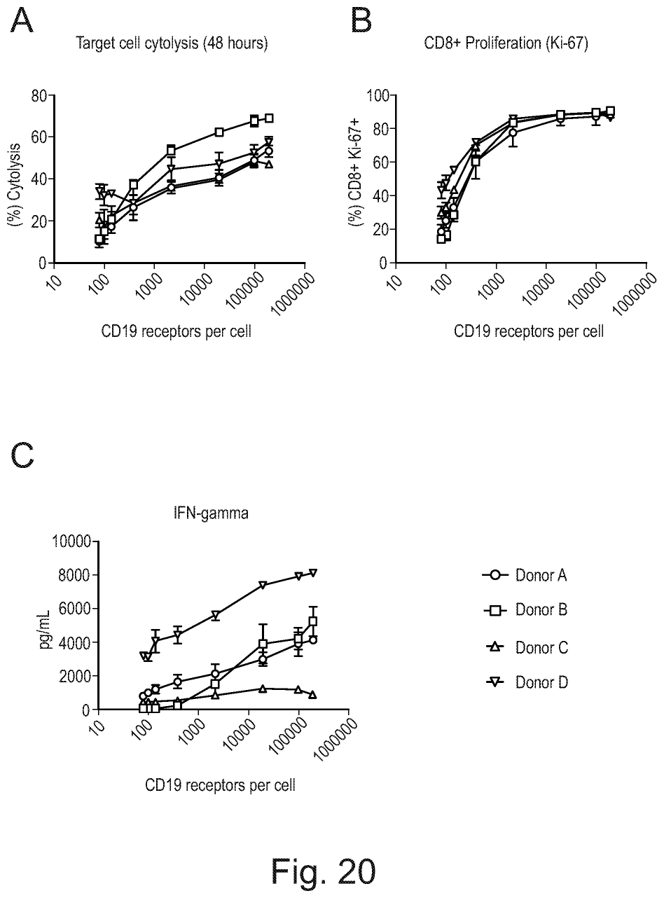

As shown in FIG. 20, TCE-mediated cytotoxicity (FIG. 20A), T cell proliferation as measured by Ki-67+ staining (FIG. 20B), and IFNγ production (FIG. 20C) all clearly correlated with CD19 target antigen expression.

Having successfully demonstrated the applicability of the system using a single CD19×CD3 TCE, the inventors tested the utility of the system to elucidate differences in the biological activities of two different CD3×CD19 TCEs across a range of antigen densities. Such an assay is useful for, for example, finding TCEs that are biologically active in killing target cells at low target antigen levels. This is because CD19 (expressed by healthy and malignant B cells) has been targeted by various therapeutics including TCEs and CAR-Ts. However, one of the main mechanisms of resistance to these therapies is downregulation of the CD19 receptor, resulting in escape of CD19-downregulated or CD19^{Low} cancer cells. In such cases, TCEs that are biologically and therapeutically active at low receptor densities would be considered of higher therapeutic potential.

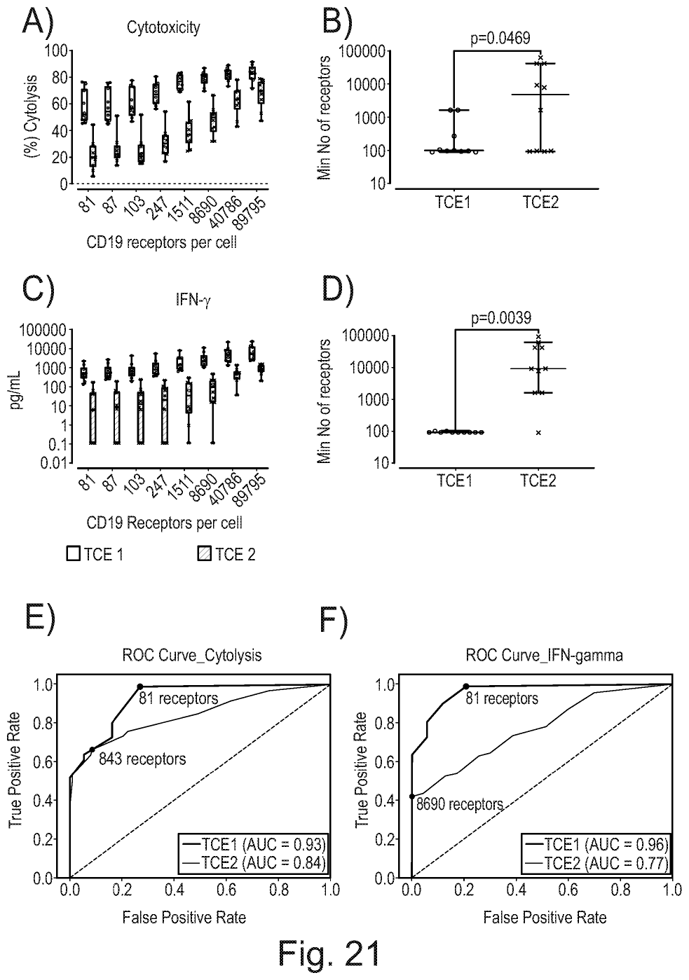

The inventors employed primary T cells derived from a cohort of 10 healthy donors, a single clinically relevant dose of each of TCE 1 and TCE 2, and performed a T-cell mediated cytotoxicity TDCC assay across a range of CD19 expression levels employing the CHO-K1 ABA activator CD19 cell line, as described hereinabove, as target cells. The inventors compared the ability of the two bispecific TCE, CD19×CD3 TCE1 and CD19×CD3 TCE2, to induce target cell killing and IFN-gamma production across seven levels of CD19 expression, as shown in FIGS. 21A and 21C. The inventors then estimated the minimum number of receptors at which an observable biological effect, namely target cell cytolysis or cytokine (IFN-gamma) production, was evident for each donor-derived primary T cell. This was estimated as the minimum number of receptors at which the observable biological effects of target cell cytolysis or IFN-gamma production were greater or equal to 3 standard deviations (SDs) above background (B+3SD), where background was defined as the biological effect of T cells observed against the wild type CHO control which are assumed as having no human CD19 expression (background).

CHO-K1 ABA CD19 cells were seeded in xCELLigence RTCA plates and treated with a concentration series of ABA for 24 hours to induce CD19 expression. CD19 expression was quantified by staining with PE anti-CD19 and plotted on a curve generated using Quanti-Brite PE receptor quantification kit. T cells were isolated from donor PBMCs (n=8) and added to target cells at a 3:1 ratio in the presence of 225 pM CD19×CD3 TCE bispecifics 1 or 2. Target cell death, as determined by impedance loss due to cell detachment, was determined after a further 72 hours.

As shown in FIGS. 21B and 21C, TCE 1 could elicit T cell mediated cytotoxicity and produce IFN-γ at much lower threshold target antigen (CD19) concentrations compared to TCE 2, suggesting a potentially more beneficial therapeutic profile.

To ascertain the threshold number of receptors required to elicit the biological activity of TCE 1 and TCE 2, namely their cytotoxic and interferon-gamma (IFN-γ) producing activities, for the cohort of 10 healthy donors, the receiver operating characteristic (ROC) analysis was used. ROC is a widely accepted method in the art for evaluating the performance of diagnostic tests by distinguishing between positive and negative outcomes, and can also be employed to define a threshold above which an effect is considered to be real (i.e., true). Specifically, Youden's Index (J) was employed to establish this threshold, corresponding to the point on the ROC curve that maximizes the distinction between the true positive rate (sensitivity) and the false positive rate (1—specificity), where sensitivity, specificity and J are defined as:

Sensitivity=true positives/(true positives+false negatives) Roc curve formula

Specificity=true negatives/(true negatives+false positives)

J=sensitivity+specificity−1 Youden's index formula

Again CHO-K1 ABA CD19 cells were cultured in xCELLigence RTCA plates and exposed to a range of ABA concentrations for 24 hours to induce CD19 expression. CD19 expression levels were quantified by staining with PE anti-CD19 and plotted on a curve created using the Quanti-Brite PE receptor quantification kit. T cells were isolated from donor peripheral blood mononuclear cells (PBMCs; n=8) and combined with target cells at a 3:1 ratio in the presence of 225 pM CD19×CD3 bispecific molecules 1 or 2 (TCE1 and TCE2). Target cell lysis and interferon-gamma (IFNγ) production were assessed using xCELLigence RTCA and enzyme-linked immunosorbent assay (ELISA), respectively. ROC curves were generated for each readout and molecule. Youden's Index was calculated for each molecule and readout, and the receptor activation threshold was estimated based on these values. For classification of the results as positive, a threshold of B+3SD was employed, where B is the biological effect observed against WT CHO (no CD19 receptor expression), and SD is the Standard Deviation of the mean of the background.

As shown for cytolysis and IFN-γ in FIGS. 21E and 21F, respectively, by implementing this approach, the inventors discerned that for the donor cohort examined, each effector function exhibited a distinct threshold number of receptors required for TCE 1 and TCE 2 to enact a biological effect. Such information is crucial when evaluating the therapeutic potential of each molecule.

### 3 CIP Assay for Screening Chimeric Antigen Receptor (CAR)-T Cell Efficacy

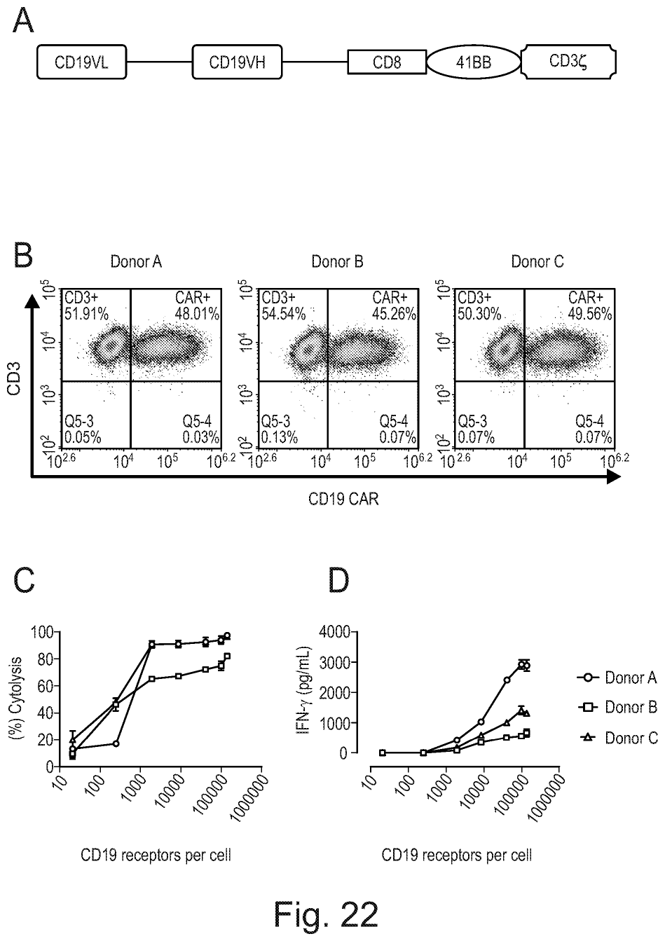

As effector cells, the inventors employed primary T cells from three donors, which were transduced with a lentivirus carrying a FMC63 scFv based CD19-targeting CAR otherwise known as Kymriah™ (depicted as a cartoon diagram in FIG. 22A, and shown in SEQ ID NO: 50) and the ABA CD19 CHO-K1 cell line as target cells. As in previous examples, the assay format utilized the xCELLigence RTCA system and the readout was target cell death as measured by impendence loss due to target cell detachment.

Frozen PBMCs were thawed and cultured for 24 hours before being transduced with CD19 CAR expressing lentivirus. The cells were then expanded for a further 10 days in the presence of IL-5 and IL-17. CD19 CAR expression was detected in CD3 positive T cells using an anti-FMC63 scFv antibody via flow cytometry. CHO-K1 ABA CD19 cells were seeded in xCELLigence RTCA plates and treated with a concentration series of ABA for 24 hours to induce CD19 expression. CD19 expression quantified by staining with PE anti-CD19 and plotted on a curve generated using Quanti-Brite PE receptor quantification kit. CD19/CD22 CAR positive T cells were added at an effector to target ratio of 5:1 and target cell death, as determined by impedance loss due to cell detachment, was determined after a further 24 hours. IFN-γ release by the effector cells was determined via ELISA following 24 incubation with the target cells. n=3, graphs report mean±SD.

As confirmed by flow cytometry, CD19 CAR expression of the resultant CAR cells was relatively consistent across the three donors, ranging from ˜45-50% (FIG. 22B).

As shown in FIGS. 22C and 22D, CAR-T cell-mediated cytolysis and IFN-γ release both correlated with target cell CD19 expression increased in the target cells.

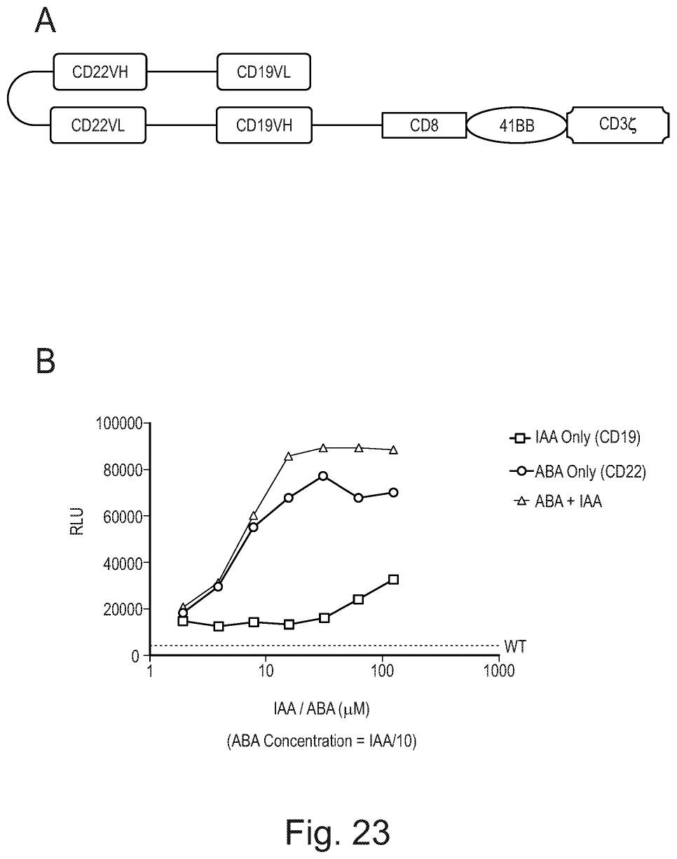

Next, the inventors assessed a bispecific CAR therapeutic targeting two different target antigens, namely a dual CD19/CD22 targeting CAR based on ‘LoopCAR 6’ (FIG. 4A) from https://www.cell.com/molecular-therapy-family/oncolytics/fulltext/S2372-7705 (18) 30030-5?_returnURL=https % 3A %2F %2Flinkinghub.elsevier.com %2Fretrieve %2Fpii %2FS2372770 518300305% 3Fshowall %3Dtrue (depicted as a cartoon diagram in FIG. 23A) and shown in SEQ ID NO:51. This CAR was designed to combat antigen escape in leukaemias and thus requires engagement of either CD19 or CD22 target antigens. We used the dual inducible IAA CD19/ABA CD22 CHO-K1 cells (used previously in Example 4) as target cells in a reporter assay with Jurkat NFAT luciferase reporter cells expressing the CD19/CD22 dual targeting CAR as the effector cells. The readout from the assay was luminescence from the NFAT luciferase reporter, which in turn was driven by CAR signalling (i.e., CAR-T cell activation).

CD19 expression was induced with a concentration series of idole-3-acetic acid (IAA) for 48 hours. CD22 expression induced with concentration series of abscisic acid (ABA) for 24 hours prior to effector cell addition. CD19/CD22 CAR Jurkat expressing NFAT luciferase reporter cells were added at an effector to target ratio of 10:1 and NFAT reporter luciferase activity was measured via luminescence signal approximately 24 hours after effector cell addition.

As shown in FIG. 23B, clear ABA (CD22) and IAA (CD19) dependent increases in luciferase signal (CAR signalling) were observed in the reporter cells. CD19 dependent CAR signalling was lower than CD22 dependent signalling, but this may have largely been due to CD22 expression level being considerably higher than CD19 expression at the same concentration of inducer molecule, as previously shown in FIG. 18 (Example 4). There also appeared to have been an additive effect on CD19/CD22 CAR-T signalling when expression of both CD19 and CD22 is induced (ABA+IAA condition).

Next, a similar experiment was performed but where the dual CD19/CD22 CAR-T effector cells were made from peripheral blood mononuclear cell (PBMC)-derived primary CD3 positive T cells from three different donors. The same dual CD19/CD22 targeting CAR construct was used to transduce T cells (FIG. 23A). The inventors assayed CAR-T activity by measuring target cell cytolysis using xCELLigence RTCA with the readout being cytotoxicity as determined by target cell detachment.

Frozen PBMCs were thawed and cultured for 24 hours before being transduced with CD19/CD22 CAR expressing lentivirus. The cells were then expanded for a further 10 days in the presence of IL-5 and IL-17. CD19/CD22 CAR expression was detected in CD3 positive T cells using an anti-FMC63 antibody via flow cytometry. CD19 and CD22 expression were induced in the target cells either separately or together with concentration series of idole-3-acetic acid (IAA) for 48 hour and a concentration series of abscisic acid (ABA) for 24 hours prior to effector cell addition. Expression quantified by staining with PE anti-CD19 or anti-CD22 and plotted on a curve generated using Quanti-Brite PE receptor quantification kit. CD19/CD22 CAR positive T cells expanded from three different PBMC donors were added at an effector to target ratio of 5:1 and target cell death, as determined by impedance loss due to cell detachment, was determined after a further 24 hours. Donor n=3, error bars report mean±SD.

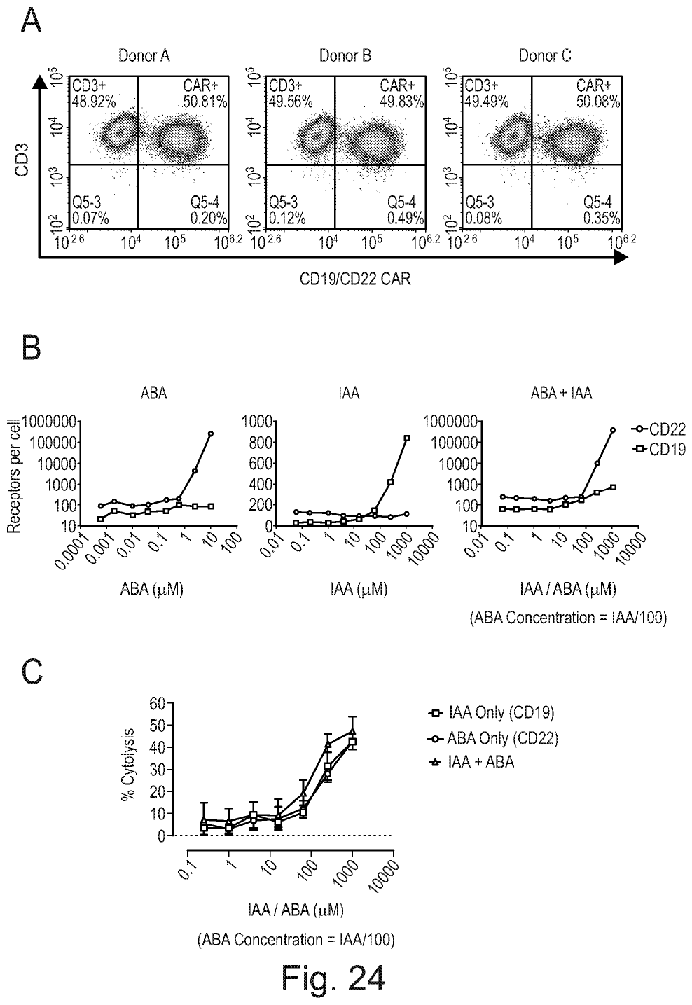

As shown in FIG. 24A, CD19/CD22 CAR expression was relatively consistent across the three donors, at approximately 50%. As shown in FIG. 24B, CD19 and CD22 expression were induced in the IAA CD19/ABA CD22 CHO-K1 cells independently and without cross talk (i.e., without ABA inducing IAA-activated CD 19 expression, and without IAA inducing ABA-activated CD22 expression). As shown in FIG. 24C, cytolysis of the target cells through the cytotoxic activity of the CAR-T effector cells increased with target cell CD19 and CD22 expression levels, with a slight additive effect was observed when the expression of both receptors was induced in the same target cell line (ABA+IAA condition).

4 CIP Assay for Screening Monoclonal Antibody (mAb) Efficacy

Pertuzumab and trastuzumab are monoclonal antibodies targeting different epitopes on HER2, which is a protein overexpressed in some breast cancers. Used as a combination therapy, their primary mechanism of action is antibody-dependent cell-mediated cytotoxicity (ADCC), recruiting immune cells to eliminate HER2-overexpressing tumour cells, thereby enhancing the anti-tumour response and improving clinical outcomes.

The inventors employed the previously used ABA inducible CHO-HER2 cell line as described herein, to assess the impact of HER2 antigen levels on the ADCC activity of either pertuzumab or trastuzumab, or a combination of both. Eight levels of HER2 antigen expression were induced by ABA treatment and the target cells were treated with 20 μg/ml of either trastuzumab or pertuzumab, or 20 μg/mL of a combination of both trastuzumab and pertuzumab. As effector cells, PBMCs from three heathy donors were employed. Target cell cytolysis was monitored with the xCELLigence RTCA system.

Inducible-HER2 CHO were plated in an xCELLIgence E-plate and expression was induced by treatment with a concentration series of ABA. HER2 levels were assessed by flow cytometry and quantified using the Quantibrite™ beads. PBMC from three healthy donors were employed at an effector to target ration of 10:1 and the cocultures were treated with 20 μg/mL of the individual or combination agents. Target cell cytolysis was assessed with the xCELLIgence RTCA system. Each data represents a PBMC donor. Data represented as mean±SD.

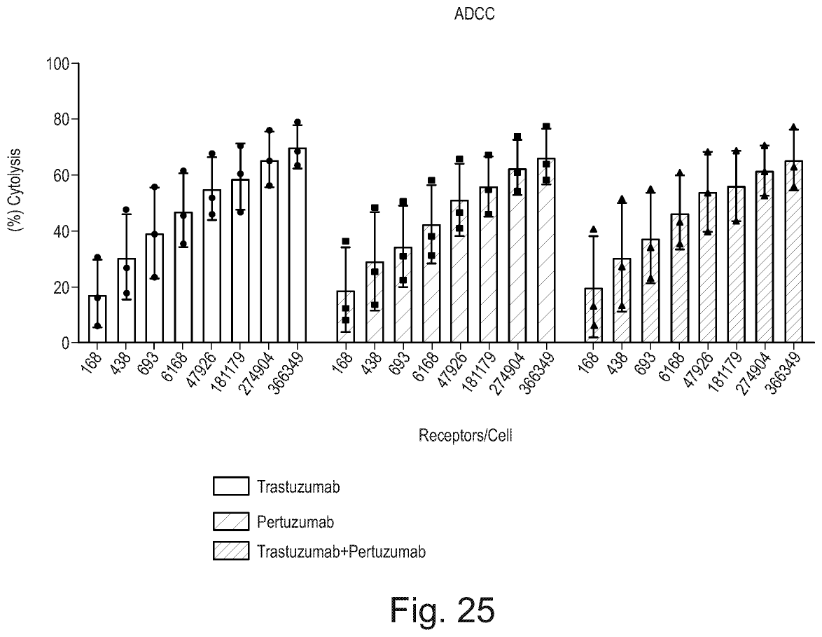

As shown in FIG. 25, both single and combination treatments induced target cell cytolysis in a HER2 receptor concentration-dependent manner, clearly demonstrating the utility of the CIP system to screen candidate agents of interest. There was no observable difference in the ability of the different treatments to induce ADCC in response to the different levels of HER2 receptor expression.

### Example 6: CIP Systems Inducible with Caffeine, Mandipropamid and Giberellin

### 1 Caffeine

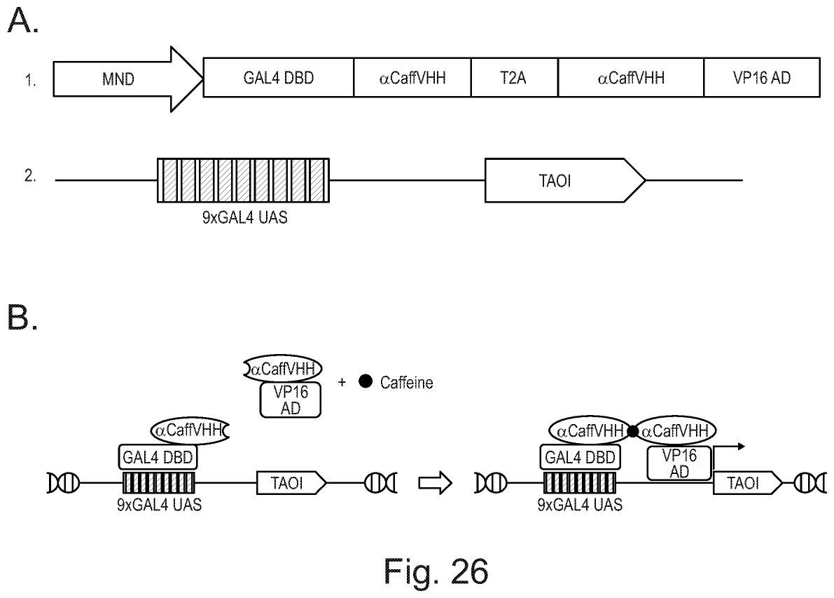

Ladenson et al (2006) described an anti-caffeine heavy-chain antibody fragment (herein referred to as αCaffVHH) isolated from Llama (Lama glama)⁶. Subsequently, it was found that the caffeine binding stoichiometry was two aCaffVHH domains for every caffeine molecule⁷. This dimerization upon caffeine binding allowed the use of aCaffVHH as basis of a caffeine inducible gene switch⁸. In FIG. 26, the inventors demonstrate the use of aCaffVHH in conjunction with the Gal4 DNA binding domain and the VP16 transactivation domain to create a CIP system driven by caffeine. It is expected that this alternative CIP system will function in the same manner as the ABA and IAA CIP systems described herein, and could be interchangeable therewith.

### 2 Mandipropamid (Mandi)

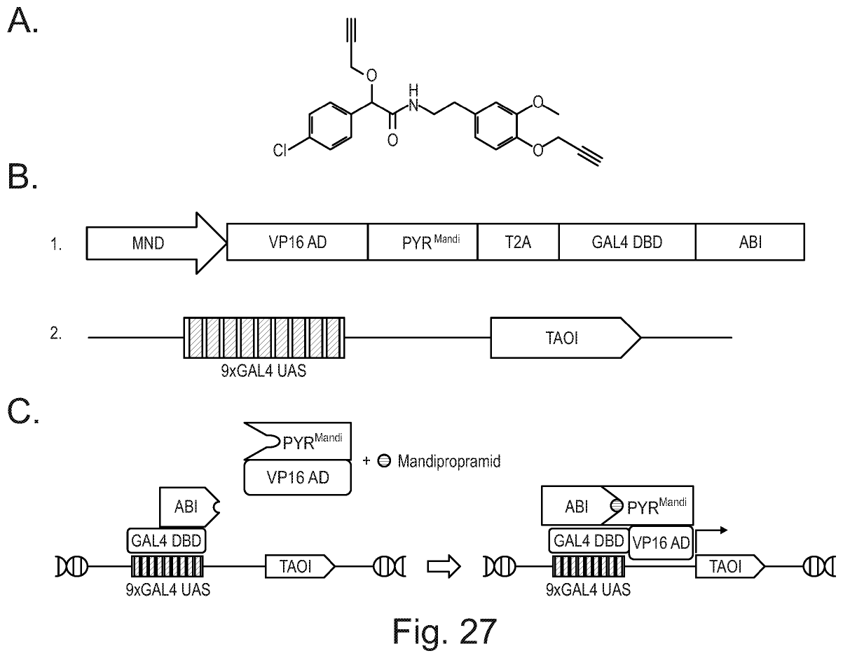

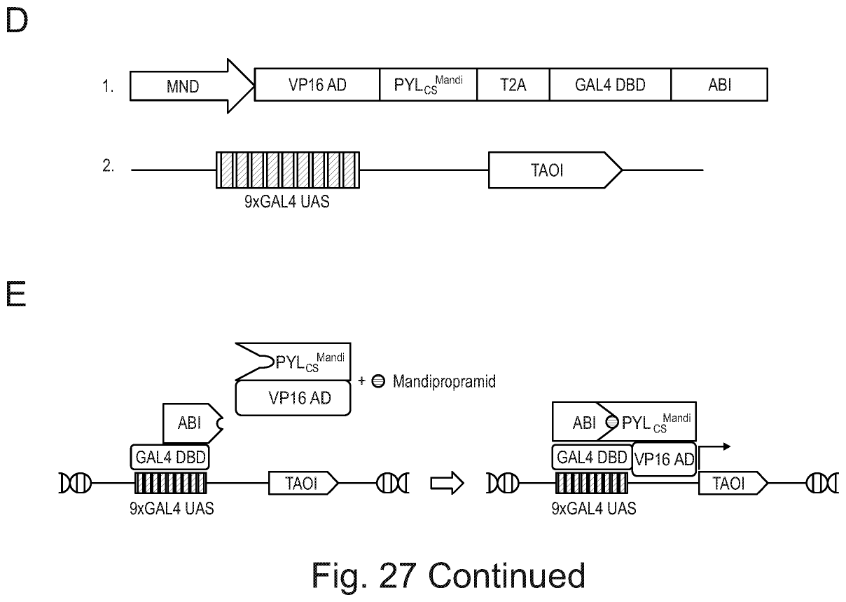

Engineered abscisic acid (ABA) receptors have been reported for agrochemical control of water use in plants⁹. The modified receptors do not respond to the phytohormone ABA but to the agrochemical Mandipropamid (Mandi, FIG. 27A), a fungicide extensively used in agriculture. A hextuple mutant PYR^{Mandi} of the Arabidopsis thaliana ABA receptor PYR1 was identified that specifically binds Mandi⁹, replacing the natural ABA response in plants. The use of PYR^{Mandi} and Mandi as a chemical inducer of proximity for the purposes of controlling subcellular translocation in mammalian cells has been described¹⁰. The inventors transposed the 6 amino acid substitutions in PYR^{Mandi} onto complementary surface (CS) region of Arabidopsis thaliana PYR1-like (PYL) to create PYLcs^{Mandi}. The inventors demonstrate the use of PYR^{Mandi} (FIG. 27B and C) and PYLcs^{Mandi} (FIG. 27C and D) in the ABA induced proximity system as described herein. It is expected that these alternative modified ABA CIP systems will function in the same manner as the ABA CIP system described herein, and could be interchangeable therewith.

### 3 Gibberellin

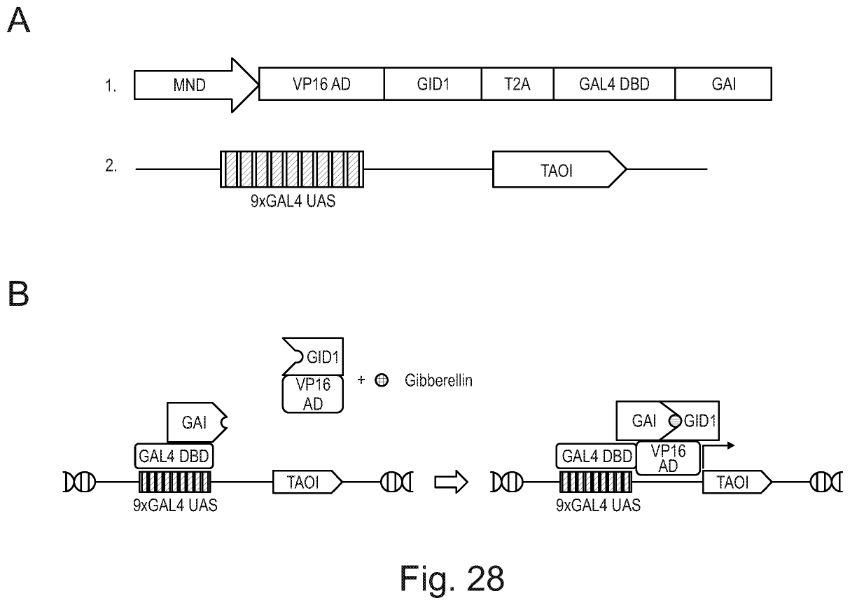

In FIG. 28, the inventors demonstrate a CIP system based on the plant hormone gibberellin, where gibberellin causes dimerization between two Arabidopsis thaliana proteins, gibberellin insensitive dwarf 1 (GID1) and nucleotides 1-276 (amino acids 1-92) of gibberellin insensitive (GAI) 11. It is expected that this alternative CIP system will function in the same manner as the ABA and IAA CIP systems described herein, and could be interchangeable therewith.

### Summary

The examples demonstrate the generation of Mammalian cells expressing the molecular components of a novel IAA CIP system, that is, the so-called IAA activator cassette which comprises the herpes simplex virus VP16 transactivation domain (VP16AD) fused to TIR1 and a catalytically inactive I-Scel homing endonuclease (dl-Scel) fused to AIDΔ34.

Following the introduction of reporter constructs containing EGFP downstream of varying numbers of the i-Scel binding site, it was determined that expression of EGFP could be induced by the addition of IAA to the cell culture media for 24 hours, with the 10× i-Scel binding site reporter providing the largest level of expression induction.

Further to the EGFP expression data, IAA dose dependent induction of expression of CD19 has been demonstrated in CHO-K1 cells expressing the IAA (T2A) activator cassette and the 10× i-Scel CD19 reporter construct. The CD19 expression level was quantified and found to correlate with the levels found in Ramos cells and B cells from PBMCs.

Similar experiments have been carried out in mammalian cells expressing the molecular components of a novel ABA CIP system, that is the so-called ABA-activator cassette which comprises the herpes simplex virus VP16 transactivation domain (VP16AD) fused to a PYL1 protein, and a GAL4 DNA binding domain fused to an ABI1 protein.

Proof of the compatibility of the IAA and ABA system promoters has been demonstrated, with ABA having no effect on the IAA system and vice versa.

Proof of concept has been achieved in a relevant bispecific T cell engager activity assay using Jurkat NFAT luciferase reporter cells and the CD19×CD3 bispecific Blincyto.

Proof of concept has been achieved using the novel IAA and ABA CIP systems to inducibly express CD19 and CD22, respectively, demonstrating that two CIP systems are capable of being independently operated without crosstalk within a single cell.

Proof of concept experiments have shown that titratable expression of a target antigen of interest using the novel CIP system can be used in assays to screen for the biological activities of candidate agents. The biological activities of antibody drug conjugates (ADCs), T cell engagers (TCEs) in combination with T cells, chimeric antigen receptor (CAR)-T cells, and monoclonal antibodies (mAbs) were screened. Biological effects of these agents including target cell cytolysis i.e., cytotoxicity (incl. T-cell mediated cytolysis), TCE-mediated T cell proliferation, IFN-γ, CAR-T signalling i.e., activation, and antibody-dependent cellular cytotoxicity (ADCC) were screened. Demonstrated variations in the biological effects of the various therapeutic agents indicate the utility of CIP system to determine their therapeutic potential.

Proof of concept has been achieved using CD3×CD19 TCEs and T cells showing that the CIP system can be used to determine the minimum level of expression of a target antigen of interest required for the measurable biological activity of said TCE and T cells.

Therefore the inventors have successfully demonstrated two chemically induced proximity systems—auxin (IAA) induced and ABA induced-which may effectively be used to control expression of any described protein of interest such as an antigen. The two systems may be combined to control expression of two different proteins of interest having multiple applications in drug discovery and testing.

### Exemplary Protocols Used in the Studies Described in the Examples

### Construct Design for Auxin (IAA) System

| Construct | Type | FIG. |
|---|---|---|
| IAA Activator | Lentivirus | 14A |
| 1 × iScel EGFP | Lentivirus | 14A |
| 5× iScel EGFP | Lentivirus | 14A |
| 10× iScel EGFP | Lentivirus | 14A |
| 10× iScel CD19 | Lentivirus | 14A |

### Construct Design for Abscisic Acid System

| Construct | Type | FIG. |
|---|---|---|
| ABA Activator | Lentivirus | 14B |
| 9× Gal4 UAS - tagBFP | Lentivirus | 14B |
| 9× Gal4 UAS - CD19 | Lentivirus | 14B |

### Cell Culture

HEK 293 cells were cultured in Dulbecco's modified Eagle's medium (DMEM) (Gibco) with 10% FBS (ThermoFisher Scientific) and 1× penicillin/streptomycin (Pen/Strep; Gibco). CHO-K1 cells were cultured in RPMI 1640 (Gibco) with 10% FBS, 1× glutamate, and 1× Pen/Strep.

### Integration of IAA Activator Cassette

Lentiviral delivery of the IAA or ABA activator cassettes was used. The constructs were designed with a neomycin resistance gene to allow selection of stably transduced cells. HEK293 and CHO cells were seeded into 6 well plates and incubated for a period of 24 hrs before lentivirus containing the IAA or ABA activator cassette was added at an MOI of 15 in the presence of 8 μg/ml polybrene. Following a further incubation period of 48 hours, media containing G418 (Gibco) was added and the cells cultured for a period of 1 week in the selection medium. VP16 expression was measured using flow cytometry (VP16 Antibody (1-21) Alexa Fluor® 488) to ensure the cells were expressing the IAA or ABA activator cassette. The cells were stored at −180° C. for use as a base for further experiments.

### Integration of Reporter Constructs

Once expression of the IAA activator was previously established in HEK293 and CHO-K1, these cells were seeded 6 well plates and incubated overnight before lentivirus carrying an EGFP reporter under the control of varying numbers of the i-Sce-I binding site, and a blasticidin resistance cassette. Lentivirus was added at an MOI of 15 in the presence of 8 μg/ml polybrene. Following a further incubation period of 48 hours, media containing blasticidin (Gibco) was added and the cells cultured for a period of 1 week in the selection medium.

Once expression of the ABA activator was previously established in HEK293 and CHO-K1, these cells were seeded 6 well plates and incubated overnight before lentivirus carrying either CD19 or tagBFP under the control 9 copies of the Gal4 UAS and a hygromycin resistance cassette was added at an MOI of 15 in the presence of 8 μg/ml polybrene. Following a further incubation period of 48 hours, media containing hygromycin (Gibco) was added and the cells cultured for a period of 2 weeks in the selection medium.

Induction of Expression of EGFP with IAA

HEK293 and CHO-K1 cells containing the IAA activator cassette and the EGFP reporter constructs were seeded in 6 well plates. Following overnight incubation, the media was replaced with fresh media containing the indicated concentrations of IAA and the cells incubated for a further 24 hours. EGFP expression was detected using a NovoCyte flow cytometer (ACEA).

Induction of Expression of tagBFP and CD19 with ABA

HEK293 and CHO-K1 cells containing the IAA activator cassette and the EGFP reporter constructs were seeded in 6 well plates. Following overnight incubation, the media was replaced with fresh media containing the indicated concentrations of ABA and the cells incubated for a further 24 hours. BFP expression was detected using a NovoCyte flow cytometer (ACEA).

### Effect of IAA on Proliferation of T Cells

Peripheral Blood Mononuclear Cells (PBMCs) from two donors were labelled with eBioscience™ Cell Proliferation Dye eFluor™ 670 (ThermoFisher #65-0840-90). 2×10⁵ labelled cells were seeded in a 96-well plated and stimulated with Dynabead Human T-Activator CD3/CD28 (ThermoFisher, #111.32D) at 1:1 bead:cell ratio in the presence of dilution series of IAA or ABA. The cells were incubated for 96 hrs in a humidified incubator at 37° C./6% CO2 at which point proliferation of CD4+ and CD8+ T cells was assessed by flow cytometry. In parallel, supernatants were collected and the levels of IFNγ was analysed by ELISA (Human IFN-gamma DuoSet ELISA, R&D systems #DY285B).

### Aba Dose Response Assessment

CHO-K1 cells containing the ABA activator cassette and the CD19 reporter constructs were seeded in 96 well plates. Following overnight incubation, the media was replaced with fresh media containing the indicated concentrations of ABA and the cells were incubated for a further 48 hours. CD19 expression was detected by anti-CD19 staining (Biolegend #302254) and flow cytometry analysis. The number of CD19 receptors per cell was quantified using a Quantibrite PE bead fluorescence quantification kit (BD Biosciences #340495).

### Iaa Dose Response Assessment

CHO-K1 cells containing the IAA activator cassette and a 10× i-Scel CD19 reporter construct were seeded overnight in 96 well plates. IAA was added at the indicated concentrations and the cells were incubated for a further 72-hours. CD19 expression was assess by flow cytometry and quantified for receptor number per cell. By means of comparison, the CD19 expression level of the CD19 positive B cells found in peripheral blood mononuclear cells (PBMCs) and Ramos cells were calculated in the same experiment. The number of CD19 receptors per cell was quantified using a Quantibrite PE bead fluorescence quantification kit (BD Biosciences #340495).

IAA and ABA Dose Response Assessment within a Single Cell

CFHO-K1 cells containing both ABA and IAA activator cassettes as well as ABA-inducible CD22 and IAA-inducible CD19 reporters constructs were seeded and treated with a concentration series of IAA for 48 hours, and ABA for 24 hours. CD22 and CD19 expression was measured by flow cytometry and mean fluorescence intensity corresponding to CD22 and CD19 expression was plotted.

### Screening ADCs Dose-Dependence by Titratable HER2 Expression

CHO-K1 ABA activator HER2 cells were created by lentiviral transduction as described for other constructs. seeded and treated with a concentration series of ABA for approximately 24 hours. HER2 expression was quantified via flow cytometry by staining with PE anti-HER2 and expressed in units of HER2 receptors per cell using the Quantibrite PE receptor quantification kit. After 24 hours, HER2 expressing cells were treated with 50 μg/mL of anti-HER2 ADCs Trastuzumab Emtansin or Trastuzumab Deruxtecan for a further 96 hours. Cell cytolysis was measured using the xCELLigence RTCA instrument and percent cytolysis was calculated by normalising data to untreated target only cells which were not treated with ADCs, and plotted against Her2 expression.

The xCELLigence RTCA instrument use biosensors to continuously monitor cell behaviour in a label-free manner. The method employs 96-well plates lined with gold electrodes that conduct a small electric current, which is impeded when a cell adheres on the plate. Upon targeting with a cytotoxic agent, the cells change morphology and de-attach resulting in a loss of signal.

### T Cell Engager (TCE) Experiments

CHO-K1 ABA CD19 cells were seeded and treated with a concentration series of ABA for 24 hours to induce CD19 expression. CD19 expression quantified by staining with PE anti-CD19 and expressed in units of CD19 receptors per cell using the Quantibrite PE receptor quantification kit. CD8+ T cells were isolated from PBMCs and added to target cells at a 3:1 ratio in the presence of 225 pM CD19×CD3 bispecific. Target cell death measured using the xCELLigence RTCA instrument was determined after a further 48 hours. CD8+ T cell proliferation was measured via Ki-67 staining and flow cytometry analysis. IFN-γ production by the effector cells was measured via ELISA after 48-hour co-incubation with the target cells.

For experiments involving a comparison of two CD19×CD13 TCEs, the protocol was as follows: CHO-K1 ABA CD19 cells were seeded in xCELLigence RTCA plates and treated with a concentration series of ABA for 24 hours to induce CD19 expression. CD19 expression quantified by staining with PE anti-CD19 and plotted on a curve generated using Quanti-Brite PE receptor quantification kit. T cells were isolated from ten donor PBMCs (n=10) and added to target cells at a 3:1 ratio in the presence of 225 pM CD19×CD3 bispecifics 1 or 2, and target cell death, as determined by impedance loss due to cell detachment, was determined after a further 72 hours. Comparison of T cell mediated cytotoxicity and IFN-gamma production induced by the two CD19×CD3 bispecific T cell engagers against CHO-K1 ABA CD19 cells across a range of CD19 receptors per cell. Each data point represents a single T cell donor. Data show median (N=10)+/−SD. B and D)

The minimum number of receptors for which a biologic effect (either cytotoxicity or IFN-gamma production) was observed and was estimated as the number of receptors at which an effect higher than the mean background effect +3 standard deviations was observed, where the background effect was the biological effect observed when wild type CHOs were used as target cells, which were assumed as having 0 number of target CD19 receptors. The Wilcoxon matched-pairs signed rank test was employed to compare the two treatments.

### Cd19 Chimeric Antigen Receptor (CAR)-T Cell Experiments

Frozen PBMCs were thawed and cultured for 24 hours before being transduced with the CD19 CAR expressing lentivirus depicted in FIG. 22A. The cells were then expanded for a further 10 days in the presence of IL-5 and IL-17. CD19 CAR expression was detected in CD3 positive T cells using an anti-FMC63 scFv antibody via flow cytometry. CHO-K1 ABA CD19 cells were seeded in xCELLigence RTCA plates and treated with a concentration series of ABA for 24 hours to induce CD19 expression. CD19 expression quantified by staining with PE anti-CD19 and expressed in units of CD19 receptors per cell using the Quantibrite PE receptor quantification kit. CD19 CAR positive T cells were added at an effector to target ratio of 5:1 and target cell death, as determined by impedance loss due to cell detachment, was determined using the xCELLigence RTCA instrument after a further 24 hours. IFN-γ release by the effector T cells was determined via ELISA following 24 incubation with the target cells.

### Dual CD19/CD22 CAR-T Cell Experiments

CHO-K1 cells containing both ABA and IAA activator cassettes as well as ABA-inducible CD22 and IAA-inducible CD19 reporters constructs were seeded and CD19 expression was induced with a concentration series of idole-3-acetic acid (IAA) for 48 hours while CD22 expression induced with concentration series of abscisic acid (ABA) for 24 hours prior to effector cell addition. In one set of experiments (FIG. 23), CD19/CD22 CAR Jurkat expressing NFAT luciferase reporter cells were added at an effector to target cell ratio of 10:1 and NFAT reporter luciferase activity was measured via luminescence signal approximately 24 hours after effector cell addition. In another set of experiments (FIG. 24), frozen PBMCs from three donors were thawed and cultured for 24 hours before being transduced with the CD19/CD22 CAR expressing lentivirus. Cells were then expanded for a further 10 days in the presence of IL-5 and IL-17. CD19/CD22 CAR expression was detected in CD3 positive T cells using an anti-FMC63 antibody via flow cytometry. CD19 and CD22 expression were induced in the target cells either separately or together with concentration series of idole-3-acetic acid (IAA) for 48 hour and a concentration series of abscisic acid (ABA) for 24 hours prior to effector cell addition. Expression quantified by staining with PE anti-CD19 or anti-CD22 and plotted on a curve generated using Quanti-Brite PE receptor quantification kit. Then, CD19/CD22 CAR positive T cells expanded from three different PBMC donors were added at an effector to target cell ratio of 5:1 and target cell death, as determined by impedance loss due to cell detachment, was determined using the xCELLigence RTCA instrument after a further 24 hours.

Monoclonal Antibody (mAb) Experiments

Inducible-HER2 CHO were plated in an xCELLigence plates and HER2 expression was induced by treatment with a concentration series of ABA. HER2 levels were assessed by flow cytometry and quantified using the Quantibrite beads. PBMC from three healthy donors were employed at an effector to target ration of 10:1 and the cocultures were treated with 20 μg/mL of the individual or combination agents. Target cell cytolysis was assessed with the xCELLIgence RTCA system.

### Sequence Information

IAA related DNA sequences
i-SceI recognition site (SEQ ID NO: 1)
TAGGGATAACAGGGTAAT
IAA activator cassette (T2A linker) (SEQ ID NO: 2)
atgggcagtgtcgagctgaatctgagggagactgagctgtgtcttggtcttcccggtggagatacagtggctccggtaaccggaa
acaagagagggttctcagagacggttgatctgaagctaaatctgaataatgagcctgcaaacaaggaaggatctacgactcat
gacgtcgtgacttttgattccaaggagaagagtgcttgtcctaaagatccagccaaacctccggccaaggcacaagttgtgggat
ggccaccggtgagatcataccggaagaacggtggcggaagcggagggggatcgatgagcaccgccccccccaccgacgt
gagcctgggcgacgagctgcacctggacggcgaggacgtggccatggcccacgccgacgccctggacgacttcgacctgga
catgctgggcgacggcgacagccccggccccggcttcaccccccacgacagcgccccctacggcgccctggacatggccga
cttcgagttcgagcagatgttcaccgacgccctgggcatcgacgagtacggcggcaggaggaagaggggaagcggagagg
gcaggggaagtcttctaacatgcggggacgtggaggaaaatcccggccccatgaagaacatcaagaagaaccaggtgatg
aacctgggccccaacagcaagctgctgaaggagtacaagagccagctgatcgagctgaacatcgagcagttcgaggccggc
atcggcctgatcctgggcagcgcctacatcaggagcagggacgagggcaagacctactgcatgcagttcgagtggaagaac
aaggcctacatggaccacgtgtgcctgctgtacgaccagtgggtgctgagccccccccacaagaaggagagggtgaaccac
ctgggcaacctggtgatcacctggggcgcccagaccttcaagcaccaggccttcaacaagctggccaacctgttcatcgtgaac
aacaagaagaccatccccaacaacctggtggagaactacctgacccccatgagcctggcctactggttcatggacgccggcg
gcaagtgggactacaacaagaacagcaccaacaagagcatcgtgctgaacacccagagcttcaccttcgaggaggtggagt
acctggtgaagggcctgaggaacaagttccagctgaactgctacgtgaagatcaacaagaacaagcccatcatctacatcga
cagcatgagctacctgatcttctacaacctgatcaagccctacctgatcccccagatgatgtacaagctgcccaacaccatcagc
agcgagaccttcctgaagggtggcggaagcggagggggatcgatgacctacttccccgagaaggtggtgaagcacatcttca
gcttcctgcccgcccagagggacaggaacaccgtgagcctggtgtgcaaggtgtggtacgagatcgagaggctgagcagga
ggggcgtgttcgtgggcaactgctacgccgtgagggccggcagggtggccgccaggttccccaacgtgagggccctgaccgt
gaagggcaagccccacttcgccgacttcaacctggtgccccccgactggggcggctacgccggcccctggatcgaggccgcc
gccaggggctgccacggcctggaggagctgaggatgaagaggatggtggtgagcgacgagagcctggagctgctggccag
gagcttccccaggttcagggccctggtgctgatcagctgcgagggcttcagcaccgacggcctggccgccgtggccagccact
gcaagctgctgagggagctggacctgcaggagaacgaggtggaggacaggggccccaggtggctgagctgcttccccgac
agctgcaccagcctggtgagcctgaacttcgcctgcatcaagggcgaggtgaacgccggcagcctggagaggctggtgagca
ggagccccaacctgaggagcctgaggctgaacaggagcgtgagcgtggacaccctggccaagatcctgctgaggaccccc
aacctggaggacctgggcaccggcaacctgaccgacgacttccagaccgagagctacttcaagctgaccagcgccctggag
aagtgcaagatgctgaggagcctgagcggcttctgggacgccagccccgtgtgcctgagcttcatctaccccctgtgcgcccag
ctgaccggcctgaacctgagctacgcccccaccctggacgccagcgacctgaccaagatgatcagcaggtgcgtgaagctgc
agaggctgtgggtgctggactgcatcagcgacaagggcctgcaggtggtggccagcagctgcaaggacctgcaggagctga
gggtgttccccagcgacttctacgtggccggctacagcgccgtgaccgaggagggcctggtggccgtgagcctgggctgcccc
aagctgaacagcctgctgtacttctgccaccagatgaccaacgccgccctggtgaccgtggccaagaactgccccaacttcacc
aggttcaggctgtgcatcctggagcccggcaagcccgacgtggtgaccagccagcccctggacgagggcttcggcgccatcgt
gagggagtgcaagggcctgcagaggctgagcatcagcggcctgctgaccgacaaggtgttcatgtacatcggcaagtacgcc
aagcagctggagatgctgagcatcgccttcgccggcgacagcgacaagggcatgatgcacgtgatgaacggctgcaagaac
ctgaggaagctggagatcagggacagccccttcggcgacgccgccctgctgggcaacttcgccaggtacgagaccatgagg
agcctgtggatgagcagctgcaacgtgaccctgaagggctgccaggtgctggccagcaagatgcccatgctgaacgtggagg
tgatcaacgagagggacggcagcaacgagatggaggagaaccacggcgacctgcccaaggtggagaagctgtacgtgtac
aggaccaccgccggcgccagggacgacgcccccaacttcgtgaagatcctgtaa
IAA activator cassette (IRES linker)
AIDΔ34-VP16-Myc (SEQ ID NO: 3)
atgggcagtgtcgagctgaatctgagggagactgagctgtgtcttggtcttcccggtggagatacagtggctccggtaaccggaa
acaagagagggttctcagagacggttgatctgaagctaaatctgaataatgagcctgcaaacaaggaaggatctacgactcat
gacgtcgtgacttttgattccaaggagaagagtgcttgtcctaaagatccagccaaacctccggccaaggcacaagttgtgggat
ggccaccggtgagatcataccggaagaacggtggcggaagcggagggggatcgatgagcaccgccccccccaccgacgt
gagcctgggcgacgagctgcacctggacggcgaggacgtggccatggcccacgccgacgccctggacgacttcgacctgga
catgctgggcgacggcgacagccccggccccggcttcaccccccacgacagcgccccctacggcgccctggacatggccga
cttcgagttcgagcagatgttcaccgacgccctgggcatcgacgagtacggcggcggtggcggaagcggagggggatcgga
acaaaaactcatctcagaagaggatctgtaa
IRES (SEQ ID NO: 4)
cccctctccctcccccccccctaacgttactggccgaagccgcttggaataaggccggtgtgcgtttgtctatatgttattttccaccat
attgccgtcttttggcaatgtgagggcccggaaacctggccctgtcttcttgacgagcattcctaggggtctttcccctctcgccaaag
gaatgcaaggtctgttgaatgtcgtgaaggaagcagttcctctggaagcttcttgaagacaaacaacgtctgtagcgaccctttgc
aggcagcggaaccccccacctggcgacaggtgcctctgcggccaaaagccacgtgtataagatacacctgcaaaggcggc
acaaccccagtgccacgttgtgagttggatagttgtggaaagagtcaaatggctctcctcaagcgtattcaacaaggggctgaag
gatgcccagaaggtaccccattgtatgggatctgatctggggcctcggtgcacatgctttacatgtgtttagtcgaggttaaaaaaa
cgtctaggccccccgaaccacggggacgtggttttcctttgaaaaacacgatgataatatggccacaacc
3xNLS-dI-SceI-TIR1-V5 (SEQ ID NO: 5)
atggccacaaccatggatccaaaaaagaagagaaaggtagatccaaaaaagaagagaaaggtagatccaaaaaagaag
agaaaggtaatgaagaacatcaagaagaaccaggtgatgaacctgggccccaacagcaagctgctgaaggagtacaaga
gccagctgatcgagctgaacatcgagcagttcgaggccggcatcggcctgatcctgggcagcgcctacatcaggagcaggga
cgagggcaagacctactgcatgcagttcgagtggaagaacaaggcctacatggaccacgtgtgcctgctgtacgaccagtgg
gtgctgagccccccccacaagaaggagagggtgaaccacctgggcaacctggtgatcacctggggcgcccagaccttcaag
caccaggccttcaacaagctggccaacctgttcatcgtgaacaacaagaagaccatccccaacaacctggtggagaactacct
gacccccatgagcctggcctactggttcatggacgccggcggcaagtgggactacaacaagaacagcaccaacaagagcat
cgtgctgaacacccagagcttcaccttcgaggaggtggagtacctggtgaagggcctgaggaacaagttccagctgaactgct
acgtgaagatcaacaagaacaagcccatcatctacatcgacagcatgagctacctgatcttctacaacctgatcaagccctacct
gatcccccagatgatgtacaagctgcccaacaccatcagcagcgagaccttcctgaagggtggcggaagcggagggggatc
gatgacctacttccccgagaaggtggtgaagcacatcttcagcttcctgcccgcccagagggacaggaacaccgtgagcctgg
tgtgcaaggtgtggtacgagatcgagaggctgagcaggaggggcgtgttcgtgggcaactgctacgccgtgagggccggcag
ggtggccgccaggttccccaacgtgagggccctgaccgtgaagggcaagccccacttcgccgacttcaacctggtgccccccg
actggggcggctacgccggcccctggatcgaggccgccgccaggggctgccacggcctggaggagctgaggatgaagagg
atggtggtgagcgacgagagcctggagctgctggccaggagcttccccaggttcagggccctggtgctgatcagctgcgaggg
cttcagcaccgacggcctggccgccgtggccagccactgcaagctgctgagggagctggacctgcaggagaacgaggtgga
ggacaggggccccaggtggctgagctgcttccccgacagctgcaccagcctggtgagcctgaacttcgcctgcatcaagggcg
aggtgaacgccggcagcctggagaggctggtgagcaggagccccaacctgaggagcctgaggctgaacaggagcgtgag
cgtggacaccctggccaagatcctgctgaggacccccaacctggaggacctgggcaccggcaacctgaccgacgacttcca
gaccgagagctacttcaagctgaccagcgccctggagaagtgcaagatgctgaggagcctgagcggcttctgggacgccagc
cccgtgtgcctgagcttcatctaccccctgtgcgcccagctgaccggcctgaacctgagctacgcccccaccctggacgccagc
gacctgaccaagatgatcagcaggtgcgtgaagctgcagaggctgtgggtgctggactgcatcagcgacaagggcctgcagg
tggtggccagcagctgcaaggacctgcaggagctgagggtgttccccagcgacttctacgtggccggctacagcgccgtgacc
gaggagggcctggtggccgtgagcctgggctgccccaagctgaacagcctgctgtacttctgccaccagatgaccaacgccgc
cctggtgaccgtggccaagaactgccccaacttcaccaggttcaggctgtgcatcctggagcccggcaagcccgacgtggtga
ccagccagcccctggacgagggcttcggcgccatcgtgagggagtgcaagggcctgcagaggctgagcatcagcggcctgc
tgaccgacaaggtgttcatgtacatcggcaagtacgccaagcagctggagatgctgagcatcgccttcgccggcgacagcgac
aagggcatgatgcacgtgatgaacggctgcaagaacctgaggaagctggagatcagggacagccccttcggcgacgccgc
cctgctgggcaacttcgccaggtacgagaccatgaggagcctgtggatgagcagctgcaacgtgaccctgaagggctgccag
gtgctggccagcaagatgcccatgctgaacgtggaggtgatcaacgagagggacggcagcaacgagatggaggagaacc
acggcgacctgcccaaggtggagaagctgtacgtgtacaggaccaccgccggcgccagggacgacgcccccaacttcgtga
agatcctgggtggcggaagcggagggggatcgggtaagcctatccctaaccctctcctcggtctcgattctacgtaa
LexA Operator Sequence (SEQ ID NO: 6)
CTGTATATATATACAG
10 x i-SceI recognition site + minimal promoter (SEQ ID NO: 7)
Tctagaaactctagggataacagggtaatcttagggataacagggtaatcttagggataacagggtaatcttagggataacagg
gtaatcttagggataacagggtaattctagggataacagggtaatcttagggataacagggtaatcttagggataacagggtaat
cttagggataacagggtaatcttagggataacagggtaatggggggctataaaagggggtgggggcgttcgtcctcactctaga
tctgcgatctaagtaagcttggcaatccggtactgttggtaa
IAA Related Amino acid sequences
IAA activator cassette (T2A linker) (SEQ ID NO: 8)
MGSVELNLRETELCLGLPGGDTVAPVTGNKRGFSETVDLKLNLNNEPANKEGSTTHDVVTF
DSKEKSACPKDPAKPPAKAQVVGWPPVRSYRKNGGGSGGGSMSTAPPTDVSLGDELHLD
GEDVAMAHADALDDFDLDMLGDGDSPGPGFTPHDSAPYGALDMADFEFEQMFTDALGID
EYGGRRKRGSGEGRGSLLTCGDVEENPGPMKNIKKNQVMNLGPNSKLLKEYKSQLIELNIE
QFEAGIGLILGSAYIRSRDEGKTYCMQFEWKNKAYMDHVCLLYDQWVLSPPHKKERVNHL
GNLVITWGAQTFKHQAFNKLANLFIVNNKKTIPNNLVENYLTPMSLAYWFMDAGGKWDYNK
NSTNKSIVLNTQSFTFEEVEYLVKGLRNKFQLNCYVKINKNKPIIYIDSMSYLIFYNLIKPYLIPQ
MMYKLPNTISSETFLKGGGSGGGSMTYFPEKVVKHIFSFLPAQRDRNTVSLVCKVWYEIER
LSRRGVFVGNCYAVRAGRVAARFPNVRALTVKGKPHFADFNLVPPDWGGYAGPWIEAAA
RGCHGLEELRMKRMVVSDESLELLARSFPRFRALVLISCEGFSTDGLAAVASHCKLLRELDL
QENEVEDRGPRWLSCFPDSCTSLVSLNFACIKGEVNAGSLERLVSRSPNLRSLRLNRSVSV
DTLAKILLRTPNLEDLGTGNLTDDFQTESYFKLTSALEKCKMLRSLSGFWDASPVCLSFIYPL
CAQLTGLNLSYAPTLDASDLTKMISRCVKLQRLWVLDCISDKGLQVVASSCKDLQELRVFPS
DFYVAGYSAVTEEGLVAVSLGCPKLNSLLYFCHQMTNAALVTVAKNCPNFTRFRLCILEPGK
PDVVTSQPLDEGFGAIVRECKGLQRLSISGLLTDKVFMYIGKYAKQLEMLSIAFAGDSDKGM
MHVMNGCKNLRKLEIRDSPFGDAALLGNFARYETMRSLWMSSCNVTLKGCQVLASKMPM
LNVEVINERDGSNEMEENHGDLPKVEKLYVYRTTAGARDDAPNFVKIL*
IAA activator cassette (IRES linker)
AIDΔ34-VP16-Myc (SEQ ID NO: 9)
MGSVELNLRETELCLGLPGGDTVAPVTGNKRGFSETVDLKLNLNNEPANKEGSTTHDVVTF
DSKEKSACPKDPAKPPAKAQVVGWPPVRSYRKNGGGSGGGSMSTAPPTDVSLGDELHLD
GEDVAMAHADALDDFDLDMLGDGDSPGPGFTPHDSAPYGALDMADFEFEQMFTDALGID
EYGGGGGSGGGSEQKLISEEDL*
3xNLS-dI-SceI-TIR1-V5 (SEQ ID NO: 10)
MATTMDPKKKRKVDPKKKRKVDPKKKRKVMKNIKKNQVMNLGPNSKLLKEYKSQLIELNIE
QFEAGIGLILGSAYIRSRDEGKTYCMQFEWKNKAYMDHVCLLYDQWVLSPPHKKERVNHL
GNLVITWGAQTFKHQAFNKLANLFIVNNKKTIPNNLVENYLTPMSLAYWFMDAGGKWDYNK
NSTNKSIVLNTQSFTFEEVEYLVKGLRNKFQLNCYVKINKNKPIIYIDSMSYLIFYNLIKPYLIPQ
MMYKLPNTISSETFLKGGGSGGGSMTYFPEKVVKHIFSFLPAQRDRNTVSLVCKVWYEIER
LSRRGVFVGNCYAVRAGRVAARFPNVRALTVKGKPHFADFNLVPPDWGGYAGPWIEAAA
RGCHGLEELRMKRMVVSDESLELLARSFPRFRALVLISCEGFSTDGLAAVASHCKLLRELDL
QENEVEDRGPRWLSCFPDSCTSLVSLNFACIKGEVNAGSLERLVSRSPNLRSLRLNRSVSV
DTLAKILLRTPNLEDLGTGNLTDDFQTESYFKLTSALEKCKMLRSLSGFWDASPVCLSFIYPL
CAQLTGLNLSYAPTLDASDLTKMISRCVKLQRLWVLDCISDKGLQVVASSCKDLQELRVFPS
DFYVAGYSAVTEEGLVAVSLGCPKLNSLLYFCHQMTNAALVTVAKNCPNFTRFRLCILEPGK
PDVVTSQPLDEGFGAIVRECKGLQRLSISGLLTDKVFMYIGKYAKQLEMLSIAFAGDSDKGM
MHVMNGCKNLRKLEIRDSPFGDAALLGNFARYETMRSLWMSSCNVTLKGCQVLASKMPM
LNVEVINERDGSNEMEENHGDLPKVEKLYVYRTTAGARDDAPNFVKILGGGSGGGSGKPIP
NPLLGLDST*
Effector Domain and Binding Domain Sequences
VP16 transactivation domain (SEQ ID NO: 11)
MSTAPPTDVSLGDELHLDGEDVAMAHADALDDFDLDMLGDGDSPGPGFTPHDSAPYGAL
DMADFEFEQMFTDALGIDEYGG
Wild type I-SceI (SEQ ID NO: 12)
MKNIKKNQVMNLGPNSKLLKEYKSQLIELNIEQFEAGIGLILGDAYIRSRDEGKTYCMQFEW
KNKAYMDHVCLLYDQWVLSPPHKKERVNHLGNLVITWGAQTFKHQAFNKLANLFIVNNKKT
IPNNLVENYLTPMSLAYWFMDDGGKWDYNKNSTNKSIVLNTQSFTFEEVEYLVKGLRNKFQ
LNCYVKINKNKPIIYIDSMSYLIFYNLIKPYLIPQMMYKLPNTISSETFLK*
Modified dI-SceI (D44S, D145A) (SEQ ID NO: 13)
MKNIKKNQVMNLGPNSKLLKEYKSQLIELNIEQFEAGIGLILGSAYIRSRDEGKTYCMQFEWK
NKAYMDHVCLLYDQWVLSPPHKKERVNHLGNLVITWGAQTFKHQAFNKLANLFIVNNKKTI
PNNLVENYLTPMSLAYWFMDAGGKWDYNKNSTNKSIVLNTQSFTFEEVEYLVKGLRNKFQ
LNCYVKINKNKPIIYIDSMSYLIFYNLIKPYLIPQMMYKLPNTISSETFLK*
Wild Type osTIR1 (SEQ ID NO: 14)
MTYFPEEVVEHIFSFLPAQRDRNTVSLVCKVWYEIERLSRRGVFVGNCYAVRAGRVAARFP
NVRALTVKGKPHFADFNLVPPDWGGYAGPWIEAAARGCHGLEELRMKRMVVSDESLELLA
RSFPRFRALVLISCEGFSTDGLAAVASHCKLLRELDLQENEVEDRGPRWLSCFPDSCTSLV
SLNFACIKGEVNAGSLERLVSRSPNLRSLRLNRSVSVDTLAKILLRTPNLEDLGTGNLTDDF
QTESYFKLTSALEKCKMLRSLSGFWDASPVCLSFIYPLCAQLTGLNLSYAPTLDASDLTKMI
SRCVKLQRLWVLDCISDKGLQVVASSCKDLQELRVFPSDFYVAGYSAVTEEGLVAVSLGCP
KLNSLLYFCHQMTNAALVTVAKNCPNFTRFRLCILEPGKPDVVTSQPLDEGFGAIVRECKGL
QRLSISGLLTDKVFMYIGKYAKQLEMLSIAFAGDSDKGMMHVMNGCKNLRKLEIRDSPFGD
AALLGNFARYETMRSLWMSSCNVTLKGCQVLASKMPMLNVEVINERDGSNEMEENHGDL
PKVEKLYVYRTTAGARDDAPNFVKIL*
Modified osTIR (E7K, E10K) (SEQ ID NO: 15)
MTYFPEKVVKHIFSFLPAQRDRNTVSLVCKVWYEIERLSRRGVFVGNCYAVRAGRVAARFP
NVRALTVKGKPHFADFNLVPPDWGGYAGPWIEAAARGCHGLEELRMKRMVVSDESLELLA
RSFPRFRALVLISCEGFSTDGLAAVASHCKLLRELDLQENEVEDRGPRWLSCFPDSCTSLV
SLNFACIKGEVNAGSLERLVSRSPNLRSLRLNRSVSVDTLAKILLRTPNLEDLGTGNLTDDF
QTESYFKLTSALEKCKMLRSLSGFWDASPVCLSFIYPLCAQLTGLNLSYAPTLDASDLTKMI
SRCVKLQRLWVLDCISDKGLQVVASSCKDLQELRVFPSDFYVAGYSAVTEEGLVAVSLGCP
KLNSLLYFCHQMTNAALVTVAKNCPNFTRFRLCILEPGKPDVVTSQPLDEGFGAIVRECKGL
QRLSISGLLTDKVFMYIGKYAKQLEMLSIAFAGDSDKGMMHVMNGCKNLRKLEIRDSPFGD
AALLGNFARYETMRSLWMSSCNVTLKGCQVLASKMPMLNVEVINERDGSNEMEENHGDL
PKVEKLYVYRTTAGARDDAPNFVKIL*
Modified osTIR (E7K, E10K, F74G) (SEQ ID NO: 29)
MTYFPEKVVKHIFSFLPAQRDRNTVSLVCKVWYEIERLSRRGVFVGNCYAVRAGRVAARFP
NVRALTVKGKPHGADFNLVPPDWGGYAGPWIEAAARGCHGLEELRMKRMVVSDESLELL
ARSFPRFRALVLISCEGFSTDGLAAVASHCKLLRELDLQENEVEDRGPRWLSCFPDSCTSL
VSLNFACIKGEVNAGSLERLVSRSPNLRSLRLNRSVSVDTLAKILLRTPNLEDLGTGNLTDDF
QTESYFKLTSALEKCKMLRSLSGFWDASPVCLSFIYPLCAQLTGLNLSYAPTLDASDLTKMI
SRCVKLQRLWVLDCISDKGLQVVASSCKDLQELRVFPSDFYVAGYSAVTEEGLVAVSLGCP
KLNSLLYFCHQMTNAALVTVAKNCPNFTRFRLCILEPGKPDVVTSQPLDEGFGAIVRECKGL
QRLSISGLLTDKVFMYIGKYAKQLEMLSIAFAGDSDKGMMHVMNGCKNLRKLEIRDSPFGD
AALLGNFARYETMRSLWMSSCNVTLKGCQVLASKMPMLNVEVINERDGSNEMEENHGDL
PKVEKLYVYRTTAGARDDAPNFVKIL*
Wild Type AID (SEQ ID NO: 16)
MGSVELNLRETELCLGLPGGDTVAPVTGNKRGFSETVDLKLNLNNEPANKEGSTTHDVVTF
DSKEKSACPKDPAKPPAKAQVVGWPPVRSYRKNVMVSCQKSSGGPEAAAFVKVSMDGAP
YLRKIDLRMYKSYDELSNALSNMFSSFTMGKHGGEEGMIDFMNERKLMDLVNSWDYVPSY
EDKDGDWMLVGDVPWPMFVDTCKRLRLMKGSDAIGLAPRAMEKCKSRA*
AIDΔ34 (SEQ ID NO: 17)
MGSVELNLRETELCLGLPGGDTVAPVTGNKRGFSETVDLKLNLNNEPANKEGSTTHDVVTF
DSKEKSACPKDPAKPPAKAQVVGWPPVRSYRKN*
LexA (SEQ ID NO: 21)
MKALTARQQEVFDLIRDHISQTGMPPTRAEIAQRLGFRSPNAAEEHLKALARKGVIEIVSGA
SRGIRLLQEEEEGLPLVGRVAAGEPLLAQQHIEGHYQVDPSLFKPNADFLLRVSGMSMKDI
GIMDGDLLAVHKTQDVRNGQVVVARIDDEVTVKRLKKQGNKVELLPENSEFKPIVVDLRQQ
SFTIEGLAVGVIRNGDWL*
Gal4 DNA binding domain (SEQ ID NO: 22)
MKLLSSIEQACDICRLKKLKCSKEKPKCAKCLKNNWECRYSPKTKRSPLTRAHLTEVESRLE
RLEQLFLLIFPREDLDMILKMDSLQDIKALLTGLFVQDNVNKDAVTDRLASVETDMPLTLRQH
RISATSSSEESSNKGQRQLTVS*
mAID (SEQ ID NO: 23)
KEKSACPKDPAKPPAKAQVVGWPPVRSYRKNVMVSCQKSSGGPEAAAFVKVSMDGAPYL
RKIDLRMYK
ABI1 (SEQ ID NO: 24)
MEEVSPAIAGPFRPFSETQMDFTGIRLGKGYCNNQYSNQDSENGDLMVSLPETSSCSVSG
SHGSESRKVLISRINSPNLNMKESAAADIVVVDISAGDEINGSDITSEKKMISRTESRSLFEFK
SVPLYGFTSICGRRPEMEDAVSTIPRFLQSSSGSMLDGRFDPQSAAHFFGVYDGHGGSQV
ANYCRERMHLALAEEIAKEKPMLCDGDTWLEKWKKALFNSFLRVDSEIESVAPETVGSTSV
VAVVFPSHIFVANCGDSRAVLCRGKTALPLSVDHKPDREDEAARIEAAGGKVIQWNGARVF
GVLAMSRSIGDRYLKPSIIPDPEVTAVKRVKEDDCLILASDGVWDVMTDEEACEMARKRILL
WHKKNAVAGDASLLADERRKEGKDPAAMSAAEYLSKLAIQRGSKDNISVVVVDLKPRRKLK
SKPLN
Modified ABI1 complementary surfaces ABIcs (SEQ ID NO: 25)
VPLYGFTSICGRRPEMEDAVSTIPRFLQSSSGSMLDGRFDPQSAAHFFGVYDGHGGSQVA
NYCRERMHLALAEEIAKEKPMLCDGDTWLEKWKKALFNSFLRVDSEIESVAPETVGSTSVV
AVVFPSHIFVANCGDSRAVLCRGKTALPLSVDHKPDREDEAARIEAAGGKVIQWNGARVFG
VLAMSRSIGDRYLKPSIIPDPEVTAVKRVKEDDCLILASDGVWDVMTDEEACEMARKRILLW
HKKNAVAGDASLLADERRKEGKDPAAMSAAEYLSKLAIQRGSKDNISVVVVDLK
PYL1 (SEQ ID NO: 26)
MANSESSSSPVNEEENSQRISTLHHQTMPSDLTQDEFTQLSQSIAEFHTYQLGNGRCSSLL
AQRIHAPPETVWSVVRRFDRPQIYKHFIKSCNVSEDFEMRVGCTRDVNVISGLPANTSRER
LDLLDDDRRVTGFSITGGEHRLRNYKSVTTVHRFEKEEEEERIWTVVLESYVVDVPEGNSE
EDTRLFADTVIRLNLQKLASITEAMNRNNNNNNSSQVR
PYL1 complementary surface PYLCS (SEQ ID NO: 27)
TQDEFTQLSQSIAEFHTYQLGNGRCSSLLAQRIHAPPETVWSVVRRFDRPQIYKHFIKSCNV
SEDFEMRVGCTRDVNVISGLPANTSRERLDLLDDDRRVTGFSITGGEHRLRNYKSVTTVHR
FEKEEEEERIWTVVLESYVVDVPEGNSEEDTRLFADTVIRLNLQKLASITEAMN
αCaffVHH DNA sequence (SEQ ID NO: 30)
GAGGTGCAGCTGCAGGCCTCCGGAGGAGGCCTGGTGCAGGCCGGAGGGAGCCTGAG
ACTGAGCTGTACTGCCAGCGGGAGGACAGGAACAATCTACTCAATGGCCTGGTTCCGC
CAGGCCCCCGGCAAGGAAAGGGAGTTTCTGGCCACCGTGGGCTGGAGCTCTGGCATC
ACATACTACATGGACTCAGTGAAGGGTAGGTTCACCATCAGCAGAGACAACGCCAAGA
ATTCTGCCTACCTGCAGATGAATAGCCTGAAACCAGAGGATACCGCCGTGTACTACTGT
ACTGCTACAAGGGCCTATAGTGTGGGTTATGATTACTGGGGACAGGGCACCCAGGTGA
CCGTGAGCCAC
αCaffVHH amino acid sequence (SEQ ID NO: 31)
EVQLQASGGGLVQAGGSLRLSCTASGRTGTIYSMAWFRQAPGKEREFLATVGWSSGITYY
MDSVKGRFTISRDNAKNSAYLQMNSLKPEDTAVYYCTATRAYSVGYDYWGQGTQVTVSH
Arabidopsis thaliana PYR^{Mandi} DNA sequence (SEQ ID NO: 32)
ATGCCCAGCGAGCTGACCCCCGAGGAGAGGAGCGAGCTGAAGAACAGCATCGCCGAG
TTCCACACCTACCAGCTGGACCCCGGCAGCTGCAGCAGCCTGCACGCCCAGAGGATC
CACGCCCCCCCCGAGCTGGTGTGGAGCATCGTGAGGAGGTTCGACAAGCCCCAGACC
CACAGGCACTTCATCAAGAGCTGCAGCGTGGAGCAGAACTTCGAGATGAGGGTGGGC
TGCACCAGGGACATCATCGTGATCAGCGGCCTGCCCGCCAACACCAGCACCGAGAGG
CTGGACATCCTGGACGACGAGAGGAGGGTGACCGGCGCCAGCATCATCGGCGGCGA
GCACAGGCTGACCAACTACAAGGGCGTGACCACCGTGCACAGGTTCGAGAAGGAGAA
CAGGATCTGGACCGTGGTGCTGGAGAGCTACGTGGTGGACATGCCCGAGGGCAACAG
CGAGGACGACACCAGGATGCTGGCCGACACCGTGGTGAAGCTGAACCTGCAGAAGCT
GGCCACCGTGGCCGAGGCCATGGCCAGGAACAGCGGCGACGGCAGCGGCAGCCAGG
TGACCTAA
Arabidopsis thaliana PYR^{Mandi} amino acid sequence (SEQ ID NO: 33)
MPSELTPEERSELKNSIAEFHTYQLDPGSCSSLHAQRIHAPPELVWSIVRRFDKPQTHRHFI
KSCSVEQNFEMRVGCTRDIIVISGLPANTSTERLDILDDERRVTGASIIGGEHRLTNYKGVTT
VHRFEKENRIWTVVLESYVVDMPEGNSEDDTRMLADTVVKLNLQKLATVAEAMARNSGDG
SGSQVT*
Arabidopsis thaliana PYLcs^{Mandi} DNA sequence (SEQ ID NO: 34)
ACCCAGGACGAGTTCACCCAGCTGAGCCAGAGCATCGCCGAGTTCCACACCTACCAGC
TGGGCAACGGCAGGTGCAGCAGCCTGCTGGCCCAGAGGATCCACGCCCCCCCCGAG
ACCGTGTGGAGCGTGGTGAGGAGGTTCGACAGGCCCCAGATCCACAGGCACTTCATC
AAGAGCTGCAACGTGAGCGAGGACTTCGAGATGAGGGTGGGCTGCACCAGGGACATC
AACGTGATCAGCGGCCTGCCCGCCAACACCAGCAGGGAGAGGCTGGACCTGCTGGAC
GACGACAGGAGGGTGACCGGCGCCAGCATCACCGGCGGCGAGCACAGGCTGAGGAA
CTACAAGGGCGTGACCACCGTGCACAGGTTCGAGAAGGAGGAGGAGGAGGAGAGGAT
CTGGACCGTGGTGCTGGAGAGCTACGTGGTGGACGTGCCCGAGGGCAACAGCGAGG
AGGACACCAGGCTGCTGGCCGACACCGTGATCAGGCTGAACCTGCAGAAGCTGGCCA
GCATCACCGAGGCCATGAACTAA
Arabidopsis thaliana PYLcs^{Mandi} amino acid sequence (SEQ ID NO: 35)
TQDEFTQLSQSIAEFHTYQLGNGRCSSLLAQRIHAPPETVWSVVRRFDRPQIHRHFIKSCNV
SEDFEMRVGCTRDINVISGLPANTSRERLDLLDDDRRVTGASITGGEHRLRNYKGVTTVHR
FEKEEEEERIWTVVLESYVVDVPEGNSEEDTRLLADTVIRLNLQKLASITEAMN*
Arabidopsis thaliana GID1 DNA sequence (SEQ ID NO: 36)
ATGGCCGCCAGCGACGAGGTGAACCTGATCGAGAGCAGGACCGTGGTGCCCCTGAAC
ACCTGGGTGCTGATCAGCAACTTCAAGGTGGCCTACAACATCCTGAGGAGGCCCGACG
GCACCTTCAACAGGCACCTGGCCGAGTACCTGGACAGGAAGGTGACCGCCAACGCCA
ACCCCGTGGACGGCGTGTTCAGCTTCGACGTGCTGATCGACAGGAGGATCAACCTGCT
GAGCAGGGTGTACAGGCCCGCCTACGCCGACCAGGAGCAGCCCCCCAGCATCCTGGA
CCTGGAGAAGCCCGTGGACGGCGACATCGTGCCCGTGATCCTGTTCTTCCACGGCGG
CAGCTTCGCCCACAGCAGCGCCAACAGCGCCATCTACGACACCCTGTGCAGGAGGCT
GGTGGGCCTGTGCAAGTGCGTGGTGGTGAGCGTGAACTACAGGAGGGCCCCCGAGAA
CCCCTACCCCTGCGCCTACGACGACGGCTGGATCGCCCTGAACTGGGTGAACAGCAG
GAGCTGGCTGAAGAGCAAGAAGGACAGCAAGGTGCACATCTTCCTGGCCGGCGACAG
CAGCGGCGGCAACATCGCCCACAACGTGGCCCTGAGGGCCGGCGAGAGCGGCATCG
ACGTGCTGGGCAACATCCTGCTGAACCCCATGTTCGGCGGCAACGAGAGGACCGAGA
GCGAGAAGAGCCTGGACGGCAAGTACTTCGTGACCGTGAGGGACAGGGACTGGTACT
GGAAGGCCTTCCTGCCCGAGGGCGAGGACAGGGAGCACCCCGCCTGCAACCCCTTCA
GCCCCAGGGGCAAGAGCCTGGAGGGCGTGAGCTTCCCCAAGAGCCTGGTGGTGGTG
GCCGGCCTGGACCTGATCAGGGACTGGCAGCTGGCCTACGCCGAGGGCCTGAAGAAG
GCCGGCCAGGAGGTGAAGCTGATGCACCTGGAGAAGGCCACCGTGGGCTTCTACCTG
CTGCCCAACAACAACCACTTCCACAACGTGATGGACGAGATCAGCGCCTTCGTGAACG
CCGAGTGCTAA
Arabidopsis thaliana GID1 amino acid sequence (SEQ ID NO: 37)
MAASDEVNLIESRTVVPLNTWVLISNFKVAYNILRRPDGTFNRHLAEYLDRKVTANANPVDG
VFSFDVLIDRRINLLSRVYRPAYADQEQPPSILDLEKPVDGDIVPVILFFHGGSFAHSSANSAI
YDTLCRRLVGLCKCVVVSVNYRRAPENPYPCAYDDGWIALNWVNSRSWLKSKKDSKVHIF
LAGDSSGGNIAHNVALRAGESGIDVLGNILLNPMFGGNERTESEKSLDGKYFVTVRDRDWY
WKAFLPEGEDREHPACNPFSPRGKSLEGVSFPKSLVVVAGLDLIRDWQLAYAEGLKKAGQ
EVKLMHLEKATVGFYLLPNNNHFHNVMDEISAFVNAEC*
Arabidopsis thaliana GAI DNA nucleotides 1-276 (SEQ ID NO: 38)
ATGAAGAGGGACCACCACCACCACCACCACCAGGACAAGAAGACCATGATGATGAACG
AGGAGGACGACGGCAACGGCATGGACGAGCTGCTGGCCGTGCTGGGCTACAAGGTGA
GGAGCAGCGAGATGGCCGACGTGGCCCAGAAGCTGGAGCAGCTGGAGGTGATGATGA
GCAACGTGCAGGAGGACGACCTGAGCCAGCTGGCCACCGAGACCGTGCACTACAACC
CCGCCGAGCTGTACACCTGGCTGGACAGCATGCTGACCGACCTGAAC
Arabidopsis thaliana GAI (UniProt Q9LQT8) amino acids 1-92 (SEQ ID NO: 39)
MKRDHHHHHHQDKKTMMMNEEDDGNGMDELLAVLGYKVRSSEMADVAQKLEQLEVMMS
NVQEDDLSQLATETVHYNPAELYTWLDSMLTDLN
Arabidopsis thaliana full length GAI (UniProt Q9LQT8) amino acid sequence (SEQ ID
NO: 48)
MKRDHHHHHHQDKKTMMMNEEDDGNGMDELLAVLGYKVRSSEMADVAQKLEQLEVMMS
NVQEDDLSQLATETVHYNPAELYTWLDSMLTDLNPPSSNAEYDLKAIPGDAILNQFAIDSAS
SSNQGGGGDTYTTNKRLKCSNGVVETTTATAESTRHVVLVDSQENGVRLVHALLACAEAV
QKENLTVAEALVKQIGFLAVSQIGAMRKVATYFAEALARRIYRLSPSQSPIDHSLSDTLQMHF
YETCPYLKFAHFTANQAILEAFQGKKRVHVIDFSMSQGLQWPALMQALALRPGGPPVFRLT
GIGPPAPDNFDYLHEVGCKLAHLAEAIHVEFEYRGFVANTLADLDASMLELRPSEIESVAVN
SVFELHKLLGRPGAIDKVLGVVNQIKPEIFTVVEQESNHNSPIFLDRFTESLHYYSTLFDSLE
GVPSGQDKVMSEVYLGKQICNVVACDGPDRVERHETLSQWRNRFGSAGFAAAHIGSNAF
KQASMLLALFNGGEGYRVEESDGCLMLGWHTRPLIATSAWKLSTN*
Arabidopsis thaliana full length GAI nucleotide sequence (human codon optimized)
(SEQ ID NO: 49)
ATGAAGAGGGACCACCACCACCACCACCACCAGGACAAGAAGACCATGATGATGAACG
AGGAGGACGACGGCAACGGCATGGACGAGCTGCTGGCCGTGCTGGGCTACAAGGTGA
GGAGCAGCGAGATGGCCGACGTGGCCCAGAAGCTGGAGCAGCTGGAGGTGATGATGA
GCAACGTGCAGGAGGACGACCTGAGCCAGCTGGCCACCGAGACCGTGCACTACAACC
CCGCCGAGCTGTACACCTGGCTGGACAGCATGCTGACCGACCTGAACCCCCCCAGCA
GCAACGCCGAGTACGACCTGAAGGCCATCCCCGGCGACGCCATCCTGAACCAGTTCG
CCATCGACAGCGCCAGCAGCAGCAACCAGGGGGGGGGGGGCGACACCTACACCACCA
ACAAGAGGCTGAAGTGCAGCAACGGCGTGGTGGAGACCACCACCGCCACCGCCGAGA
GCACCAGGCACGTGGTGCTGGTGGACAGCCAGGAGAACGGCGTGAGGCTGGTGCAC
GCCCTGCTGGCCTGCGCCGAGGCCGTGCAGAAGGAGAACCTGACCGTGGCCGAGGC
CCTGGTGAAGCAGATCGGCTTCCTGGCCGTGAGCCAGATCGGCGCCATGAGGAAGGT
GGCCACCTACTTCGCCGAGGCCCTGGCCAGGAGGATCTACAGGCTGAGCCCCAGCCA
GAGCCCCATCGACCACAGCCTGAGCGACACCCTGCAGATGCACTTCTACGAGACCTGC
CCCTACCTGAAGTTCGCCCACTTCACCGCCAACCAGGCCATCCTGGAGGCCTTCCAGG
GCAAGAAGAGGGTGCACGTGATCGACTTCAGCATGAGCCAGGGCCTGCAGTGGCCCG
CCCTGATGCAGGCCCTGGCCCTGAGGCCCGGCGGCCCCCCCGTGTTCAGGCTGACC
GGCATCGGCCCCCCCGCCCCCGACAACTTCGACTACCTGCACGAGGTGGGCTGCAAG
CTGGCCCACCTGGCCGAGGCCATCCACGTGGAGTTCGAGTACAGGGGCTTCGTG
ABA related DNA Sequences
ABA activator cassette (T2A linker) (SEQ ID NO: 18)
ATGAGCACCGCCCCCCCCACCGACGTGAGCCTGGGCGACGAGCTGCACCTGGACGG
CGAGGACGTGGCCATGGCCCACGCCGACGCCCTGGACGACTTCGACCTGGACATGCT
GGGCGACGGCGACAGCCCCGGCCCCGGCTTCACCCCCCACGACAGCGCCCCCTACG
GCGCCCTGGACATGGCCGACTTCGAGTTCGAGCAGATGTTCACCGACGCCCTGGGCA
TCGACGAGTACGGCGGCGGTGGCGGAAGCGGAGGGGGATCGACCCAGGACGAGTTC
ACCCAGCTGAGCCAGAGCATCGCCGAGTTCCACACCTACCAGCTGGGCAACGGCAGG
TGCAGCAGCCTGCTGGCCCAGAGGATCCACGCCCCCCCCGAGACCGTGTGGAGCGTG
GTGAGGAGGTTCGACAGGCCCCAGATCTACAAGCACTTCATCAAGAGCTGCAACGTGA
GCGAGGACTTCGAGATGAGGGTGGGCTGCACCAGGGACGTGAACGTGATCAGCGGCC
TGCCCGCCAACACCAGCAGGGAGAGGCTGGACCTGCTGGACGACGACAGGAGGGTG
ACCGGCTTCAGCATCACCGGCGGCGAGCACAGGCTGAGGAACTACAAGAGCGTGACC
ACCGTGCACAGGTTCGAGAAGGAGGAGGAGGAGGAGAGGATCTGGACCGTGGTGCTG
GAGAGCTACGTGGTGGACGTGCCCGAGGGCAACAGCGAGGAGGACACCAGGCTGTTC
GCCGACACCGTGATCAGGCTGAACCTGCAGAAGCTGGCCAGCATCACCGAGGCCATG
AACGGCATCAGGAGGAAGAGGAGCGTGAGCCACGGAAGCGGAGAGGGCAGGGGAAG
TCTTCTAACATGCGGGGACGTGGAGGAAAATCCCGGCCCCATGAAGCTACTGTCTTCT
ATCGAACAAGCATGCGATATTTGCCGACTTAAAAAGCTCAAGTGCTCCAAAGAAAAACC
GAAGTGCGCCAAGTGTCTGAAGAACAACTGGGAGTGTCGCTACTCTCCCAAAACCAAA
AGGTCTCCGCTGACTAGGGCACATCTGACAGAAGTGGAATCAAGGCTAGAAAGACTGG
AACAGCTATTTCTACTGATTTTTCCTCGAGAAGACCTTGACATGATTTTGAAAATGGATT
CTTTACAGGATATAAAAGCATTGTTAACAGGATTATTTGTACAAGATAATGTGAATAAAG
ATGCCGTCACAGATAGATTGGCTTCAGTGGAGACTGATATGCCTCTAACATTGAGACAG
CATAGAATAAGTGCGACATCATCATCGGAAGAGAGTAGTAACAAAGGTCAAAGACAGTT
GACTGTATCGGGTGGCGGAAGCGGAGGGGGATCGATGACGCGTGTGCCTTTGTATGG
TTTTACTTCGATTTGTGGAAGAAGACCTGAGATGGAAGCTGCTGTTTCGACTATACCAA
GATTCCTTCAATCTTCCTCTGGTTCGATGTTAGATGGTCGGTTTGATCCTCAATCCGCC
GCTCATTTCTTCGGTGTTTACGACGGCCATGGCGGTTCTCAGGTAGCGAACTATTGTAG
AGAGAGGATGCATTTGGCTTTGGCGGAGGAGATAGCTAAGGAGAAACCGATGCTCTGC
GATGGTGATACGTGGCTGGAGAAGTGGAAGAAAGCTCTTTTCAACTCGTTCCTGAGAG
TTGACTCGGAGATTGAGTCAGTTGCGCCGGAGACGGTTGGGTCAACGTCGGTGGTTGC
CGTTGTTTTCCCGTCTCACATCTTCGTCGCTAACTGCGGTGACTCTAGAGCCGTTCTTT
GCCGCGGCAAAACTGCACTTCCATTATCCGTTGACCATAAACCGGATAGAGAAGATGAA
GCTGCGAGGATTGAAGCCGCAGGAGGGAAAGTGATTCAGTGGAATGGAGCTCGTGTTT
TCGGTGTTCTCGCCATGTCGAGATCCATTGGCGATAGATACTTGAAACCATCCATCATT
CCTGATCCGGAAGTGACGGCTGTGAAGAGAGTAAAAGAAGATGATTGTCTGATTTTGG
CGAGTGACGGGGTTTGGGATGTAATGACGGATGAAGAAGCGTGTGAGATGGCAAGGA
AGCGGATTCTCTTGTGGCACAAGAAAAACGCGGTGGCTGGGGATGCATCGTTGCTCGC
GGATGAGCGGAGAAAGGAAGGGAAAGATCCTGCGGCGATGTCCGCGGCTGAGTATTT
GTCAAAGCTGGCGATACAGAGAGGAAGCAAAGACAACATAAGTGTGGTGGTGGTTGAT
TTGAAGCATATGTCATGA
9x Gal4 UAS recognition site + minimal promoter (SEQ ID NO: 19)
GAGTTTCTAGACGGAGTACTGTCCTCCGAGCGGAGTACTGTCCTCCGACTCGAGCGGA
GTACTGTCCTCCGAGCGGAGTACTGTCCTCCGAGCGGAGTACTGTCCTCCGAGCGGA
GTACTGTCCTCCGAGCGGAGTACTGTCCTCCGAGCGGAGTACTGTCCTCCGAGGAATT
CCGGAGTACTGTCCTCCGAAGACGCTAGCGGGGGGCTATAAAAGGGGGTGGGGGCGT
TCGTCCTCACTCTAGATCTGCGATCTAAGTAAGCTTGGCAATCCGGTACTGTTGGTAA
Gal4 UAS recognition site (SEQ ID NO: 28)
CGGAGTACTGTCCTCCG
ABA Related Amino Acid Sequences
ABA activator cassette (T2A linker) (SEQ ID NO: 20)
MSTAPPTDVSLGDELHLDGEDVAMAHADALDDFDLDMLGDGDSPGPGFTPHDSAPYGAL
DMADFEFEQMFTDALGIDEYGGGGGSGGGSTQDEFTQLSQSIAEFHTYQLGNGRCSSLLA
QRIHAPPETVWSVVRRFDRPQIYKHFIKSCNVSEDFEMRVGCTRDVNVISGLPANTSRERL
DLLDDDRRVTGFSITGGEHRLRNYKSVTTVHRFEKEEEEERIWTVVLESYVVDVPEGNSEE
DTRLFADTVIRLNLQKLASITEAMNGIRRKRSVSHGSGEGRGSLLTCGDVEENPGPMKLLSS
IEQACDICRLKKLKCSKEKPKCAKCLKNNWECRYSPKTKRSPLTRAHLTEVESRLERLEQLF
LLIFPREDLDMILKMDSLQDIKALLTGLFVQDNVNKDAVTDRLASVETDMPLTLRQHRISATS
SSEESSNKGQRQLTVSGGGSGGGSMTRVPLYGFTSICGRRPEMEAAVSTIPRFLQSSSGS
MLDGRFDPQSAAHFFGVYDGHGGSQVANYCRERMHLALAEEIAKEKPMLCDGDTWLEKW
KKALFNSFLRVDSEIESVAPETVGSTSVVAVVFPSHIFVANCGDSRAVLCRGKTALPLSVDH
KPDREDEAARIEAAGGKVIQWNGARVFGVLAMSRSIGDRYLKPSIIPDPEVTAVKRVKEDDC
LILASDGVWDVMTDEEACEMARKRILLWHKKNAVAGDASLLADERRKEGKDPAAMSAAEY
LSKLAIQRGSKDNISVVVVDLKHMS*
Caffeine related DNA and amino acid sequences
Caffeine activator cassette DNA sequence (T2A linker) (SEQ ID NO: 40)
ATGAAACTGCTGAGCAGCATCGAGCAGGCCTGTGACATCTGTAGACTGAAGAAGCTGA
AGTGCAGCAAGGAGAAGCCCAAATGCGCCAAATGTCTGAAGAACAATTGGGAGTGCAG
GTACTCCCCCAAGACTAAACGCTCCCCCCTGACCAGAGCTCACCTGACAGAGGTGGAG
AGCCGGCTGGAAAGGCTGGAGCAGCTGTTCCTGCTGATTTTCCCAAGGGAAGACCTGG
ATATGATCCTGAAGATGGATAGCCTGCAGGATATCAAGGCTCTTCTGACTGGCCTCTTC
GTGCAGGACAATGTGAACAAGGACGCTGTGACAGACCGACTGGCCTCCGTGGAGACA
GACATGCCTCTGACACTCCGGCAGCACAGGATCAGCGCCACATCTTCTAGCGAGGAGT
CCTCTAACAAAGGCCAACGGCAGCTGACAGTGTCTGGGGGGGGCAGCGGCGGGGGC
AGCGAGGTGCAGCTGCAGGCCTCCGGAGGAGGCCTGGTGCAGGCCGGAGGGAGCCT
GAGACTGAGCTGTACTGCCAGCGGGAGGACAGGAACAATCTACTCAATGGCCTGGTTC
CGCCAGGCCCCCGGCAAGGAAAGGGAGTTTCTGGCCACCGTGGGCTGGAGCTCTGGC
ATCACATACTACATGGACTCAGTGAAGGGTAGGTTCACCATCAGCAGAGACAACGCCA
AGAATTCTGCCTACCTGCAGATGAATAGCCTGAAACCAGAGGATACCGCCGTGTACTAC
TGTACTGCTACAAGGGCCTATAGTGTGGGTTATGATTACTGGGGACAGGGCACCCAGG
TGACCGTGAGCCACGGGATCCGCAGAAAAAGAAGCGTGTCTCACGGCTCCGGCGAGG
GCAGAGGGTCCCTGCTGACCTGTGGCGATGTGGAGGAGAACCCTGGCCCTGAGGTGC
AGCTGCAGGCCAGTGGGGGCGGCCTGGTTCAGGCAGGCGGAAGTCTGCGGCTTAGCT
GTACCGCCTCCGGCCGGACCGGAACTATTTACTCAATGGCCTGGTTCCGGCAGGCCC
CCGGGAAAGAGAGGGAGTTCCTGGCCACCGTGGGATGGTCCTCCGGGATTACCTACT
ACATGGACTCCGTGAAGGGGAGGTTCACCATCTCTCGCGATAACGCCAAGAATAGCGC
CTATCTGCAGATGAACAGCCTGAAACCCGAGGACACAGCTGTGTACTACTGTACGGCC
ACTCGGGCTTATAGCGTGGGCTACGACTACTGGGGCCAGGGCACACAGGTGACCGTG
TCCCATGGCGGCGGCAGCGGCGGAGGCAGTATGAGCACCGCCCCCCCAACCGATGTC
AGCCTGGGCGATGAGCTCCACCTGGACGGCGAGGACGTGGCCATGGCCCATGCCGAT
GCCCTGGACGACTTCGATCTGGACATGCTGGGCGACGGCGATAGTCCCGGCCCAGGC
TTCACCCCTCATGATTCCGCTCCATACGGTGCCCTGGATATGGCCGACTTCGAGTTTGA
GCAGATGTTCACAGACGCCCTGGGCATCGATGAGTACGGCGGCTAA
Caffeine activator cassette amino acid sequence (T2A linker) (SEQ ID NO: 41)
MKLLSSIEQACDICRLKKLKCSKEKPKCAKCLKNNWECRYSPKTKRSPLTRAHLTEVESRLE
RLEQLFLLIFPREDLDMILKMDSLQDIKALLTGLFVQDNVNKDAVTDRLASVETDMPLTLRQH
RISATSSSEESSNKGQRQLTVSGGGSGGGSEVQLQASGGGLVQAGGSLRLSCTASGRTG
TIYSMAWFRQAPGKEREFLATVGWSSGITYYMDSVKGRFTISRDNAKNSAYLQMNSLKPE
DTAVYYCTATRAYSVGYDYWGQGTQVTVSHGIRRKRSVSHGSGEGRGSLLTCGDVEENP
GPEVQLQASGGGLVQAGGSLRLSCTASGRTGTIYSMAWFRQAPGKEREFLATVGWSSGIT
YYMDSVKGRFTISRDNAKNSAYLQMNSLKPEDTAVYYCTATRAYSVGYDYWGQGTQVTVS
HGGGSGGGSMSTAPPTDVSLGDELHLDGEDVAMAHADALDDFDLDMLGDGDSPGPGFTP
HDSAPYGALDMADFEFEQMFTDALGIDEYGG*
Mandipropamid (Mandi) related DNA and amino acid sequences
PYLcs^{Mandi} activator cassette DNA sequence (T2A linker) (SEQ ID NO: 42)
ATGAGCACCGCCCCCCCCACCGACGTGAGCCTGGGCGACGAGCTGCACCTGGACGG
CGAGGACGTGGCCATGGCCCACGCCGACGCCCTGGACGACTTCGACCTGGACATGCT
GGGCGACGGCGACAGCCCCGGCCCCGGCTTCACCCCCCACGACAGCGCCCCCTACG
GCGCCCTGGACATGGCCGACTTCGAGTTCGAGCAGATGTTCACCGACGCCCTGGGCA
TCGACGAGTACGGCGGCGGTGGCGGAAGCGGAGGGGGATCGACCCAGGACGAGTTC
ACCCAGCTGAGCCAGAGCATCGCCGAGTTCCACACCTACCAGCTGGGCAACGGCAGG
TGCAGCAGCCTGCTGGCCCAGAGGATCCACGCCCCCCCCGAGACCGTGTGGAGCGTG
GTGAGGAGGTTCGACAGGCCCCAGATCCACAGGCACTTCATCAAGAGCTGCAACGTGA
GCGAGGACTTCGAGATGAGGGTGGGCTGCACCAGGGACATCAACGTGATCAGCGGCC
TGCCCGCCAACACCAGCAGGGAGAGGCTGGACCTGCTGGACGACGACAGGAGGGTG
ACCGGCGCCAGCATCACCGGCGGCGAGCACAGGCTGAGGAACTACAAGGGCGTGAC
CACCGTGCACAGGTTCGAGAAGGAGGAGGAGGAGGAGAGGATCTGGACCGTGGTGCT
GGAGAGCTACGTGGTGGACGTGCCCGAGGGCAACAGCGAGGAGGACACCAGGCTGC
TGGCCGACACCGTGATCAGGCTGAACCTGCAGAAGCTGGCCAGCATCACCGAGGCCA
TGAACGGCATCAGGAGGAAGAGGAGCGTGAGCCACGGAAGCGGAGAGGGCAGGGGA
AGTCTTCTAACATGCGGGGACGTGGAGGAAAATCCCGGCCCCATGAAGCTACTGTCTT
CTATCGAACAAGCATGCGATATTTGCCGACTTAAAAAGCTCAAGTGCTCCAAAGAAAAA
CCGAAGTGCGCCAAGTGTCTGAAGAACAACTGGGAGTGTCGCTACTCTCCCAAAACCA
AAAGGTCTCCGCTGACTAGGGCACATCTGACAGAAGTGGAATCAAGGCTAGAAAGACT
GGAACAGCTATTTCTACTGATTTTTCCTCGAGAAGACCTTGACATGATTTTGAAAATGGA
TTCTTTACAGGATATAAAAGCATTGTTAACAGGATTATTTGTACAAGATAATGTGAATAAA
GATGCCGTCACAGATAGATTGGCTTCAGTGGAGACTGATATGCCTCTAACATTGAGACA
GCATAGAATAAGTGCGACATCATCATCGGAAGAGAGTAGTAACAAAGGTCAAAGACAGT
TGACTGTATCGGGTGGCGGAAGCGGAGGGGGATCGATGACGCGTGTGCCTTTGTATG
GTTTTACTTCGATTTGTGGAAGAAGACCTGAGATGGAAGCTGCTGTTTCGACTATACCA
AGATTCCTTCAATCTTCCTCTGGTTCGATGTTAGATGGTCGGTTTGATCCTCAATCCGCC
GCTCATTTCTTCGGTGTTTACGACGGCCATGGCGGTTCTCAGGTAGCGAACTATTGTAG
AGAGAGGATGCATTTGGCTTTGGCGGAGGAGATAGCTAAGGAGAAACCGATGCTCTGC
GATGGTGATACGTGGCTGGAGAAGTGGAAGAAAGCTCTTTTCAACTCGTTCCTGAGAG
TTGACTCGGAGATTGAGTCAGTTGCGCCGGAGACGGTTGGGTCAACGTCGGTGGTTGC
CGTTGTTTTCCCGTCTCACATCTTCGTCGCTAACTGCGGTGACTCTAGAGCCGTTCTTT
GCCGCGGCAAAACTGCACTTCCATTATCCGTTGACCATAAACCGGATAGAGAAGATGAA
GCTGCGAGGATTGAAGCCGCAGGAGGGAAAGTGATTCAGTGGAATGGAGCTCGTGTTT
TCGGTGTTCTCGCCATGTCGAGATCCATTGGCGATAGATACTTGAAACCATCCATCATT
CCTGATCCGGAAGTGACGGCTGTGAAGAGAGTAAAAGAAGATGATTGTCTGATTTTGG
CGAGTGACGGGGTTTGGGATGTAATGACGGATGAAGAAGCGTGTGAGATGGCAAGGA
AGCGGATTCTCTTGTGGCACAAGAAAAACGCGGTGGCTGGGGATGCATCGTTGCTCGC
GGATGAGCGGAGAAAGGAAGGGAAAGATCCTGCGGCGATGTCCGCGGCTGAGTATTT
GTCAAAGCTGGCGATACAGAGAGGAAGCAAAGACAACATAAGTGTGGTGGTGGTTGAT
TTGAAGCATATGTCATAA
PYLcs^{Mandi} activator cassette amino acid sequence (T2A linker) (SEQ ID NO: 43)
MSTAPPTDVSLGDELHLDGEDVAMAHADALDDFDLDMLGDGDSPGPGFTPHDSAPYGAL
DMADFEFEQMFTDALGIDEYGGGGGSGGGSTQDEFTQLSQSIAEFHTYQLGNGRCSSLLA
QRIHAPPETVWSVVRRFDRPQIHRHFIKSCNVSEDFEMRVGCTRDINVISGLPANTSRERLD
LLDDDRRVTGASITGGEHRLRNYKGVTTVHRFEKEEEEERIWTVVLESYVVDVPEGNSEED
TRLLADTVIRLNLQKLASITEAMNGIRRKRSVSHGSGEGRGSLLTCGDVEENPGPMKLLSSI
EQACDICRLKKLKCSKEKPKCAKCLKNNWECRYSPKTKRSPLTRAHLTEVESRLERLEQLF
LLIFPREDLDMILKMDSLQDIKALLTGLFVQDNVNKDAVTDRLASVETDMPLTLRQHRISATS
SSEESSNKGQRQLTVSGGGSGGGSMTRVPLYGFTSICGRRPEMEAAVSTIPRFLQSSSGS
MLDGRFDPQSAAHFFGVYDGHGGSQVANYCRERMHLALAEEIAKEKPMLCDGDTWLEKW
KKALFNSFLRVDSEIESVAPETVGSTSVVAVVFPSHIFVANCGDSRAVLCRGKTALPLSVDH
KPDREDEAARIEAAGGKVIQWNGARVFGVLAMSRSIGDRYLKPSIIPDPEVTAVKRVKEDDC
LILASDGVWDVMTDEEACEMARKRILLWHKKNAVAGDASLLADERRKEGKDPAAMSAAEY
LSKLAIQRGSKDNISVVVVDLKHMS*
PYR^{Mandi} activator cassette DNA sequence (T2A linker) (SEQ ID NO: 44)
ATGAGCACCGCCCCCCCCACCGACGTGAGCCTGGGCGACGAGCTGCACCTGGACGG
CGAGGACGTGGCCATGGCCCACGCCGACGCCCTGGACGACTTCGACCTGGACATGCT
GGGCGACGGCGACAGCCCCGGCCCCGGCTTCACCCCCCACGACAGCGCCCCCTACG
GCGCCCTGGACATGGCCGACTTCGAGTTCGAGCAGATGTTCACCGACGCCCTGGGCA
TCGACGAGTACGGCGGCGGTGGCGGAAGCGGAGGGGGATCGATGCCCAGCGAGCTG
ACCCCCGAGGAGAGGAGCGAGCTGAAGAACAGCATCGCCGAGTTCCACACCTACCAG
CTGGACCCCGGCAGCTGCAGCAGCCTGCACGCCCAGAGGATCCACGCCCCCCCCGA
GCTGGTGTGGAGCATCGTGAGGAGGTTCGACAAGCCCCAGACCCACAGGCACTTCAT
CAAGAGCTGCAGCGTGGAGCAGAACTTCGAGATGAGGGTGGGCTGCACCAGGGACAT
CATCGTGATCAGCGGCCTGCCCGCCAACACCAGCACCGAGAGGCTGGACATCCTGGA
CGACGAGAGGAGGGTGACCGGCGCCAGCATCATCGGCGGCGAGCACAGGCTGACCA
ACTACAAGGGCGTGACCACCGTGCACAGGTTCGAGAAGGAGAACAGGATCTGGACCG
TGGTGCTGGAGAGCTACGTGGTGGACATGCCCGAGGGCAACAGCGAGGACGACACCA
GGATGCTGGCCGACACCGTGGTGAAGCTGAACCTGCAGAAGCTGGCCACCGTGGCCG
AGGCCATGGCCAGGAACAGCGGCGACGGCAGCGGCAGCCAGGTGACCGGCATCAGG
AGGAAGAGGAGCGTGAGCCACGGAAGCGGAGAGGGCAGGGGAAGTCTTCTAACATGC
GGGGACGTGGAGGAAAATCCCGGCCCCATGAAGCTACTGTCTTCTATCGAACAAGCAT
GCGATATTTGCCGACTTAAAAAGCTCAAGTGCTCCAAAGAAAAACCGAAGTGCGCCAAG
TGTCTGAAGAACAACTGGGAGTGTCGCTACTCTCCCAAAACCAAAAGGTCTCCGCTGA
CTAGGGCACATCTGACAGAAGTGGAATCAAGGCTAGAAAGACTGGAACAGCTATTTCTA
CTGATTTTTCCTCGAGAAGACCTTGACATGATTTTGAAAATGGATTCTTTACAGGATATA
AAAGCATTGTTAACAGGATTATTTGTACAAGATAATGTGAATAAAGATGCCGTCACAGAT
AGATTGGCTTCAGTGGAGACTGATATGCCTCTAACATTGAGACAGCATAGAATAAGTGC
GACATCATCATCGGAAGAGAGTAGTAACAAAGGTCAAAGACAGTTGACTGTATCGGGT
GGCGGAAGCGGAGGGGGATCGATGACGCGTGTGCCTTTGTATGGTTTTACTTCGATTT
GTGGAAGAAGACCTGAGATGGAAGCTGCTGTTTCGACTATACCAAGATTCCTTCAATCT
TCCTCTGGTTCGATGTTAGATGGTCGGTTTGATCCTCAATCCGCCGCTCATTTCTTCGG
TGTTTACGACGGCCATGGCGGTTCTCAGGTAGCGAACTATTGTAGAGAGAGGATGCAT
TTGGCTTTGGCGGAGGAGATAGCTAAGGAGAAACCGATGCTCTGCGATGGTGATACGT
GGCTGGAGAAGTGGAAGAAAGCTCTTTTCAACTCGTTCCTGAGAGTTGACTCGGAGATT
GAGTCAGTTGCGCCGGAGACGGTTGGGTCAACGTCGGTGGTTGCCGTTGTTTTCCCGT
CTCACATCTTCGTCGCTAACTGCGGTGACTCTAGAGCCGTTCTTTGCCGCGGCAAAACT
GCACTTCCATTATCCGTTGACCATAAACCGGATAGAGAAGATGAAGCTGCGAGGATTGA
AGCCGCAGGAGGGAAAGTGATTCAGTGGAATGGAGCTCGTGTTTTCGGTGTTCTCGCC
ATGTCGAGATCCATTGGCGATAGATACTTGAAACCATCCATCATTCCTGATCCGGAAGT
GACGGCTGTGAAGAGAGTAAAAGAAGATGATTGTCTGATTTTGGCGAGTGACGGGGTT
TGGGATGTAATGACGGATGAAGAAGCGTGTGAGATGGCAAGGAAGCGGATTCTCTTGT
GGCACAAGAAAAACGCGGTGGCTGGGGATGCATCGTTGCTCGCGGATGAGCGGAGAA
AGGAAGGGAAAGATCCTGCGGCGATGTCCGCGGCTGAGTATTTGTCAAAGCTGGCGAT
ACAGAGAGGAAGCAAAGACAACATAAGTGTGGTGGTGGTTGATTTGAAGCATATGTCAT
GA
PYR^{Mandi} activator cassette amino acid sequence (T2A linker) (SEQ ID NO: 45)
MSTAPPTDVSLGDELHLDGEDVAMAHADALDDFDLDMLGDGDSPGPGFTPHDSAPYGAL
DMADFEFEQMFTDALGIDEYGGGGGSGGGSMPSELTPEERSELKNSIAEFHTYQLDPGSC
SSLHAQRIHAPPELVWSIVRRFDKPQTHRHFIKSCSVEQNFEMRVGCTRDIIVISGLPANTST
ERLDILDDERRVTGASIIGGEHRLTNYKGVTTVHRFEKENRIWTVVLESYVVDMPEGNSEDD
TRMLADTVVKLNLQKLATVAEAMARNSGDGSGSQVTGIRRKRSVSHGSGEGRGSLLTCGD
VEENPGPMKLLSSIEQACDICRLKKLKCSKEKPKCAKCLKNNWECRYSPKTKRSPLTRAHL
TEVESRLERLEQLFLLIFPREDLDMILKMDSLQDIKALLTGLFVQDNVNKDAVTDRLASVETD
MPLTLRQHRISATSSSEESSNKGQRQLTVSGGGSGGGSMTRVPLYGFTSICGRRPEMEAA
VSTIPRFLQSSSGSMLDGRFDPQSAAHFFGVYDGHGGSQVANYCRERMHLALAEEIAKEK
PMLCDGDTWLEKWKKALFNSFLRVDSEIESVAPETVGSTSVVAVVFPSHIFVANCGDSRAV
LCRGKTALPLSVDHKPDREDEAARIEAAGGKVIQWNGARVFGVLAMSRSIGDRYLKPSIIPD
PEVTAVKRVKEDDCLILASDGVWDVMTDEEACEMARKRILLWHKKNAVAGDASLLADERR
KEGKDPAAMSAAEYLSKLAIQRGSKDNISVVVVDLKHMS*
Gibberellin (GA) related DNA and amino acid sequences
Gibberellin activator cassette nucleotide sequence (T2A linker) (SEQ ID NO: 46)
ATGAGCACCGCCCCCCCCACCGACGTGAGCCTGGGCGACGAGCTGCACCTGGACGG
CGAGGACGTGGCCATGGCCCACGCCGACGCCCTGGACGACTTCGACCTGGACATGCT
GGGCGACGGCGACAGCCCCGGCCCCGGCTTCACCCCCCACGACAGCGCCCCCTACG
GCGCCCTGGACATGGCCGACTTCGAGTTCGAGCAGATGTTCACCGACGCCCTGGGCA
TCGACGAGTACGGCGGCGGTGGCGGAAGCGGAGGGGGATCGATGGCCGCCAGCGAC
GAGGTGAACCTGATCGAGAGCAGGACCGTGGTGCCCCTGAACACCTGGGTGCTGATC
AGCAACTTCAAGGTGGCCTACAACATCCTGAGGAGGCCCGACGGCACCTTCAACAGGC
ACCTGGCCGAGTACCTGGACAGGAAGGTGACCGCCAACGCCAACCCCGTGGACGGCG
TGTTCAGCTTCGACGTGCTGATCGACAGGAGGATCAACCTGCTGAGCAGGGTGTACAG
GCCCGCCTACGCCGACCAGGAGCAGCCCCCCAGCATCCTGGACCTGGAGAAGCCCGT
GGACGGCGACATCGTGCCCGTGATCCTGTTCTTCCACGGCGGCAGCTTCGCCCACAG
CAGCGCCAACAGCGCCATCTACGACACCCTGTGCAGGAGGCTGGTGGGCCTGTGCAA
GTGCGTGGTGGTGAGCGTGAACTACAGGAGGGCCCCCGAGAACCCCTACCCCTGCGC
CTACGACGACGGCTGGATCGCCCTGAACTGGGTGAACAGCAGGAGCTGGCTGAAGAG
CAAGAAGGACAGCAAGGTGCACATCTTCCTGGCCGGCGACAGCAGCGGCGGCAACAT
CGCCCACAACGTGGCCCTGAGGGCCGGCGAGAGCGGCATCGACGTGCTGGGCAACA
TCCTGCTGAACCCCATGTTCGGCGGCAACGAGAGGACCGAGAGCGAGAAGAGCCTGG
ACGGCAAGTACTTCGTGACCGTGAGGGACAGGGACTGGTACTGGAAGGCCTTCCTGC
CCGAGGGCGAGGACAGGGAGCACCCCGCCTGCAACCCCTTCAGCCCCAGGGGCAAG
AGCCTGGAGGGCGTGAGCTTCCCCAAGAGCCTGGTGGTGGTGGCCGGCCTGGACCTG
ATCAGGGACTGGCAGCTGGCCTACGCCGAGGGCCTGAAGAAGGCCGGCCAGGAGGT
GAAGCTGATGCACCTGGAGAAGGCCACCGTGGGCTTCTACCTGCTGCCCAACAACAAC
CACTTCCACAACGTGATGGACGAGATCAGCGCCTTCGTGAACGCCGAGTGCGGCATCA
GGAGGAAGAGGAGCGTGAGCCACGGAAGCGGAGAGGGCAGGGGAAGTCTTCTAACAT
GCGGGGACGTGGAGGAAAATCCCGGCCCCATGAAGCTACTGTCTTCTATCGAACAAGC
ATGCGATATTTGCCGACTTAAAAAGCTCAAGTGCTCCAAAGAAAAACCGAAGTGCGCCA
AGTGTCTGAAGAACAACTGGGAGTGTCGCTACTCTCCCAAAACCAAAAGGTCTCCGCT
GACTAGGGCACATCTGACAGAAGTGGAATCAAGGCTAGAAAGACTGGAACAGCTATTT
CTACTGATTTTTCCTCGAGAAGACCTTGACATGATTTTGAAAATGGATTCTTTACAGGAT
ATAAAAGCATTGTTAACAGGATTATTTGTACAAGATAATGTGAATAAAGATGCCGTCACA
GATAGATTGGCTTCAGTGGAGACTGATATGCCTCTAACATTGAGACAGCATAGAATAAG
TGCGACATCATCATCGGAAGAGAGTAGTAACAAAGGTCAAAGACAGTTGACTGTATCGG
GTGGCGGAAGCGGAGGGGGATCGATGAAGAGGGACCACCACCACCACCACCACCAG
GACAAGAAGACCATGATGATGAACGAGGAGGACGACGGCAACGGCATGGACGAGCTG
CTGGCCGTGCTGGGCTACAAGGTGAGGAGCAGCGAGATGGCCGACGTGGCCCAGAA
GCTGGAGCAGCTGGAGGTGATGATGAGCAACGTGCAGGAGGACGACCTGAGCCAGCT
GGCCACCGAGACCGTGCACTACAACCCCGCCGAGCTGTACACCTGGCTGGACAGCAT
GCTGACCGACCTGAACTAA
Gibberellin activator cassette amino acid sequence (T2A linker) (SEQ ID NO: 47)
MSTAPPTDVSLGDELHLDGEDVAMAHADALDDFDLDMLGDGDSPGPGFTPHDSAPYGAL
DMADFEFEQMFTDALGIDEYGGGGGSGGGSMAASDEVNLIESRTVVPLNTWVLISNFKVA
YNILRRPDGTFNRHLAEYLDRKVTANANPVDGVFSFDVLIDRRINLLSRVYRPAYADQEQPP
SILDLEKPVDGDIVPVILFFHGGSFAHSSANSAIYDTLCRRLVGLCKCVVVSVNYRRAPENPY
PCAYDDGWIALNWVNSRSWLKSKKDSKVHIFLAGDSSGGNIAHNVALRAGESGIDVLGNILL
NPMFGGNERTESEKSLDGKYFVTVRDRDWYWKAFLPEGEDREHPACNPFSPRGKSLEGV
SFPKSLVVVAGLDLIRDWQLAYAEGLKKAGQEVKLMHLEKATVGFYLLPNNNHFHNVMDEI
SAFVNAECGIRRKRSVSHGSGEGRGSLLTCGDVEENPGPMKLLSSIEQACDICRLKKLKCS
KEKPKCAKCLKNNWECRYSPKTKRSPLTRAHLTEVESRLERLEQLFLLIFPREDLDMILKMD
SLQDIKALLTGLFVQDNVNKDAVTDRLASVETDMPLTLRQHRISATSSSEESSNKGQRQLTV
SGGGSGGGSMKRDHHHHHHQDKKTMMMNEEDDGNGMDELLAVLGYKVRSSEMADVAQ
KLEQLEVMMSNVQEDDLSQLATETVHYNPAELYTWLDSMLTDLN*
FMC63 scFv based CD19-targeting CAR used in examples (SEQ ID NO: 50)
MALPVTALLLPLALLLHAARPDIQMTQTTSSLSASLGDRVTISCRASQDISKYLNWYQQKPD
GTVKLLIYHTSRLHSGVPSRFSGSGSGTDYSLTISNLEQEDIATYFCQQGNTLPYTFGGGTK
LEITGGGGSGGGGSGGGGSEVKLQESGPGLVAPSQSLSVTCTVSGVSLPDYGVSWIRQP
PRKGLEWLGVIWGSETTYYNSALKSRLTIIKDNSKSQVFLKMNSLQTDDTAIYYCAKHYYYG
GSYAMDYWGQGTSVTVSSTTTPAPRPPTPAPTIASQPLSLRPEACRPAAGGAVHTRGLDF
ACDIYIWAPLAGTCGVLLLSLVITLYCKRGRKKLLYIFKQPFMRPVQTTQEEDGCSCRFPEEE
EGGCELRVKFSRSADAPAYKQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR*
Dual CD19/CD22 targeting CAR used in examples (SEQ ID NO: 51)
MPLLLLLPLLWAGALAGSGEQKLISEEDLGGGSGGGSDIQMTQTTSSLSASLGDRVTISCRA
SQDISKYLNWYQQKPDGTVKLLIYHTSRLHSGVPSRFSGSGSGTDYSLTISNLEQEDIATYF
CQQGNTLPYTFGGGTKLEITGGGGSQVQLQQSGPGLVKPSQTLSLTCAISGDSVSSNSAA
WNWIRQSPSRGLEWLGRTYYRSKWYNDYAVSVKSRITINPDTSKNQFSLQLNSVTPEDTA
VYYCAREVTGDLEDAFDIWGQGTMVTVSSGSTSGSGKPGSGEGSTKGDIQMTQSPSSLSA
SVGDRVTITCRASQTIWSYLNWYQQRPGKAPNLLIYAASSLQSGVPSRFSGRGSGTDFTLTI
SSLQAEDFATYYCQQSYSIPQTFGQGTKLEIKGGGGSEVKLQESGPGLVAPSQSLSVTCTV
SGVSLPDYGVSWIRQPPRKGLEWLGVIWGSETTYYNSALKSRLTIIKDNSKSQVFLKMNSL
QTDDTAIYYCAKHYYYGGSYAMDYWGQGTSVTVSSAAAIEVMYPPPYLDNEKSNGTIIHVK
GKHLCPSPLFPGPSKPFWVLVVVGGVLACYSLLVTVAFIIFWVRSKRSRLLHSDYMNMTPR
RPGPTRKHYQPYAPPRDFAAYRSRVKFSRSADAPAYKQGQNQLYNELNLGRREEYDVLD
KRRGRDPEMGGKPRRKNPQEGLYNELQKDKMAEAYSEIGMKGERRRGKGHDGLYQGLS
TATKDTYDALHMQALPPR*

### BIBLIOGRAPHY

1. Weijers, D. & Wagner, D. Transcriptional Responses to the Auxin Hormone. Annu. Rev. Plant Biol. 67, 539-74 (2016).
2. Nishimura, K., Fukagawa, T., Takisawa, H., Kakimoto, T. & Kanemaki, M. An auxin-based degron system for the rapid depletion of proteins in nonplant cells. Nat. Methods 6, 917-22 (2009).
3. Zhao, W. et al. A chemically induced proximity system engineered from the plant auxin signaling pathway. Chem. Sci. 9, 5822-5827 (2018).
4. Cutler, S. R., Rodriguez, P. L., Finkelstein, R. R. & Abrams, S. R. Abscisic acid: emergence of a core signaling network. Annu. Rev. Plant Biol. 61, 651-79 (2010).
5. Liang, F. Sen, Ho, W. Q. & Crabtree, G. R. Engineering the ABA Plant stress pathway for regulation of induced proximity. Sci. Signal. 4, rs2 (2011).
6. Ladenson, R. C., Crimmins, D. L., Landt, Y. & Ladenson, J. H. Isolation and characterization of a thermally stable recombinant anti-caffeine heavy-chain antibody fragment. Anal. Chem. 78, 4501-4508 (2006).
7. Sonneson, G. J. & Horn, J. R. Hapten-induced dimerization of a single-domain VHH camelid antibody. Biochemistry 48, 6693-6695 (2009).
8. Bojar, D., Scheller, L., Hamri, G. C.-E., Xie, M. & Fussenegger, M. Caffeine-inducible gene switches controlling experimental diabetes. Nat. Commun. 9, 2318 (2018).
9. Park, S. Y. et al. Agrochemical control of plant water use using engineered abscisic acid receptors. Nature 520, 545-548 (2015).
10. Ziegler, M. J. et al. Mandipropamid as a chemical inducer of proximity for in vivo applications. Nat. Chem. Biol. 18, 64-69 (2022).
11. Miyamoto, T. et al. Rapid and orthogonal logic gating with a gibberellin-induced dimerization system. Nat. Chem. Biol. 8, 465-470 (2012).

1. A method of screening a candidate binding molecule for a biological effect comprising:
(a) Providing a cell comprising a first inducible system operable to express a first protein of interest, and optionally a second inducible system operable to express a second protein of interest;
(b) Exposing the cell to an effective concentration of a first inducer to induce a desired level of expression of the first protein of interest, and optionally exposing the cell to an effective concentration of a second inducer to induce a desired level of expression of the second protein of interest;
(c) Contacting the cell with a candidate binding molecule;
(d) Determining whether the candidate binding molecule enacts a biological effect on the cell expressing the first and optionally the second protein of interest.

2. A method determining the minimum level of expression of at least one protein of interest in a cell at which a candidate binding molecule enacts a biological effect, the method comprising:
(a) Providing a cell comprising a first inducible system operable to express a first protein of interest, and optionally a second inducible system operable to express a second protein of interest;
(b) Exposing the cell comprising the first inducible system, and optionally the second inducible system, to a plurality of different concentrations of a first inducer to induce a plurality of different levels of expression of the first protein of interest, and optionally exposing the cell to a plurality of different concentrations of a second inducer to induce a plurality of different levels of expression of the second protein of interest;
(c) Contacting the cell with the candidate binding molecule;
(d) Determining whether the candidate binding molecule enacts a biological effect on the cell expressing the first and optionally the second protein of interest at each level of expression of the first and optionally the second protein of interest;
(e) Determining the minimum level of expression of the first and optionally the second protein of interest at which the candidate binding molecule enacts a biological effect on the cell expressing the first and optionally the second protein of interest.

3. A method according to claim 1 or 2, wherein the biological effect comprises binding to the first and optionally the second protein of interest.

4. A method according to claim 1, 2, or 3, wherein the candidate binding molecule is selected from: a fusion protein, an antibody (e.g. a monoclonal antibody, an antibody drug conjugate, a nanobody, scFv, di-scFv, Fab, sdAb, F(ab)₂, a 185glycol-engineered antibody) or a binding fragment thereof, a fusion protein, an antibody-drug conjugate, an aptamer, an ankyrin, a designed ankyrin repeat protein (DARPin), a peptide, a bicyclic peptide, a vaccine, a cytokine, a chemokine, a hormone, an Oncolytic virus, and a bacterium, preferably the binding molecule is an immunotherapy.

5. A method of screening a candidate therapeutic agent for a biological effect comprising:
(a) Providing a cell comprising first inducible system operable to express a first protein of interest, and optionally a second inducible system operable to express a second protein of interest, and an immune cell;
(b) Exposing the cell comprising the first inducible system, and optionally the second inducible system, to an effective concentration of a first inducer to induce a desired level of expression of the first protein of interest, and optionally exposing the cell to an effective concentration of a second inducer to induce a desired level of expression of the second protein of interest;
(c) Contacting the immune cell with the candidate therapeutic agent;
(d) Determining whether the contacted immune cell enacts a biological effect on the cell expressing the first and optionally the second protein of interest.

6. A method of determining the minimum level of expression of at least one protein of interest in a cell at which an immune cell enacts a biological effect in the presence of a candidate therapeutic agent the method comprising:
(a) Providing a cell comprising a first inducible system operable to express a first protein of interest, and optionally a second inducible system operable to express a second protein of interest, and an immune cell;
(b) Exposing the cell comprising the first inducible system and optionally the second inducible system to a plurality of different concentrations of a first inducer to induce a plurality of different levels of expression of the first protein of interest, and optionally exposing the cell to a plurality of different concentrations of a second inducer to induce a plurality of different levels expression of the second protein of interest;
(c) Contacting the immune cell with the candidate therapeutic agent;
(d) Determining whether the contacted immune cell enacts a biological effect on the cell expressing the first and optionally the second protein of interest at each level of expression of the first and optionally the second protein of interest;
(e) Determining the minimum level of expression of the first and optionally the second protein of interest at which the contacted immune cell enacts a biological effect on the cell expressing the first and optionally the second protein of interest.

7. A method according to any of claim 5 or 6, wherein the biological effect comprises targeting the cell expressing the first and optionally the second protein of interest.

8. A method according to any of claim 5, 6 or 7, wherein the candidate therapeutic agent is a biologic, preferably the candidate therapeutic agent is an immunotherapy, more preferably selected from a fusion protein, an antibody (e.g. a monoclonal antibody, an antibody drug conjugate, a nanobody, scFv, di-scFv, Fab, sdAb, F(ab)₂, a 186glycol-engineered antibody) or a binding fragment thereof, a fusion protein, an antibody-drug conjugate, an aptamer, an ankyrin, a designed ankyrin repeat protein (DARPin), a peptide, a bicyclic peptide, a vaccine, a cytokine, a chemokine, a hormone, an Oncolytic virus, and a bacterium.

9. A method according to any of claims 5 to 8, wherein the immune cell is selected from a T cell, an NK cell, a B cell, a lymphocyte, a dendritic cell, and a mesenchymal cell, or immortalised cells thereof.

10. A method of screening a candidate engineered immune cell for a biological effect comprising:
(a) Providing a cell comprising a first inducible system operable to express a first protein of interest, and optionally a second inducible system operable to express a second protein of interest;
(b) Exposing the cell comprising the first inducible system and optionally the second inducible system to an effective concentration of a first inducer to induce a desired level of expression of the first protein of interest, and optionally exposing the cell to an effective concentration of a second inducer to induce a desired level of expression of the second protein of interest;
(c) Contacting the cell with the candidate engineered immune cell;
(d) Determining whether the candidate engineered immune cell enacts a biological effect on the cell expressing the first and optionally the second protein of interest.

11. A method of determining the minimum level of expression of at least one protein of interest in a cell at which a candidate engineered immune cell enacts a biological effect, the method comprising:
(a) Providing a cell comprising a first inducible system operable to express a first protein of interest, and optionally a second inducible system operable to express a second protein of interest;
(b) Exposing the cell comprising the first inducible system, and optionally the second inducible system, to a plurality of different concentrations of a first inducer to induce a plurality of different levels of expression of the first protein of interest, and optionally exposing the cell to a plurality of different concentrations of a second inducer to induce a plurality of different levels of expression of the second protein of interest;
(c) Contacting the cell with the candidate engineered immune cell;
(d) Determining whether the candidate engineered immune cell enacts a biological effect on the cell expressing the first and optionally the second protein of interest at each level of expression of the first and optionally the second protein of interest
(e) Determining the minimum level of expression of the first and optionally the second protein of interest at which the candidate engineered immune cell enacts a biological effect on the cell expressing the first and optionally the second protein of interest.

12. A method according to claim 10 or 11, wherein enacting a biological effect may comprise targeting the cell expressing the first and optionally the second protein of interest.

13. A method according to any of claims 10-12, wherein the candidate engineered immune cell is selected from a cell expressing a CAR or a T-cell receptor (TCR), preferably selected from a CAR T-cell, a TCR T cell, a CAR NK cell, a CAR macrophage, and a CAR B cell.

14. A method according to any of claim 1, 3-5, 7-10, or 12-13, wherein the effective concentration of first or second inducer comprises between 0.001 μM to 2000 μM.

15. A method according to any of claim 1, 3-5, 7-10, or 12-14, comprising exposing the cell to a plurality of different concentrations of first inducer to induce a plurality of different levels of expression of the first protein of interest, and optionally exposing the cell to a plurality of different concentrations of second inducer to induce a desired level of expression of the second protein of interest.

16. A method according to any of claim 2-4, 6-9, 11-13 or 15 wherein the plurality of different concentrations of first inducer and optionally the second inducer range from 0.001 μM to 2000 μM, preferably the plurality of different concentrations of first inducer and optionally the second inducer are selected from: 0.001 μM, 0.002 μM, 0.003 μM, 0.004 μM, 0.005 μM, 0.006 μM, 0.007 μM, 0.008 μM, 0.009 μM 0.01 μM, 0.02 μM, 0.03 μM, 0.04 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 10 μM, 50 μM, 100 μM, 200 μM, 500 μM, 750 μM, 1000 μM, and 2000 μM.

17. A method according to any of claim 2-4, 6-9, or 11-13 or 16 wherein the cell is exposed to at least two different concentrations of first and optionally the second inducer, preferably at least a low concentration within the range of 0.001-50 μM and a high concentration in the range of 50-2000 μM.

18. A method according to any of claims 1-17, wherein the biological effect is selected from: binding, targeting, engulfing, trogocytosis, endocytosis, phagocytosis, Antibody-Dependent Cellular Cytotoxicity (ADCC), cytolysis, T-cell mediated cytolysis, Antibody Dependent Cellular Phagocytosis (ADCP), perforation, cytotoxicity, cytokine/chemokine activity or release, proliferation, cell activation, upregulation or downregulation of surface receptors.

19. A method according to any preceding claim, wherein the first and the second inducible systems are different.

20. A method according to any preceding claim, wherein the cell comprises both a first and a second inducible system.

21. A method according to any preceding claim, wherein the first inducible system does not interact with the second inducible system.

22. A method according to any preceding claim, wherein the first and second inducible systems may be selected from: a hypoxia inducible system, a forskolin inducible system, a temperature inducible system, a pH inducible system, an osmolarity inducible system, a carbon source inducible system, an alcohol inducible system, an amino acid inducible system, a steroid inducible system, a tetracycline inducible system, a cumate inducible system, a 4-hydroxytamoxifen (OHT) inducible system, a gas inducible system, a Riboswitch system, a Ribozyme system, an Aptazyme system, a Metallothionein inducible system, a rapamycin inducible system, a rheoswitch, a CRISPR system, and a chemically induced proximity system.

23. A method according to claim 22, wherein the first and second inducible systems are chemically induced proximity systems (CIP systems).

24. A method according to claim 23, wherein the first and second inducible systems are plant hormone or plant hormone analogue inducible proximity systems.

25. A method according to claim 24, wherein the plant hormone or plant hormone analogue inducible proximity systems are selected from: auxin, abscisic acid, gibberellin, ethene, cytokinin, salicylic acid, jasmonate, brassinosteroid, peptide, and caffeine proximity inducible systems.

26. A method according to claim 24 or 25, wherein at least one of the inducible systems is an abscisic acid inducible proximity system.

27. A method according to any of claims 24 to 26, wherein the first inducible system is selected from a from a caffeine inducible proximity system, a mandipropamid inducible proximity system, and a gibberellin inducible proximity system, and the second inducible system is an abscisic acid inducible proximity system.

28. A method according to any of claim 2-4, 6-9, 11-13, or 16-27 wherein the minimum level of expression of the first and optionally the second protein of interest at which the candidate enacts a biological effect on the cell expressing the first and optionally the second protein of interest is the level of expression of the first and optionally the second protein of interest at which a biological effect higher than the background biological effect is achieved.

29. A method according to claim 28 wherein the minimum level of expression of the first and optionally the second protein of interest at which the candidate enacts a biological effect on the cell expressing the first and optionally the second protein of interest is the level of expression of the first and optionally the second protein of interest at which a biological effect of at least 3, 4, 5, 6, 7, 8, 9, or 10 standard deviations above the background biological effect is achieved.

30. A method according to claim 28 or 29 wherein the background biological effect is the biological effect of the candidate on a control cell, preferably wherein the control cell is a cell which does not express the first or second proteins of interest.

31. A method according to any of claims 28 to 30, wherein the minimum level of expression of the first and optionally the second protein of interest at which the candidate enacts a biological effect on the cell expressing the first and optionally the second protein of interest is the activation threshold of the first and optionally the second protein of interest.

32. A method according to any of claims 28 to 31 wherein the minimum level of expression of the first and optionally the second protein of interest at which the candidate enacts a biological effect on the cell expressing the first and optionally the second protein of interest, or the activation threshold, is calculated using receiver operator characteristic (ROC) curve analysis, preferably using Youdon's index or Youden's J statistic.

33. A cell comprising (a) a first chemically inducible proximity system and/or (b) a second chemically inducible proximity system,
wherein the first chemically inducible proximity system (a) comprises:
(i) a first construct comprising a promoter operably linked to a nucleic acid sequence encoding:
a first chimeric protein, and
a second chimeric protein,
wherein the first and second chimeric proteins each comprise a first inducer binding domain and an effector domain;
wherein each first inducer binding domain is operable to bind to a first inducer;
wherein the effector domains comprise a transactivation domain and a first DNA binding domain;
wherein the effector domain of the first and second chimeric proteins is different; and
(ii) a second construct comprising a nucleic acid sequence encoding: one or more first DNA binding domain binding sites operably linked to a nucleic acid sequence encoding a first protein of interest;
wherein the second chemically inducible proximity system (b) comprises:
(i) a third construct comprising a promoter operably linked to a nucleic acid sequence encoding:
a third chimeric protein, and
a fourth chimeric protein,
wherein the third and fourth chimeric proteins each comprise a second inducer binding domain and an effector domain;
wherein each second inducer binding domain is operable to bind to a second inducer; wherein the effector domains comprise a transactivation domain or second DNA binding domain;
wherein the effector domain of the third and fourth chimeric proteins is different; and
(ii) a fourth construct comprising a nucleic acid sequence encoding: one or more second DNA binding domain binding sites operably linked to a nucleic acid sequence encoding a second protein of interest;
wherein the first chemically inducible proximity system does not interact with the second chemically inducible proximity system, and wherein one of the first or second DNA binding domains is a dl-Scel DNA binding domain.

34. A cell according to claim 33, wherein the first and/or second chemically inducible proximity systems are plant hormone or plant hormone analogue inducible proximity systems.

35. A cell according to claim 33 or 34, wherein the plant hormone or plant hormone analogue inducible proximity systems are selected from: auxin, abscisic acid, gibberellin, ethene, cytokinin, salicylic acid, jasmonate, brassinosteroid, peptide, and caffeine proximity inducible systems.

36. A method according to any of claims 33 to 35, wherein at least one of the chemically inducible proximity systems is an abscisic acid inducible proximity system.

37. A method according to any of claims 33 to 35, wherein at least one of the chemically inducible proximity systems is selected from an auxin inducible proximity system, a caffeine inducible proximity system, a mandipropamid inducible proximity system, and a gibberellin inducible proximity system.

38. A method according to any of claims 33 to 37, wherein the first chemically inducible proximity system is selected from a from a caffeine inducible proximity system, a mandipropamid inducible proximity system, and a gibberellin inducible proximity system, and the second chemically inducible proximity system is an abscisic acid inducible proximity system.

39. A method according to any of claims 35 to 38, wherein the auxin inducible proximity system comprises a first construct comprising a promoter operably linked to a nucleic acid sequence encoding:
a first chimeric protein, and
a second chimeric protein,
wherein the first and second chimeric proteins each comprise an auxin binding domain and an effector domain;
wherein the auxin binding domain is selected from Transport Inhibitor Response 1 protein (TIR1) or Auxin/indole-3-acetic acid protein (AID); wherein the effector domain is selected from a transactivation domain or a catalytically inactive I-Scel endonuclease DNA binding domain (dl-Scel); and wherein the auxin binding domain and the effector domain of the first and second chimeric proteins are different.

40. A method according to any of claims 35 to 38, wherein the caffeine inducible proximity system comprises a first construct comprising a promoter operably linked to a nucleic acid sequence encoding:
a first chimeric protein, and
a second chimeric protein,
wherein the first and second chimeric proteins each comprise a caffeine binding domain and an effector domain;
wherein the caffeine binding domain is an Anti-caffeine heavy-chain antibody fragment (aCaffVHH); wherein the effector domain is selected from a transactivation domain or a DNA binding domain selected from Gal4 DNA binding domain and a catalytically inactive I-Scel endonuclease DNA binding domain (dl-Scel); and wherein the effector domain of the first and second chimeric proteins is different.

41. A method according to any of claims 35 to 38, wherein the mandipropamid inducible proximity system comprises a first construct comprising a promoter operably linked to a nucleic acid sequence encoding:
a first chimeric protein, and
a second chimeric protein,
wherein the first and second chimeric proteins each comprise a Mandipropamid (Mandi) binding domain and an effector domain;
wherein the Mandipropamid binding domain is selected from a modified pyrobactin receptor (PYR^{Mandi}), a modified pyrobactin-like receptor (PYLcs^{Mandi}), and abscisic acid insensitive 1 protein (ABI); wherein the effector domain is selected from a transactivation domain or a DNA binding domain selected from Gal4 DNA binding domain and a catalytically inactive I-Scel endonuclease DNA binding domain (dl-Scel);
and wherein the Mandipropamid binding domain and effector domain of the first and second chimeric proteins is different.

42. A method according to any of claims 35 to 38, wherein the gibberellin inducible proximity system comprises a first construct comprising a promoter operably linked to a nucleic acid sequence encoding:
a first chimeric protein, and
a second chimeric protein,
wherein the first and second chimeric proteins each comprise a gibberellin binding domain and an effector domain;
wherein the gibberellin binding domain is selected from gibberellin insensitive dwarf 1 protein (GID1) and gibberellin insensitive (GAI) protein or a fragment thereof; wherein the effector domain is selected from a transactivation domain or a DNA binding domain selected from Gal4 DNA binding domain and a catalytically inactive I-Scel endonuclease DNA binding domain (dl-Scel);
and wherein the gibberellin binding domain and effector domain of the first and second chimeric proteins is different.

43. A cell according to any of claims 33 to 42 wherein the second construct comprises a nucleic acid sequence encoding: one or more dl-Scel binding sites or one or more Gal4 upstream activation sequences operably linked to a nucleic acid sequence encoding a first protein of interest.

44. A cell according to any of claims 35 to 43, wherein the abscisic acid inducible proximity system comprises a third construct comprising a promoter operably linked to a nucleic acid sequence encoding:
a third chimeric protein, and
a fourth chimeric protein,
wherein the third and fourth chimeric proteins each comprise an abscisic acid binding domain and an effector domain;
wherein the abscisic acid binding domain is selected from ABI1 or pyrobactin resistance-like protein PYL1; wherein the effector domain is selected from a transactivation domain or a DNA binding domain selected from Gal4 DNA binding domain and a catalytically inactive I-Scel endonuclease DNA binding domain (dl-Scel);
and wherein the abscisic acid binding domain and the effector domain of the third and fourth chimeric proteins are different.

45. A cell according to any of claims 33 to 44 wherein the fourth construct comprises a nucleic acid sequence encoding: one or more dl-Scel binding sites or one or more Gal4 upstream activation sequences operably linked to a nucleic acid sequence encoding a second protein of interest.

46. A cell according to any of claim 43 or 45 wherein the one or more dl-Scel binding sites comprise between 1 to 15 I-Scel DNA binding sites, preferably ten I-Scel DNA binding sites, preferably wherein the I-Scel DNA binding sites are in tandem.

47. A cell according to any of claim 43 or 45 wherein the one or more Gal4 upstream activation sequences comprise between 1 and 15 GAL4 upstream activation sequences, preferably nine GAL4 upstream activation sequences, preferably wherein the GAL4 upstream activation sequences are in tandem.

48. A cell according to any of claims 39 to 47, wherein the or each transactivation domain is selected from: Gal4, Oaf1, Leu3, Rtg3, Pho4, Gln3, Gcn4 in yeast, and p53, NFAT, NF-κB, VP16 or VP34, preferably wherein the or each transactivation domain is VP16.

49. A cell according to any of claims 33 to 48, wherein the cell is a mammalian cell, preferably a HEK293 or CHO-K1 cell.

50. A method according to any of claims 1 to 32, or a cell according to any of claims 33 to 49, wherein the first and/or the second protein of interest is an antigen, preferably an antigen which is a therapeutic target.

51. A method or cell according to claim 50, wherein the antigen is associated with a disease, preferably wherein the antigen is a tumour associated antigen (TAA).

52. A method or cell according to claim 50 or 51, wherein the antigen is selected from: CD19, BCMA, CD123, mesothelin, GD2, CD20, CD33, CD47, HER2, CD22, CD13, PSMA, EGFR vIII, EGFR, CD38, EpCAM, PSCA, CEA, HIV, Glypican-3, FLT3, NKG2D, claudin 18.2, DLL3, CS1, MUC16, CD3, PD-L1, 4-1BB, PD-1, LAG3, CTLA-4, MUC1, 5T4, CD40, CD155, OX-40, NY-ESO, ROR1, TROP2, VEGFR1, VEGFRII, CLL, CD30, CD70, CD133, TIM-3, L1CAM, ICOS, DLL4, Fralpha, WT1, IL13Ralpha, Lewis-Y or cMET.
